(19) United States
(12) Patent Application Publication
(10) Pub. No.: US 2010/0286791 A1
(43) Pub. Date: Nov. 11, 2010

(54) Integrated system for the ballistic and nonballistic infixion and retrieval of implants

(76) Inventor: David S. Goldsmith, Atlanta, GA (US)

Correspondence Address: David S. Goldsmith, 3567 Kingsboro Road NE, Atlanta, GA, 30319, US

(21) Appl. No.: 11/986,021

(22) Filed: Nov. 19, 2007

Related U.S. Application Data
(60) Provisional application No. 60/860,392, filed on Nov. 21, 2006.

(51) Int. Cl. A61F 2/04; A61M 36/12; A61M 25/00; A61M 31/00; A61B 18/04; A61B 17/50; A61B 17/28; A61F 13/00

(52) U.S. Cl. 623/23.7; 600/8; 604/523; 604/93.01; 606/27; 604/524; 606/211; 606/205; 602/43

(57) ABSTRACT

Provided are methods and apparatus for the use of magnetic traction to maintain the patency of a tubular anatomical structure, whether a vessel, duct, the trachea, bronchus, bile duct, ureter, vas deferens, fallopian tube, or portions of the digestive tract, as to constitute means for extraluminal stenting. An extraluminal stent consists of a perimedial or medial intravascular and an extravascular component. The intravascular component consists of ferromagnetic spherules implanted aeroballistically or stays implanted by means of a special hand tool, while the extravascular component consists of a pliant jacket or mantle that has magnets mounted about its outer surface. A catheter adapted for use as the barrel of a gas-operated implant insertion gun is so devised that it can be used independently to perform an angioplasty and thereafter have its free or extracorporeal end inserted into the airgun to initiate implantation of the intravascular component without the need for withdrawal and reinsertion through the introducer sheath. When the implants must be spaced too closely together to be controlled by hand, a positional control system is used to effect discharge automatically. Spherules that consist entirely of medication or that have a radiation emitting seed as the core can be implanted with the same apparatus. A glossary of terms follows the specification.

### CROSS REFERENCE TO RELATED APPLICATION

This nonprovisional application follows and claims the benefit of Disclosure Document 565662, filed on 26 Nov. 2004, and Provisional Patent Application 60/860,392, filed on 21 Nov. 2006 under 35 U.S.C. 119(e). The entire disclosure of the above-referenced application is incorporated by reference herein.

### BACKGROUND OF THE INVENTION

1. Field of the Invention

The apparatus and methods to be described are intended for use by veterinary specialists, laryngologists, pulmonologists, interventional radiologists, interventional cardiologists, gastroenterologists, and urologists to preserve the integrity of and sustain the movement of contents through a bodily conduit (vas, vessel, canal, ductus, duct), that has collapsed, become constricted, or has been alleviated of constriction (stenosis) or blockage (occlusion). Endoluminal stents are used, for example, by pulmonologists and laryngologists to alleviate the symptoms of stricture or collapse in the airway (trachea and bronchi), by interventional cardiologists and radiologists to preserve the patency of blood vessels, and by urologists to maintain the patency of the ureters and the ducts that transport gametes.

The apparatus and methods to be described are intended to apply to all vasa or ducti in all vertebrates sufficient in diameter or gauge to accept the apparatus employed. All stents are remedial or palliative, none constitutes a means for actually curing underlying disease, and no such claims are made for the means to be described; however, the preservation of patency is always essential to sustain function and is often essential to sustain life, making the capability to do so with less risk to the patient of long-term sequelae of vital importance.

2. Summary Description of the Invention

An extraluminal stent consists of an intravascular and an extravascular component. Ordinarily, the intravascular component consists of bioinert ferromagnetic spherules implanted by means of a specially adapted semiautomatic gas-operated gun, or airgun, which projects the spherules to a point beneath the outer fibrous sheath of the ductus or tubular structure, the tunica adventitia, or tunica externa in vessels and tunica fibrosa in the trachea, for example. In the vascular tree, the unitized barrel-catheter and muzzle-head, or barrel-assembly, which is used without connection to the airgun while the barrel-assembly is used to perform an angioplasty, is passed transluminally to the lesion and the spherules discharged at an acute forward angle to undercut and lodge beneath the adventitia, or subadventitially (perimedially).

While the leading (distal) component through which the spherules are discharged (the muzzle-head) is placed in flush or slightly less than flush relation to the wall of the lumen, the passing pulse expands the vessel about the muzzle-head, which also has grooves to allow oxygenation albeit reduced compared to the unobstructed condition. For this reason, the barrel-assembly has built into it certain features to minimize the time required for a procedure. When the muzzle-head is of the kind that delivers a plurality of spherule implants in different directions with each discharge, such eccentricity within the lumen of the muzzle-head as occurs with the passage of the pulse has proportionately little effect upon the spherule impact force. One or more barrels or barrel-tubes housed within an gas embolism-averting gas pressure equalizing enclosure, or barrel-catheter, discharge forward and radially through a distal muzzle-probe or muzzle-head.

The nontransluminally delivered prostheses to be described herein—stent-jackets, stays, clasp-wraps, and so on, allow passage through an entry wound smaller in size than does conventional open repair. When for any reason ballistic implantation is contraindicated, a plurality of arcuate stent-stays containing ferrous metal, or a clasp-wrap, likewise used with a stent-jacket, can be manually inserted into the wall of the ductus through a local incision by means of a special hand tool to be described. A test is provided for adventitia-media delamination to determine whether the spherules must be implanted in the media. When transluminal access is best avoided, a clasp-wrap that encircles and engages the ductus is used.

The extravascular component, or stent-jacket, consists of a sleeve of elastic tubing that is slit along one side and has small neodymium iron boron magnets mounted about its outer surface which are encapsulated for bioinertness and coated, usually by means of vapor deposition or sputtering, with tantalum, tungsten, gold, molybdenum, and alloys thereof, or a biostable biocompatible radiopaque polymer for radiopacity. The stent-jacket is introduced through a small local incision and placed about the vessel or duct in surrounding, or circumvascular, relation. The magnets exert the minimum centrifugal refractive force upon the spherule implants sufficient to draw the adventitia up against the inner surface of the stent jacket with the vas or ductus relaxed in autonomic motor function. The noncollapsible, flexible, and side-slitted circumvascular stent-jacket limits contraction of the substrate vas or ductus to the normal or to the best minimum diameter as judged by the clinician while allowing the ductus to freely expand. The elastic stent-jacket thus complies with pulsatile, tonic, or peristaltic expansion and allows the discretionary treatment of eccentric lesions.

When the ductus is swollen, a stent-jacket is chosen that incorporates an absorbable or percuraeous ultrasonic lithotriptor-destructible expansion insert which allows the stent to contract or to be contracted with subsidence (regression) in the enlargement (dilatation, ectasia, ectasis) as the vas or ductus subsides. The recent advent of totally endoscopic and robotic means to minimize the trauma of exposure significantly augment the practicability and preferability of circumvascular over endoluminal stents in many locations, access to the coronary arteries, for example, no longer necessitating a median sternotomy, clamshell, or hemiclamshell thoracotomy. Retention of the wall of the ductus by means of subadventitial implants under minimal retractive force if not completely peripheral to the dynamically vital tissue of the lining of the lumen, or passageway through the tubular structure, averts compression necrosis and fistulization while leaving the lumen free and clear of any foreign object. The unobstructed lumen is less likely to restenose, and should reintervention become necessary, the lumen will be unobstructed by a stent. The catheter-based apparatus described may be used with the patient inside a tomograph gantry.

Apart from the variation in mechanical properties that may be expected from the diseased tissue as the only kind that would be implanted thus, the response of the lumen wall to the condition of a miniature ball implanted within it and subjected to a mild tractive outward force demands experimentation. While substantially sterile upon discharge, in an otherwise healthy individual, the miniball is likely to acquire some bacteria in traversing the blood or other lumen contents and in the endothelium, some of which would be squeejeed away as the miniball passes through the inner layers. Since such means are proposed for use with respect to tissue types as diverse as blood vessels, ducts, the airway, and gastrointestinal tract, this factor is widely variable, from the relatively sanitary condition inside the bloodstream to the heavy burden of bacteria in the colon. If the bacteria count is relatively small, a containment or carbuncle response is likely, whereas if the count is significant, a fistulizing response is likely, with the consequence that the miniball would be expelled, both purely mechanical outward extraction under the tractive force of the magnets and expulsion by fistulization referred to herein as ‘pull-through.’

To counteract pull-through, as is routine, systemic antibiotics are administered and the miniballs are coated with an antibiotic. The tissue adhesion and infiltration or ingrowth response at various depths into the lumen wall likewise demands experimentation. This information will then determine the surface texture, medication, value in applying phosphorylcholine, and so on, that will be optimal for a specific application. Accordingly, the various components used may be classified as transluminal, to include special catheters used as airgun barrels, some angioplasty capable; intravascular, to include miniballs and stays; or extravascular, to include stent-jackets, clasp-wraps, magnet-wraps, and stay insertion tools to be described, all variable in particulars as required by different vascular conditions. Stent-jackets may be made adaptive to the reduction in caliber of vasa with subsidence or regression in an initially enlarged condition thereof, while stays may be adaptive in partial or complete dissolution over a controllable period. Stays and miniballs can additionally be made to release medication or radiation.

3. Terminology

Because the recognized terms ‘intravascular,’ ‘extravascular,’ ‘intratubal,’ ‘extratubal,’ or, applying the term more generally than to the subarachnoid or subdural spaces, ‘intrathecal,’ do not distinguish ‘within or without a vessel (vas) or duct (ductus)’, from ‘within or without the lumen of the vessel or duct,’ here the more recently recognized (included in medical dictionaries) but long commonly used and immediately understood terms ‘endoluminal’ and ‘extraluminal’ are used. The term ‘intraluminal’ had already been accepted, but its complement, ‘extraluminal,’ had not. The terms ‘vessel’ and ‘ductus’ both denoting tubular anatomical structures, ‘vessel’ as used herein generally refers to blood vessels rather than to other type vessels.

The term ‘endoluminal’ is not widely recognized as combining a Greek prefix with a Latin stem word, but the term ‘endovascular (endoluminal vascular),’ of like composition, has more recently been accepted by default through common use as necessary and obvious in meaning. The unrecognized term ‘endostent’ as a contraction for ‘endovascular,’ or ‘endoluminal,’ ‘stent’, to denote any conventional or intraluminal stent, can be used for brevity; however, since an extraluminal stent of the kind to be described herein is not entirely extravascular, the contractions ‘extrastent’ and ‘exostent’ will remain unacceptable as ambiguous unless made clear by convention or ancillary description. An ‘extraluminal’ stent, then, consists of subadventitial (perimedial) or medial, hence, intravascular but extraluminal spherule implants, and an extravascular, specifically circumvascular or perivascular, a fortiori, extraluminal, stent-jacket, or otherwise, subcutaneous or suprapleural magnets.

The term ‘abluminal’ is indefinite as to the layer or depth into the lumen wall to be understood. The terms ‘circumluminal’ and ‘periluminal’ are neither recognized nor distinct as to denoting whether the surrounding is of the lumen inside or outside the vessel or duct. For present purposes, where no guidewire is used, the term ‘trackability’ is synonymous with ‘steerability’ as denoting the relative ease with which the barrel-assembly, rather than a catheter moved over a guidewire, can be passed through a tortuous segment of a vessel. Any tubular anatomical structure may be referred to as a vas or ductus; however, the term ‘endovascular,’ for example, because it stipulates ‘vascular,’ is more readily interpreted as relating to an arterial or a venous stent. The term ‘ablation’ as conventionally used is not limited to the removal of tissue by means of cutting, and here denotes the destruction of tissue protrusive into a lumen by means of thermal (actual cautery) or cryogenic cautery (cryocautery).

‘Endoluminal,’ indicates that the stent is not simply endovascular or intravascular but within the lumen; when dealing with stents that include a distributed collective extraluminal intravascular component but are otherwise circumvascular, the term ‘endoluminal’ provides a necessary distinction. Except in using the standard term ‘intravascular brachytherapy,’ ‘intravascular’ is used to mean within the structure, the wall, of the vessel, not through the lumen, as it were ‘intravenous.’

As used herein, ‘torqueability’ denotes the resistance of a catheter to twisting or helical deformation when rotated at the manipulated, or proximal end. Parenthetically, in the present context, when the need for steerability outweighs the need for torqueability, the pliancy required results in a loss of torqueability, necessitating the incorporation of a hard-wire remotely controlled electrical motor to rotate the distal end or muzzle-head of the transluminal component to be described, referred to as a ‘barrel-assembly.’

The term “barrel” as denoting a cylindrically form or cylindrically formed part of a larger structure is in standard use relative to guns, eyelets, and plating, all directly involved herein, the context making clear which of these meanings is intended. Use of the term ‘clip’ has been limited to the magazine clips used in the special interventional airguns to be described herein and not to refer to the clasps used, for example, in clasp-magnets and clasp-wraps. An ‘airgun’ or ‘air gun’ may discharge implants as projectiles or air, a vortex tube-based cooling or heating device referred to by the standard industrial term ‘cold air gun.’

The traditional meaning of ‘percutaneous’ as passing through without breaking the skin (transcutaneous; transdermic; diadermic) is no longer restricted in meaning thus, the very term ‘percutaneous transluminal coronary angioplasty’ for procedures that require entry by incision and arteriotomy making the point. The unrecognized terms ‘permural’ or ‘perparial’ are specific for walls of body cavities and not applicable to incisions elsewhere. The term ‘bore’ is not properly applicable to the internal diameter of the barrel-tubes, which are extruded, but is nevertheless useful to denote the lumen diameter corresponding to miniballs of slightly smaller diameter meant to use this tubing as a barrel.

The term ‘subadventitial’ as used herein denotes the depth into the lumen wall at which the material of the wall becomes the peripheral connective tissue comprised of the external elastic lamina just outside of the smooth muscle and inside the tunica adventitia or outer fibrous jacket. Until changed by adjustment of the drive controller, a stepper motor rotates by a consistent angle as would move or ‘increment’ a linear positioning table by the equivalent constant linear distance, here along the lumen of a ductus.

To distinguish between ‘increments’ as these elementary rotatory steps set by the step-angle, from the overall segment traversed as the sum of these elementary steps, the term ‘step’ is limited to the action of the stepper motor, and the term ‘increment’ applied to the transluminal segment traversed as the sum of these steps. The term ‘torquer’ is used to describe both a kind of electrical motor and knobs used to rotate catheters, the term ‘cure’ used for the setting time of an adhesive and the time to heal, and the term ‘magazine’ to indicate the container for a load queued for ejection in an airgun or a stent-stay insertion tool, to be described, the context making it plain which meaning is intended.

‘Atherectomy’ denotes a form of angioplasty that unlike the compression of plaque by a balloon, cuts the plaque away. Thermal and laser catheters ablate rather than merely crush plaque, but such removal is not by cutting action as would suggest use of the suffix—‘ectomy,’ so that these are usually referred to as forms of angioplasty. The ‘angioplasty’ barrel-catheter to be described, operates in two ways, of which the cutting action by side-sweepers is properly a form of atherectomy but the noncutting thermal or cryogenic action a form of angioplasty. By avoiding balloon injury to an artery as may result in inflammation entirely through its wall, an atherectomy performed by the means to be described facilitates treatment by the extraluminal stenting means also to be described. The latter include stent-stays and clasp-wraps which avoid the lumen altogether.

Since the term ‘angioplasty’ is applied to atherectomy but the reverse is not true, the one term that covers both actions of a barrel-assembly used as an independent plaque-removal device is ‘angioplasty,’ prompting the term ‘angioplasty-capable barrel-assembly’ or ‘angioplasty barrel-assembly’ for short. The term ‘cavitation’ in engineering denotes the generation of bubbles in a fluid medium, whereas in medical use, the same term denotes the formation of a vacuities, vacuoles, or cavities whether normal or pathological.

While the term ‘recovery electromagnets’ would comprehend both the retrieval of dropped and the extraction of mispositioned miniballs, the term ‘recovery and extraction miniball electromagnet assembly’ is used for specificity. Whereas the electronic components in positional control systems were once separate and distinct, miniaturization has led to the combination of these in small enclosures that obscure the functional distinctness of each component, namely, the positional command (set point, zero point) input device whether digital encoder or analogue (resolver, synchro), differential (comparator), servomotor (actuator), machine table, shaft, or other driven member, and output or positional difference (displacement) measuring device, usually of the same kind as the command input device, that provides the signal fed back to the differential. This has resulted in much confused terminology, such as use of the term ‘amplifier’ to denote an apparatus that actually includes the controller. Herein, the terms ‘controller’ or ‘servocontroller’ denote the controller, and the term ‘amplifier’ or ‘servoamplifier’ denotes the amplifier.

4. Description of the Prior Art and Conventional Practice in Vascular, Tracheobronchial, and Urological Interventions

Endoluminal stents offer distinct advantages over treatments that necessitate small entry wound or laparoscopic access, much less open exposure. Occasional injury from balloon overinflation or a special insertion device notwithstanding, placed transluminally, conventional, or endoluminal, stents are prosthetic liners that compared to open surgery, confer luminal patency and symptomatic palliation with little trauma. Furthermore, unlike extraluminal stents to be described, which require both transluminal access and separate entry through a small incision and dissection to allow mantling about the vessel or duct with a length of resilient tubing, an endoluminal stent can be introduced into any deep-lying vessel or duct that is fully embedded or invested within tissue and has a lumen that is large enough in diameter to admit it.

Significantly, this includes the femoral, popliteal, and tibial arteries, which larger and lower in the body, are often affected by vascular disease, and embedded would necessitate much dissection to encircle. For vessels and ducts that embedded lack circumvascular space and can be approached only with much dissection, a purely transluminal approach, with or without the introduction of an endoluminal stent, is preferable, the likelihood for adverse sequelae then more likely. For embedded structures, the extraluminal stenting to be described would only replace the sequelae associated with endoluminal stenting with the sequelae to be expected from the intrusive approach and the placement of the base-tube in complete investment within the surrounding tissue.

Significantly, an endoluminal stent can be used despite delamination or a moderate degree of malacia in the wall of a blood vessel, although this is not of particular concern in the trachea, for example. Whereas modern endoprostheses and stents are made of materials such as titanium and polymers that are nonferromagnetic or are at most weakly ferromagnetic as not to disallow the use of magnetic resonance imaging (MRI), the present means inherently demand the use of ferromagnetic materials that once applied do indeed disallow the use of magnetic resonance imaging; unless the ferromagnetic implants are first extracted, alternative forms of imaging must be used to the end of life. Compatibility with MRI notwithstanding, several studies have indicated that for recent endoluminal stents, magnetic resonance imaging poses an increased risk of restenosis.

Balloon angioplasty does not remove but rather redistributes plaque to clear a passageway or channel through the lumen by crushing the plaque between the endothelium and the internal elastic lamina, injuring both and inducing proliferation, or intimal hyperplasia (Harnek, J., Zoucas, E., Stenram, U., Cwikiel, W. 2002. “Insertion of Self-expandable Nitinol Stents without Previous Balloon Angioplasty Reduces Restenosis Compared with PTA Prior to Stenting,” Cardiovascular and Interventional Radiology 25(5):430-436), rotational atherectomy stimulating intimal hyperplasia to a significantly lesser degree (McKenna, C. J., Wilson, S. H., Camrud, A. R., Berger, P. B., Holmes, D. R. Jr., and Schwartz, R. S. 1998. “Neointimal Response Following Rotational Atherectomy Compared to Balloon Angioplasty in a Porcine Model of Coronary In-stent Restenosis,” Catheterization and Cardiovascular Diagnosis 45(3):332-336).

Furthermore, when properly introduced from outside the ductus, two classes of device to be described, stays and clasp-wraps, avoid the lumen entirely, so that when used in the vascular tree, these substantially avoid disruption to the flow of blood and the adverse consequences such disruption can induce (see, for example, Shive, M. S., Salloum, M. L., and Anderson, J. M. 2000. “Shear Stress-induced Apoptosis of Adherent Neutrophils: A Mechanism for Persistence of Cardiovascular Device Infections,” Proceedings of the National Academy of Sciences of the United States of America 97(12):6710-6715; Shive, M. S., Brodbeck, W. G., Anderson, J. M. 2002. “Activation of Caspase 3 During Shear Stress-induced Neutrophil Apoptosis on Biomaterials,” Journal of Biomedical Materials Research 62(2): 163-168).

While inflammation is often pan-arteritic (see, for example, Higuchi, M. L., Gutierrez, P. S., Bezerra, H. G., Palomino, S. A., Aiello, V. D., Silvestre, J. M., Libby, P., Ramires, J. A. 2002. “Comparison Between Adventitial and Intimal Inflammation of Ruptured and Nonruptured Atherosclerotic Plaques in Human Coronary Arteries,” Arquivos Brasileiros Cardiologia 79(1):20-24), and prior to atherectomy, adventitial inflammation might conceivably contribute to plaque instability (see, for example, Hu, C. L., Xiang, J. Z., Hu, F. F., and Huang, C. X. 2007. “Adventitial Inflammation: A Possible Pathogenic Link to the Instability of Atherosclerotic Plaque,” Medical Hypotheses 68(6):1262-1264.), avoidance of the lumen eschews the many additional complications that may arise within the lumen and is advantageous in certain conditions that involve inflammation of the intima, such as Takayasu disease (Takayasu arteritis) when in-stent restenosis could otherwise pose a problem (Mieno, S., Horimoto, H., Arishiro, K., Negoro, N., Hoshiga, M., Ishihara, T., Hanafusa, T., and Sasaki, S. 2004. “Axillo-axillary Bypass for in-Stent Restenosis in Takayasu Arteritis,” International Journal of Cardiology 94(1):131-132) and antiphospholipid (Hughes) syndrome (see, for example, Ben-Ami, D., Bar-Meir, E., and Shoenfeld, Y. 2006. “Stenosis in Antiphospholipid Syndrome: A New Finding with Clinical Implications,” Lupus 15(7):466-472).

By the early 1990s it had been determined that the critical factor for ensuing acute coronary events was not whether plaque was occlusive but rather that plaque was present within the arterial wall at all; protrusive plaque may not prove occlusive, and atheromatous plaque that had been smashed flat in the media was just as likely to induce infarction as plaque that had been allowed to remain protruding into the lumen (Waxman, S., Ishibashi, F., and Muller, J. E. 2006. “Detection and Treatment of Vulnerable Plaques and Vulnerable Patients Novel Approaches to Prevention of Coronary Events,” Circulation 114(22):2390-2411; Naghavi, M.; Libby, P; Falk, and 55 Other Authors 2003. “From Vulnerable Plaque to Vulnerable Patient: A Call for New Definitions and Risk Assessment Strategies: Part I,” Circulation 108(14):1664-1672; Libby, P. and Aikawa, M. 2002. “Stabilization of Atherosclerotic Plaques: New Mechanisms and Clinical Targets,” Nature Medicine 8(11)1257-1262, erratum 9(1):146; Moreno, P. R. 2001. “Pathophysiology of Plaque Disruption and Thrombosis in Acute Ischemic Syndromes,” Journal of Stroke and Cerebrovascular Disease 10(2 Part 2):2-9; Muller, J. E. Abela, G. S. Nesto, R. W. and Tofler, G. H. 1994. “Triggers, Acute Risk Factors and Vulnerable Plaques: The Lexicon of a New Frontier,” Journal of the American College of Cardiology 23(3):809-813, and as but one distinction between rupture and erosion, the degree of protrusion appears of secondary significance (Shah, P. K. 2002. “Pathophysiology of Coronary Thrombosis: Role of Plaque Rupture and Plaque Erosion,” Progress in Cardiovascular Diseases 44(5):357-368).

When collateral circulation is lacking, unless plaque is actually removed, balloon angioplasty appears only to add injury and the risk of thromboembolization. When collateral circulation is insufficient, reperfusion or recanalization to the Thrombolysis in Myocardial Infarction study-defined Grade III (TIMI III, normal) flow is known to produce a better consequence the more promptly it is accomplished. However, when collateral circulation is sufficient, even coronary total occlusion may be disserved by angioplasty, which given the burden of plaque can injure collateral circulation by embolization (Meier, B. 1989/2005. “Angioplasty of Total Occlusions: Chronic Total Coronary Occlusion Angioplasty,” Catheterization and Cardiovascular Diagnosis 17(4):212-217; Kahn, J. K. 1995/2005. “Collateral Injury by Total Occlusion Angioplasty: Biting the Hand that Feeds Us,” Catheterization and Cardiovascular Diagnosis 34 (3): 65-66; Ha, J. W., Cho, S. Y., Chung, N., Choi, D. H., Choi, B. J., Jang, Y., Shim, W. H., and Kim, S. S. 2002. “Fate of Collateral Circulation After Successful Coronary Angioplasty of Total Occlusion Assessed by Coronary Angiography and Myocardial Contrast Echocardiography,” Journal of the American Society of Echocardiography 15(5):389-395; Waser, M., Kaufmann, U., and Meier, B. 1999. “Mechanism of Myocardial Infarction in a Case with Acute Reocclusion of a Recanalized Chronic Total Occlusion: A Case Report,” Journal of interventional Cardiology 12 (2), 137-140. Stone, G. W., Kandzari, D. E., Mehran, R., Colombo, A., and 23 Other Authors 2005. “Percutaneous Recanalization of Chronically Occluded Coronary Arteries: A Consensus Document, Part I,” Circulation 112(15):2364-2372).

Thus, the need for stenting is often the direct result of inadequacies of balloon angioplasty, which rather than to remove, only deforms plaque and can subject the lumen wall to stretching injury that induces abrupt closure, as well as dilatation and dissections that stimulate constrictive remodeling, or arterial shrinkage, which is “the predominant factor responsible for luminal narrowing after balloon angioplasty” and the stimulant for intimal hyperplasia (Pasterkamp, G., Mali, W. P., and Borst, C. 1998. “Application of Intravascular Ultrasound in Remodelling Studies,” Seminars in Interventional Cardiology 2(1):11-18); see also Smits, P. C., Bos, L. Quarles van Ufford, M. A., Eefting, F. D., Pasterkamp, G., and Borsta, C. 1998. “Shrinkage of Human Coronary Arteries is an Important Determinant of de Novo Atherosclerotic Luminal Stenosis: An in Vivo Intravascular Ultrasound Study,” Heart 79(2):143-147; Narins, C. R., Holmes, D. R. Jr., and Topol, E. J. 1998. “A Call for Provisional Stenting: The Balloon is Back!,” Circulation 97(13):1298-1305; Teo, K. K. 1998. “Clinical Review: Recent Advances, Cardiology,” British Medical Journal 316(7135):911-915).

Since at least 1996, the application of an external stent has been known to reduce intimal thickening in vein grafts (Izzat, M. B., Mehta, D., Bryan, A. J., Reeves, B., Newby, A. C., and Angelini, G. D. 1996. “Influence of External Stent Size on Early Medial and Neointimal Thickening in a Pig Model of Saphenous Vein Bypass Grafting,” Circulation 94(7):1741-1745. Vijayan, V., Shukla, N., Johnson, J. L., Gadsdon, P., Angelini, G. D., Smith, F. C., Baird, R., and Jeremy, J. Y. 2004. “Long-term Reduction of Medial and Intimal Thickening in Porcine Saphenous Vein Grafts with a Polyglactin Biodegradable External Sheath,” Journal of Vascular Surgery 40(5):1011-1019; Jeremy, J. Y., Bulbulia, R., Johnson, J. L., Gadsdon, P., Vijayan, V., Shukla, N., Smith, F. C., and Angelini, C. D. 2004. “A Bioabsorbable (Polyglactin), Nonrestrictive, External Sheath Inhibits Porcine Saphenous Vein Graft Thickening,” Journal of Thoracic and Cardiovascular Surgery 127(6): 1766-1772.

These external stents, some absorbable, some not, were inwardly restraining but not lumen patenting and were incapable of adapting to and remaining with the substrate ductus while preserving its patency while complying with its autonomic movement during overall enlargement or reduction. By contrast, the extraluminal stents herein to be described are restraining inwardly but compliant outwardly. Specifically, the stents girdle about the ductus at its smallest normal diameter and comply with outward forces developed within and travelling through its walls.

Abrupt closure is associated with intimal and medial injury and elevation in blood creatine kinase (CK, phosphocreatine kinase, creatine phosphokinase) myocardial band (MB) isoenzyme, but a cause or effect relationship between abrupt closure and elevated CK-MB has not been determined (Cavallini, C., Rugolotto, M., Savonitto, S. 2005. “Prognostic Significance of Creatine Kinase Release After Percutaneous Coronary Intervention,” Italian Heart Journal 6(6):522-529). Its prevention is sought through the administration of antiplatelet (antithrombocyte) medication, such as aspirin and glycoprotein IIb/IIIa, anticoagulants, such as intravenous heparin, vasodilatory drugs, such as nitroglycerin and calcium channel blockers or antagonists.

Endoluminal stents alleviate but do not prevent, and by introducing dissection and thrombus may actually precipitate, abrupt closure (Cheneau, E., Mintz, G. S., Leborgne, L., Kotani, J., Satler, L. F., Ajani, A. E., Weissman, N. J., Waksman, R., and Pichard, A. D. 2004. “Intravascular Ultrasound Predictors of Subacute Vessel Closure After Balloon Angioplasty or Atherectomy,” Journal of Invasive Cardiology 16(10):572-574; Cheneau, E., Leborgne, L., Mintz, G. S., Kotani, J., Pichard, A. D., Satler, L. F., Canos, D., Castagna, M., Weissman, N. J., and Waksman, R. 2003. “Predictors of Subacute Stent Thrombosis: Results of a Systematic Intravascular Ultrasound Study,” Circulation 108(1):43-47).

Drug-eluting stents remain susceptible to thrombosis (Iakovou, I., Schmidt, T, Bonizzoni, E., Ge, L., Sangiorgi, G. M., Stankovic, G., Airoldi, F., Chieffo, A., Montorfano, M., Carlino, M., Michev, I., Corvaja, N., Briguori, C., Gerckens, U., Grube, E., and Colombo, A. 2005. “Incidence, Predictors, and Outcome of Thrombosis After Successful Implantation of Drug-eluting Stents,” Journal of the American Medical Association 293(17):2126-2130; Daemen, J., Wenaweser, P., Tsuchida, K., Abrecht, L., Vaina, S., Morger, C., Kukreja, N., Juni, P., Sianos, G., Hellige, G., van Domburg, R. T., Hess, O. M., Boersma, E., Meier, B., Windecker, S., and Serruys, P. W. 2007. “Early and Late Coronary Stent Thrombosis of Sirolimus-eluting and Paclitaxel-eluting Stents in Routine Clinical Practice: Data from a Large Two-institutional Cohort Study,” Lancet 369(9562):667-678; Urban P. and De Benedetti, E. 2007. “Thrombosis: The Last Frontier of Coronary Stenting? [Comment on Daemen et al., preceding]” Lancet 369(9562):619-621).

Drug-eluting stents can also induce thrombosis in association with allergic inflammatory reactions (Virmani, R, Guagliumi, G., Farb, A., Musumeci, G., Grieco, N., Motta, T., Mihalcsik, L., Tespili, M., Valsecchi, O., and Kolodgie, F. D. 2004′. “Localized Hypersensitivity and Late Coronary Thrombosis Secondary to a Sirolimus-Eluting Stent: Should We Be Cautious?,” Circulation 109(6):701-705.

Moreover, an endoluminal stent required for sequelae the direct result of balloon angioplasty is not only susceptible to, but makes possible and implements to itself become, precisely the factor that provokes restenosis. The action associated with the placement of a stent can produce the conditions that result in restenosis (see, for example, Anderson, H. V. and Carabello, B. A. 2000. “Provisional Versus Routine Stenting: Routine Stenting is Here to Stay,” Circulation 102(24):2910-2914). A stent without any endoluminal component cannot passively clog, much less actively stimulate a response that would clog it.

By contrast, atherectomy such as performed with an excimer laser with pharmacological follow-up, actually removes plaque or other occlusive matter, and therefore would appear to have the potential to reduce if not eliminate the need for stenting (see Sharma, S. K., Kini, A., Mehran, R., Lansky, A., Kobayashi, Y., and Marmur, J. D. 2004. “Randomized Trial of Rotational Atherectomy versus Balloon Angioplasty for Diffuse In-stent Restenosis (ROSTER),” American Heart Journal 147(1):16-22.

In practice, however, no known means for the reinstatement of patency in a vessel is free of complications. Furthermore, the presumed inability of a laser catheter to remove at least moderately calcified plaque by cavitation, thermal breakdown, and vaporization appears unwarranted (see Bilodeau, L., Fretz, E. B., Taeymans, Y., Koolen, J., Taylor K., and Hilton, D. J. 2004. “Novel Use of a High-energy Excimer Laser Catheter for Calcified and Complex Coronary Artery Lesions,” Catheterization and Cardiovascular Interventions 62(2):155-161).

Endoluminal stenting serves balloon angioplasty in covering over any dissections that may have resulted from overinflation and by retaining or ‘tacking up’ debris compressed against the lumen wall, thus preventing debris greater in diameter than 5 micrometers, which is too large to pass through capillaries, from passing downstream. By contrast, extraluminal stenting as described herein does not achieve patency by endoluminal scaffolding and therefore does not simply force debris up against the lumen wall or counteract balloon damage with its sequelae of hyperplasia, shrinkage, and spasm. Endoluminal stenting involves some insignificant clotting for endothelial recovery. For vasculopathy that calls for stenting alone, extraluminal stenting, because it leaves the lumen clear, is less susceptible to adverse sequelae. The measures employed to avert embolism are discussed below.

In order to minimize the risks of migration whenever the lumen expands and clogging, an endoluminal stent must assume the internal diameter of the lumen plus enough additional circumferential expansion to remain in place. The stent thus forces the ductus to remain at its maximum diameter as determined by the action of the smooth muscle in the wall of the lumen at all times. Whenever autonomic function would act to contract the lumen, this nonadaptive diametric maximization represents a chronic irritation to the lumen wall. As the pulse or peristaltic wave traverse the stent, the noncompliant margins of the stent ‘dig into’ and irritate the lumen wall. This stands in marked contrast to an extraluminal stent, which is placed about the outside of the ductus to fit at its minimum outer diameter, and elastic, expands with the ductus.

Less suited to masking over shortcomings of endoluminal stenting, extraluminal stenting is better paired with laser atherectomy, which does not involve the application of outward radial force against the wall of the lumen to merely compress and crush plaque or obstructive tissue and is able to remove moderately calcified lesions by actually cavitating, disintegrtating, and vaporizing the occlusive material. When plaque is so calcified that the laser cannot remove it, a preceding rotational atherectomy is indicated.

The actual removal or ablation of plaque (atheroablation) and other diseased tissue, as opposed to the mere crushing and displacement of plaque obtained with balloon angioplasty preferable, and endoluminal stenting being a multirisk-laden underpinning or crutch for this inferior method for opening the lumen, the preferred pairing of extraluminal stenting by means of photoablative laser rather than alternative means for performing the antecedent angiolasty recommends the incorporation into the extraluminal stent delivery device, or barrel-assembly, of a laser catheter as integral, wherefor embodiments that do in fact incorporate laser catheters will be described.

Used manually apart from an airgun, the barrel-assembly when equipped with side-sweepers with or without a trap-filter and a heatable turret-motor can serve as an independent apparatus for mechanical atherectomy and thermal angioplasty in lieu of balloon angioplasty and the incorporation of a laser catheter or rotational atherectomy burr allows its use for atherectomy with calcified plaque. As such, the barrel-assembly can be used to prepare the lumen for the introduction of a conventional or endoluminal stent. Combined forms that include a laser catheter or rotational atherectomy burr are more versatile in the ability to remove calcified plaque.

Nevertheless, an object of the invention is to be able to accomplish both the removal of plaque or other obstructive matter and complete implanting the intravascular component of the intra and circumvascular type stent described herein without the need to reenter the lumen. A muzzle-head designed to contain a rotational burr or laser catheter at its center is equally capable of containing a intravascular ultrasonographic probe or a cryogenic angioplasty balloon, such latter devices integrated into the barrel-assembly and not represented as inventive. Integration thus has as an object prepositioning to eliminate the need for withdrawal and reentry at times when these devices may be needed.

When not thrombogenic (thromboplastic), as when treatment lies outside the circulatory system or is inside but managable with antithrombotic medication, implantation with a thermal angioplasty barrel-catheter following thermal angioplasty can additionally be followed by thermal use of the thermal angioplasty barrel-catheter to initiate the release of medication or time-release medication from medicated miniballs, stays, or stent-jacket expansion inserts, or to accelerate dissolution of the latter two without withdrawal of the barrel-assembly. Arcuate stays and expansion inserts can not only be irradiative or coated with medication but incorporate agents to control their dissolution, which can also liberate medication.

Stays for use independently of a stent-jacket and thus lacking ferromagnetic material can be irradiating seeds. Conventional seeds imaged ultrasonographically or by means of computed tomography, seed stays not conventionally marked for radiopacity can be encapsulated with a layer that includes a gamma emitting isotope for gamma camera viewing. Within the size constraints, stays can be encapsulated with medicine. Miniballs of like purpose can be of like composition as stays. The use of the methods and apparatus described herein to implant spherules that consist solely of medication and/or serve as a substrate for the emission of radiation from a highly localized site within the wall of a tubular anatomical structure is described below. Any barrel-tube can be used to infix a medicated, multiple concentric medicated, and/or irradiative implant along the wall of a ductus thus causing the medication or radiation to be eluted or emitted in a highly localized discretionary manner rather than circulated thorughout the bloodstream.

Less traumatic to introduce notwithstanding, endoluminal stents, whether in the vascular tree, the tracheobronchial tree, the bile, or urinogenital ducts, all cover over and compress portions of the internal surface or endothelium and intima of the lumen, necessarily interfering with normal lumen wall physiology at every level from the biochemical, to the microscopic, to the gross anatomical. Broadly, mechanical expedients essential to meet the priority of maintaining patency as simply imperative, endoluminal stents are otherwise noncompliant with vessel physiology in every way.

In the vasculature, chronic contact with the luminal endothelium probably excites the subjacent layers to proliferate—“It is believed that the central role of the vascular endothelium is to maintain quiescence of the underlying media and adventitia” (Aoki, J., Serruys, P. W., van Beusekom, H., Ong, A. T., McFadden, E. P., Sianos, G., van der Giessen, W. J., Regar, E., de Feyter, P. J., Davis, H. R., Rowland, S., and Kutryk, M. J. 2005. “Endothelial Progenitor Cell Capture by Stents Coated with Antibody Against CD34: The HEALING-FIM (Healthy Endothelial Accelerated Lining Inhibits Neointimal Growth-First In Man) Registry,” Journal of the American College of Cardiology 45(10):1574-1579). By comparison, the typically 0.4 millimeter trajectories of ballistic implantation quickly seal and quickly heal, and even when recommending anti-platelet agents and anti-coagulants, do not represent a permanent source of irritation.

In the airway, secretory and mucociliary action, the movement of phagocytes, normal exposure to oxygen and moisture, other chemical interaction at the surface of the lumen, and smooth muscle action are all disrupted. Interference with the normal physiology of the tracheobronchial tree thus exerts a significant nonmechanical secondary effect in impairing immunomodulatory function. In the vascular system, an endoluminal stent facilitates the thrombogenicity associated with any antecedent angioplasty that resulted in dissection of the lumen wall promoting thrombogenesis, intimal hyperplasia, and constrictive modelling.

In the airway, a conventional stent, in marked contrast to subfibrosally implanted minispheres, can serve as a scaffold for the spread of tubercular or other infection (Bautista, M., Greenberg, A., and Weissman, P. 2002. “Expansion of a Lung Abscess after Stent Closure of a Bronchoesophageal Fistula,” Gastrointestinal Endoscopy 55(2):281-283; Benesch, M., Eber, E., Pfleger, A., and Zach, M. S. 2000. “Recurrent Lower Respiratory Tract Infections in a 14-year-old Boy with Tracheobronchomegaly (Mounier-Kuhn syndrome),” Pediatric Pulmonology 29(6):476-479).

To reduce the risk of displacement, outward radial force sufficient to overcome the propulsive or propagative action passing along the ductus wall must be exerted against the lumen wall. Mass flow through the stent undiminshed, even when expanded just enough to preclude migration, an endoluminal stent interrupts and thus interferes with the radial movements in the ductus wall at both the proximal and distal stent margins. The impulse to prevent migration by overly expanding a stent in particular results in constant restraint-irritation and injury of the ductus leading to delayed and long term sequelae. Essentially, an endoluminal stent uses the propulsive forces in the lumen wall to cause the ductus to injure itself. Thus, often practically irrecoverable, endoluminal stents can be life-saving upon insertion only to produce serious if not life-threatening complications at a later time.

Numerous problems associated with endoluminal stents arise not just from the outward radial forces imposed upon the lumen wall but from placement within the lumen adjacent to if not directly in the path of passing contents. A stent within an artery, especially one made of metal, encourages the clotting and adhesion to its surface of blood, prompting the administration of anticoagulants to dangerously high levels. A stent in a ureter acts in a roughly analogous manner to encourage the deposition of calcium oxalate, calcium phosphate, and ammonium magnesium phosphate salts. Situated thus, the contents, if not positively induced to precipitate onto the alien surface, can additionally be trapped inside and clog the stent.

In marked contrast, an elastic stent outside the lumen complies with any change in caliber of the substrate vessel or duct and therefore is far less likely to be a constant irritant. Except for those made of tantalum or platinum, endoluminal stents are poorly radiopaque, and will usually, should one be dropped from the balloon (Hubner, P. J. B. 1998. Guide to Coronary Angioplasty and Stenting, Amsterdam, Holland: Harwood Academic Publishers, page 108), prove difficult if not impossible to locate much less retrieve without open exploratory surgery.

Historically, the main problem with stenting in the vascular tree—restenosis—was to an extent ameliorated with the appearance of the Palmaz-Schatz stent. However, the central joint or articulation in this endoluminal stent, which is provided to allow some flexion for trackability, is a point of weakness that fails to adequately retain the subjacent lumen wall, which under intravascular ultrasound is seen to prolapse into the joint and constrict the lumen. In fact, “ . . . the stent has poor trackability and is best used for proximal lesions in straight vessels, free from disease down to the lesion,” (Hubner, op. cit. page 107).

In the effort to suppress the restenosis of an endovascular stent, an angiotensin receptor antagonist or blocker (see for example, Traub, X. and Shapiro, A. P. 1997, “Management of Hyertension with Particular Attention to the Renin-Angiotensin System,” in Glew, R. H. and Ninomiya, Y., Clinical Studies in Medical Biochemistry, New York, N.Y.: Oxford University Press), such as valsartan (Diovan®); an angiotensin receptor blocker that acts as a citokine gene expression inhibitor, such as tacrolimus; or candesartan cilexetil; or an angiotensin II receptor antagonist or blocker and cytostatic immunosuppressant, such as sirolimus (rapamycin, Rapamune) is often administered, even though the efficacy in long term use of immunosuppressants or anti-hypertensives for this purpose remains unproven. While relaxing the lumen wall allows transluminal access to narrower portions of the vascular tree, an extraluminal stent should not require or recommend such medication on a continued basis.

A more recent version of the Palmaz-Schatz stent, the Palmaz-Schatz Crown stent, has two spiral articulations, and while more trackable, still suffers from the common problems associated with endoluminal vascular placement, as do the most advanced endovascular stents. If obstructed, some tend to drop from the balloon. If the stent becomes stuck and an effort is made to withdraw it, or if the balloon is withdrawn, single wire coil stents may uncoil, and rarely, catheter components, to include guidewires, Rotablator® Systems, and stents, become entrapped during cardiologic interventions causing life-threatening complications and the need for emergency cardiac surgery (Alexiou, K., Kappert, U., Knaut M., Matschke, K., and Tugtekin, S. M. 2006. “Entrapped Coronary Catheter Remnants and Stents: Must They Be Surgically Removed?,” Texas Heart Institute Journal 33(2):139-142. With several endovascular stents, the delivery catheter balloon may fail to deflate, making withdrawal difficult. In some instances, this has led to serious complications requiring coronary artery bypass surgery or to death.

The blunt and slippery nose of the muzzle-head poses little risk of perforations, and made of tough, flexible, and strongly bonded polymer tubing, the entire apparatus less still of fractures. The absence of a guidewire eliminates the low incidence of mishaps associated with these, to include:

a. Perforation (see, for example Shirakabe, A., Takano, H., Nakamura, S., and thirteen other authors 2007. “Coronary Perforation During Percutaneous Coronary Intervention,” International Heart Journal 48(1):1-9; Axelrod, D. J., Freeman, H.; Pukin, L., Guller J., and Mitty, H. A. 2004. “Guide Wire Perforation Leading to Fatal Perirenal Hemorrhage from Transcortical Collaterals after Renal Artery Stent Placement,” Journal of Vascular and Interventional Radiology 15(9):985-987; Naik, M., Lau, K.-W., and Chua Y.-L. 2001. “Guidewire Perforation during PTCA with Subsequent Off-Pump Bypass Surgery,” Texas Heart Institute Journal 2001 28(1):70-71; Witzke, C. F., Martin-Herrero, F., Clarke, S. C., Pomerantzev, E., and Palacios, I. F. 2007. “The Changing Pattern of Coronary Perforation During Percutaneous Coronary Intervention in the New Device Era,” Journal of Invasive Cardiology 16(6):257-301; Kent, J. and Nedumpara, T. 2007. “Perforation of the Gall Bladder by a Peripherally Inserted Central Catheter Guidewire: ‘If it Can Happen it Will;’ Australia-New Zealand Journal of Surgery 77(3):190-191).
b. Fracture (see, for example Collins, N., Horlick, E., and Dzavik, V. 2007. “Triple Wire Technique for Removal of Fractured Angioplasty Guidewire,” Journal of Invasive Cardiology 19(8):E230-234; Vrolix, M., Vanhaecke, J., Piessens, J., and De Geest, H. 2005. “An Unusual Case of Guide Wire Fracture During Percutaneous Transluminal Coronary Angioplasty,” Catheterization and Cardiovascular Diagnosis 15(2):99-102; Gavlick, K. and Blankenship, J. C. 2005. “Snare Retrieval of the Distal Tip of a Fractured Rotational Atherectomy Guidewire Roping the Steer by its Horns,” Journal of Invasive Cardiology 17(12):E55-E58).
c. Fracture necessitating emergency surgical retrieval (see, for example, Demirsoy, E., Bodur, H. A., Arbatli, H., Ya\check{g}an, N., Yilmaz, O., Tükenmez, F., Oztürk, S., and Sönmez, B. 2005. “Surgical Removal of Fractured Guidewire with Ministernotomy,” Anadolu Kardiyoloji Dergisi/Anatolian Journal of Cardiology 5(2):145-477 (available at http://www.anakarder.com/sayilar/21/2005-145-147.pdf).
d. Entrapment necessitating emergency surgical extraction and a coronary artery bypass graft (see, for example Kim, C. K., Beom Park, C., Jin, U., and Ju Cho, E. 2006. “Entrapment of Guidewire in the Coronary Stent During Percutaneous Coronary Intervention,” Thoracic and Cardiovascular Surgeon 54(6):425-426).
e. Fracture with entrapment (see, for example Marti, V. and Markarian, L. 2007. “Atrapamiento de la guia de angioplastía después de la implantación de un stent: Descripción de dos casos y revisión de la literatura,” (“Angioplasty Guidewire Entrapment Following Implantation of a Stent: Description of Two Cases and a Review of the Literature”) Archivos de cardiologia de Mexico 77(1):54-57; Kim, C. K., Beom Park, C., Jin, U., and Ju Cho, E. 2006. “Entrapment of Guidewire in the Coronary Stent During Percutaneous Coronary Intervention,” Thoracic and Cardiovascular Surgeon 54(6):425-426; Ozkan, M., Yokusoglu, M., and Uzun, M. 2005. “Retained Percutaneous Transluminal Coronary Angioplasty Guidewire in Coronary Circulation,” Acta Cardiologica 60(6):653-654; Khambekar, S., Hudson, I., and Kovac, J. 2005. “Percutaneous Coronary Intervention to Anomalous Right Coronary Artery and Retained Piece of Guidewire in the Coronary Vasculature,” Journal of Interventional Cardiology 18(3):201-204;
f. Fracture with entrapment necessitating emergency percutaneous retrieval (see, for example Khong, P. L. and John, P. R. 1997. “Percutaneous Retrieval of a Fractured Biliary Guidewire from a Reduced Liver Graft,” Pediatric Radiology 27(3):253-254; Pande, A. K. and Doucet, S. 1998. “Percutaneous Retrieval of Transsected Rotablator Coronary Guidewire Using Amplatz “Goose-Neck Snare”,” Indian Heart Journal 50(4):439-442; Savas, V., Schreiber, T., and O'Neill, W. 1991. “Percutaneous Extraction of Fractured Guidewire from Distal Right Coronary Artery,” Catheterization and Cardiovascular Diagnosis 22(2):124-126).

In any endovascular stent, a larger mesh or grid gap improves side branch accessibility to a guidewire, but only at the risk of lumen wall prolapse due to a lack of support (Hubner, op. cit. page 114). Furthermore, situated at or beside the ostium, the grid is more thrombogenic, making a large mesh risky for spanning a side branch. Inducing patency by extravascular means would not eschew all limitations and sequelae, but it would these. Demisroy et al. provide references pertaining to the thromogenic, spasm inducing, and narrowing that can result from allowing a fractured guidewire to remain in a coronary artery.

Radially and longitudinally rigid and continuous in structure, most endoluminal stents are noncompliant to physiological changes in vascular gauge and unaccomodating of gross movement. To span or straddle branches or to bend, separate stents must be used to either side of the branch or point of flexion, presenting the multiple thrombogenic edges, or margins, of two or more stents. Furthermore, “evidence is emerging that the abrupt compliance mismatch that exists at the junction between the stent ends and the host arterial wall disturbs both the vascular hemodynamics and the natural wall stress distribution” (Berry, J. L., Manoach, E., Mekkaouri, C., Rolland, P. H., Moore, J. E., and Rachev, A. 2002. “Hemodynamics and Wall Mechanics of a Compliance Matching Stent: in Vitro and in Vivo Analysis,” Journal of Vascular and Interventional Radiology 13(1):97-105; for the effect of shear stress on in-stent restenosis, see Wentzel, J. J., Krams, R., Schuurbiers, J. C. H.; Oomen, J. A. Kloet, J., van der Giessen, W. J., Serruys, P. W., and Slager, C. J., 2001. “Relationship between Neointimal Thickness and Shear Stress after Wallstent Implantation in Human Coronary Arteries,” Circulation 103(13):1740-1745 and Sanmartin, M., Goicolea, J., Garcia, C., Garcia, J., Crespo, A., Rodriguez, J., and Goicolea, J. M. 2006. “Influencia de la tensión de cizallamiento en la reestenosis intra-stent: estudio in vivo con reconstrucción 3D y dinámica de fluidos computacional,” [Influence of Shear Stress on in-Stent Restenosis: in Vivo Study Using 3D Reconstruction and Computational Fluid Dynamics],” Revista española de cardiologia 59(1):20-27.)

Intraluminal stents are incapable of treating the eccentricities characteristic of angiosclerotic lesions discriminately, instead covering over unaffected portions of the arterial wall as well. The two critical distinctions between an endoluminal and an extraluminal stent then, are that the latter avoids the numerous complications associated with insertion within, or rather intrusion into, the lumen, and unlike the former, is compliant to muscular action in the wall of the structure.

Endoluminal stents in the trachea or esophagus can interfere with gross motility as well as smooth muscle action. A distinct irritant, endoluminal stents in the vascular tree have stimulated intimal hyperplasia leading to restenosis, accelerated atherosclerosis, and resulted in ischemiatizing intramedial protrusion sometimes leading to erosions, fistulization responsive to the chronic irritation of physiologically active tissue or infection, and interference with normal intrinsic motility, while stents in the gastrointestinal tract are noteworthy for accumulating detritus and interfering with peristaltic motility as well as voluntary movement. Whether due to primary deformity or pathological deterioration, protracted impairment in physiological function from immobilization over time further destroys normal structure and function in the lumen wall. Even though the smooth muscle has deteriorated or atrophied, a stent that is able to comply in expandability and contractility can be significant in preserving normal vascular physiology.

From the moment of placement, an endovascular stent poses the risk of causing thrombogenic turbulent flow at the edges that the thrombophilic metal surface of every practical vascular stent aggravates (see for example, Manjappa, N., Agarwal, A., and Cavusoglu, E. 2006. “Very Late Bare-metal Stent Thrombosis. A Case Report and Review of the Literature,” Journal of Invasive Cardiology 18(7):E203-E206. Endoluminal stents also conflict with physiological action in noncompliance to autonomic (vasotonic angiotonic) adjustments in lumen diameter to change the blood pressure (arterial tension) and to allow the advancing alternation in expansion and contraction of the pressure waves of the pulse. An endoluminal stent in the bloodsteam reduces the cross-sectional area of the vessel causing the rate of flow through the device to increase, centrifugally agitating platelets and other blood cells that move axially in laminar flow (see, for example, Porth, C. M. 2004. Pathophysiology: Concepts of Altered Health States, Philadelphia, Pa.: Lippincott Williams and Wilkins), and inducing the thrombogenicity associated with turbulent flow.

The tendency for the edges of endovascular stents to irritate and induce the formation of thrombi is increased when multiple, as when used to anchor the ends of an endovascular graft (see Parodi, J. C., Veith, F. J., and Marin, M. L. 1998. Endovascular Grafting Techniques, Baltimore, Md.: Williams and Wilkins, page 128, figure 15.5), or in treating either intermittent segments of vessels diseased over lengths considered too small and not sufficiently deteriorated to justify excision and anastomosis or the insertion of a graft, or segments to either side of a branch or point of flexion. That an endovascular stent is as noncompliant inwardly as outwardly makes it a distinct liability in the event of restenosis to which this noncompliance may have contributed.

Lacking a side-hole wherewith to straddle or span branches of which the orifices are notably susceptible to atherogenesis, intravascular stents necessitate the use of two stents leaving the segment of the vessel wall opposite and subtended by the opening to the branch unstented. Since the proximal ends of endoluminal stents to either, side of the branch opening usually maintain the diameter of the lumen past the opening over the distance separating the two, this usually is not problematic in itself, even when the stent is drug-eluting or irradiative. However, placing endoluminal stents to either side of a branch also results in the presentation of four thrombogenic edges to the blood that flows therethrough in positions of maximum nonlaminar flow and shear stress favorable to the formation of thrombi and atheromatous lesions.

The presence of a stent is likely to interfere with the re-treatment that will most often be due to the etiology, which no stent, even one drug-eluting or radiation-emitting, can more than palliate, and the stent may itself have aggravated if not precipitated the condition that necessitates its retrieval. Thousands of references on file at the National Library of Medicine document the fracture, fragmentation, migration, clogging, and susceptibility to act as bases for the deposition of constituents out of the passing fluid of endoluminal stents. In producing these consequences, endoluminal stents introduce mechanical as well as physiological complications that often necessitate a second procedure, often one involving open surgery, to effect their retrieval.

5. Concept of the Extraluminal Stent and the Means for its Placement

Laparoscopic techniques have made it possible to access vessels and ducts without extensive dissection, making stenting that necessitates local entry for circumvascular access less objectionable than when open exposure with large incisions was necessary. Unless deep and invested within skeletal muscle, practically any ductus can be extraluminally stented despite its extent of attachment or investment in connective tissue. Essentially, all but muscular vessels in the extremities are debarred, those in the neck and trunk usually treatable.

Dissection for circumvascular access should be adequate to minimize any compression or abrasion of the adjacent tissue. The small bar magnets mounted to the outer surface of the stent-jacket substrate tube or base-tube are magnetized at right angles or normal to their length, so that the lines of force are radial to the central axis of the ductus. Since the edges of the perpendicularly magnetized small bar magnets about the outer surface of the stent-jacket are rounded and encapsulated, unless moving independently of and abrasively in relation to the magnets, the surrounding tissue is not irritated, making the mere fact of abutment insignificant.

Magnets that would encroach upon and irritate neighboring tissue, such as when applied to a coronary artery where it abuts upon the heart, are situated aside from the side-slit opened to encircle the vessel. Provided the ductus can be sufficiently encircled to assure secure positioning, a deep or far side attachment is left intact, only the encircleable arc implanted and attracted. Dissection when unavoidable is minimized with the use of ties in conjunction with a circumferentially larger side-slit, or side-slot.

When implantation along an interface preempted, either an eccentrically discharging barrel-assembly or one radially symmetrical with the side facing the adjacent tissue blanked at the rotary magazine clip is used to implant the freed circumference of the vessel. Then dissection to free the vessel, preferably accomplished robotically to minimize the trauma of access, is limited to the freed circumference necessary for secure placement. The stent-jacket side-slit or one with a circumferentially extended slit, a side-slot, is used to clear any underlying or far side attachments.

Elsewhere, when the ductus can be undercut or separated from the underlying connective tissue to admit the stent-jacket base-tube, but the additional presence of magnets, even thin with rounded edges, would pose the risk of erosive or fistulative injury to the underlying tissue, implantation and magnets are eliminated over the contact surface. Where the normal anatomy or a condition resulting from disease makes undercutting the ductus parallel to its axis impracticable, circumvascular end-ties of nonabsorbable suture are passed beneath the ductus. Following healing and the cessation of anticoagulants, an extravascular stent is athrombogenic, and unlike endoureteral stents, an extraureteral stent is not susceptible to encrustation, bacterial colonization, or enstonement that necessitates painful and operatively difficult replacement every three months.

Vessels in adaptive tonicity and pulsation, and ducts in peristasis expand and contract around a mean diameter or caliber. Enclosing the vessel or duct with a compliant mantle while holding the lumen patent, that is, supporting the vessel wall both contractively and expansively, the extraluminal stent can deal with failures whether inward, outward, or combinations of the two. The size of the stent-jacket is selected to allow circumvascular encirclement and slit clearance or gapping to allow contraction. The stent-jacket then draws the wall of the structure toward its inner surface but not with such tractive (magnetomotive) force as prevents contractive travelling waves from passing through without significant distortion.

In the vascular tree and especially in the ureters, the endoluminal stent is precisely itself the scaffold that induces the deposition of matter out of the passing fluid. Leaving the lumen clear, an extraluminal stent used in place of an endoluminal stent following an angioplasty that resulted in dissection, for example, tends less to induce proliferative or thrombotic sequelae as contribute to the need for, complicate, and interfere with retreatment for restenosis.

Since the elastic stent-jacket has sufficient compliance to accommodate normal (tonic, pulsatile, or peristaltic) changes in caliber, the unmodified elastic stent-jacket also adjusts to reductions in ductus cross-sectional area ordinarily associated with the reduction in the size of (resolution, subsidence, shrinking, regression) or healing of an incipient aneurysm. However, if initially sized to accommodate autonomic expansion in an already enlarged condition, then following subsidence, the end internal diameter of the stent-jacket must remain too large. The stent-jacket should not remain larger in internal diameter than the outer diameter in its resting phase of the ductus once healed.

Rather than to leave the stent-jacket slightly oversized once healing is complete, when the enlargement of the vessel or duct, whether due to intrinsic inflammatory (ectasic) swelling or to swelling as the direct result of ballistic implantation, is less than 20 percent or so, a stent-jacket with slightly greater elasticity to avoid interim irritation may be chosen for the eventually normal mean diameter, meaning that pending subsidence, the stent-jacket will not fully encircle the ductus. Usually, however, and necessarily when the discrepancy is larger, a stent-jacket with an expansion insert to be described is used so that the initial diameter of the stent-jacket accommodates the enlargement in the ductus but the end diameter and pliancy following healing will fit properly over the long term.

That is, if following healing, the autonomically varying mean diameter of a vessel is expected to reduce by 20 percent or more in relation to the stent-jacket, then a specially adapted stent-jacket that effectively contracts in diameter over a predictable time that is keyed to the anticipated healing time of the diseased vessel or duct is necessary, and such embodiments will be described. Adaptability in diameter obviates a forced choice between a stent-jacket that fits initially but will be too large following healing, or worse, a stent-jacket sized for the anticipated end-caliber that would constrict and irritate the vessel or duct.

For containing a less progressed aneurysm, a stent-jacket is used without magnets or intravascular implants that while to place requires open access, does so less traumatically, which can encourage preemptive arrest before a more advanced fusiformation or sacculation, either of which can become lined with a laminated thrombus, ensue. If end-tethers for preventing migration, as will be described, are contraindicated, but sufficient lengths of healthy tissue extend beyond the ends of an affected segment, then bridging or overextension of the stent jacket or articulated stent-jacket onto the healthy tissue allows magnets and implants to be used over such nonaneurysmal or otherwise unaffected segments, and in contrast to an endoluminal stent used in a similar way, without encroachment upon the physiologically more active inner layers of the vessel.

Mounting tiny perpendicularly magnetized bar magnets longitudinally about a length of resilient tubing provides a vascular surround or sleeve referred to as a stent-jacket, that is able to expand and contract, hence, comply with changes in the diameter or gauge of a tubular structure whether tonic, pulsatile or peristaltic. While free to expand and contract and thus comply with autonomic and renin-angiotensin system regulated changes in gauge, the elastic circumvascular stent-jacket is not susceptible to permanent deformation from an accidental fall, for example.

Spontaneous adaptability in cross-sectional area to different environmental and behavioral conditions or the need to propel contents a significant aspect of normal vessel and duct physiology, an extraluminal stent, in marked and significant contrast to endoluminal stents, does afford such compliance. Resilient, an extravascular (extraluminal vascular) stent-jacket is not susceptible to deformation with ensuing thrombogenesis in response to external forces, as from a fall or the impact of a collision. An extraluminal stent can be flexible and compliant in any length or biased to provide flexibility or compliance that is eccentric.

The vessel or other duct is kept patent by a mild outward pull upon the lumen wall toward the internal surface of the surrounding circumvascular stent-jacket, which elastic, freely expands and contracts in response to changes in vessel diameter tonic pulsatile or peristaltic. This stands in marked contrast to the fixation in diameter and radial force against the lumen wall imposed by endolumnal stents.

For the coronary arteries, most other vessels, and narrow gauge ducts, the use of multibarrel radial discharge barrel-assemblies that discharge fractional millimeter miniballs, specifically when advanced by means of an automatic positional control system, as will be described, allows higher density implantation, or the implanting of numerous miniballs at smaller intervals, to allow a reduction in the magnetic force exerted over a given unit area and any one miniball, thus reducing the likelihood that any will be pulled entirely through the adventitia. While the miniball implants are encapsulated for bioinertness and small (typically 0.4 millimeters), so that even were such perforations to occur, the leakage of contents would be quickly and spontaneously truncated and the loss of miniballs within a body cavity would have no medical significance, the loss of a threshold number would impair the effectiveness of the stent from a functional standpoint.

Rotary magazine clips that provide ten or more discharges of four or more shots to the shot group per discharge support the loading requirements for high-density implantation. However, it is necessary to withdraw the barrel-assembly by quick steps that precludes distorting (drawing or stretching) the lumen wall (endothelium and intima), and such increments with proper spacing is too fine for manual placement or even visualization with the aid of imaging equipment. Broadly, applications of the apparatus to be described fall into three categories—the air handgun with a simple pipe-type barrel-assembly, the dedicated airgun with a radial discharge barrel-assembly, and the manual insertion of stays. The first combination is suitable in the airway where the anatomy is differentiated or structured and the size of the anatomy allows the apparatus to be accurately positioned. Most stenting of the airway will be veterinary or pediatric.

The second combination is used in vessels or ducts that tend to lack anatomical landmarks where the lumen may contain blood or other contents and the size of the vessel or duct is such that maneuverability and accurate positioning are difficult or impossible. Stays can be used where neither of the first two means are preferred as well as in situation unrelated to stenting, to include the highly localized placement of medication or radiation. Stays for the latter purposes may be absorbable (temporary).

Concerns with such implants include the consequences of perforating the ductus wall upon discharge, the pulling through of implants under the constant if weak attraction of the stent-jacket magnets, and when used in the vascular tree, the entry of implants into the bloodstream. Depending upon the specifics involved, the trauma that any one of these eventualities pose with one or a few small diameter implants, typically 0.4 millimeters, is preventable, discountable, or remediable without the need for reentry, much less radical measures. In locations where perforation cannot be tolerated, the stent-jacket is placed prior to initiating discharge. The control of rebound then essential to prevent entry of the implant into the lumen is discussed below under the section entitiled Double-wedge Stent jacket Bumper-rebound Directing Linings.

To the extent possible, the object is to have multiple implants together lift the lumen wall as a uniform sheet, so that even though magnet pole foci of attraction must exist, the force of attraction on any one implant, even at the apices of the foci, will not be sufficiently disproportionate to pull implants through the adventitia, so that to deliberately avoid placing implants within areas of focal field intensity is not necessary. While to generalize concerning conditions that apply to all types of ducti as the present methods and apparatus pertain is difficult, by virtue of its more general symptoms, a malacic condition that affects the outer layer of ducti seldom remains unidentified, the limitation of such a condition to the outer layers is unseen (see, for example, www.nhlbi.nih.gov/ . . . /Diseases/vas/vas_all.html), and a test for wall strength is provided in the section below entitled In situ Tissue Puncture and Penetration Test.

As an inherent consequence of the need to achieve substantial uniformity of attraction, implants are not isolated such that the force of attraction, through unequal, is limited to one or a few. A minimal but sufficient density of implants is used so that the gradual pull-through of some would not unacceptably degrade stenting function. Since to the extent possible, the stent-jacket is fitted to the substrate ductus in a length that is spanned by the passing bulge of smooth muscle and as to grasp the ductus about while relaxed then resiliently expand with the ductus, and exudates within the path torn through by the miniball would afford some adhesion, for a miniball to enter the adventitia-stent-jacket interface and migrate away from its point of entry is improbable.

Nevertheless, were this to result, irritation to the ductus should resolve itself in the short term, the bioinertly encapsulated submillimetric miniball either dropping into the surrounding cavity, or if trapped, becoming embedded in the lining of the stent-jacket under the force of constantly repeated contraction. Hence, once the stent-jacket has been placed, the gradual pull-through of one or a few miniballs into the interface separating the internal surface of the stent-jacket from the outer surface of the ductus as to persist therein and chronically irritate the ductus is improbable and unlikely to produce significant consequences either medical or with respect to stent sufficiency.

Even with no stent-jacket prepositioned to prevent perforation, the practical risk posed by a miniball of fractional millimeter diameter that perforated is nugatory. The likelihood of a perforation resulting because the operator was unaware that near certain ganglia or the head a vulnerable structure lay along the trajectory and failed to take prescribed measures for accommodating this condition is slight, the size of the projectile limits the injury that could be inflicted, and perforations tend to seal quickly. A primary reason for ballistic implantation is precisely the fact that the access path is no larger than that of the implant itself, and therefore quickly seals and quickly heals.

Furthermore, were the miniball to gain entry into the lumen of another blood vessel, for a miniball of such diameter (usually 0.4 millimeters) to embolize a vessel of which the dependent tissue is without collateral circulation is slight. To emphasize the point, due to nervous and vascular redundancy, a 0.4 millimeter miniball shot entirely through the body would be unlikely to do signficant damage. In even a worst case situation where rather than to gain lumen entry by perforation one or even several miniballs passed the continuously energized miniball recovery tractive electromagnets at the front of the muzzle-head provided precisely to prevent such an eventuality and then additionally managed to pass an external miniball recovery and extraction tractive electromagnet positioned downstream, an actual need to recover these through open surgery would be improbable.

Even where collateral circulation or nervous redundancy are lacking, provided the tissue lost was not extensive and critical such as ventricular, following atrophy and necrosis, the ischematized tissue would be replaced by scar tissue (see, for example, Frangogiannis, N. G. 2006. “The Mechanistic Basis of Infarct Healing,” Antioxidants and Redox Signaling,” 8(11-12):1907-1939). Unless lodged in an location that was itself or was behind a structure that would be acutely sensitive to small scale trauma, one or a few miniballs could, however bizarre, be recovered simply by magnetic extraction.

That is by pulling it through and through the body with a powerful external electromagnet. This would tear many capillaries, venules, and muscle and nerve fibers, but produce negligible injury over the long term. Specifically, it would Interposed vessels and organs that were perforated would quickly seal themselves and heal, and the severing of nerve fibers that produced some functional impairment internally or an area of cutaneous numbness would soon recover.

When the muzzle-head can approach the miniball at the lumen diameter where the external miniball recovery and extraction tractive electromagnet interdicted its further travel, raising the current to the inmate tractive electromagnets in the muzzle-head and deenergizing the external magnet result in the attraction of the miniball into the antemagnet chambers. When this is not possible, the diameter of the vessel will be such that collateral circulation is present or the extent of affected tissue even if necrotized is soon resolved. Since the implants are bioinert, the escape of any into the surrounding cavity would not likely prove problematic.

To prevent the miniball implants from moving and the stent breaking down over time, the tractive force applied to the miniballs is evenly distributed by positioning these in a uniform pattern. To accomplish uniformity of distance between adjacent miniballs at the small dimensions involved exceeds the precision that can be achieved by manual control and therefore necessitates an automatic positioning means. The use of a stepper motor to advance a catheter goes back to at least the mid-1970s (see, for example, Clark, J. S. and Farr, F. L 1978. “Alveolar Gas Sampling System and Method,” [U.S. Pat. No. 4,220,162]; Bradley, W. E Klatt, W. M., Kuyava, C. C., and Dreher, R. D. 1980. “Urethral Catheter Puller,” [U.S. Pat. No. 4,233,991], and a catheter-based transducer through the lumens of vessels at millimetric intervals was accomplished not later than 1994 (Matar, F. A., Mintz, G. S., Douek, P., Farb, A., Virmani, R., Javier, S. P., Popma, J. J., Pichard, A. D., Kent, K. M., Satler, L. F., Keller, M. and Leon, M. B. 1994. Coronary Artery Lumen Volume Measurement Using Three-dimensional Intravascular Ultrasound: Validation of a New Technique,” Catheterization and Cardiovascular Diagnosis 33(3):214-220; Liu, J. B., Bonn, J., Needleman, L., Chiou, H. J., Gardiner, G. A. Jr., and Goldberg, B. B. 1999. “Feasibility of Three-dimensional Intravascular Ultrasonography: Preliminary Clinical Studies,” Journal of Ultrasound in Medicine 18(7):489-495).

To accomplish this fine incremental advancement or withdrawal, the patient and barrel-assembly are positioned level with the airgun barrel and a semiautomatically operated single-axis linear positioning table to which the airgun is mounted is used to withdraw or advance the barrel-catheter by the same distance (increment, interval) with each discharge. Even when discharges follow in rapid succession, implantation must follow each triggering of the airgun by the short interval that it takes for the projectile implants to travel down the barrel-tubes and reach the trajectory end-point. Incremental movement of the linear positioning table and discharge need not be detained for each such recurrence, for simplicity, successive increments of the table and the triggering of each discharge are time to allow seating of the implants before continuing. The apparatus to be described allows varying both the anatomical distance between successive shot-groups and the interval of time separating discharges.

The availability of a less radical open procedure can encourage intervention earlier than is conventional. For example, according to accepted doctrine, an abdominal aortic aneurysm is monitored until allowed to attain a diameter of five centimeters or more, produces discomfort, or enlarges while under observation. If not arrested earlier, however, the condition can gradually progress to become markedly fusiform or saccular, often accumulating a laminated thrombus lining. The thrombus affords no protection against rupture adjacent to the sac, and once thrombosed, intervention of any kind risks embolization.

That so long as the enlargement has not spread to the common iliac arteries or become thrombosed, a resilient jacket (without intravascular implants)—and contingent upon the longitudinal extent of the enlargement, cut-outs to clear the branches—can be maneuvered into circumvascular position through an incision of a few centimeters encourages earlier intervention. While limited to aneurysms diagnosed prior to any significant expansion, a stent-jacket used to treat an aneurysm is not limited to one of any particular anatomical position, so that an abdomnal aortic aneurysm need not be infrarenal, or the segments of the aorta superior and inferior to the aneurysm not less than 1.5 centimeters.

While the realization is emerging that endovascular treatment should be performed more promptly (Zarins, C. K., Crabtree, T., Bloch, D. A., Arko, F. R., Ouriel, K., and White, R. A. 2006. “Endovascular Aneurysm Repair at 5 Years: Does Aneurysm Diameter Predict Outcome?,” Journal of Vascular Surgery 44(5):920-931.), an endovascular stent-graft causes as many problems as it alleviates (below and see, for example, Rutherford, R. B. 2006. “Randomized EVAR Trials and Advent of Level I Evidence: A Paradigm Shift in Management of Large Abdominal Aortic Aneurysms?,” Seminars in Vascular Surgery 19(2):69-74). Segmented (articulated, jointed) stent-jackets that allow flexion and the clearing of side branches without resistance, bulging, or buckling regardless of length can be used as compliant stent-grafts.

In veterinary practice, a less radical if open procedure for the repair of tracheal collapse would likewise encourage intervention before this invariably progressive condition resulted in hypoxia if not suffocation. A stent-jacket used as a circumvascular or circumtracheal stent-graft can be inserted through one or two incisions that are much smaller than the incisions required by conventional open procedures and secured by means of end-tethers (below). To seal a leaking ductus whether fistulized or accidently incised without interference with its autonomic function, a stent-jacket can be used to seal the defect or perforation.

Even though circumvascular placement averts numerous drawbacks to endoluminal placement, in the case of an aneurysm, unless diagnosed before progressed to the point that placement is difficult if not dangerous, the use of an extravascular stent is to be avoided. Unlike an endoluminal stent graft, use of a circumvascular stent to contain an aneurysmal artery becomes more difficult, less feasible, and less effective as the condition progresses. The larger the diameter of the aneurysm, the more difficult is opening a stent-jacket of suitable diameter to surround it, the greater the risk of rupture, and reducing a thrombus within that could result in complete obstruction and the liberation of embolizing debris. If there is no accumulation within, then the wall becomes too slack and subject to obstructive infolding if reduced from about.

The threshold of difficulty for surrounding an aneurysm with a stent-jacket precedes that at which the risk of slack upon reduction would pose a risk of obstruction when involuted into the lumen. Seepage demanding, a stent-jacket or an articulated stent-jacket or stent-graft can be bonded to the substrate ductus long enough to allow it to heal. The decision to resect an advanced aneurysm and anastomose the ends for sealing encirclement with an circumvascular prosthesis because there results relatively little loss in motile compliance rather than to place a conventional (endoluminal) stent graft, which is neither compliant nor clears branches, must be left to clinical judgment.

Involving neither suture nor incision, infection is less likely and continued enlargement (see Dubenec, S. R., White, G. H., Pasenau, J., Tzilalis, V., Choy, E., and Erdelez, L. 2003. “Endotension. A Review of Current Views on Pathophysiology and Treatment,” Journal of Cardiovascular Surgery (Turin) 44(4):553-557) is counteracted regardless of whether its cause is endoleakage or ‘endotension,’ hypothesized to result from either an imperceptible endoleak (Lin, P. H., Bush, R. L., Katzman, J. B., Zemel, G., Puente, O. A., Katzen, B. T., Lumsden, A. B. 2003. “Delayed Aortic Aneurysm Enlargement Due to Endotension After Endovascular Abdominal Aortic Aneurysm Repair,”Journal of Vascular Surgery 38(4):840-842) or increased graft permeability (Kougias, P., Lin, P. H., Dardik, A., Lee, W. A., El Sayed, and H. F., Zhou, W. 2007. “Successful Treatment of Endotension and Aneurysm Sac Enlargement with Endovascular Stent Graft Reinforcement,” Journal of Vascular Surgery 46(1): 124-127).

It is improbable that an aneurysm would ever heal so as to regain sufficient wall strength, and not merely shrink as occurs upon complete endovascular exclusion, that an absorbable stent-jacket with a liner typically medicated with steroid and antibiotic drugs could be used to contain it for only so long as this strength took to reappear. Circumvascular stents, with or without implants or circumvascular bonding but with side-slit and thus compliant with smooth muscle action, can be used to contain fistulae, and contingent upon early diagnosis and intervention, aneurysms and false (pseudo) aneurysms. Furthermore, while the aneurysmal wall recovers strength to some extent with an endovascular liner (endograft, internal bypass graft; endoprosthesis) in place, recovery is without exposure to the flow of blood, and the stent is practically irrecoverable with the covered over or adumbrated tissue remaining isolated from blood.

To reduce the risk of migration, the stent-jacket 1. Resiliently grasps about the vessel or duct, 2. Is generally longer than the equivalent intraluminal stent, 3. Has a greater contact area, 4. May have nonincisive projections or 5. Be bonded in whole or part to the outer surface of the substrate ductus on its inner surface, 6. Can have an internal surface that is textured or lined with gauze, which also increases the surface area for tissue integration by infiltration and for the adhesion of a film coating of a sealant, antibiotic, other medication, or an adhesive, as will be described, and 7. Have wrap-around straps or ties at one or both ends. Side branches can be spanned without prolapse of the lumen wall through a mesh that is too large. The spherules and/or the internal surface of the stent-jacket can be medicated or irradiative. Adhesives for bonding the gauze within the stent-jacket are specified below in the section entitled Stent-jacket Anti-migration Lining.

Articulated stent-jackets can span static or dynamic bends and can allow closer compliance to passing smooth muscle waves. Means for creating an artificial outer layer about a vas or ductus of sufficient strength when that intrinsic is weak, means for recovering loose and for extracting misplaced spherules, for testing airgun control settings for the force of impact necessary to seat the spherule implants in the diseased tissue encountered in situ quickly through direct observation, and motorization of the muzzle-head are described.

Since compared to healthy tissue, the mechanical properties of diseased tissue vary over a much wider range, a means for actually testing the tissue considered for treatment in situ is imperative. For example, most vascular disease atherosclerotic, degradation in elasticity of the arterial wall due to infiltration by white blood cells that activate collagen and elastin-degrading proteases, and the prognosis for the continuation of this degradation with the administration of protease resistant medication (see, for example, Nichols, T. C., Busby, W. H., Merricks, E., Sipos, J., Rowland, M., Sitko, K., and Clemmons, D. R. 2007. “Protease Resistant IGFBP-4 Inhibits IGF-I Actions and Neointimal Expansion in a Porcine Model of Neointimal Hyperplasia,” Endocrinology (prepublication copy available at http://endo.endojournals.org/cgi/rapidpdf/en.2007-0571v1), that does not interfere with the clearing of fibrin (see, for example, Sachs, B. D., Baillie, G. S., McCall, J. R., Passino, M. A., and 13 other authors 2007. “p75 Neurotrophin Receptor Regulates Tissue Fibrosis Through Inhibition of Plasminogen Activation via a PDE4/cAMP/PKA Pathway,” Journal of Cell Biology 177(6):1119-1132) is a central concern in deciding whether to proceed with implantation and if so, by what means.

Since elevated protease levels are suspected to contribute to the rupture of plaque leading to acute cardiovascular events (Chen, J., Tung, C. H., Mahmood, U., Ntziachristos, V., Gyurko, R., Fishman, M. C., Huang, P. L., and Weissleder, R. 2002. “In Vivo Imaging of Proteolytic Activity in Atherosclerosis,” Circulation 105(23):2766-2771), at least when angioplasty and stenting are elective, medication that moderates the action of proteases may have been initiatied well beforehand. Whether or how to stent the artery by the various methods to be described, whether medication would allow a retention in elasticity, and so on, can determine whether such treatment should proceed. That plaque tends to become progressively calcified is meaningful according to how much of it remains following the steps taken to eliminate it.

An optional side-sweeping feature, consisting of two semicircularly tipped brushes (écouvillonages) of opposite direction is limited to the removal of occlusive material that is softer than calcified plaque. Either or both side-sweeping brushes can be deployed during manual or linear table advancement or withdrawal of the barrel-assembly. To preclude human error as might result in downstream embolization by debris liberated by the side-sweepers, the same switch that deploys and retracts the side-sweepers also controls a trap-filter stored within a concavity or silo in the nose of the muzzle-head when not deployed.

Most studies indicate that despite increased procedural time, the risk of special complications, and additional expense, distal embolic protective filters are withal beneficial (see Sprouse, L. R., Peeters, P., Bosiers, M. 2005. “The Capture of Visible Debris by Distal Cerebral Protection Filters During Carotid Artery Stenting: Is It Predictable?,”. Journal of Vascular Surgery 41(6):950-955; Wholey, M. H., Jannolowski, C. R., Wholey, M. and Eles, G. R 2003. “Carotid Artery Stent Placement—Ready for Prime Time?,” Journal of Vascular and Interventional Radiology 14(1):1-10), whereas others have shown that by causing intimal abrasion and denudation, distal filters actually generate much thromboembolic debris (Müller-Hülsbeck, S., Stolzmann, P., Liess, C, Hedderich, J., Paulsen, F., Jahnke, T., and Heller M. 2005. “Vessel Wall Damage Caused by Cerebral Protection Devices: Ex Vivo Evaluation in Porcine Carotid Arteries,” Radiology 235(2):454-460; Maleux, G., Demaerel, P., Verbeken, E., Daenens, K., Heye, S., Van Sonhoven, F., Nevelsteen, A., and Wilms, G. 2006. “Cerebral Ischemia After Filter-protected Carotid Artery Stenting is Common and Cannot be Predicted by the Presence of Substantial Amount of Debris Captured by the Filter Device,” American Journal of Neuroradiology 27(9):1830-1833).

In light of these findings and the fact that the incorporation of a trap-filter, even without the additional incorporation of a side-sweeper module, reduces working depth down the vascular tree, the selection of a barrel-assembly that incorporates a trap-filter must rest upon clinical judgment made on a case by case basis through preliminary imaging of the specific condition to be treated. Since the apparatus described herein are not limited to use in the vascular tree, distal protection is not appropriate in every embodiment. However, the use of side-sweeping brushes in the vascular tree would generate debris that an improved filter should eliminate without generating debris of its own. Thus, at least the location and method for incorporating a filter into different embodiments to be described should be shown, regardless of whether a filter is actually incorporated into any one specimen. Accordingly, this unresolved controversy is resolved by providing for the incorporation of a trap-filter in each of the various embodiments to be described.

A laser catheter incorporated into the barrel-assembly as described in the section on combined forms below is used in a forward direction or distad without deployment of the side-brushes with trap-filter. Side-brushes, filter, and optionally the turret-motor or miniball recovery and extraction tractive electromagnets for thermal angioplasty are then actuated when the direction is reversed or proximad. Accomplished thus, the forward pass addresses plaque more central in the lumen, while the return pass addresses that more peripheral. When the laser and side-brushes are actuated together, the simultaneous deployment of the trap-filter with the side-brushes is overriden. Otherwise, the trap-filter is separately actuable for deployment as desired.

While the muzzle-head, and the muzzle-head with side-sweepers can accomplish the removal of soft plaque, combination-forms to be described incorporate a laser catheter or rotational atherectomy burr which, depending upon the degree and distribution of calcification as revealed by electron beam or helical (spiral) computed tomography, will allow harder prominences to be removed from the lumen center, while side-sweepers of a bristle hardness or tip type as will be described selected for the specific plaque will reduce the plaque at the lumen periphery. As will be described, combination-form barrel-asemblies use an edge-discharge muzzle-head that unlike a center-discharge muzzle-head, makes the central canal available. Such a barrel-assembly can thus either incorporate as built into it or effectively serve midprocedurally as a guiding catheter for an original equipment manufacturer laser or a rotational blade with aspiration or burr atherectomy cable, or as a passageway through which a hose outlet from a vortex tube can be passed to direct either cryogenic or thermal air against the backside of the nose-cap for performing a precautionary angioplasty, for example.

To allow the cold gas or the hot or cold air to reach out to the periphery of the inner (rear, proximal) surface of the nose-cap heat-window and circulate back into the central canal, the nose-cap heat-window is separated from the outer surface of the ejection head and the delivery tube is smaller in diameter than the central canal. A single vortex tube can be mounted to provide cold or hot air of the required temperature as preferred. This midprocedural capability makes possible angioplasty or atherectomy ‘touch-ups’ by the method preferred following the initiation of stenting implantation. Interchangeability thus requires that the muzzle-head have a distal slit valve as an end seal to exclude lumen contents from entry. When not so occupied, the distal end of the canal can be used to house a trap-filter silo by channeling recursive gas through gas-return paths that are outside (peripheral, radial) in relation to the barrel-tube or tubes.

The internal design of the barrel-assembly incorporates gas pressure relief means so that the pressure of discharge is contained within the barrel-assembly; discharge (release of propulsive carbon dioxide gas) with the barrel-assembly in the vasculature, even when the airgun is not loaded, will not introduce gas into the bloodstream. The generally torpedo-shaped distal component of the barrel-assembly or muzzle-head supports the walls of a stenotic vessel or duct and is usually jointed to flex and grooved or fluted to allow blood to pass. The longitudinally centrad apices of these longitudinal concavities or blood grooves pass midway between and longidudinally past the muzzle-ports. The invention is intended not only to stent but to accomplish angioplasty and stenting in a single operation, eliminating the need for multiple withdrawal and reentry and thus entry wound complications.

Not expandable as is a balloon, for angioplasty, the body or shell of the muzzle-head muzzle-head would be limited to lumens that match or slightly exceed it in diameter and are occluded by soft atheromatous lesions. Although care has been exercised to provide paths for blood to pass the muzzle-head when these diameters match, obstruction to the flow of blood will often prove unavoidable. Accordingly, minimizing intracorporeal time is even more important than it is in conventional intervention, and several features have been incorporated to accomplish this. Whether to increase the force applied to the lumen wall for angioplasty or to pull the muzzle-head aside to allow more blood to pass, an external hand-held electromagnet can be used to attract the turret-motor and miniball recovery and extraction tractive electromagnet cores so that the muzzle-head is drawn against the near side of the lumen wall.

This approach is limited to a lumen that matches the muzzle-head in diameter, to plaque that is not calcified, by the need to avoid excessive force as might injure the lumen wall, and by the fact that miniballs already implanted on the opposite side of the vessel or duct could be extracted. To ameliorate the limitations imposed by the restrictedness of the lumen diameter and the hardness of some lesions, the barrel-assembly incorporates several means for accomplishing angioplasty. When the bristle tips are not sharply pointed as would perforate the lumen wall, the deployment of one side-sweeping brush (see below) also allows the muzzle-head to be nudged off-center in the diametrical direction.

Brushes retracted into recesses in the muzzle-head are available to brush the sides of the lumen wall. Based upon a preliminary determination of the disribution and character of the plaque (usually by computed tomography), brushes having bristles of suitable hardness and tip conformation are mounted in the recesses. The side-sweepers, whether brushes or subminiature cutting tools, can be used with the barrel-assembly manually moved transluminally, and since the brushes can be rotated at any level with the turret-motor, the wider sweep of the length rather than the width of the brushes can used. Rotation enables the bringing to bear of alternating bundles of bristles having different hardnesses or tool configurations in each brush.

Using a rechargeable lithium-ion polymer electrolyte (lithium polymer, Li-poly, LiPo) battery pack, preferably of the thin film kind for increased charge-discharge cycles, to supply power to the electrical components within the barrel-assembly allows the manual operation of all but the turret-motor as a rotatory mover, which is, however, generally used thus only during discharge with the barrel-assembly removed from the airgun barrel without a dangling power cord. Angioplasty with the barrel-assembly is performed manually, the proximal end inserted into the airgun for stenting only after the angioplasty is completed and the muzzle-head as been brought to the position at which stenting is to be initiated.

Provided the barrel-assembly is not fully intracorporeal so that the slack needed for transluminal movement can be obtained by simply moving the airgun closer to the patient, angioplasty can be resumed without detaching the barrel-assembly after it has already been inserted into the airgun. Otherwise, with the barrel-assembly not removed from the airgun in order to free the proximal end for ease of manipulation by hand, the airgun linear positioning table can be used to move the muzzle-head.

The linear positioning table typically allows continuous movement of 15 centimeters even though such a large distance is not often needed to move from one lesion to the next over the affected segments of a vessel. This situation more often arises when the benefit of additional angioplasty becomes apparent only after stenting has already been intitiated. Upon resuming discharge, the barrel-assembly must be returned to a substantially level profile, and means in the form of a leveling linkage will be described for this purpose. If the area for secondary treatment proves more extensive, the barrel-assembly is removed from the airgun freeing it for independent movement as if airgun insertion had not been interim used.

For these reasons, the controls for heating the turret-motor stator, deploying the side-sweeper brushes, and other components used during preparatory or end-purpose angioplasty while the barrel-assembly is used as an independent device and remains separate from the airgun are duplicated in a miniature control panel that is directly attached to the proximal end of the barrel-assembly short of the length to be inserted into the airgun. Implantation demands greater precision than angioplasty, recommending controlled machine support. Except for repositioning of the muzzle-head over distances greater than 15 centimeters, once stenting has been initiated, the control of transluminal positioning is by means of a linear positioning table, and rotatory positioning by means of the turret-motor, as will be described.

Because while used as a separate device, an ablation and angioplasty barrel-assembly demands free and independent movement, the miniature control panel for ablative and angioplastic functions is directly mounted at the proximal end of the barrel-assembly short of the length that will be inserted into the airgun. The preparatory or end-purpose ablation and angioplasty controls include those for heating the turret-motor stator and either or both recovery electromagnet windings, deploying the side-sweeper brushes, directing (rotating) an eccentric (slot or slit shaped) turret-motor heat-window and/or side-sweeper brush. By the same token, the controls for discharge positioning and discharge are mounted to the airgun. Some original equipment manufacturer components, such as a vortex tube based cold air gun attached at the back of an ablation and angioplasty barrel-assembly may include controls as well.

To the extent that angioplasty necessitates positional control, this is duplicated in the barrel-assembly and the airgun. Implantation demands greater precision than angioplasty, recommending controlled machine support. Except for repositioning of the muzzle-head over distances greater than 15 centimeters, once stenting has been initiated, the control of transluminal positioning is by means of a linear positioning table, and rotatory positioning by means of the turret-motor, as will be described. Whether used as the primary or secondary source of power, a battery-powered barrel-assembly not only imparts freedom of movement, but requires no emergency backup power source. The airgun, however, incorporates an uninterruptible power source. Manually side-sweeping the lumen walls transluminally, the operator can stop and use the turret-motor to remotely rotate the brushes in order to better access the sides of the lumen not well covered because the barrel-assembly could not be rotated manually.

The powered components within the barrel-assembly always include tractive electromagnets to recover stray miniballs, almost always a turret-motor, and in even a basic barrel-assembly intended for angioplasty, side-sweepers with trap-filter. With combined forms that include additional electrically operated components for transluminal use, such as a laser catheter or atherectomy burr, these too can be operated with the barrel-assembly removed from the airgun for manual use, in which application, the barrel-assembly constitutes a means for angioplasty to include atherectomy quite apart from stenting.

Accordingly, the turret-motor can be used to rotate in order to aim an eccentrically discharging muzzle-head, to rotate the tractive electromagnets to recover a mispositioned or stray miniball, or to rotate the side-sweepers with downstream trap-filter to assist in angioplasty with the barrel-assembly removed from the airgun barrel for manual use. One or both tractive electromagnets in a radial discharge muzzle-head can likewise serve a second function as temperature controlled heating elements for thermal angioplasty when sent heating current before insertion into the airgun, and then used to recover errant implants once the barrel-assembly has been engaged for stenting.

The bristle bundles are inserted into the brush handle with sufficient separation to allow blood to flow through, and deploying one side-sweeper can be used to press the muzzle-head against the lumen wall along any preferred angle, circumferentially when the side-sweepers are mounted normal to the longitudinal axis of the muzzle-head, and longitudinally when the side-sweepers are mounted radially. When the latter, the passage of blood past the device is significantly improved.

In a ductus ablation or angioplasty-capable barrel-assembly, the turret-motor has three separate and distinct modes of operation. The first is to rotate the barrel-assembly, which may be in manually controlled or automated use. So that its extracorporeal end can be freely moved, except when some followup thermal angioplasty is desired after the barrel-assembly has already been inserted into the airgun, angioplasty with the barrel-assembly is manual with the barrel-assembly separate from (electrically and mechanically independent from, not inserted into) the airgun. While in use to perform an angioplasty, only angioplasty-capable barrel-assemblies that require insertion into the airgun to positionally control the turret-motor are dependent thus on the airgun. The proximal end is thus unconnected and freely movable.

For untethered freedom of movement, while used to perform an angioplasty, an angioplasty-capable barrel-assembly is independent of an airgun for power. In more basic angioplasty barrel-assemblies, the on-board power cannot be used to control the turret-motor for positioning. Thus, with more basic embodiments, should use of the turret-motor become desired for use during the angioplasty, positional control of the turret-motor requires connection through insertion into the airgun. By contrast, a more capable embodiment is independent for positional control of the turret-motor, which likewise is powered by the on-board battery pack.

Transluminal precision of the kind essential to implant miniballs not required for an angioplasty, no means for control of the linear table is incorporated into an angioplasty-capable barrel-assembly of the linear table for transluminal movement) Once the barrel-assembly is inserted into the airgun to initiate stenting, the independent capability of the barrel-assembly is supplemented by indirect connection to the power supply within the airgun cabinet. That is, battery power is not concurrently disconnected leaving the barrel-assembly dependent for power.

The second mode of operation is as a temperature controlled heating element for thermal angioplasty whereby the turret-motor at the rear of the muzzle-head at stall (and/or the tractive electromagnets toward the front of the muzzle-head) are designed to accept a surge amperage that quickly raises winding temperatures from body temperature at 36.8±0.7 degrees centigrade by 53 degrees centigrade or 98.2±1.3 degrees Fahrenheit higher by 96 degrees Fahrenheit past the intervening thrombogenic range of temperatures to 90 degrees centigrade or 194 degrees Fahrenheit as having been determined optimal for thermal angioplasty (Post et al. 1996, Thrombosis and Haemostasis 75(3):515-519 cited below) or another temperature optimal for the endoluminal ablation of a specific kind of tissue. This temperature is considered not only optimal for thermal angioplasty, but sufficient to destoy the debris that passage of the muzzle-head could liberate, namely, lipid, macrophages, T cells, proteoglycans, smooth muscle cells, and collagen and calcified plaque particulates.

In an angioplasty-capable barrel-assembly, to control the turret-motor and recovery electromagnets as heating elements independently requires that three heat servocontrol microcircuits be provided in the hand-grip with angioplasty control panel at the proximal end. In some instances, momentarily heating the turret-motor or one of the recovery electromagnets can be use to assist in locating the muzzle-head. Thermal ablation in ducti other than vascular is not limited to 90 degrees centigrade, so that different temperature settings are provided from 50 to 100 degrees centigrade in ten increments of five degrees centigrade each.

When used upon initial passage through an artery known or suspected to harbor vulnerable plaque, the risk of rupture or the dislodgement of debris at points about the muzzle-head where it first makes contact with the surrounding lumen wall is minimized by effectively subjecting the wall to a preemptive thermal angioplasty. Such is not an angioplasty in the conventional sense, but rather a continuous pass over the lumen wall whereby the cold or heat is used to reduce the risk of rupture and embolism. For such an artery, only a barrel-assembly with heatable front end may be used and only with the windings of both recovery electromagnets heated. The nondiscretionary angioplasty with use of a minimally angioplasty-capable barrel-assembly, or a barrel-assembly in which only the coils of the reovery electromagnets at the front of the muzzle-head are heatable, is for the purpose of precluding ruptures by contact with the muzzle-head and should not precipitate intimal hyperplasia and restenosis.

Where the latter is a special concern, a preparatory cryogenic angioplasty, which is reputed to reduce restenosis, can be performed; however, this assumes that the heat pretreatment will induce stenosis in portions of the artery unaffected by the stenting to follow. This circumstance arises both when a minimally angioplasty-capable barrel-assembly is used for stenting without an antecedent discretionary angioplasty performed as a separate procedure and when a fully angioplasty-capable barrel-assembly is used to perform a proper angioplasty as a separate procedure without regard to whether the proximal end of the barrel-assembly will thereafter be inserted into an airgun for stenting.

In barrel-assemblies for either of these uses, a heat-window that fully encircles the recovery electromagnets and except for portions occupied by the distal embolic protective trap-filter and, if installed, a laser or atherectomy burr, extends over to cap or envelop the entire nose of the muzzle-head, is used as a leading heating element, the nose or nose-cap heat-window, to heat the surrounding wall of the lumen. Thus, while the winding of either electromagnet is separately heatable, when used upon initial introduction and transluminal passage through an artery known or suspected to harbor vulnerable plaque, the windings of both recovery electromagnets are used to generate heat.

Following initial contact and while continuing to heat before switching to tractive use during discharge throughout which both electromagnets must remain energized, either winding can be used independently to concentrate the heat directed toward an eccentric lesion. Since plaque is usually eccentric, and the turret-motor is at a distance from the points where the muzzle-head makes intial contact with the lumen wall, most turret-motors will have circumferentially delimited slits, slots, or an elongated rectanglar heat-window for directing the heat over a certain level and arc (direction).

Since temperatures above and below 90 degrees centigrade tend to be thrombogenic (Post et al. 1996, Thrombosis and Haemostasis 75(3):515-519 cited below, heat windows, whether enveloping, as at the nose (nose-cap, nose-dome), or circumferentially delimited, as at the turret-motor, must to the extent possible be temperature isolated from the surrounding surface of the muzzle body. This is accomplished by making the window of silver and thus creating a considerable differential in thermal conductivity between the heat-window and the regions bounding it. However, even when the areas adjacent to the heat-windows represent zones along a gradient of decreasing temperature, unless located beyond the level to which the focus of heat is moved, these adjacent areas are exposed to temperatures below 90 degrees centrigrade only momentarily before exposed to the full 90 degree temperature over the window.

Another time that thrombogenic temperatures will occur is when the recovery electromagnets at the front of the muzzle-head are switched from the heating to the tractive mode upon initiating discharge. At this time, the magnets, usually along with the trap-filter, are kept on (energized) instead to interdict and recover any miniballs that would otherwise pass downstream. The interval during the return from 90 degrees centrigrade back down to body temperature is minimized by prepositioning a cooling catheter in the central canal or a spare barrel-tube (service channel) that is used to conduct chilled air generated by vaporization from a small, typically 12 to 20 gram cartridge of liquified carbon (CO₂) or nitrous oxide (dinitrogen (mon)oxide), (NO₂) gas or high purity 1,1,1,2-tetrafluoroethane (R134a) cryogen spray.

By directing a stream of cold air at the back side of the muzzle-assembly nose-cap, the same apparatus can be used to perform a precautionary angioplasty. Whether to conduct heat produced by means of a vortex tube or cold produced by a vortex tube (cold air gun) or by a CO₂ or NO₂ cartridge connected at the back of the barrel-assembly, the use of a cooling catheter is necessary only in barrel-assemblies that would otherwise release gas into the bloodstream. This is avoided in an edge-discharge muzzle-head of which the central canal is used to house only a trap-filter silo with enclosed back at its distal end.

A gas vaporization cold generating apparatus is described by Sellinger, M. S. and Currie, R. B. 1971. “Cryogenic Biological Apparatus,” U.S. Pat. No. 3,630,203, incorporation into a barrel-assembly necessitating miniaturization of the parts that course through the barrel-assembly central canal. The use of such portable cartridges, which are commonly used in airguns to provide the propulsive force for propelling the projectiles (CO₂) and in aerosol cans to dispense whipped cream (NO₂) for example, allows the cryogenic gas to be carried on-board the angioplasty-capable barrel-assembly, which thus remains free of a hose that would be needed for connection to a separate supply tank.

While temperature inconstant over time and limited in charge and thus duration, such a free-standing flash expansion vaporization chilling means would rarely fail to meet the present needs. A discharged cartridge can be replaced with a charged one, but where constancy of temperature without interruption is desired, a vortex tube-based cold air gun, which necessitates connection to the end-plate at the back of the barrel-assembly by a small supply line or hose to a tank (cylinder, canister) of compressed air, is used (below).

Connection by a small hose notwithstanding, connection at the back (proximal end) of the barrel-assembly to a highly pliant hose leading to a supply of air is not as hindering as would be the need to manipulate an angioplasty-capable barrel-assembly that is relatively inflexible and inserted into the airgun. Both because it is not connected to function thus and is contained within the airgun cabinet necessitating connection to the barrel-assembly through a supply line, the cartridge used to power the airgun is not also used cryogenically. Another reason for prepositioning a cooling catheter in the muzzle-head is to cool the push type solenoid in the nose of the muzzle-head that is used to deploy a distal embolic protective trap-filter when optimal materials notwithstanding, the duty cycle with consequent buildup of heat would damage to the solenoid.

Ideally, heat sunk, and through use of the cooling catheter, the heat liberated by the solenoid is constrained to 90 degrees centigrade, any thermal thrombogenic effect thereby averted when used in the vascular tree. The ability to deliver cold air, typically at −10 degrees centigrade (14 degrees Fahrenheit), through a cooling catheter, allows the quick return to body temperature of the electromagnet and turret-motor coils when used for thermal angioplasty, and of the solenoid coil when energized to deploy the trap-filter. The cooling catheter is closed off at its distal terminus, and the central canal and barrel-tubes are perforated to allow gas contained within the barrel-assembly to be internally recirculated. Gas is thus prevented from entry into the bloodstream, and the cryogenic gas is quickly returned to body temperature.

The barrel-assemblies described herein are neither configured nor intended to be capable of cryogenic angioplasty through the use of a cryoplasty balloon. Such balloons accomplish angioplasty when expanded in apposition to the surrounding wall of the lumen when left in place for 20 seconds at −10 degrees centigrade. While such a balloon could be deployed from the otherwise unoccupied central canal of an edge-discharge muzzle-head as described below, a process that required advancement of the balloon in inflation and deflation increments would take too much time.

A cryogenic approach therefore directs nitrous oxide vaporized from a small cartridge of nitrous or carbon dioxide temporarily attached at the back (behind the end-plate) of the barrel-assembly while still detached from the airgun toward the internal or back side of an efficiently temperature conducting nose-cap window of the same kind as is used for a heat-window (below). The barrel-assembly can then be slowly and continuously advanced or withdrawn without the need to deploy and stow it incrementally, which process is tedious and takes too much time.

Thus, in a precautionary angioplasty whereby the muzzle-head is slowly moved over the wall of a lumen suspected or known to harbor vulnerable plaque rather than directed at specific lesions, the operator must arrive at a clinical judgment as to whether the somewhat greater speed of using heat and reduced susceptibility of extraluminal stenting to intimal hyperplasia outweighs the relative freedom from consequent intimal hyperplasia of using cold. The outlet temperature of a vortex tube-based cold air gun is typically 70 degrees Fahrenheit lower than the inlet temperature, so that supplied with compressed air at 75 degrees Fahrenheit, for example, a vortex tube-based cold air gun can deliver air at 5 degrees Fahrenheit.

The barrel-assembly effectively serves as a kind of guide (guiding) catheter that once introduced through the introducer sheath, allows the entry wound to be opened but once for both angioplasty and stenting. Unless a procedure will be completed in too little time for the inlet temperature to change, merely to prerefrigerate or preheat a canister of air or another suitable gas which is then allowed to gradually return to the ambient temperature in the operating room is unacceptable. Alternative means of providing gas of constant temperature are possible.

Placing the compressed gas cylinder within a refrigeration or heating mantle or enclosure can be used separately or with a vortex tube to achieve colder temperatures. However, the cylinder must be close to the barrel-assembly end-plate, since moving through a long supply line will similarly allow gradual change to room temperature. Thus, except occasionally for connection to a cold air gun, all angioplasty-capable barrel-assemblies, to include minimally thermal angioplasty (lumen wall priming-searing-) capable barrel-assemblies and fully angioplasty (lumen wall priming-searing-) capable barrel-assemblies, are used separately and independently of an airgun and inserted into the airgun only afterwards to initiate stent-implantation. Left connected throughout the angioplasty would seldom if ever impede the freedom of transluminal movement. Small in gauge and pliant, the cold air supply line can be left connected until the barrel-assembly must be inserted into the airgun to commence stent implantation.

Since a minimally angioplasty-capable barrel-assembly is used to preclude ruptures through a preliminary searing of the lumen wall, insertion into an airgun, requiring but the momentary disconnection of the supply line, will always follow. A fully angioplasty-capable barrel-assembly may not be followed by stenting that uses the barrel-assembly. A side-access entry portal for introducing a cooling catheter into the central canal is not contemplated. As barrel-assemblies are not limited to use in the arterial system, and these heating and chilling features can be used for thermal (but not cryogenic) ablation in other type ducti, barrel-assemblies other than minimally angioplasty-capable contain controls to allow the selection of temperatures (winding currents) other than 90 degrees centigrade, usually from 50 to 100 degrees centrigrade in ten increments of five degrees.

Since the vortex tube cold air gun emits hot air at the opposite outlet, it can be used as an alternate source of heat for thermal angioplasty as well. With the qualification that the volume of air delivered by the vortex ‘cold’ air gun diminishes in proportion to the extremity of the temperature demanded, the overall range of temperatures that such a gun can deliver is dependent upon the temperature of the air supplied from the compressed air cylinder (tank, canister) and to a much lesser extent, the ambient temperature, but generally ranges from 15 to 250 degrees Fahrenheit, which range encompasses the temperatures used for ablation and angioplasty whether cryogenic or thermal.

The degree of capability is also based upon whether each such heating element is independently controllable by a separate temperature servocontrol microcircuit in the hand-grip. A more basic embodiment is distinguished from one of greater capability in that the latter also incorporates a positional servocontrol microcircuit in the hand-grip, positional use of the turret-motor while disconnected from the airgun limited to the support of angioplasty. Use of the turret-motor both as a heating element and mover necessitates the coordinated control of winding temperature. Cooling back down to body temperature past the thrombogenic range is accelerated by means of passing a special cooling catheter or rapid cooling catheter down the barrel-assembly or spare adjacent barrel-tube (service channel) to the turret-motor and/or electromagnets, as will be described.

The third mode of operation is oscillatory, obtained either by detuning the turret-motor drive controller velocity loop, in which case the oscillation is random, or if the movement is to be controllable, then by programming the reciprocal motion according to sinusoidal profiles which can be selected, s-ramping used to obtain smooth performance if desired. While in most instances, one or two frequencies and arcuate strokes (excursion) to either side are satisfactory, some side-sweeper tool tips that function efficiently at a certain frequency or stoke may require more setting.

Oscillatory movement is used to free the muzzle-head following delivery of a lubricant through a service channel or to vibrate the side-sweeping brushes, but never in an attempt to pass a tortuous stretch of a vessel, where such action can result in stretching injury or even perforation. Fever tends to reduce thrombogenicity and may be disregarded for the present purpose (see, for example, Groza, P., Artino-R\check{a}dulescu, M., Nicolescu, E., Munteanu, A., and Lungu, D. 1987. “Blood Coagulation and Fibrinolysis in Hyperthermic Rats,” Physiologic 24(4):213-220).

The use of silver wire achieves the maximum electrical and thermal conductivity in both the turret-motor and tractive electromagnets, which in barrel-assemblies designed for thermal as well as mechanical angioplasty, also serve as temperature-controlled heating elements. High electrical conductivity equating to low resistance with less heat generated for a given level of current, silver wire windings require proportionally more current to be raised to 90 degrees Celsius; however, in no instance should an electrically separate outer coil of nichrome wire be needed as the heating element. Consistent with the use of insulation in electrical motors and magnets, the insulation must be effective electrically but thermally conductive.

The subminiature dimensions of the turret-motor necessitate that to achieve a reasonable service life, the winding varnish be made as thick as space will allow (see, for example, Grise, W. R. and Zargari, A. 1997. “Delamination and Cracking Failures in High-voltage Stator Winding Coatings,” Electrical Insulation Conference and Electrical Manufacturing and Coil Winding Technology Conference, 1997, Proceedings, 22-25 Sep. 1997, pages 835-839); however, for ablation or angioplasty, the electrical insulation should minimally interfere with thermal conductivity (see, for example, Speer, D. R., Jr. 1997. “Thermal Conductivity Improvements for Electric Motors,” Electrical Insulation Conference and Electrical Manufacturing and Coil Winding Technology Conference, 1997, Proceedings, 22-25 Sep. 1997, pages 723-725). Finally, lesion removal and stenting in a single operation is significantly augmented with the incorporation into the barrel-assembly of conventionally independent means for the removal of highly calcified plaque, to include a rotational atherectomy burr or laser catheter.

Provided the parameters appurtenant of the action are closely determined and controlled, the miniballs bioinert, and sterility achieved, the implantation by ballistic means of ferromagnetic miniballs just inside the tunica adventitia, subadventitially (perimedially), or medially is safe (for a histological description of the tracheal adventitia, see Ohkimoto K, Mouri M, Amatsu M, and Teraoka M. 1997, “Histological Study of the Tracheal Adventitia, Perichondrium and Annular Ligament (in Japanese), Nippon Jibiinkoka Gakkai Kaiho 100(11):1394-1400.

Abrupt closure, whether during angioplasty, atherectomy, or following the stenting of an artery, usually results from thrombogenesis. This can arise as the result of dissection due to balloon overinflation during angioplasty or with directional atherectomy, where injury due to the bulkiness of the device has been hypothesized to result in an increased rate of distal embolization (Abdelmeguid, A. E., Whitlow, P. L., Sapp, S. K., Ellis, S. G., and Topol, E. J. 1995. “Long-term Outcome of Transient, Uncomplicated, In-Laboratory Coronary Artery Closure Circulation 91(11):2733-2741, whose attribution to spasm as weakly predictive of acute sequelae compared to elevation in serum muscle enzyme levels is at odds with the findings of Piana, R. N., Ahmed, W. H., Chaitman, B., Ganz, P., Kinlay, S., Strony, J., Adelman, B., and Bittl, J. A. 1999. “Effect of Transient Abrupt Vessel Closure During Otherwise Successful Angioplasty for Unstable Angina on Clinical Outcome at Six Months,” Journal of the American College of Cardiology 33(1):79-81.) in both instances especially when insufficient glycoprotein IIb/IIIa antagonist (inhibitor) has been administered to deter platelet-rich thrombi. Since the object of the procedure is to stent the vessel, it is only abrupt closure at levels not to be stented that poses a risk.

Here, however the tiny circumscribed punctures through the intima and the trajectories through the media are quite unlike dissection in extent or form, the trajectories of the implants is external to the lumen, the barrel-assembly while larger in diameter than an uninflated balloon is fully rounded and smooth surfaced as not to gouge, nor is it so large as to seize onto or stretch the lumen wall. Abrupt closure should not result for conventional reasons. Absent dissection, balloon (compressive, atheroma-crushing) angioplasty still injures the endothelium, and “endothelial dysfunction can promote both restenosis and coronary spasm” (Chandrasekar, B., Nattel, S., and Tanguay, J. F. 2001. “Coronary Artery Endothelial Protection After Local Delivery of 17 Beta-Estradiol During Balloon Angioplasty in a Porcine Model: A Potential New Pharmacologic Approach to Improve Endothelial Function,” Journal of the American College of Cardiology 38(5): 1570-1576).

Negligible endothelial injury due to balloon (compressive, atheroma-crushing) angioplasty may induce vasospasm even in an untreated artery (Lauribe, P., Benchimol, D., Duclos, F., Benchimol, A., Bonnet, J., Levy, S., and Bricaud, H. 1993. “Spasme occlusif d'une artère coronaire non abordée au cours d'une angioplastie. A propos d'une observation” (“Occlusive Spasm of a Coronary Artery Not Treated During Angioplasty. Apropos of a Case”), Annales de cardiologie et d'angéiologie 42(2):89-92) by imparting hyper-reactivity to acetylcholine (Nishijima, H. Meno, H., Higashi, H., Yamada, K., Hamanaka, N., and Takeshita, A. 1996. “Coronary Vasomotor Response to Acetylcholine Late After Angioplasty,” Japanese Circulation Journal 60(10):789-796; Osborn, L. A. and Reynolds, B. 1998. “Vagally Mediated Multivessel Coronary Artery Spasm During Coronary Angiography,” Catheterization and Cardiovascular Diagnosis 44(4):423-426), perhaps by relation to the superoxide radical (see for example, Laurindo, F. R., da Luz, P. L., Uint, L., Rocha, T. F., Jaeger, R. G., and Lopes, E. A. 1991. “Evidence for Superoxide Radical-dependent Coronary Vasospasm After Angioplasty in Intact Dogs,” Circulation 83(5):1705-1715; Ferrer, M., Tejera, N. Marin, J. and Balfagon, G. 1999. “Androgen Deprivation Facilitates Acetylcholine-induced Relaxation by Superoxide Anion Generation,” Clinical Science 97(6): 625-631; Rubanyi, G. M. and Vanhoutte, P. M. 1986. “Superoxide Anions and Hyperoxia Inactivate Endothelium-derived Relaxing Factor,” American Journal of Physiology 250(5 Part 2):H822-H827). This is the likely explanation for the occurrence of vasospasmodic response with angioplasty even in another artery, much less when dissection has not occurred (see Fischell, T. A. 1990. “Coronary Artery Spasm After Percutaneous Transluminal Angioplasty: Pathophysiology and Clinical Consequences,” Catheterization and Cardiovascular Diagnosis 19(1): 1-3).

Conventional causes for abrupt closure aside, the appearance of vasospasm as a reflex response to the shock of sudden unnatural penetration should be deterrable through the preprocedural inception of arterial antispasmodic drugs, such as nitrovasodilators (glyceryl trinitrate, nitroglycerin), verapamil (Isoptin®, Verelan®, Calan®, Bosoptin®), diltiazem calcium antagonist, intracoronary infusion of isosorbide dinitrate, or a dilute intravenous solution of papaverine and nicardipine, as well as platelet glycoprotein IIb/IIIa antagonist.

That abrupt closure cannot simply be attributed to reflex response as might be induced by the sudden impact of a projectile is additionally suggested by evidence that heparin anticoagulation as measured by the activated clotting time appears to reduce the risk of abrupt closure during angioplasty in proportion to the dosage without increasing the risk of major bleeding complications (Narins, C. R., Hillegass, W. B., Jr, Nelson, C. L., Tcheng, J. E., Harrington, R. A., Phillips, H. R., Stack, R. S., and Califf, R. M. 1996. “Relation Between Activated Clotting Time During Angioplasty and Abrupt Closure,” Circulation 93:667-671). In addition to conventional delivery paths, the miniballs can be coated with thromobolytic, anti-inflammatory, or intimal hyperplasia-attenuating medication as hereinafter described. The risk of mortality and complications, to include cerebral hemorrhage, has been determined to be reduced the earlier coronary reperfusion is initiated (Cannon, C. P. 2001. “Importance of TIMI-3 Flow,” Circulation 104(6):624-626).

Should such a response occur, miniballs with an outer coating to deliver these drugs in situ are used, with any collateral intravenous or oral dosage restricted to subhypotensive levels. Counterintuitively, implantation by such means is minimally traumatizing as instantaneous, clean, bloodless, and limited to the tissue within and immediately surrounding the trajectory, secondary swelling is moderate and medically manageable.

To minimize the risk of rebounding from, penetration, or puncture of the fibrous outer layer or sheath of the vessel or duct while taking advantage of the elasticity of the lumen wall, miniball discharge is at a 45 degree or less, i.e., acute, angle. Compared to a trajectory perpendicular to the lumen surface, penetration at an acute angle aids implantation by undercutting the media with less risk of perforating the tunica adventitia. The longer trajectory extends the resultant trauma and inflammation; however, the reobstruction if not abrupt closure that appears to vary with the extent of vessel wall damage by balloon over-inflation and dissection is quite different in form and much more extensive than is the suddenly introduced and highly circumscribed penetration of the intima and media by the miniature balls.

Success is optimized by efficient technique that minimizes operating and thus anesthetization time, and the apparatus for implanting the miniballs is devised to accomplish the action required in the least amount of time in patients for whom the angioplasty has recovered adequate blood flow. The time of a procedure is largely determined by the need to position the muzzle-head and place the miniball implants. Superior steerability, rotatability, ability to place multiple implant miniballs per discharge, and clear viewability of the angular displacement of the muzzle-head allow efficient positioning. Hence the incorporation into the muzzle-head of a turret-motor.

Whenever preceded by a conventional (balloon) angioplasty, access, whether percutaneous or open, is preferably at the same groin (inguinal, femoral) entry wound as was used for the angioplasty, with the administration of heparin having been stopped. This is because 1. Proximal, meaning brachial or ‘axillary’ (high brachial), much less radial access poses a greater risk of complications, 2. Groin (femoral artery or vein) compression closure aids such as the FemoStop®Plus, Angio-Seal®STS Plus and Millenium platforms, and Perclose®A-T have become available to deal with oozing or hematoma, 3. Stenting almost always follows balloon angioplasty or rotational (rotablation) or directional atherectomy, and so can aggravate the entry wound, 4. The muzzle-head at the end of the barrel-assembly is generally 8-10 French, recommending an entry wound of larger size, 5. A point of entry other than inguinal increases the possibilities for puncture site complications and postoperative morbidity. When the angioplasty is accomplished using an angioplasty-capable barrel-assembly, removal of one catheter and insertion of another is unnecessary.

The nonmagnetic muzzle-head is preferably made of a nonmagnetic austenitic stainless steel, such as 18-8, 304, or 316 amenable of hardening in smaller thicknesses. The core of the tractive electromagnets at the distal end of the muzzle-head and components of the turret-motor if present, however, allows the use of an external hand-held electromagnet to expedite steering, and in a larger vessel, make possible the quick positioning of the round tip in stable abutting relation to at particular position along the lumen wall. When the brushes are separately controllable, the side-sweeping feature described below can also be used to nudge the muzzle-head eccentrically within a lumen, but only in a lumen little greater in diameter than the muzzle-head itself, and with the barrel-assembly stationary. Limited thus, the potential utility of an external hand-held electromagnet stands.

The muzzle-head could also be made of any nonferrous metal of suitable hardness and tissue compatibility or a resin by transfer-molding. The amperage to the electromagnet is controlled by a precision multiturn digital potentiometer that allows the muzzle-head to be nudged against the lumen wall without such force as could injure the wall or so compress the tunica media as to preclude undercutting it for subadvential placement. Alternatively, the muzzle-head spindle can be made of polytetrafluoroethylene by transfer-molding.

While balloon angioplasty alone can return the lumen to substantial concentricity, the sites of harder lesions experience greater radial force, sometimes resulting in dissections leading to increased arterial shrinkage, or constrictive remodelling, and intimal hyperplasia. A preliminary reduction of prominences by means of cutter balloon or rotational, directional, thermal, cryogenic, ultrasonic, or laser-catheter atherectomy reduces the risk of this eventuality, and this has encouraged the development of catheter-based devices that cut and then reduce rather than merely smash plaque whether a stent is used to hold it in place. Especially when equipped with side-sweepers or as combination-forms that incorporate a laser or cutting tool, ablation and angioplasty-capable barrel-assemblies can be used to perform an angioplasty or atherectomy independently of an airgun.

Using either an edge- or center-discharge muzzle-head, these differ from the use of balloons in ways that as an alternative technology and methodology, offer advantages and disadvantages as compared to the use of balloons. Not inflated and not over-inflatable, provided it is properly sized, the muzzle-head is less prone to cause dissections and does not require to be withdrawn through the introducer sheath for reentry to introduce a stent. Instead, stenting is initiated without a second radial inflation against the lumen wall in order to expand an endoluminal stent with sufficient force to prevent migration. Instead, to initiate stenting, the free end of the barrel-assembly is plugged into the airgun. By the same token, the muzzle-head is not deflatable, which capability of a balloon reduces the risk of tears and ischemia.

That deflatability enhances passage through stenotic or tortuous stretches is indissociable from the need for a guide wire and the risks associated with the use of a guide wire. A further integration of function as would allow one device that not only cuts and compresses but also stents would further reduce operative time and the need to repeatedly withdraw and reenter, risking entry wound complications. Catheter-based devices currently in use do not lend themselves to such a combination of functions and less still to an ability to place multiple stents. Apart from difficulties in integrating the devices mechanically, some are not reusable. A distinguishing attribute of the apparatus to be described is the possibility of combining these various functions. Unlike endoluminal stents, extraluminal stents allow intervention that is discretionary with respect to lesion eccentricity, which is usual.

Eccentric and pathologically distinct lesions within the same vessel or duct can also be dealt with through the use of differently medicated or irradiative miniball implants. Eccentric and pharamacologically differential treatment is best accomplished without the need to withdraw one barrel-assembly and replace it with another having a differently configured muzzle-head. Not only does this reduce procedural time, but the duration of transient ischemia due to blockage of the vessel by the apparatus and of injury to the inguinal or brachial point of entry is significantly reduced. Eccentric lesions other than atheromatous, such as result from congenital variants, dissections, and infection can appear in vessels that nonmuscular and nonintracranial, afford sufficient perivascular space to surround with a stent-jacket. (Russo, C. P. and Smoker, W. R. 1996. “Nonatheromatous Carotid Artery Disease,” Neuroimaging clinics of North America 6(4):811-830)

In a muscular artery, for example, the layers of the vessel wall, to include the smooth muscle of the tunica media, the internal elastic lamina inside the tunica media, and the external elastic lamina outside the tunica media, cooperate with the tunica adventitia or outer fibrous layer of the vessel to provide a range of subpuncture impact force values in response to a miniball forcibly projected against the inner surface of the lumen wall. By cushioning and dissipating the force of impact by outward displacement, the layers make it possible to shoot a miniball at an acute angle at the lumen wall so that it comes to rest just beneath the tunica adventitia.

Even allowing for variation among conspecific individuals due to genetics, size, age, and behavioral distinctions, specific disease-free tissues and tissue laminates, such as that of a certain artery, adhere to a substantially consistent and characteristic range of values in mechanical properties, to include resistance to puncture. Among vertebrate classes, type tissues can exhibit less consistency in mechanical properties. When the outer layer of a ductus lacks sufficient strength to sustain the negligible tractive force used, a ferromagnetic wrap-surround is engaged by means of prongs or clasps in the media to provide a prosthetic adventitia.

However, by definition, only tissue that is diseased would ever recommend the application of an exogenous source of retractive tension to restore it to functional conformation, and diseased tissue is not consistent in mechanical properties as is healthy tissue. The alteration undergone by various tissues in disease covers the full spectrum of pathological alteration from the progressive hardening of vascular plaque sclerotization in other tissues by calcification or ossification.

Numerous conditions prompt hyperplasia, and previous radiotherapy or chemotherapy can induce hardening. Fluid infiltration, hypervascularization, atrophy, and pyogenic arteritis can produce softening. Continuous variability characterizing diseased tissue, the need for nonpresumptive testing that evaluates the puncture resistance of the actual lesion in the specific patient without presumption is evident. Neither is diseased tissue uniform in its distribution; an improved stent should be able to deal with eccentricities discriminately.

To test diseased tissue of the same kind and state of progress in subjects immediately upon death as to precede autolysis affords an indication as to the force of impact needed. While such results significantly reduce the uncertainty and serve as the basis for initial control settings, individual variation prevents uncertainty from being eliminated. Tissue adjacent to a lesion will not exhibit the same properties as that affected. Furthermore, while the miniballs are bioinert, the presence of surrounding vessels, nerves, or other structures will recommend against a preliminary test discharge that could result in a puncture and injury to such a neighboring structure. Accordingly, a method an apparatus for testing the tissue to be implanted is necessary.

While the subadventitial implantation of smooth-edged or blunted iron filings would best distribute the tension on tissue by the pulling force of a magnet, iron in the body is readily oxidized, dispersed, and absorbed. The fibrous jacket of vessels and ducts are not inert, but like all connective tissue, to include tooth enamel, constantly replaced. Consequently, a blunt object placed subadventitally would not, with the pulling forces used, be likely to pull entirely through the tunica adventitia. To this end, neodymium magnets are used not to obtain forceful fields, but rather to minimize the size of the magnets necessary to produce the usually mild pulling force required.

The miniballs are biochemically inert, and may be treated with anti-inflammatory agents, antibiotics, a coagulant, other medication, or irradiative. For vascular applications, a radial discharge muzzle-head that includes from one to four barrels, but usually either two or four, referred to as two-way or four-way, is connected to the end of the barrel-catheter containing the miniball airgun barrel-tubes to form a unitized whole or barrel-assembly. As explained below, a barrel-assembly to be used for placing implants at intervals that are too fine to be controlled by hand is automatically controlled.

Whether intended for exclusively manual or for manual and automatic use, a barrel-assembly usually incorporates a forward drive stabilizer as described below about the barrel-catheter. One to three-way barrel-assemblies with barrel exit ports at different angles are used with vessels or other tubular structures that intimately attached to their substrate, resist dissection round and about without open surgery as would allow the vessel or duct to be completely surrounded by a full rather than a partial stent-jacket, whereas the four-way apparatus is used with structures readily freed from the substrate, allowing a full stent jacket to completely surround or jacket the structure.

Vascular treatment by the method to be described warrants special caution for patients presenting a susceptibility to spontaneous rupture of the internal elastic lamina or when the antecedent angioplasty has resulted in baloon overstretch injury. By situating sterile implants subadventitially and using magnetic force to draw these away from the more vital superjacent tissue, the tendency toward fistulization is minimized. By contrast, an intravascular stent, for example, sits upon the intima and compresses the subjacent tissue. Intraluminal stents in adjacent relation place structures in rubbing relation, whereas extraluminal stent-jackets prevent rubbing.

An adapted semiautomatic repeat action gas-operated airgun and single or multiple barrel barrel-assembly affects miniball implantation under a tightly controllable impact force too suddenly for the target tissue to displace or deform, and the diameter of the trajectory through the tissue is that of the miniball; stent implantation by such means should affect only the tissue in and immediately surrounding the trajectory, which immediately closes in to contain the miniball implant and commences the healing process. For use in the bloodstream, resistance to the advancement of the miniballs by the air trapped in the barrels demands pressure relief to prevent air or propulsive gas, normally CO₂, from entering the bloodstream during discharge in conflict with the need minimize or prevent the inflow of blood through the muzzle exit ports during the intervals between discharges.

In vascular applications, extraluminal stenting replaces endoluminal stenting following angioplasty or atherectomy whether thermal, cryogenic, or mechanical, often followed by angioplasty. The methods described herein thus see the perioperative management that has been undertaken with the primary angioplasty. This includes the means for managing the sequelae normally associated with cather-based procedures, such as the administration of a calcium antagonist to reduce risk of coronary artery spasm. Accordingly, medical management as such is not a part of the present invention.

Using the apparatus to be described in vasa not open to the exterior, clinical judgment must determine the relative value of stenting that eludes the foregoing complications but which will disallow the use of magnetic resonance imaging, possibly interfere with older technology implanted electronic devices such as an implantable pacemaker or a cardioverter defibrillator, necessitate exposure through a small incision and the need to dissect a vessel from its substrate or supporting structure in order to allow its partial or complete encirclement by the stent-jacket or extravascular component of an extraluminal stent.

The diameter of the apparatus that can be used in a vessel or other ductus is limited by the diameter of the lumen, which is simply given by nature. As a generalization, the internal diameter of the coronary arteries in adults, for example, varies between two and three millimeters, imposing a demand for severe miniaturization. This factor makes application for pediatric use especially difficult, barrel-assemblies suited to such use generally limited to a single barrel-tube.

Not requiring transluminal access to implant miniballs from within the lumen and applicable to a ductus of insufficient wall strength to allow ballistic implantation, a clasp-wrap or alternative means for introducing ferromagnetic implants in the wall of a ductus as will be described, can be applied to a duct or vessel that is smaller in diameter and can be fully surrounded. Other means for ‘grabbing hold of’ vessels walls that lack sufficient strength to retain small spherical implants will be described.

Access to coronary vessels is through a minimal incision, such as is used in minimally invasive direct coronary artery bypass (MIDCAB; limited access coronary artery bypass; keyhole incision heart surgery) to expose the left anterior descending artery and its diagonal branches on the front of the heart. The same totally endoscopic and robotic technology that make it possible to access a ductus without the need for an extended incision also allows a stent-jacket, magnet-surround, or miniball-surround to be placed. Robotically assisted coronary artery bypass (RACAB), for example, allows access to the coronary arteries without the need for a sternotomy that significantly increases trauma and healing time.

The size and mass of magnet-wraps and stent-jackets are minimized through the use of high energy product neodymium iron boron (Nd₂Fe₁₄B; NIB; NdFeB, “neo”) magnets. Sintered neodymium iron boron magnets are commonly available with an outer plating of nickel, which is often gold plated. In ducti open to the exterior, no concentrically jacketing component is used to surround the duct, entirely eliminating the need for percutaneous access. For ducti such as vessels not open to the exterior, the stent-jacket is inserted through a minimal incision with minimal dissection.

Unlike balloons, solid catheter-based devices such as a rotary burr, laser, or barrel-assembly cannot simply be deflated to allow resumption in the flow of blood past the device. This factor imposes a severe demand for miniaturization in the diametrical extension of the parts within the barrel-assembly, hence, the number and caliber of barrels. The smaller the implants, the greater must be the distribution density to achieve a uniformity of pulling force that reduces to an acceptable level the risk of implants being gradually pulled through the adventitia.

Balloon-based deflatable or otherwise collapsible and re-extendible muzzle-heads and muzzle-heads having collapsible and re-extendible chambers would make possible the use of larger caliber implants, but would introduce much additional structural, materials, and bonding complexity where the embodiment would have to be fully dependable. More significantly, a collapsible embodiment would unavoidably and unacceptably compromise the distal electromagnet assembly essential to trap loose and extract improperly positioned miniballs.

For this reason, a deflatable or mechanical linkage-based collapsible muzzle-head is discounted, flow past the muzzle-head being achieved by keeping the muzzle-head diameter to a minimum not simply for this part of the barrel-assembly as a whole, but at each longitudinal level along its length and by providing pathways in the form of external blood grooves and through and through tunnels that allow blood and contrast dye essential to confirm the reinstatement of patency to pass.

Apparatus and methods, summarization of requirements:

1. In the coronary arteries, minimizing the risk of a midoperative crisis due to obstruction of the lumen, whether by the apparatus or as the result of abrupt closure.
2. Especially in the arterial tree:

a. Minimizing the risk of ischemia due to obstruction of the lumen.

b. Minimizing thrombogenicity and the risk of remote embolization.

c. Means, to include adjuvant, for reducing the risk of and for responding to abrupt closure or the inducement of arterial spasm as may result from ballistic implantation of the arterial wall.

d. Adjuvant means for reducing inflammation due to ballistic implantation as posing a risk of occlusion.

3. In the vascular system more generally:

a. Means for equalizing differences in internal pressure within the barrel-assembly during discharge to prevent gas from entering the bloodstream.

b. Design to minimize to insignificant the ingression of blood through the muzzle-ports and into airgun discharge pressure relief channels as not to significantly affect the performance, much less foul the mechanism, of the barrel-assembly when introduced into and positioned within the vasculature.

c. Trackability of the barrel-assembly through a vessel that is the same or slightly smaller or larger in diameter than the muzzle-head.

d. Means for preventing the entry of a spherule into the bloodstream.

e. Optimization in the reach of implantation access by minimizing the forward extension of the muzzle-head, which may include at its front and center any of various means for the removal of diseased tissue, to include a rotational atherectomy cutter that must be recessed within the nose during transluminal movement or a laser photo-ablater.

f. A turret-motor stator and tractive electromagnet coils that function secondarily as heating elements for thermal angioplasty.

g. Providing means so that by attachment of a vortex tube, the muzzle-head can be used for either thermal or cryogenic angioplasty.

h. Except for attachment of a vortex tube or where an ablative or atherectomy cable is incorporated, complete independence of the barrel-assembly from the airgun until the proximal or extracorporeal end of the barrel-assembly must be inserted in the airgun to initiate stenting implantation.

i. Means for medially or permedially stent implanting a vas for retraction by a circumvascular stent-jacket that are introduced other than ballistically.

j. Automated means for closely spacing apart ferromagnetic spherule implants (miniature balls, miniballs) to provide a more even distribution of the magnetic tractive force.

k. Muzzle-head lubricity and torque as to preclude endothelial cling.

4. In the trachea:

a. A muzzle-head without the torpedo-shaped containing envelope that is needed for transluminal advancement within a smaller and structurally undifferentiated ductus which allows implants to be aimed into the structurally differentiated wall of the lumen.

b. Means for retrieving a mispositioned or dropped spherule.

4. In any ductus:

a. The ability to ablate matter obstructive of the lumen either by mechanical means or thermally or cryogenically by attaching a vortex tube at the rear (necessitating connection to a tank of compressed air), or thermally by sending current to the inmate windings of the electromechanical actuators within the muzzle-head that are primarily used for positional control and implant recovery respectively, or cryogenically by attaching a nitrous oxide or carbon dioxide cartridge to the back (proximal, extracorporeal end) of the barrel-assembly. An angioplasty-capable barrel-assembly is distinguished from such function only in the temperature applied.

b. The administration of adjuvant anti-inflammatory, antibiotic, and antithrombotic medication in moderation understood, the ability to commence stenting implantation immediately upon completion of ablation or angioplasty by insertion of the free (proximal) end of the barrel-assembly in the airgun.

c. Means for recovering by extraction a spherule that was misplaced upon implantation.

d. Means for preventing a loose spherule from passing down the ductus.

e. Means for checking the insertion of a mispositioning stay.

5. Interventional airguns must:

a. Discharge by quick semiautomatic operation, that is, in rapid succession without the need for frequent reloading.

b. Except for use in the airway, be capable of delivering multiple implants per discharge.

c. If based upon airguns that are commercially marketed, be capable of modification for interventional use to the same standard of safety as pertain to dedicated or special-purpose interventional airguns.

6. The materials used must be:

a. Bioinert if implanted.

b. Approved for use in the body.

c. Disposible or sterilizable.

7. Means for testing the penetration and puncture resistance of tissue to be implanted whereby all computation and calibration precede use of the apparatus, so that midprocedural testing may proceed by quick and direct observation, or empirically.
8. Means for testing lumen-muzzle head adhesion and for delivering lubricant to the muzzle-head midprocedurally as necessary.
9. In any bodily system:

a. Extraluminal stents must afford flexibility so as not to resist smooth muscle adjustment in vessel or duct caliber, whether pulsatile, tonic, or peristaltic.

b. Extraluminal stenting must minimize the risk of pull-through, or the gradual penetration and eventual perforation of the tunica adventitia by the implants under sustained magnetic traction.

c. There must be a reliable means for recovering loose miniballs.

d. There must be a reliable means for extracting misplaced miniballs.

10. Some mechanism to compensate for the inability of the operator to distance the implants along the length of a vessel or duct must be provided to accomplish this action automatically, and
11. That mechanism must be variable to allow the incremental distance and time between successive shot groups to be varied.
12. Increasing the intrinsic self-adjustment of the stent-jacket to assume the caliber of the treated vessel or duct.
13. Structuring the stent-stay insertion tool so that insertion is minimally interfering to imaging equipment necessary to confirm substantial concentricity of insertion.
14. Means for retracting stent-stays that have are mispositioning or entering the ductus other than substantially concentric thereto.
Other matters, such as the integration into the barrel-assembly of an excimer laser or rotatory atherectomy burr, are not of a fundamental nature but do go to the scope of practical utility of the apparatus to be described.

In the airway embodiment, a simple pipe or single barrel (monobarrel) barrel-assembly is used to implant miniballs adjacent to collapsed cartilage rings along the dorsal tracheal membrane, or trachealis muscle. Collapse of the tracheal ceiling is most often presented by toy breed dogs at mid-life. By contrast, the tracheomalacia encountered in human neonates is almost always due to immature development with consequent lack of resilience in the cartilage rings by the time of birth, which spontaneously resolves itself.

In the dog, however, tracheal collapse results when cartilage maintenance expressed as resilience begins to fail in middle age only to grow progressively worse. Accordingly, in man, pending spontaneous correction, the threat of suffocation in a severe case may warrant the temporary placement of an intraluminal stent. Except where tracheobronchial constriction or collapse is permanent if not progressive, the procedure to be described for use in the airway is intended for veterinary application.

Because the advancement of collapse is due primarily to increased ring infirmity and secondarily to stretching of the dorsal membrane from respiration that is made possible by and thus increases in proportion to the primary degradation in cartilage resilience, and because early intervention truncates stretching, an initial response should seek not to respond to the extreme collapse to which the condition would invariably progress were there no intervention, but rather to the condition as it exists.

Numerous conditions can result in constriction of the airway in man, and some, to include tracheomegaly (tracheaectasy), tracheobronchomegaly (tracheabronchoectasy, as in Mounier-Kuhn syndrome), which may be associated with pulmonary tuberculosis and diabetes, may produce tracheobronchomalacia and tracheal collapse even in the adult (Sudou, A., Hashimoto, T., Nakamura, H., Sarashina, G., Shimizudani, N., Yagyuu, H., and Matsuoka, T. 2003. [in Japanese] “Specific Notch in Early Expiration Region of a Flow-volume Curve in a Case of Mounier-Kuhn Syndrome,” Nihon Kokyuki Gakkai Zasshi 41(5):361-364).

While no stent can go to underlying etiology but only remedy the occlusion whether due to collapse of a vessel or duct mechanically, supporting the tracheheobroncial ceiling will interdict continued stretching of the collapsing dorsal membrane that the tidal flow of respiration, or constant push-pull action of breathing, would otherwise constantly aggravate. This action considerably increases collapse, which is primarily caused by a progressive loss of resilience in the cartilage rings due to a genetic defect expressed as an inadequacy of cartilage synthesis. Unless stopped, tracheal collapse eventually leads to inflammation and infection. For this reason, to persist in purely medical palliation with no mechanical intervention while the patient becomes more debilitated, and moreover, to then perform a radical procedure that may even include a thoracotomy represents poor management.

Annoyance from extrinsic forces on the trachea and the probability of stimulating the cough reflex must be weighed against the understanding that once advanced, frequent suffocation leading to a loss of consciousness and even death is prevented and that the same medication that is used when a mechanical intervention has not been performed should allow some remediation. A definite error in the treatment of tracheal collapse is the detention in mechanical intervention and continued dependency upon medication that lacks the efficacy to terminate further progress of the condition. Surgery tends to be detained until the condition is advanced and as a result, the patient much impaired, and intervention with existing tracheal stents is properly detained because the stent itself creates complications by interfering with normal physiology at the lumen surface.

In a dog with Grade I or II tracheal collapse, the sagging dorsal membrane is suspended by means of small neodymium permanent magnets positioned subcutaneously on the outer investing layer of the deep or muscle fascia overlying the implanted miniballs or suprapleurally, i.e., upon the serous membrane overlying the lungs. At incipient grades of collapse, significant closure of the airway as the result of a diagonal folding flat of the trachea when the head is raised as revealed by fluoroscopic observation is unlikely, allowing an initial intervention that is minimal.

If having already progressed to Grade III or IV collapse, the slack is pulled away laterally between ferromagnetic miniball implants in the ceiling of the trachea and magnets secured beneath the esophagus by means of a magnet-wrap as shown in FIGS. 10 and 11. The very malacia that demands correction renders the tracheal ceiling sufficiently compliant to move with the peristalsis of the esophagus during deglutition (swallowing) and without interference to the mucociliary function of the trachea. Since tautening the dorsal membrane alleviates the constant stretching action of breathing, it should seldom be necessary to retrieve the subcutaneous or suprapleural clasp magnets by means of an electromagnet and institute the second option.

However, such a conversion from the first option, directed to less progressed or lower grade collapse and the second to more progressed or higher grade collapse may be accomplished at a later date. Both procedures, placing fascial magnets and esophageal tacking, avert the extremely traumatic but nevertheless recommended procedures taught in every textbook of veterinary surgery at a time when the patient has become impaired by secondary sequelae, commonly ventricular and atrial enlargement and increased density of lung tissue, and is least likely to survive open surgery that may sever a thyroid artery or recurrent laryngeal nerve.

Expansion in the area treated at a later date is then made subject to actual eventualities, allowing trauma and risk to be minimized. Since an initial procedure can always be expanded upon at a later date when progress in the condition need not be presupposed, and an interval for recuperation is gained, the concept of extension for prevention is set aside. Furthermore, following any intervention, an interval should be allowed for the patient to learn to adapt to the new condition.

If certain postures, such as raising the head past a certain angle, initiate the characteristic ‘goose-honk’ cough, then the patient is likely to associate this posture and coughing, and learn to avoid the posture. Unless movement is restricted unacceptably, or coughing on drinking or eating do not subside over time, reintervention is deferred. Rather than to actually stent the structure it surrounds, a surrounding jacket can mount magnets to exert patenting traction upon miniballs implanted in an adjacent structure, notably by the esophagus upon miniballs implanted along dorsolateral longitudinal lines running along the ceiling of the collapsed trachea, for example.

When a structure is not merely to support magnets for exerting force upon a neighboring structure, but is itself to be stented, except that the endogenous outer layer of the structure to be stented lacks sufficient elasticity and strength to withstand puncture or retention of the miniballs, as may be true, for example, of diseased ducti and the normal esophagus, then reinforcement with an artificial or prosthetic ‘adventitia’ of the required properties is necessary.

Such a clasp-wrap or alternative means for introducing ferromagnetic implants in the wall of a ductus may be acted upon by either a more local stent-jacket or a magnet-wrap supported by a neighboring structure. However, a wall diseased as to retain little shear or tensile strength will present no substance to ‘grab hold of’ and will simply delaminate and collapse beneath the artificial adventitia. A structure so lacking in strength should be replaced with a graft or prosthesis.

Placed outside the tubular structure, such a wrap-surround must be tissue compatible but requires no immunosuppressive drugs as would pertain to a homograft or xenograft. When the miniballs can be mounted to a clasp-wrap (miniball wrap-surround) or alternative means for introducing ferromagnetic implants in the wall of a ductus, the need for implantation is eliminated and since lumen diameter need not be sufficient for transluminal access, ducti smaller in diameter than those implantable can be treated. However, to achieve continuous adhesion over the outer surface of the ductus that resists the traction of the magnets and yet complies with the intrinsic movement within the walls of the ductus, much less avoids interfering with such action, is elusive as not to eliminate the need for implantation capability.

Because all bodily tissue, even the enamel of the teeth, is constantly replaced, long-term adhesion is a problem. The use of a clasp-wrap is considered a relatively short-term solution unsuited to use in younger patients with a long life expectancy. Furthermore, the use of a wrap-surround, whether a clasp-wrap or a magnet-wrap, is limited to structures that are readily encircleable with few if any attachments that necessitate extensive dissection, and preferably with no more than loose surrounding fascia. The interposition of an artificial adventitia precludes the use of medication on the inner surface of the outer or magnet-mounting component or stent-jacket. However, the medication is then applied to the inside of the reinforcing wrap in contact with the structure.

Integration with the host tissue is not a desirable means for obtaining the adhesion of such an artificial adventitia to the outside of the ductus, because it requires an antecedent procedure, necessitates some negligible surface preparation scoring injury to the intrinsic adventitia of the ductus requiring time to heal, and usually results in bonding of insufficient strength to resist dislodgement by the tractive force exerted by the magnets over time, making adhesion undependable over the long term. Moreover, the need for treatment is usually urgent, making a procedure completed in a single operation imperative.

The methods described herein are intended to avoid open surgery, some avoiding incision entirely, but do nothing to preclude reversal and the application of alternative treatment should results prove inadequate. All of the procedures described herein for the repair of tracheal collapse are practically reversible, and neither in performance nor reversal nearly so traumatic or hazardous as are the standard procedures.

If dysphagia or discomfort continues for more than 15 days following the esophageal tacking procedure, then the miniball magnets are retrieved from the tracheal ceiling and esophageal floor by means of an electromagnet and intraluminal stents inserted in the trachea and bronchi as necessary. Once implanted, imaging other than magnetic resonance must be used, and heart pacing circuitry may be disrupted by proximity to magnets.

An extraluminal stent, because it completely mantles or surrounds and can seal an artery is better able to prevent rupture with hemorrhage than is an intraluminal stent having the form of an open mesh or grid. Studies of the consequences of small puncture wounds to the internal elastic lamina have so far been limited to those produced by microsurgical needles and microelectrodes with no opportunity for healing. By comparison, the longitudinal segmentation of stent-jacket bar magnets allows an extraluminal stent to comply with tonic (angiotonic), pulsatile, and peristaltic changes in gauge regardless of the anatomical tube treated or the length of the stent.

Because the stent-jacket is compliant and the distance slight, that the magnets act in a bistable way as to abruptly seize or ‘yank’ a ferromagnetic object when the field strength meets a certain value does not mean that an extraluminal stent interferes with the normal motility intrinsic in the ductus wall. Essentially, the lumen wall is drawn little or no farther outward than in normal function and under less and less rapidly changing force, so that the risks of the adventitia separating or delaminating from the media or of stretching injury are slight to nonexistent.

To be certain that the normal quiescent diameter of the ductus is not significantly exceeded by the internal diameter of the stein-jacket to be applied, the ductus can be measured with a caliper and the reading matched to the internal diameter specified on the stent-jacket package. In basic contrast with endoluminal stents, the extraluminal stent moves with the walls of the lumen. From the moment of insertion, the extraluminal stent is immediately and instantly compliant in a way that a slowly and limitedly shape adaptive nitinol stent cannot approach.

Between its longitudinal magnets, the base tubing of an extraluminal stent-jacket can be slitted, perforated, or slotted to enhance compliance with smooth muscle action, and perforation or slotting will also serve to expose the outer surface of the ductus to its normal chemical environment. Small, delimited, and distantly spaced punctures of the internal elastic lamina do not represent injury equivalent to the running dissection of a vessel as the result of balloon overinflation which can lead to shrinkage, intimal hyperplasia, and restenosis, and is certainly not equivalent to rupture. Stress relief afforded by an extraluminal stent (see Berry, et al. 2002, op cit.) is not approachable by an intraluminal stent.

In contrast to this least initial trauma of endoluminal stents, the extraluminal stenting to be described requires not only transluminal access to place an intravascular component subadventitially, but extravascular entry through a separate incision or entry wound to allow permural access for placement of an extravascular component, or stent-jacket. The intravascular component consists of miniature ferromagnetic balls that implanted ballistically, produce some tearing and bruising that can result in inflammation, which is, however, medically manageable and short lived. The detailed responses of the lumen wall to ballistic implantation of internal origin are distinct from the form of injury, edematous swelling, and ensuing inflammation that are seen following injury to tissue exposed to the environment where dermal and muscle cells are crushed in depth and many tiny vessels torn.

This situates extraluminal stenting on the trauma scale as intermediate between intraluminal stenting and open surgery. Essentially, conventional or intraluminal stenting trades initial placement with relatively little trauma but the probability of complications that will increase in severity over time for short-term inflammation as the result of some cell-crushing, tearing and bruising, edematous swelling, in larger vessels, some vas vasorum bleeding, and the need for permural entry to place the stent-jacket, but thereafter, as with high-quality dental restorations, relative freedom from long-term complications. In fact, just as might the methods described herein, conventional methods occasionally result in unpredictable injury and adverse sequelae, no procedure known being capable of avoiding this prospect. Since medical surveillance is close while the patient is still in the hospital and immediately following discharge, the earlier unavoidable sequelae appear, the more will there be the opportunity for successful management.

This object of freedom from complications over a long-term equates to suitability for permanent as opposed to temporary repair. Stent-jacket expansion inserts made of layers that are spontaneously absorbable, or are absorbable when warmed by a thermal angioplasty capable barrel-catheter with or without the need for reentry, or when to remain over a longer term as to necessitate stony materials that are destructible by means of lithotripsy or ultrasonic lithotresis, which does require a follow-up procedure, for example, are limited to achieving reconformation made necessary by a changing condition in precedence to an end condition that should prove relatively stable.

Exceptionally, where the transluminal placement of a conventional (endoluminal) absorbable stent to temporarily support an enlarged or collapsed ductus pending subsidence is impossible and entry by means of an angiotomy would result in greater trauma, completely absorbable stays used without a circumvascular stent-jacket, as will be described, may be preferred. Any instance of absorption can also involve the liberation of medication.

When a separate incision is necessary to access the outside of a tubular structure that is otherwise inaccessible from outside the body, the minimization of trauma requires that the incision be small and that the component to be inserted be practically manipulable through the incision. Compared to the length of the incision needed to suture prosthetic rings at intervals about the trachea, for example, that needed to insert and place a stent-jacket is small. Operative and general anesthetic time to be minimized, larger inserts must readily unfold as desired, and any use of adhesives not result in unintended sticking.

### Accommodation of Lumen Contents and Autonomic Function

With the barrel-assembly substantially occluding the lumen, the force of discharge is clearly sufficient to discount counteracting variables, such as intervening lumen contents, the pulse, and muscular action in the lumen wall. These factors tend to be minimized, because discharge will usually be with a muzzle-head that has been selected precisely so that the muzzle port or ports will remain in contact with the lumen wall. The adverse consequences of occlusion, notably ischemia in the arterial tree, are minimized by devising the apparatus to require relatively little time to use and by providing passages for contents, here blood, to flow past, albeit much restrictedly.

Even when lumen contents do intervene or action in the lumen wall is present, the force of discharge will rarely be proportionally insufficient to necessitate its increase, so that except in exceptional circumstances, contents, pulse, and smooth muscle action may be discounted. Automatic adjustment in exit velocity, which is readily adjustable manually, is thus seen as a needless expense. However, since ejection is forward at an acute angle, the alternate mediolateral-lateromedial reciprocation of the wall at the permedial, medial, or equivalent target site distal to the muzzle-port due to the pulse or peristalsis can result in implantation too shallow (medial) or distant.

Drugs allow slowing the pulse or peristalsis, and opioids can stop peristalsis. Nevertheless, for use in an artery, the incorporation of a means for automatically synchronizing discharge to the movement of the wall during manual or automatic discharge when unviewable or not dependably implantable under manual control is unnecessary. Instead, the entry wound for inserting the stent-jacket is made prior to discharge and a Halsted mosquito forceps or hemostat used to compress the artery ahead of (distal to, downstream of) the target site to suppress the pulse. This is more readily obvious in the use of a stay insertion tool, which requires local entry ab initio.

### OBJECTS OF THE INVENTION

1. The primary object of the invention is to reduce in frequency the need to place a foreign object endovascular lining or prosthesis within a diseased lumen. Situated therein, artificial liners, or stents, clog, fracture, migrate, irritate, interfere with normal function, and may necessitate adjunctive medication such as thrombolytic in dangerous dosages. The primary object of the invention is thus to provide means for extraluminal or circumvascular stenting which respond to an alternative set of medical criteria and offer options for treatment not previously available for any and every condition currently treated by means of endoluminal stenting. Another major object of the invention is to augment the treatment options for the many conditions that stenose, weaken, or collapse tubular anatomical structures.
Further objects include the provision of:
2. Implantation through a trajectory that, no wider than the implants themselves, minimizes trauma and quickly seals.
3. Implantation with such suddenness that tissue displacement, hence, implant misplacement or loss, are minimized,
4. Implantation with repeatable control over the force of impact, so that allowing for the variability in mechanical properties of diseased tissue, a consistency in depth of penetration can be achieved that to attempt to duplicate with a hand tool would pose difficulty, hesitancy, and variability resulting in increased injury.
5. Practical testing and operative means, that empirical and intuitive, eliminate complications or the need for computation, and thus minimize the chances for human error.
6. Apparatus that is as intuitive and simple as possible to use and thereby minimizes the chances for human error.
7. Independent angioplasty catheters that do not use a balloon but unlike prior art catheters, additionally capable of insertion into an airgun to initiate stenting immediately without the need to withdraw it and introduce a second stent delivering balloon catheter, thus averting repeated passage and irritation to the entry wound.
8. Independent angioplasty catheters which can additionally incorporate a prior art catheter of either the laser or rotational burr type to cope with more protrusive highly calcified plaque toward the center of the lumen, while unique components allow atheroablation and not just a smashing of plaque up within the lumen wall, thus yielding an overall result critically improved compared to prior art angioplasty and equal to alternative methods of atherectomy.
9. Systems and methods as allow the subadventitial implantation of ferromagnetic spherules as makes it possible to alleviate the symptoms of collapsed trachea in dogs, averting the need for an open surgical procedure in which polypropylene prosthetic cartilage rings cut from syringe cases are sutured about the trachea, demanding extensive incision and dissection and jeopardizing the tracheal vasculature at a time when the patient is at greatest operative risk and least able to withstand such trauma.
10. In the alleviation of collapsed trachea in dogs, elimination of the need for a thoracotomy through use of a new form of stent that is placed circumtracheally (peritracheally), that is, about, not inside, the airway, and thus is not susceptible to the accumulation of mucus as necessitates frequent reinspection, withdrawal, and replacement.
11. Systems and methods that reduce or eliminate the sequelae associated with endoluminal stenting, to include restenosis in vessels and reocclusion in other tubular anatomical structures to which the stent is itself a contributing factor if not the sole cause, fracture, fragmentation, migration intact or following breakage, clogging, and erosive irritation to the portions of the lumen in contact with the stent thereby achieving stenting action that remains trouble-free to the end of life.
12. Interventional procedures that while more traumatic than endoluminal stenting to implant yield relatively inconsequential problems thereafter and are less, usually significantly less, traumatic than open surgery.
13. Interventional procedures that while more traumatic than endoluminal stenting upon insertion are less traumatizing and risk laden than open surgery but which are nonetheless readily practicable.
14. Limited-purpose interventional airguns at relatively low cost through modification of off the shelf airguns.
15. Specialized interventional airguns to provide finer control resolution, with redundant control points to circumvent criticality in adjustment of a single control to allow exactitude in setting the exit velocity or force of penetration sought.
16. Interventional apparatus and procedures as allow angioplasty, atherectomy, and stenting in a single operation, minimizing intracorporeal time, and necessitating luminal entry once with minimal entry wound and other complications or trauma.
17. Apparatus for use in the vascular system which affords operative speed and blood bypass paths to minimize perfusion defects or ischemia and does not involve installing a thrombogenic metal surface object within the lumen.
18. Means of extraluminal stenting that comply with the intrinsic motility of and neither mechanically nor chemically irritate the ductus in any manner that is avoidable.
19. Stents and like devices that positioned outside of the lumen will not occlude the ductus as might an endoluminal stent that failed, migrated, or both.

Apparatus for use in the vascular system that encourage shorter intravenous infusion times of platelet inhibitor-type antithrombotic or aggregation counteractant, such as glycoprotein IIb/IIIa receptor blockade or antagonist (abciximab), and lower doses of thrombolytic drugs, thus reducing the risk of bleeding complications (Lenderink, T., Boersma, E., Ruzyllo, W., Widimsky, P., Ohman, E. M., Armstrong, P. W., Wallentin, L., Simoons, M. L. 2004. “Bleeding Events with Abciximab in Acute Coronary Syndromes Without Early Revascularization: An Analysis of GUSTO IV-ACS,” American Heart Journal 147(5):865-873). In higher doses, these drugs increase the risk of bleeding complications (see, for example, Cote, A. V, Berger, P. B., Holmes, D. R., Scott, C. G., and Bell, M. R. 2001. “Hemorrhagic and Vascular Complications after Percutaneous Coronary Intervention with Adjunctive Abciximab,” Mayo Clinic Proceedings 76(9):890-896; Jong, P., Cohen, E. A., Batchelor, W., Lazzam, C., Kreatsoulas, C., Natarajan, M. K., and Strauss, B. H. 2001. “Bleeding Risks with Abciximab After Full-dose Thrombolysis in Rescue or Urgent Angioplasty for Acute Myocardial Infarction,” American Heart Journal 141(2):218-225). At the same time, in lower doses these drugs are of critical value perioperatively (see, for example, Tcheng, J. E., Kandzari, D. E., Grines, C. L., and 11 other authors 2003. “Benefits and Risks of Abciximab Use in Primary Angioplasty for Acute Myocardial Infarction The Controlled Abciximab and Device Investigation to Lower Late Angioplasty Complications (CADILLAC) Trial,” Circulation 108(11):1316-1323).

These and other objects and advantages of the invention will become apparent from the following specification and accompanying drawings. Further scope in the applicability of the invention will become apparent from the detailed descriptions of the preferred combinations of components, assemblies, and methods, or the embodiments, given herein. Since various modifications in keeping with the concept and scope of the invention will be evident, the detailed specification is given only by way of illustration. The invention accordingly comprises the features of construction, combination of elements, and arrangement of parts exemplified in the constructions hereinafter set forth, and the scope of the invention will be indicated in the claims.

### SUMMARY OF THE INVENTION

The objects are accomplished in accordance with the invention by the provision of apparatus for implanting ferromagnetic miniature balls, or miniballs, peripheral to most of the physiologically more active inner layers or tunics that line the lumen. Tiny permanent magnets, which for specific uses are magnetized miniballs to be implanted by the same means, are placed outside the periphery of the structure in surrounding relation as to exert a mild outward pull on the miniballs that is too slight to cause the miniballs to penetrate, puncture, or fistulize through the outer fibrous sheath but sufficient to maintain the lumen patent. The miniballs are drawn outward toward the circumferential and inwardly directed like poles of the magnets, each of whose circuit encompasses the spherules closest to it. Alternative means and methods for introducing ferromagnetic implants into the walls of failing vasa or ducti are also presented.

### BRIEF DESCRIPTION OF THE DRAWINGS

A more complete understanding of the invention can be obtained by reference to the accompanying drawings of which:

FIG. 1 is a diagrammatic (histological detail lacking) longitudinal sectional view of a vas or ductus lumen with ferromagnetic spherules or miniature balls implanted just inside the fibrous outermost layer, or tunica adventitia, and the trachea mantled about by a circumvascular stent-jacket.

FIG. 2 is a diagrammatic longitudinal sectional view of a vessel mantled about by a full-circle or slotted circumvascular stent-jacket showing the higher density of shot-like smaller ferromagnetic spherules or miniball implants used just inside the fibrous outermost layer (tunica adventitia, tunica fibrosa) of a vas or ductus as laid down by automatic discharge to more uniformly distribute the magnetic traction and thereby reduce the risk of perforation.

FIG. 3. is a diagrammatic cross sectional view taken along line A-N in FIGS. 1 and 2 of the lumen with ferromagnetic spherules or miniballs implanted just inside the fibrous outermost layer, or tunica adventitia, which has been mantled about by a full-round circumvascular stent-jacket as shown in longitudinal section in FIGS. 1 and 2.

FIG. 4 is an angular perspective view of the full-round extravascular stent-jacket component of an extraluminal stent with gas-exchange or breathing perforations for a vessel or duct that can be fully encircled as shown in FIGS. 1 and 2.

FIG. 5 is an angular perspective view of a stent-jacket with circumferentially expanded side-slit, or side-slot, for clearing a subjacent ligamentous or mesenteric attachment, or a branch or branches of the treated vessel that plunge into deeper tissue to form a t-joint.

FIG. 6 is an angular perspective view of a stent-jacket having an absorbable temporary expansion insert along one longitudinal edge to allow the progressive approximation of the edges as swelling of the ductus subsides.

FIG. 7 is a detailed cross-sectional view of a stent-jacket multilayered expansion insert that is divided between both facing side-slit edges of the stent-jacket base-tube taken along line B-B′ in FIG. 6.

FIG. 8 is a cross sectional view of a stent-jacket multilayered expansion insert with maximum expansion divided between both facing side-slit edges of the stent-jacket base-tube taken along line B-B′ in FIG. 6.

FIG. 9 is a full-face view of a tweezers or tongs-type stent-jacket base-tube slit-expansion and mantling hand tool for use with deep lying ducts or vessels, in which the restorative force of the structure and material serve to pull open the stent-jacket for placement about the vessel or duct when compression between the thumb and index finger is released.

FIG. 10 is a full-face view of a tweezers or tongs-type stent-jacket base-tube slit-expansion and mantling hand tool for use with ducts or vessels that lie close to the surface of the body, in which the restorative force of the structure and material serve to pull open the stent-jacket for placement about the vessel or duct when compression between the thumb and index finger is released.

FIG. 11 is a full-face view of a forcept or scissors-configured stent-jacket base-tube slit-expansion tool for use with ducts or vessels that lie close to the surface of the body.

FIG. 12 is a full-face view of a forcept or scissors-configured stent-jacket base-tube slit-expansion tool for use with ducts or vessels that lie deep to the surface of the body.

FIG. 13 is a full-face view of an open magnet-wrap.

FIG. 14 is a left edge-on sectional view of an open magnet-wrap taken along line C-C′ in FIG. 12 with the plies separated.

FIG. 15 is a full-face view of an open clasp-wrap.

FIG. 16 is a detailed overhead or plan view of a single clasp in the clasp-wrap shown in FIG. 15 as indicated therein.

FIG. 17 is a detailed sectional view along line D-D′ of the individual clasp shown in FIG. 16.

FIG. 18 is a full face overhead or plan view of a patch-magnet suitable for subcutaneous or suprapleural attachment.

FIG. 19 is cross sectional view of a patch-magnet suitable for subcutaneous or suprapleural attachment taken along line E-E′ in FIG. 18.

FIG. 20 is a cross-section through a miniball.

FIG. 21 is a cross-section through a miniball having additional layers to chemically isolate the core, release medication, and/or emit radiation.

FIG. 22 is a full-face view of a 7-shot rotary magazine clip for use in a single barrel (monobarrel) radial discharge barrel-assembly.

FIG. 23 is a full-face view of a 10-shot rotary magazine clip for use in a four barrel or four-way radial discharge barrel-assembly.

FIG. 24 is a lateral longitudinal sectional view of a simple pipe barrel-assembly suitable for use in the tracheobroncial tree wherein the anatomy is structurally differentiated, requiring aiming.

FIG. 25 is a lateral longitudinal sectional view of a simple pipe-type barrel-assembly similar to that shown in FIG. 24, but equipped with a bounce-plate for reflecting and so reversing the trajectory at an equal angle in the opposite direction.

FIG. 26 is a longitudinal sectional detailed view showing the miniball recovery electromagnet within its enclosure affixed to the underside of the distal concavity in a simple pipe type barrel-assembly such as those shown in FIGS. 24 and 25.

FIG. 27 is a detailed perspective view partly in section of the muzzle-head of the simple pipe barrel-assembly with bounce-plate shown in the overall view of FIG. 25.

FIG. 28 is a mid-longitudinal section through the barrel-catheter and outside view of the muzzle-head of a single barrel radial-discharge barrel-assembly to show the various parts.

FIG. 29 is a mid-longitudinal section through the four-barrel barrrel-catheter and muzzle-head of an ablation and angioplasty-incapable (plain discharge, limited-purpose) radial-discharge barrel-assembly that lacks a convoluted section and side-sweeper modules to show the parts within the muzzle-head

FIG. 30 is a cross-section through a blood tunnel of an ablation and angioplasty-incapable four-barrel radial discharge barrel-assembly taken along line F-F′ in FIG. 29.

FIG. 31 is a full-face cross-section view through a centering device of a center-discharge ablation and angioplasty-incapable four-barrel radial discharge barrel-assembly when the barrel-catheter is relatively large in diameter taken along line G-G′ in FIGS. 29 and 34.

FIG. 32 is a full-face cross-sectional view through centering device of an alternative centering device in an ablation and angioplasty-incapable four-barrel radial discharge barrel-assembly and one suitable for use in an edge-discharge barrel-assembly or in a barrel-assembly that places the barrel-tubes more distant radially from the longitudinal axis of a relatively large diameter barrel-catheter taken along line G-G′ in FIGS. 29 and 34.

FIG. 33 is a full-face cross-sectional view through a centering device of a center-discharge ablation and angioplasty-incapable four-barrel radial discharge barrel-assembly that places the barrel-tubes less distant radially from the longitudinal axis of a relatively small diameter barrel-catheter taken along line G-G′ in FIGS. 29 and 34.

FIG. 34 is a mid-longitudinal-sectional detailed view of the internal structure of barrel-catheter as shown in FIG. 29.

FIG. 35 is a cross-sectional view through the magnet chambers behind the nose in the muzzle-head of the barrel-assembly taken along line H-H′ in FIG. 29.

FIG. 36 is a perspectival view of an airgun valve-body that incorporates a sliding pressure-bleeding valve.

FIG. 37 is a detail view of the sliding pressure-bleeding valve shown in FIG. 36.

FIG. 38 is a mid-longitudinal section through a basic ablation and angioplasty-incapable center-discharge muzzle-head.

FIG. 39 is a mid-longitudinal section through an ablation and angioplasty-capable center-discharge muzzle-head with side-sweeping modules and recovery electromagnets chambered normal to the long axis of the barrel-assembly and equipped with a trap-filter that is stowed in the silo within the extended nose and push-solenoid deployed and retracted.

FIG. 40 is a detail view of the trap-filter and if deploying and retracting push-type solenoid as shown in FIG. 39 in the deployed state.

FIG. 41 is a detail of the mid-longitudinal section through the side-sweeping modules in the ablation and angioplasty-capable center-discharge muzzle-head shown in FIG. 39.

FIG. 42 is a detailed view showing four differently configured tips for the bristles or aristae in the side-sweepers located as shown in FIGS. 39 and 41.

FIG. 43 is a full face external view of the center-discharge ablation and angioplasty-capable center-discharge muzzle-head shown in FIG. 39 to show the heat-windows for the turret-motor and recovery electromagnets.

FIG. 44 is a mid-longitudinal section of center-discharge ablation and angioplasty-capable center-discharge muzzle-head equipped with side-sweepers and a trap-filter that is stowed in the silo within the less extended nose and push-solenoid deployed and retracted having recovery electromagnets parallel to the long axis of the muzzle-head.

FIG. 45 is a mid-longitudinal section of a cooling catheter.

FIG. 46 is a cross-section through the cooling catheter taken along line I-I′ in FIG. 45.

FIG. 47 is a mid-longitudinal section through a combination-form ablation and angioplasty-capable edge-discharge muzzle-head with side-sweeper modules, trap-filter, and incorporating an excimer laser through the central canal.

FIG. 48 is a cross-section through an ablation and angioplasty-capable edge-discharge muzzle-head taken along line J-J′ in FIG. 47 showing the eccentric (off-axis, lateral) location of the trap-filter silo when the recovery electromagnets are installed parallel to the longitudinal axis of the muzzle-head.

FIG. 49 is a detailed view of the (proximal) end-plate in a two-barrel radial discharge barrel-assembly showing the electrical connection as integrated into the mechanical connection of the barrel-assembly within the barrel of the airgun.

FIG. 50 is a detailed perspective view of the twist-to-lock connector (connecting flange) used to connect a barrel-catheter of any type, but here shown fixed to a disconnected simple-pipe type barrel-catheter, and the stop-and-lock ring affixed to the muzzle of the airgun into which the twist-to-lock connector is engaged.

FIG. 51 is a longitudinal sectional view of the barrel-catheter of FIG. 50 locked in position within an airgun.

FIG. 52 is a side view partly in section and partly external of an alternative electrical connection to that shown in FIG. 49 but separate from (distal to) the mechanical connection of the barrel-assembly to the barrel of the airgun.

FIG. 53 is a full face longitudinal view partly in section of an ablation and angioplasty-capable barrel-assembly with onboard battery pack and control panel, thus able to function independently of the airgun for an angioplasty, but since engaged in an airgun to initiate implantation discharge.

FIG. 54 is a detailed view of a control panel on an ablation and angioplasty-capable barrel-assembly with heatable turret-motor stator, directional heat-windows necessitating the ability to rotate the turret-motor independently of the airgun power supply, brush-type side-sweeper modules, independently heatable recovery electromagnet windings, and an excimer laser.

FIG. 55 is a detailed view of a forward drive stabilizing linkage that extends over the barrel-catheter to maintain it in a straight condition.

FIG. 56 is a cross-section through a forward drive stabilizing linkage taken along line K-K′ in FIG. 55.

FIG. 57 is a break-out view of the components contained within the hand-grip that includes the battery pack and control panel in an ablation and angioplasty-capable barrel-assembly showing the wiring scheme.

FIG. 58 is a sectional view of an interventional airgun that is limited to manual discharge and is suitable for procedures involving the treatment of different tissues to different depths in quick succession with redundant points of control to adjust the exit velocity at a rate adequate for numerous procedures.

FIG. 59 is a sectional view of an interventional airgun that is limited to manual discharge but suitable for procedures involving the treatment of different tissues to different depths in quick succession with redundant points of control to adjust the exit velocity

FIG. 60 is a diagrammatic representation of an interventional airgun with additional exit velocity control points compared to the airgun of FIG. 59 for quick midprocedural adjustments, using rotary magazine clips to allow the use of multiple barrel-tube barrel-assemblies, and provided with an automatic positional control system that allows uniform close formation implantation suitable for use to implant the wall of a blood vessel.

FIG. 61 is a diagrammatic representation of the airgun of FIG. 60 showing the coordination between the timing and positional control in the automatic laying down of a pattern of implants with uniformity and sufficient accuracy for use to implant the wall of a blood vessel.

FIG. 62 is a detailed view of the control panel for the airgun shown in FIG. 60.

FIG. 63 is a detailed view of the joystick control seen in FIG. 62.

FIG. 64 is a side view partly in section of a spring back to inject after depressed (pusher type, passive type) stay insertion tool that allows the force of insertion to be set by the restorative force of the plunger or plunger-slide return spring but allows force to be added by the operator if necessary.

FIG. 65 is a full face front view of the upper portion of a spring back to inject after depressed (pusher type, passive type) stay insertion tool shown in FIG. 64.

FIG. 66 is a longitudinal section through an active or direct pull trigger to inject type stay insertion tool that allows the operator to control the force of insertion.

FIG. 67 is a detailed view of the stay injecting or working end of the types of stay insertion tool shown both in FIGS. 64 and 66.

FIG. 68 is a detailed view of the injection tip at the stay injecting or working end of the types of stay insertion tool shown both in FIGS. 64 and 66.

FIG. 69 shows the tip of plunger blade at the stay injecting or working end of the types of stay insertion tool shown both in FIGS. 64 and 66.

FIG. 70 is a diagrammatic view of stays implanted within the wall of a duct or vessel whether the stays are ferromagnetic for encirclement by a stent-jacket, consist purely of medication, or represent stay-shaped radiation seeds.

FIG. 71 is a mid-longitudinal cross-section through a stent-stay (containing a ferromagnetic core) with various coatings.

FIG. 72 is a diagrammatic view of a stay showing coating that can be applied in greater detail than is depicted in FIG. 71.

### DESCRIPTION OF THE PREFERRED EMBODIMENTS

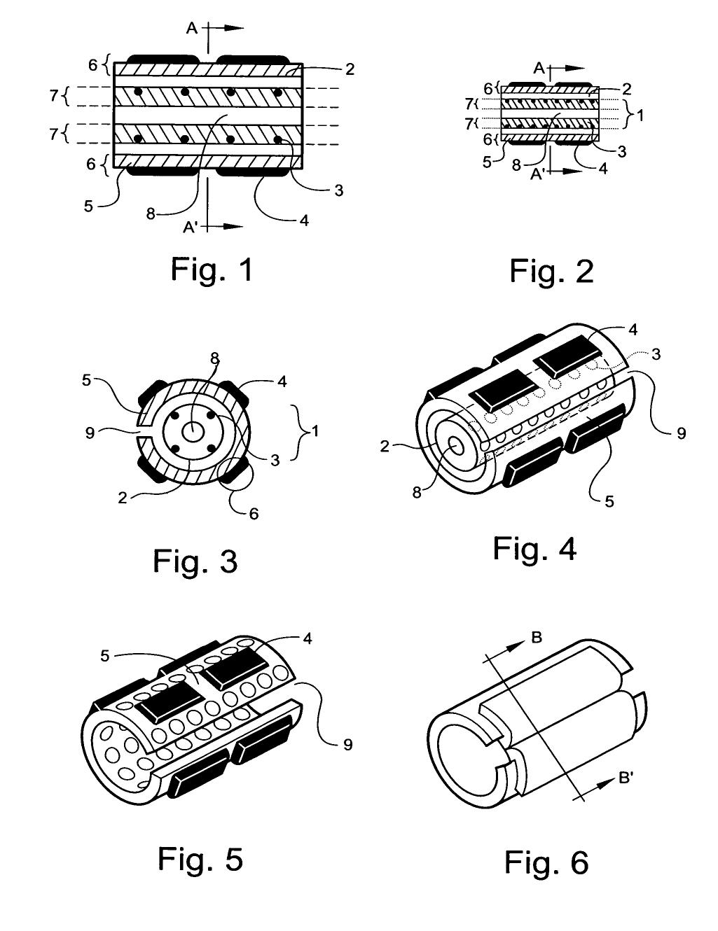

Referring now to FIGS. 1 thru 3, shown is one end-condition sought through use of the means and methods to be described. To apply to almost any tubular anatomical structure, viz. any vas or ductus, the figures are intentionally diagrammatic in omitting histological detail. Accordingly, FIGS. 1 and 3 may be taken to represent the lumen of a ureter, the esophagus, colon, trachea, or bronchus, and FIGS. 2 and 3 the lumen of an artery or vein following treatment in accordance with the implantation apparatus and method to be described where FIGS. 1 and 2 use the same part numbers for equivalent parts and FIG. 3 has not been redrawn merely to show the implants as slightly smaller in FIG. 2 than in FIG. 1.

FIGS. 1 and 2 show the stent-jacket about the vas (vessel) or ductus (duct) in longitudinal section, while FIG. 3 shows this in cross section. With a simple barrel-assembly, an artery is stented following conventional angioplasty or atherectomy, whereas with a barrel-assembly that incorporates a laser and side-sweepers as described below, depending upon the specific medical condition, clearing and stenting an artery is possible without the need to reenter and withdraw a second time.

Vessel or other ductus 1 has implanted just inside its fibrous outer layer, or tunica adventitia 2, ferromagnetic spherules or miniballs 3. Acted upon by the tractive force of the longitudinally positioned small bar neodymium iron boron magnets 4 mounted to the outer surface of a surrounding length of pliant tubing, or base-tube or base-tubing 5 this extravascular component as a unit hereinafter referred to as a stent-jacket 6, the miniballs 3 pull the ductus wall 7 outward and so maintain the passageway or channel that courses through the ductus or lumen 8 open, or patent.

In longer stent-jackets, the bar magnets are segmented to allow flexibility. The stent-jacket can be made to any length, but where flexion is required, base-tube 5 is also segmented into sections joined by an articulated Palmaz-Schatz-type connection as described below under the section entitled Jointed Stent Jackets. Four magnets are shown for simplicity; for some eccentric lesions, only quadrants superjacent to the affected arc of the ductus need be drawn outwards thus, and some lesions may necessitate the use of more than one magnet in each quadrant. The magnetic field strength is strong enough to urge the adventitia 2 into contact with it but not to significantly interfere with the smooth muscle action in the ductus wall 7.

The stent-jacket base-tubing 5 is pliant and slit 9 longitudinally along one side but is not so pliant that the lumen 8 can revert to its former stenosed or constricted condition. The object is not merely to reinstate passability through the lumen of air, blood, food, urine or some other bodily or reproductive substance that would otherwise be obstructed, but to do so with the least trauma essential to obtain results that will not pose complications over a longer term than does conventional or intraluminal stenting.

Several variants of this basic conformation are possible. The base-tubing 5 of a stent-jacket 6, described below, can be slit, slotted, perforated, or made of compound tubing, or a compound extrusion (coextrusion). Perforation of the base-tube other than to adjust its restorative force, such as to ‘breathe’ or to allow tissue infiltration to avert migration, will affect its restorative force, which must not require a thickness that with magnets unacceptably encroaches upon the adjacent tissue. Perforated base-tubes can be lined with a gas-permeable (breathing) layer for direct contact with the outer surface of the ductus, and ongoing experiments for encouraging tissue adhesion and fixation conducted on endothelial progenitor or precursor cells show promise (see, for example, Hoffmann, J., Paul, A., Harwardt, M., Groll, J., Reeswinkel, T., and seven Other Authors 2007. “Immobilized DNA Aptamers Used as Potent Attractors for Porcine Endothelial Precursor Cells,”. Journal of Biomedical Materials Research, Part A (in press).

To achieve the desired restorative force within the desired dimensions, selection of the base-tube material on the basis of intrinsic elasticity and resilience must therefore consider the most effective internal surface conformation, which may incorporate undercuts or deeper texture for increased bonding surface area of an expansion insert, texturing or embossing for tissue infiltration and integration, perforations for gas exchange, for better adhesion of a surface film of medication, adhesive, perforation sealant, and so on, as described below.

Stent-jackets that are placed before initiating discharge in order to interdict continued travel by the risk of a perforating miniball (discussed below in the section entitled Stent-jacket Linings for Containing or Preventing Perforations and for Reducing the Momentum and Misdirection of Rebound) but whose presence risks miniball rebound toward the lumen are made with a softer stent-jacket internal surface layer that is textured to serve any of the purposes indicated, and is backed by a flat-faced layer of greater resilience which is inclined distoradially to direct rebounds subadventitially or medially and thus away from the lumen and toward a functional subadventitial or medial location for stenting purposes.

That is, a more resilient layer, which becomes thinner moving distad, is placed subjacent (outside, lateral, centrifugal) to a superjacent (inner) softer layer, which becomes thicker moving distad, so that these layers together constitute one continuous layer of which the halves are complementary to jointly form a layer of consistent thickness. At such fine thicknesses, the ‘soft’ inner layer would seem relatively ‘hard’ in a nonsubminiature or readily manipulable size.

A miniball rebounded off of the distally receding harder backing layer thus does so at an angle that is more acute in relation to the central axis of the lumen than were the reflecting surface parallel to the axis. The prepositioning of a stent-jacket to insulate a site for thermal angioplasty is unnecessary, and the use of a an angioplasty barrel-assembly for thermal angioplasty when stenting is to be endovascular needlessly introduces a noncontributory step that requires an incision and takes time. Now, a miniball that strikes the more resilient layer through the softer layer and rebounds at the equal and opposite angle will have been directed laterally or centrifugally of the angle that would result were the surface rebounded off of parallel to the central axis of the lumen.

Depending upon the extent of the area to be treated, such an extraluminal stent-jacket, whether consisting of a plurality of separate sections connected or articulated, as indicated above, by articulated joints or Palmaz-Schatz type connections, can be made in any length. A variant to the use of a surrounding stent-jacket with magnets mounted about its outer surface to be described below is the use of a bandage wrap-surround, or magnet-wrap, placed about a neighboring structure with magnets directed toward the miniballs. Unlike the stent-jacket, which draws the ductus outwards all around, this variant is intended for eccentric lesions on one side or to suspend a collapsed trachea.

Another variant to be described below is the use about a ductus of an artificial adventitia that includes spaced miniballs. Such a clasp-wrap or alternative means for introducing ferromagnetic implants in the wall of a ductus is used when the ductus to be treated lacks an intrinsic tougher outer layer and so is incapable of retaining implanted miniballs, or is too small in lumen diameter and thus inaccessible transluminally. While the use of a clasp-wrap or alternative means for introducing ferromagnetic implants in the wall of a ductus allows dispensing with implantation, this artificial adventitia is limited in service life to the time that it remains adherent to the outer surface of the ductus and is therefore not recommended for use in younger and otherwise healthy patients.

A clasp-wrap or alternative means for introducing ferromagnetic implants in the wall of a ductus can be used with either a stent jacket for all-around dilatation or a magnet-wrap for unidirectional expansion of a stenotic or collapsed vessel or duct when intervening tissue in abuting relation to that drawn serves to limit the excursion of the miniballs under magnetic traction. Directional force is suited to eccentric rather than radially symmetrical or circumferentially uniform lesions. By contrast, a stent-jacket inherently establishes a limit for excursion of the miniballs, hence, the resultant expansion or dilatation of the ductus, eliminating any risk of stretching injury.

Magnets can also be placed subcutaneously or suprapleurally, as described below. Once implanted with miniballs, the collapsed dorsal membrane of a trachea, for example, can be drawn to and thus lifted out of sagging obstruction to the lumen by magnets in a stent-jacket immediately surrounding the trachea, or situated along ventrolateral lines of a magnet-wrap surrounding the esophagus, or placed subcutaneously along the neck and shoulders, or suprapleurally above the ceiling of a collapsed bronchus.

An example of the combined use of these variants is the application of jointed (articulated, segmented) stent-jackets to support the dorsal membrane of the trachea anterior to the thoracic inlet with subcutaneously or suprapleurally placed magnets used to support the ceiling when collapse extends into the bronchi where these are embedded in lung tissue and no longer encircleable by a stent-jacket.

### Stent-Jackets

Stent-jackets can be used to maintain the patency of any tubular anatomical structure that unembedded and without extensive attachment, provides sufficient wrap-around access for the stent-jacket to grasp it about. A stent-jacket consisting of a single segment of side-slit tubing is referred to here as simple, whereas one consisting of two or more segments connected by a joint is referred to as jointed or articulated, the term ‘compound’ avoided as ambiguously denoting either made with plural segments or made of compound tubing material. Both simple and jointed stent-jackets can be full round to encircle the ductus, or when the ductus is attached along a line, partially round to avoid attachment to substrate or adjacent tissue, or some segments can be full and others partially round.

The restorative force of the stent-jacket base-tube, which is the product of the intrinsic elastomeric properties of the material, or if a coextrusion, the combined materials, and its thickness, or the relative thickness of each material, is selected for close compliance with the smooth muscle action passing through the ductus and not so resistant to such action that the margins (end rims) dig into the outer surface of the ductus. In some instances, articulated stent-jackets are used to achieve this action.

An extravascular, extraureteric, or other stent-jacket must not present any discontinuity on its inner surface to the outer surface of the vessel or at its margins as would pose chronic irritation, and must possess elasticity so as to yield to the excursion at and about the point of impact when placed prior to implantation of the miniballs. In most instances, deformation in the cross section of a vessel is best avoided as promoting turbulent flow. The tendency for the magnetic traction to distort the cross section from circular to angular is limited by the stent-jacket, which encircling the vessel, usually in flush relation, is circular.

Regardless of the detailed formation of magnets mounted to its surface, the base tubing of an extraluminal stent jacket can be slit, perforated, or slotted between the magnets to enhance compliance with smooth muscle action, and expose the outer surface of the ductus to its normal chemical environment. Signficant porosity has been shown essential for the suppression of hyperplasia in vein grafts (George, S. J., Izzat, M. B., Gadsdon, P., Johnson, J. L., Yim, A. P., Wan, S., Newby, A. C., Angelini, G. D., and Jeremy, J. Y. 2001. “Macro-porosity is necessary for the reduction of neointimal and medial thickening by external stenting of porcine saphenous vein bypass grafts,” Atherosclerosis 155(2):329-336). Provided the condition is diagnosed early, since stent-jackets can be articulated by connecting a train of otherwise separate stent-jackets with stainless steel wires or strip-wires on either side similarly to older Palmaz-Schatz intravascular stents to allow flexion with no buckling, and the extravascular stent can itself constitute a compliant jacket to contain, for example, an incipient aneurysm.

The absorbable cements specified herein can also serve to release medication. This includes both hydrogels (see, for example, Yin, L., Fei, L., Cui, F., Tang, C., and Yin, C. 2007. “Superporous Hydrogels Containing Poly(acrylic acid-co-acrylamide)/O-carboxymethyl Chitosan Interpenetrating Polymer Networks,” Biomaterials 28(6):1258-1266; Ishihara, M., Fujita, M., Obara, K., Hattori, H., Nakamura, S., Nambu, M., Kiyosawa, T., Kanatani, Y., Takase, B., Kikuchi, M., and Maehara, T. 2006. “Controlled Releases of FGF-2 and Paclitaxel from Chitosan Hydrogels and Their Subsequent Effects on Wound Repair, Angiogenesis, and Tumor Growth,” Current Drug Delivery 3(4):351-358; Ishihara, M., Obara, K., Nakamura, S., Fujita, M., Masuoka, K., Kanatani, Y., Takase, B., Hattori, H., Morimoto, Y., Ishihara, M., Maehara, T., and Kikuchi, M. 2006. “Chitosan Hydrogel as a Drug Delivery Carrier to Control Angiogenesis,” Journal of Artificial Organs 9(1):8-16; Serra, L., Domenech, J., and Peppas, N. A. 2006. “Design of Poly(ethylene Glycol)-tethered Copolymers as Novel Mucoadhesive Drug Delivery Systems,” European Journal of Pharmaceutics and Biopharmaceutics 63(1):11-18; Hu, B. H. and Messersmith, P. B. 2005. “Enzymatically Cross-linked Hydrogels and Their Adhesive Strength to Biosurfaces,” Orthodontics and Craniofacial Research 8(3):145-149; Roorda, W. E., Bodde, H. E., de Boer, A. G., Bouwstra, J. A., and Junginger, H. E. 1986. “Synthetic Hydrogels as Drug Delivery Systems,” Pharmacy World and Science [Pharmaceutisch Weekblad. Scientific Edition] 8(3):165-189) and cyanoacrylates, (see, for example, “Local Delivery of Vancomycin for the Prophylaxis of Prosthetic Device-related Infections,”; Eskandari M M, Ozturk O G, Eskandari H G, Balli E, and Yilmaz C. 2006. “Cyanoacrylate Adhesive Provides Efficient Local Drug Delivery,” Clinical Orthopaedics and Related Research 451:242-250).

A full-round stent-jacket is shown in FIGS. 1-4, the parts thereof already enumerated above, and a partial stent-jacket, or partially round or slotted stent-jacket, the parts thereof already enumerated above, is shown in FIG. 5. A full round stent-jacket is used when the vessel or duct is completely encircleable, allowing miniballs implanted at intervals entirely about the interior wall of the tunica adventitia to be attracted by extravascular magnets, or in the case of the trachea, subcutaneous or suprapleural magnets, aligned to these. Less than fully circumferential discharge is accomplished, as is explained below, by blanking out one or more of the barrel holes in the rotary magazine clip used with a fully circumferentially discharging barrel-assembly or by means of a barrel-assembly with a muzzle-head having ports at angles confined to only a portion of the circumference.

Whether discharge is fully or omni-radial or circumferential or partially, radial, a completely or full-round stent-jacket is used to surround a fully encircleable vessel, and a partial stent-jacket used about a partially encircleable vessel. The circumferential positions of miniballs implant beneath the tunica adventitia and bar magnets in the stent-jacket are aligned. If eccentricity of the lesion or other condition of the vessel wall recommends avoiding implantation in certain arcs about the vessel circumference, provided the vessel wall can be predicted to recover in strength, such areas of the wall can temporarily be kept patent by bonding to the inner surface of the stent-jacket with, for example, surgical cyanoacrylate cement. If the condition is aneurysmal, i.e., failure is outward rather than inward, bonding is omitted.

When diagnosed early, an adequately anchored circumvascular stent-graft applied, for example, to an incipiently aneurysmal abdominal aorta is not subject to endoleak. Furthermore, it having been demonstrated in an endovascular stent-graft that is bifurcated for extension down into the iliac arteries, that the ability to flex improves the outcome of an abdominal aortic aneurysm (Arko, F. R., Lee, W. A., Hill, B. B., Cipriano, P., Fogarty, T. J., and Zarins, C. K. 2001. “Increased Flexibility of AneuRx Stent-graft Reduces Need for Secondary Intervention Following Endovascular Aneurysm Repair,” Journal of Endovascular Therapy 8(6):583-591), a circumvascular stent-graft that is adequately anchored without the need for such extension need flex to a much lesser extent, and if necessary, can be articulated. Where a vessel requiring treatment must flex, articulated stent-jackets in number as to minimize compression at the end-margins of each stent-jacket in the train impart flexibility without constriction.

A barrel-assembly designed for eccentric discharge typically provides muzzle-ports at smaller circumferential distances than does a conventional multibarrel muzzle-head with adjacent muzzle-ports at equal distances about the circumference. Depending upon the extent of eccentricity, eccentric lesions can be treated with a single barrel radial discharge barrel-assembly, especially when a turret-motor is used for lateral coverage at each level implanted. In a multiple barrel-tube (barrel) barrel-assembly, the barrel-tubes are rotated by the distal ejection head into which these engage. That is, the barrel-tubes, unlike the barrel-catheter, which is divided by a rotary joint so that the turret-motor can rotate the distal segment, are continuous, and rotated not at the turret-motor but rather at the distal end which is rotated by the turret-motor.

The distal termini of the barrel-tubes, or muzzle-ports, can thus be remotely rotated through an arc while intraluminal to discharge at an angle to either side of a reference or 0 degree index at the center of the overall working arc. When the circumference or the arcuate extent to either side of the center line of the lumen wall in enfilade is not overextended, a noneccentric multiple radial discharge barrel-assembly, that is, one with muzzle-ports equidistant entirely about the circumference, typically four) using fewer than all of the barrel-tubes can be used for eccentric lesions. To do this, the barrel-assembly is supplied miniballs from a rotary magazine clip that lacks holes for the undesired barrel-tubes. The same approach is used to skirt or straddle a portion of the vessel along the line of its attachment to by connective tissue.

The resilience of the tubing used as the base-tubing for longitudinally mounting the tiny bar magnets varies with the material; its thickness, and length. The tubing material from which the base-tube is cut may be simple or compound (coextruded), affording a wide range of restorative forces. The addition of an internal layer within a stent-jacket to moderate rebound when the stent-jacket is placed prior to discharge in order to prevent perforation and escape of a miniball is discussed below. Prior to placement about the implanted site, the inner surface of the stent-jacket can be wetted with a coagulant, antibiotic, anti-inflammatory, adhesive, or other medication.

Unless encapsulated within a bioinert plastic resin that blunts the exposed edges of the bar magnets, the edges are rounded prior to plastic encapsulation or plating and replating, or plating and Microfusion®, a proprietary plasma-based ion deposition or physical vapor thin film vacuum coating form of metastable phase synthesis available from Implant Sciences Corporation, Wakefield, Mass., which may be used to cover over any microfractures that remain following plating, as well as to impart radiopacity (see Sahagian, R. 1999. “Critical Insight: Marking Devices with Radiopaque Coatings,” Medical Device and Diagnostic Industry Magazine, Canon Communications, May 1999 available at http://www.devicelink.com/mddi/archive/99/05/011.html and http://www.implantsciences.com/pdf/orthodontic.pdf). Microfusion® is an outgrowth of nonplasma (discrete, directed beam) ion deposition (see, for example, Hirvonen, J. K. 1991. “Ion Beam Assisted Deposition,” Material Science Reports 6 (6):215-274; Nastasi, M. A., Mayer, J. W., and Hirvonen, J. K. 1996. Ion-Solid Interactions: Fundamentals and Applications, New York, N.Y.: Cambridge University Press). Both plating and ion deposition are convenient methods for increasing implants to a preferred mass.

Compared to replating, microfusion adds less mass, and with a primary object of providing high radiopacity markings, microfusion can be used for this purpose in lieu of crimped metal banding, the inclusion in components of a metal powder such as barium or tungsten, electroplating, chemical vapor desposition, ion beam assisted deposition, high vacuum thin film coating, cold process physical vapor deposition, or sputter-coating where the marking of components described herein is necessary.

A conical stent-jacket is preferably made by suturing the ends of segments of different diameters together without an adhesive, any gaps at the ends filled by a ring made of sheeting or cut from tubing of intervening diameter and wall thickness. To allow the stent-jacket to open to admit and close about the vessel and thereafter allow expansion and contraction in response to tonic, pulsatile, or peristaltic changes in gauge, such a sectional stent-jacket must have a common side-slit. The difference in diameter among the sections of such a compound stent is not such as to induce turbulent flow to any significant or thrombogenic extent.

Vascular disease tending to favor openings to side branches, or ostia, and longitudinal bifurcations along the lumen wall as exposed to more turbulent flow, the stent-jacket expansion slit can be enlarged to admit a branch or bifurcation by cutting mating semicircles midway along the slit, thus allowing the branch or bifurcation to be spanned. In order to enclose a trunk and branch together, stent-jackets can be made in shapes other than tubular, such as in the form of a tee. A branch that exits at a 90 degree angle must be accessed separately.

The extraluminal stent-jacket base-tube, which depending upon the application may be lined (below), supports bar magnets on its outer surface that typically are tantalum coated for viewability, is made of implantable resilient polymeric tubing that withstands the intracorporeal environment for at least many years if not to the end of life. Tubing that is able to withstand the salinity of the intracorporeal environment can be extruded from pellets or diced Polymer Technology Group, Inc., Berkeley, Calif. Bionate®, (formerly manufactured under the tradename Corethane® Polycarbonate by Corvita, Inc.), polycarbonate-urethane copolymer (see, for example, Ward, R. S. 2000. “Thermoplastic Silicone-Urethane Copolymers: A New Class of Biomedical Elastomers,” Medical Device and Diagnostic Industry 22(4):68-77). Other polymers from the same company include Biospan®, Elasthane®, PurSil®, and Carbosil®, and from Thoratec, Inc., Thoralon®. One such material exhibits a durometer D-scale per ASTM D2240-02 test, Shore A reading at 15 seconds of 55.

Silicone-urethane copolymers are antioxidative, elastic, and resilient at body temperature as to exhibit ‘memory,’ have a coefficient of friction that combined with the other countermeasures to be described are consistent with resistance to migration, and have already met the federally mandated criteria for implantable material. Bioinert polymeric materials suitable for base-tubes are numerous, and include silicone, expanded polytetrafluororethylene, polyfluoroethylene, other fluoropolymers, polyetherurethane, polycarbonateurethane, polysiloxaneurethane, silicone-polyurethane copolymers and hydrogenated poly(styrene-butadiene) copolymer. Recent improvements in these materials mean that replacement should not become necessary for years. Clearance by the neighboring anatomy permitting, varying the restorative force required to fit the ductus treated is easily accomplished merely by changing the wall thickness of the tubing.

For embedded ducti, barring the presumably greater longevity of more recent materials not yet available in the form of tubing (see, for example Pinchuk, L. 1998. “Biostable Elastomeric Polymers Having Quaternary Carbons,” U.S. Pat. No. 5,741,331), compared to tenuous contact with surrounding tissue within a body cavity, complete investment within tissue, which is discouraged, is likely to accelerate the cracking that significantly reduces the service life of implanted polymers. Implanted Elast-Eon 2 80A synthesized using poly(hexamethylene oxide) (PHMO) and poly(dimethylsiloxane) (PDMS) macrodiols has been demonstrated to stand up well and with tolerable alteration in mechanical properties over time (see Simmons, A., Hyvarinen, J., Odell, R. A., Martin, D. J., Gunatillake, P. A., Noble, K. R., and Poole-Warren, L. A. 2004. “Long-term in Vivo Biostability of Poly(dimethylsiloxane)/Poly(hexamethylene Oxide) Mixed Macrodiol-based Polyurethane Elastomers,” Biomaterials 25(20):4887-4900; Martin, D. J., Warren, L. A., Gunatillake, P. A., McCarthy, S. J., Meijs, G. F., and Schindhelm, K. 2000. “Polydimethylsiloxane/Polyether-mixed Macrodiol-based Polyurethane Elastomers: Biostability,” Biomaterials 21(10):1021-1029).

However, the degradation of even embedment within skeletal muscle is less than that to be expected of a plastic material for use in an endovascular stent. Nevertheless, the means described herein are not intended to supplant the treatment by endoluminal stenting of ducti embedded in muscle. It also warrants stating that the base-tube is usually separated from the adventitia by a lining, while the tiny bar magnets mounted to its outer surface are encapsulated for bioinertness and have rounded if any corners to preclude abrasive or probing injury. Otherwise, tubes can be made of individual or different materials laminated to produce stent-jacket magnet platform materials that differ in restorative force. The inner diameter of the tubing should match that of the outer diameter of the vessel and longitudinally slit along one side to allow it to encircle a tubular anatomical structure and radially expand. To increase the surface area in contact with the outer surface of the vessel, the extraluminal stent is somewhat longer than the equivalent intraluminal stent.

In order to encircle (girdle, jacket or grasp about) a vessel of which the deep side is inaccessible without dissection that would demand open surgery, a longitudinal strip is cut from the otherwise fully cylindrical of complete extraluminal partial stent-jacket to create a partial stent-jacket. This side-slot constitutes an enlarged side-slit or longitudinal expansion gap that due to the anatomy is situated at the deep side. When the deep side of a fully encircleable vessel gives rise to a branch, the slit is enlarged to afford clearance for the branch. When the deep side of a vessel that is not fully encircleable gives rise to a branch larger in diameter than the width of the strip cut out that plunges even deeper, then the strip cut from the side of the base-tube 5 is enlarged to afford clearance for the branch. By contrast, existing intravascular stents are unable to retain structural integrity when a void is introduced and therefore unable to span a branch. Parenthetically, as will also be explained, when the branch is approached, rather than to withdraw the radially discharging barrel-assembly in order to replace it with a semiradially discharging muzzle assembly, the rotary magazine clip is simply replaced with one that blanks out the barrels directed toward the opening to the branch.

In treating eccentric lesions, note is taken of the positions of the lesions throughout the course of the vascular system to be treated and the choice of a fully radially discharging muzzle assembly with occasional blanked out rotary magazine clips or partially radially discharging muzzle assembly made on this basis. Minimizing the duration of the procedure and any further trauma to the inguinal or brachial point of entry, the choice is based upon avoiding the need to withdraw one barrel-assembly and replace it with another. To keep the size and mass of the stent-jacket to a minimum as least to encroach upon or rub against adjacent tissue, the magnets should be as diminutive as possible consistent with the magnetic force required. To this end, small sintered neodymium iron boron (Nd₂Fe₁₄B) permanent bar magnets of megagauss oersted (MGO) 50 or higher grade material are used. The magnets are magnetized parallel to their thickness, or normal to their plane.

Readily corroded, neodymium iron boron magnets are often available already nickel plated in a length that spans the implanted miniballs lengthwise, or running parallel to the axis of the tubing. However, neither neodymium iron boron nor nickel is biocompatible (see International Standards Organization standard series 10993, Biological Evaluation of Medical Devices. Bioinertness is attained by overlayment or encapsulation of the bar magnets in gold, tantalum, titanium, or any of the large number of nondegrading bioinert plastic polymer resins. The use of other noble metals, such as platinum, rhodium, and alloys of platinum and rhodium are considered unnecessarily expensive. The magnets are drilled through toward each of their ends or perimeters prior to being coated or encapsulated for bioinertness, then fastened to the base-tube with rivets or eyelets. Since placing the stent-jacket about the ductus with the insertion tool to be described may necessitate the application of lateral force to the sides the magnets with end of a probe, wider flange rivets or eyelets are used. Tantalum coating of the bar magnets affords radiopacity that enhances radiopacity making radiological visualization easier.

When the diseased tissue, stent-jacket, and bar magnets are proportionally long in relation to the peak excursion of the passing pulse, and the bar magnets must be thin for clearance, and moreover, the encapsulated layer added for bioinertness such as gold microfused to a substrate of nickel plate does not add sufficient ultimate strength or resistance to breaking stress for the bending load, the brittleness of sintered neodymium iron boron requires that long and thin bar magnets of this material be longitudinally divided or segmented to avoid fracture. Greater pliancy is attained by using separate stent-jackets, with a continuous base-tube 5 mounting longitudinally divided bar magnets giving intermediate compliance without fracture compared to a continuous stent-jacket spanned from end to end by bar magnets.

Variability in compliance to smooth muscle action is obtained through the use of different stent jacket base-tube 5 materials, which may be compounded by coextrusion or lamination, by varying the relative thicknesses of the layers, the dimensions of the bar magnets, by using separate stent-jackets or a continuous stent jacket with longitudinally discontinuous bar magnets, and by selecting the material or materials of encapsulation and the thickness of each for strength, the suitability of gold, tantalum, titanium, and a bioinert plastic polymer resin, alone or in combinatinon posing a spectrum of choices. For example, where disease has eccentrically weakened the arterial wall, longitudinal segmentation of the magnets on a continuous base-tube 5 offers greater compliance to the passing pulse while fully jacketing about the vessel to prevent its rupture. By comparison, intravascular stents reduce the risk of rupture, but are noncompliant.

Flat flanged straight barrel aluminum eyelets are used to fasten the bar magnets to the plastic tubing. The flanges should not be so large in diameter as to significantly flatten the wall of the base-tube 5. Aluminum has the advantages over the brass used in most eyelets of lower weight and anodizability. When the tubing is large enough in diameter and the wall thickness is sufficient, the edges of the flanges may be drawn flush into the elastic material to the inner surface of the tubing as not to protrude into the outer surface of the vessel. If not, then the edges of the flanges are beveled to preclude irritating the outer surface of the vessel.

It should not be necessary to rout the tube facing flat side of the magnets to create a depressed circle to accommodate the flange in an inlaid or nonprotrusive position. Since closing the eyelets to fasten the magnets to the tubing could cause gold electroplate to break away exposing the non-biocompatible aluminum substrate, plating is not a suitable coating. The eyelets are instead gold anodized, which precedent is long established in the making of dental crowns. Consistent with the need for flexion, longitudinally continuous magnets are preferred to a formation of separate magnets along the length of the tube as reducing the criticality of magnet positioning relative to the miniball implants. The magnets are placed in position about the outer surface of the plastic tube lengthwise along the center of each quadrant, that is, parallel to the long axis of the tubing, planar or wide side down, south poles facing toward or normal to the central axis of the tube, and the holes drilled in the magnets are extended through the tubing.

The gold anodized eyelets are inserted through the drill holes with the flanges inside the tubing, barrels directed outwards, through the holes in the magnets, and the eyelets crimped or closed to bond the magnets to the surface of the tubing. An adhesive suitable for bonding, sealing, or smoothing any discontinuities between the gold surface of the bar magnets, eyelets, and tubing is Loctite Hysol Cool Melt®, which melts at 250 degrees Fahrenheit, well below the Curie temperature of neodymium iron boron magnets. Tubing materials approved for medical use by the Food and Drug Administration suitable for such use are Polymer Technology Group, Incorporated Bionate® polycarbonate based polyurethane copolymer of durometer D-scale 55 and silicone-urethane copolymers. It is considered obvious that the attracted and attracting parts, herein magnets and ferromagnetic pieces, such as by implanting magnetized miniballs and using biocompatibly encapsulated soft iron bars on the stent-jacket, could be reversed in position to obtain a similar or identical result.

Expansion inserts are shown in FIGS. 6-8. When an expansion insert is to be bonded to the base tube, portions of the surface of the base-tube to which the insert is to be bonded are scored with surface undercutting for more secure adhesion. Stent-jackets and articulated stent jackets placed in locations that pose conditions with inordinate potential to displace these have end-tethers as described below as an additional precaution against displacement.

### Fully Absorbable Stent-Jackets

Placing a magnetless nonabsorbable stent-jacket for a temporary time, for example, to circumvascularly deliver medication over only a delimited or localized segment of a ductus, is discounted as requiring permural access to implant and then again to recover the stent, that is, as doubly invasive. However, a fully absorbable magnetless stent-jacket with the elasticity to comply with the autonomic action of the ductus eliminates the need for reentry at a later date. Stays that consist exclusively of medication and are completely absorbed also allow spot-sourcing of medication without the need for reentry. The drugs used with absorbable stent-jackets and absorbable medication stays would typically consist of steroids and antibiotics.

Inasmuch as the reduction or shrinking that usually follows treatment of an aneurysm is not associated with an eventual recovery of wall strength that would justify removal or reversal of the conventional therapeutic means employed, a stent graft when not surgically repaired, the irreversibility of current methods for the treatment of an aneurysm is probably not a disadvantage compared to the gradual dissolution of an absorbable magnetless stent-jacket. Permanent stein-jackets with circumsurfacial magnets and a special lining to prevent perforations by placement prior to initiating the ballistic implantation of permanent miniball implants is discussed below.

Where the implantation of fully absorbed medication or permanent radiation seed-miniballs is desirable at localized diseased loci or segments of a ductus that is not malacic as to require the prevention of perforations by a prepositioned double-wedge stent-jacket (below) but the overall length of the ductus having such lesions is so extensive that the number of permural entries required to place pure medication stays is considered unacceptable, implantation by transluminal ballistic means without the use of a stent jacket is considered. If a similar extent of ductus is malacic so that perforations are a certainty, and no alternative therapy is considered as effective as localized medication or radiation, then implantation with the aid of prepositioned magnetless stent-jackets incorporating rebound-directing double-wedge internal surfaces as described below may be justified.

The decision to use permanent or absorbable stent-jackets depends upon the prospects for the recovery of wall strength. A stent-jacket is placed prior to initiating discharge when the implants are to be permanent. Because it has no long-term function and requires separate entry to place and retrieve, an absorbable stent-jacket is not placed prior to initiating the discharge of absorbable implants to prevent the perforation of a malacic ductus. When the absorbable stent-jacket does not present the correct degree of elasticity, narrower and thus more compliant joints are incorporated.

Use an Absorbable Stent-Jacket with a Medicated Lining

The use an absorbable stent-jacket with a medicated lining may be indicated where the localized circumvascular release of medication may be therapeutic. Given the emerging involvement of the adventitia in different vascular disease processes confirmed and hypothesized, this is probable (see, for example, Xu, X., Lin, H., Lv, H., Zhang, M., and Zhang, Y. 2007. “Adventitial Lymphatic Vessels—An Important Role in Atherosclerosis,” Medical Hypotheses 69(6):1238-1241; Stern, N. and Marcus, Y. 2006. “Perivascular Fat: Innocent Bystander or Active Player in Vascular Disease?,” Journal of the Cardiometabolic Syndrome 1(2):115-120; Plekhanova, O. S., Stepanova, V. V., Ratner, E. I., Bobik, A., Tkachuk, V. A., and Parfyonova, Y. V. 2006. “Urokinase Plasminogen Activator in Injured Adventitia Increases the Number of Myofibroblasts and Augments Early Proliferation,” Journal of Vascular Research 43(5):437-446). Wilcox, J. N., Okamoto, E. I., Nakahara, K. I., and Vinten-Johansen, J. 2001. “Perivascular Responses After Angioplasty Which May Contribute to Postangioplasty Restenosis: A Role for Circulating Myofibroblast Precursors?,” Annals of the New York Academy of Sciences 947:68-92; Wilcox, J. N. and Scott, N. A. 1996. “Potential Role of the Adventitia in Arteritis and Atherosclerosis,” International Journal of Cardiology 54 Supplement:S21-35;

Pathology that results from balloon overinflation injury may not pertain to the angioplasty apparatus described herein (see, for example, Wallner, K., Sharifi, B. G., Shah, P. K., Noguchi, S., DeLeon, H., and Wilcox, J. N. 2001. “Adventitial Remodeling After Angioplasty is Associated with Expression of Tenascin mRNA by Adventitial Myofibroblasts,” Journal of the American College of Cardiology 37(2):655-661). For this reason, the internal surface of stent-jackets may be coated or impregnated with medication, which may be time-released, allowing a higher concentration at the affected location in support of the systemic medication essential in the treatment of systemic disorders. Panarteritis (polyarteritis nodosa, periarteritis nodosa, necrotizing arteritis), segmental arterial mediolysis, and similar diseases in the elderly who are too impaired to undergo radical surgery or the risk of a systemic overdose may prove palliable for a time.

Given that bacteria such as Chlamydia pneumoniae can play a role in arteritis and atherosclerosis (see, for example, Hu, C. L., Xiang, J. Z., Hu, F. F., and Huang, C. X. 2007. “Adventitial Inflammation: A Possible Pathogenic Link to the Instability of Atherosclerotic Plaque,” Medical Hypotheses 68(6):1262-1264.), medication can consist of or include antibiotics. The means for avoiding complete enclosure that would obstruct gas and other chemical exchange at the vessel outer surface is discussed above. When the adventitia is diseased or injured, the stent-jacket can be placed prior to discharge or stays consisting of medication implanted instead. Absorbable stein-jackets are formed by transfer molding of materials specified under the section below entitled Stent jacket expansion-insert materials with relatively short breakdown times.

### Stent-Jacket Linings for Containing or Preventing Perforations and for Reducing the Momentum and Misdirection of Rebound

Stent-jacket linings are devised to incorporate anti-migration, tissue infiltration or integration encouraging, and perforation preventing features in one and the same lining, a disproportionate requirement for one of these arising infrequently. As discussed below in the section entitled Sequence of Stent-jacket Placement and Implantation, when the stein-jacket is placed prior to initiating discharge, an especially resilient lining is needed. When in order to prevent a perforation where the risk is high, the stent is positioned prior to initiating discharge, the material properties and perforation value for a given ductus result from the combined properties of the interfaced adventitial and prosthetic lining backup layers as a physical system, which unpredictable with diseased tissue, necessitates preliminary in situ testing as described in the section below entitled In situ tissue puncture and penetration test.

When a localized source or sources of medication and/or radiation within the ductus wall would be advantageous over systemic or nonlocalized treatment alone, and exclusively transluminal approach is desired as less invasive, miniballs that consist purely of medication can be implanted ballistically. These are absorbed over time within the wall, and can not be released downstream when partially absorbed, as would an absorbable endoluminal stent. As is the case with prostatic brachytherapy, radiation spherules (seed miniballs, miniball seeds) are allowed to remain permanently, the thrombogenicity and invasiveness of a second procedure to remove these (through use of the recovery electromagnets) foregone. If the luminal wall is malacic and a perforation could result in serious injury, then treatment by a transluminal approach is precluded. Since to implant medication stays requires only permural access while ballistic implantation requires access both transluminally and permurally, the former is preferable.

When not lined by gauze, the internal surface of the stent-jacket, with or without the aid of a lining, is textured to avert migration, encourage tissue adhesion by ingrowth, provide more surface area, and retain more of a film coating that can consist of a ductus perforation sealant, antibiotic, or adhesive. While the overall thickness of a lining intended to assist in the control of rebounds is unaffected, the texture must meet the primary desiderata of reducing the momentum of and directing or steering rebounds, which calls for certain relations between the material and surface geometry of the lining. This is accomplished by means of a layer that consists of two sublayers at complementary angles as described below.

A nontemporary (nonabsorbable) stent-jacket is placed prior to initiating discharge in order to avert 1. Perforation (puncture, through-and-through penetration of the ductus wall), if perforation occurs, 2. The continued travel of a miniball following perforation by acting as a mechanical barrier or shield, or 3. the miniball from striking a vulnerable adjacent structure by protrusion at the point where the elastic adventitia protrudes upon outward displacement. Other reasons for placing the stent-jacket prior to discharge are to 4. Take advantage of the permanent magnets mounted to the outer surface of the stent-jacket in order to prevent the dislodging of an implanted miniball, especially when the need arises to pass an implanted miniball with the tractive recovery electromagnets energized, to 5. Take advantage of a tantalum coated stent-jacket as an imaging marker of high radiopacity to make locating the work site less difficult.

### Double-Wedge Stent-Jacket Bumper-Rebound Directing Linings

A stent-jacket that is placed prior to discharge in order to block a perforating miniball from continued travel should have a lining that either prevents or reduces the rebound from it. Rebound can be prevented by using a less resilient stent-jacket lining that allows the miniball to fully perforate and lodge (become embedded) within the lining. However, perforation results in the loss of refractive function that was the purpose in introducing the miniball in the first place. Perforation is advantageous only when it serves to avert rebound that would introduce a miniball into the lumen. The permanent magnets mounted to the outer surface of the stent-jacket exert retentive force to reduce rebound, but not before lending some additional force to expedite perforation; the need to balance magnet strength and stent-jacket liner resilience for moderating rebound is evident.

Rebound is preferably reduced to a usable level, meaning the miniball trajectory terminates at a usable depth within the wall of the ductus, by using a more resilient liner that absorbs the force of impact and thus causes the miniball to rebound with reduced momentum, regardless of whether the miniball perforated at the point where it has been stopped by contact with the lining. Controlling rebound makes it possible to place the miniball a short distance distad to the point of initial contact with the stent-jacket. Retractive function preserved, the latter is plainly preferable. The need for more than two layers, an inner to absorb and an outer to redirect, is discounted, as is the supposition that the intervening adhesive over the interface between the complementary wedge halves warrants special consideration in terms of dynamic consequences.

Normally, the elasticity intrinsic in the wall of the ductus allows the adventitia to stretch and retain the miniball. However, this property varies from instant to instant according to the phase in smooth muscle action in the wall of the ductus, such that elasticity is reduced during distension and recovered during relaxation. The more resilient is the internal layer of the stent-jacket and the less is the adventitia (or media), the greater will be the tendency toward both perforation and rebound. When the wall of the ductus is diseased—which is the only circumstance that would prompt treatment in the first place—the possible combinations in elasticity of the adventitia, whether such recommends the preliminary placement of a stent-jacket to prevent perforation and the risk of escape, and if so, the resilience of the stent-jacket lining suitable for reducing rebound to a degree that would land the miniball in a functional position become too variable for any solution but in situ testing as provided for below.

The ductus is tested without preliminary placement of a stent-jacket, and if perforated in testing, then with stent-jackets having linings that differ in resilience, the rebound imparted to the test rod translated into equivalent rebound of the miniball proposed for use. To minimize the number of perforations that result by testing, a stent jackethaving the properties presumed most effective is tested first. The rebound layer is compounded of two complementary tapered sublayers that interface along a common inclined junction, which is directed distally and outwards (away from the lumen, centrifugal).

### Specification of Cyanoacrylate Tissue Sealants and Bonding Agents

The citation of short or long-chain cyanoacrylate cement herein is subject to continued research with regard to histotoxicity and carcinogenicity. Some have described long-chain plastic glues such as butyl 2-cyanoacrylate and octyl-cyanoacrylate as more slowly degraded with some incision line encrustation or microcrystallization residue remaining but less histotoxic than short-chain glues such as methyl- and ethyl-cyanoacrylate (Toriumi, D. M., Raslan, W. F., Friedman, M. and Tardy, M. E. 1990. “Histotoxicity of Cyanoacrylate Tissue Adhesives. A Comparative Study,” Archives of Otolaryngology—Head and Neck Surgery 116(5):546-550; Levrier, O., Mekkaoui, C., Rolland, P. H., Murphy, K., Cabrol, P., Moulin, G., Bartoli, J. M., and Raybaud, C. 2003. “Efficacy and Low Vascular Toxicity of Embolization with Radical Versus Anionic Polymerization of n-butyl-2-cyanoacrylate (NBCA). An Experimental Study in the Swine,” Journal of Neuroradiology 30(2):95-102; Haber, G. B. 2004. “Tissue Glue for Pancreatic Fistula,” Gastrointestinal Endoscopy 59(4):535-537; Pachulski, R., Sabbour, H., Gupta, R., Adkins, D., Mirza, H., and Cone, J. 2005. “Cardiac Device Implant Wound Closure with 2-octyl Cyanoacrylate,” Journal of Interventional Cardiology 18(3):185-187.). Long-chain advocates do, however, reject isobutyl-2-cyanoacrylate (bucrylate) as a potential carcinogen (Vinters, H. V. Balil, K. A., Lundie, M. J. and Kaufmann, J. C. 1985. “The Histotoxicity of Cyanoacrylates,” Neuroradiology 27(4):279-291; Vinters, H. V., Debrun, G., Kaufmann, J. C., and Drake C. G. 1981. “Pathology of Arteriovenous Malformations Embolized with Isobutyl-2-cyanoacrylate (Bucrylate). Report of Two Cases,” Journal of Neurosurgery 55(5):819-825).

Other researchers, however, contradict the indictment of short-chain or absorbable cyanoacrylates as tissue adhesives stating that ethyl 2-cyanoacrylate is excreted from the body intact, with no mention of degradation or the liberation of formaldehyde as does Haber as cited in the preceding paragraph (see, for example, Kaplan, M., Oral, B., Rollas, S., Kut, M. S., and Demirtas, M. M. 2004. “Absorption of Ethyl 2-cyanoacrylate Tissue Adhesive,” European Journal of Drug Metabolism and Pharmacokinetics 29(2):77-81). Moreover, the same group has asserted that ethyl 2-cyanoacrylate can be used in vascular, myocardial and pulmonary surgery without concern for toxicity (Kaplan, M., Bozkurt, S., Kut, M. S., Kullu, S., and Demirtas, M. M. 2004. “Histopathological Effects of Ethyl 2-cyanoacrylate Tissue Adhesive Following Surgical Application: An Experimental Study,” European Journal of Cardio-thoracic Surgery 25(2):167-172). In small amounts used superficially, as when closing following the placement of subcutaneous clasp magnets, butyl 2-cyanoacrylate would appear to be relatively risk free (see, for example, Canonico, S., Campitiello, F., Santoriello, A., Canonico, R., Ciarleglio, F. A., and Russo, G. 2001. “Sutureless Skin Closure in Varicose Vein Surgery Preliminary Results,” Dermatologic Surgery 27(3):306-308), while for closing stay insertion tool adventitial or medial incisions, insistence upon a longer chain adhesive such as octyl-cyanoacrylate appears warranted until such time as a cyanoacrylate cement which is absorbable and nonencrusting becomes available (Seifman, B. D., Rubin, M. A., Williams, A. L., and Wolf, J. S. 2002. “Use of Absorbable Cyanoacrylate Glue to Repair an Open Cystotomy,” Journal of Urology 167(4):1872-1875).

Given contradictory information, the adhesive used to bond the lining to the internal surface of the base-tube, which is fully cured during manufacture and as cured involves little tissue contact, is based upon the materials of which the components to be bonded are made. Since octyl-cyanoacrylate and N-butyl-2-cyanoacrylate are generally agreed upon as posing the least risk, these, along with tissue sealants of different chemistry specified in the section below entitiled Sealant Cartridges and Sealants (Adhesives) are preferred for all applications described herein that involve sealing tissue rather than an extracorporeal assembly of components with cyanocrylate cements not brought into extended or intimate contact with tissue.

### Provisional Status of Preferences Concerning Cyanoacrylate Tissue Sealants

Small-chain cyanoacrylates absorbed but suspected to be toxic and long-chain cyanoacrylates considered less toxic but slowly and not fully absorbed, ongoing work on investigational atoxic absorbable surgical cyanoacrylate cements seeking atoxicity and absorption may have the effect of changing the preferences stated herein, which must be chosen from among existing cyanoacrylate cements (see, for example, Schenk, W. G. 3rd, Spotnitz, W. D., Burks, S. C., Lin, P. H., Bush, R. L., and Lumsden A. B. 2005. “Absorbable Cyanoacrylate as a Vascular Hemostatic Sealant: A Preliminary Trial,” American Surgeon 71(8):658-661; Lumsden, A. B. and Heyman, E. R. 2006. “Prospective Randomized Study Evaluating an Absorbable Cyanoacrylate for Use in Vascular Reconstructions,” Journal of Vascular Surgery 44(5):1002-1009; Ellman, P. I., Brett Reece, T., Maxey, T. S., Tache-Leon, C., Taylor, J. L., Spinosa, D. J., Pineros-Fernandez, A. C., Rodeheaver, G. T., and Kern, J. A. 2005. “Evaluation of An Absorbable Cyanoacrylate Adhesive as a Suture Line Sealant,” Journal of Surgical Research 125(2):161-167; Seifman, B. D., Rubin, M. A., Williams, A. L., and Wolf, J. S. 2002. “Use of Absorbable Cyanoacrylate Glue to Repair an Open Cystotomy,” Journal of Urology 167(4): 1872-1875).

### Stent-Jacket Lining Materials Suitable for Rebound-Directing Linings (Double Wedge Stent-Jacket Linings)

Stent-jackets for placement before initiating discharge in order to prevent a perforation from injuring adjacent anatomy must exclude any lining material that could prove injurious if implanted in the wall of the ductus through rebound. Silicone-urethane copolymers that resist degradation in resilience due to the cracking within the internal environment is preferred. The inner (adventitia contacting) layer of the double wedge rebound or bumper lining should exhibit a hardness on the order of 70 Shore durometer A scale at normal body temperature, while the outer layer is polyurethane of hardness 55 Shore durometer D scale (Yu, J-H., Dillard, D. A., and Lefebvre, D. R. 2001. “Pressure and Shear Stress Distributions of an Elastomer Constrained by a Cylinder of Finite Length,” International Journal of Solids and Structures 38(38-39):6839-6849). Permanently implantable polyurethane, preferably double bond polyurethane, available from Griffith Polymers Incorporated, can also be used. The inner layer may additionally require an anti-migration surface treatment (below).

Since the double-wedge liner is placed prior to initiating discharge, its internal surface must not become damaged by or allow the deposition onto the surface of the rebounding miniball of any substance that potentially harmful would then rebound to become embedded within the wall of the ductus. To reduce the risk of migration, the internal surface of the double-wedge base-tube insert can be embossed or textured but only to the extent that such relief or depression exercises little if any effect upon the direction of rebound. In manufacture, running rectangular blocks of each material precut to an overall length that is a sum of lengths of the stent-jacket to be lined are incrementally advanced through a grid of vertical and angled razor-edged stainless steel blades and then cleaved into segments of stent-jacket length by a vertically reciprocating shear having a razor-edged stainless steel blade.

The extent of displacement at the interface when the wedge halves are rolled following curing of the adhesive that is used to join the two together is too slight to require that the two be bonded only after having been rolled into a tube shape. The complementary halves must be bonded with an adhesive that upon curing will not fragment if subjected to relatively high density implantation by a multiple barrel radial discharge barrel-assembly in automatic discharge mode, such as biocompatibly plasticized 2-octylcyanoacrylate, or n-butyl cyanoacrylate cement. Biocompatible plasticizers include acetyl tri-n-butyl citrate, acetyl trihexyl citrate, butyl benzyl phthalate, dibutyl phthalate, dioctyl phthalate, n-butryl tri-n-hexyl citrate, and diethylene glycol dibenzoate (Greff, R. J. and Byram, M. M. 1998. “Cyanoacrylate Compositions Comprising an Antimicrobial Agent,” U.S. Pat. No. 5,783,177).

The base-tube is not opened flat to insert the double-wedge lining. Instead, the bonded double-wedge lining is manually rolled around a mandrel that is slightly larger in diameter than the internal diameter of the stent-jacket, and is held in place in mandrel surrounding relation with a tab of pressure-sensitive adhesive coated tape. The outer surface of the double-wedge is then coated with adhesive, and the base-tube with side-slit having been cut is opened just enough to place it over the lining so that the ends of the lining and base-tube line up. The tab of tape is immediately removed, and the plies are bonded together by closing a hand around it for a few seconds.

Before sealing in its individual sterile pack, or if not dispensed thus, then upon insertion, the individually sterile-packed stent-jacket is coated with an antibiotic or an antiseptic that is acceptable for intracorporeal use to avert infection (Visai, L., Rindi, S., Speziale, P., Petrini, P., Fare, S., and Tanzi, M. C. 2002. “In Vitro Interactions of Biomedical Polyurethanes with Macrophages and Bacterial Cells,” Journal of Biomaterials Applications 16(3):191-214) or degradation of the base-tube material by adherent macrophages (Jones, J. A., Dadsetan, M., Collier, T. O., Ebert, M., Stokes, K. S., Ward, R. S., Hiltner, P. A., and Anderson, J. M. 2004. “Macrophage Behavior on Surface-modified Polyurethanes,” Journal of Biomaterials Science, Polymer Edition 15(5):567-584).

### Stent-Jacket Anti-Migration Lining

When the risk of migration is a concern, as when retention in place by adjacent tissue and the force of magnetic attraction are thought inadequate and end-ties are not wanted, nonallergenic gossamer grade woven surgical gauze is bonded to the inner surface of the base tubing of the stent-jacket with an adhesive. To avoid brittleness that would result in interference with autonomic compliance, cause the gauze become stiff and therefore more prone to irritate the surface of the ductus, and gradually undo the bond between the gauze and the base-tube through an accumulation of microfractures, a surgically acceptable polyurethane adhesive that remains pliable after curing is used (see, for example, Marois, Y. and Guidoin, R. 2001. “Biocompatibility of Polyurethanes,” in Vermette, P., Griesser, H. J., Laroche, G. and Guidoin, R. (eds.), Biomedical Applications of Polyurethanes, Austin, Tex.: Landes Bioscience). A coating of the adhesive that is viscous and thin enough that it does not ooze to fill the holes in the gause up to the surface of the gauze to be placed against the adventitia is rolled onto the side of the gauze that is to interface with the internal surface of the base-tube. The delayed healing that has been observed with polyurethanes is of nugatory pertinence, since with the stent-jacket placed after implantation, stay insertion tool incision flaps would already have been bonded shut by the tissue bonding agent that is dispensed by the stay insertion tool, and any perforation that might have occurred during ballistic implantation would be tangential to the internal surface of the stent-jacket and already have sealed.

The external surface of the ductus is otherwise intact before the stent-jacket is placed. The gauze resists sidewise displacement, which is enhanced by tissue infiltration, and when the base-tube is provided with perforations for the purpose, does not interfere with exposure to intracavitary gas. When the potential consequences of a perforation prompt placing the stent jacket before the airgun is discharged, a rebound-directing or double-wedge lining, as described in the preceding section entitled Double-wedge Stent-jacket Bumper-rebound Directing Linings, is used. Anti-migration features in a double-wedge stent-jacket are incorporated into the internal surface of the inner layer by relief or embossing. Thus, when the stent-jacket is prepositioned, the materials with which it is lined would not be rebounded off of by a perforating miniball that would then reenter the wall of the ductus. Alternatively, since produced by extrusion, the internal surface of the tubing cannot be roll embossed, a stent-jacket with a slip resistant ribbed or ridged inner surface is produced by gluing a grid lining to the inner Surface of the stent-jacket. Stent-jacket base-tubes to which an expansion insert is to be added should eliminate or minimize lapping of the insert onto the internal surface.

When the base-tube of the stent-jacket is made, for example, of Bionate® thermoplastic polycarbonate urethane copolymer tubing and a rebound lining as described above is not to be inserted, for example, heavy gauze is glued to the inner surface of the base-tube with a long carbon chain cyanoacrylate cement, such as 2-octylcyanoacrylate, 4 Meta/MMA-TBB® 4-metacryloyloxyethyl trimellitate anhydride (4-META) and methylmetacrylate (MMA) as monomers and tri-n-butyl borane (TBB) as an initiator, Histoacril® 2-cyanobutylacrylate, and Bucrylate® isobutylcyanoacrylate. The use of a stent-jacket with a textured internal surface when, in order to prevent perforation and escape of a miniball, the stent-jacket must be placed prior to discharge implantation, should contemplate the effect of the internal pattern on the rebound and trapping of the miniballs. Such a texture can be impressed by heat stamping or micromilling into the inner polyurethane layer of a double wedge lining. Cyanoacrylate cement used to seal adventitial entry incisions produced by stay insertion (below) are absorbable and can be drug-releasing.

### Stent Jacket End-Tethers (End-Straps; Side-Straps; End-Tethers; End-Straps)

When the location for placement poses an inordinate potential for causing migration, end-tethers are added at either end of the stent jacket. End-tethers are outrigger stabilizers consisting of hook and loop straps with a backing of loosely braided multifilament spandex fabric specially woven for breathability. The straps are connected to the ends of the base-tube by non-absorbable soft braided multifilament suture, such as Neobond®, coated polyester, or Durasil® silk suture. Silk suture exhibits good elasticity but should be avoided in silk hypersensitive or allergic patients. The use of suture without flat straps about the ductus is discouraged as less secure, prone to cause injury by snagging or strangulation if single or few stranded, or demanding constricting multiple winds at the outer ends. The use of end-tethers may necessitate widening the entry wound and take additional time to place with the aid of forceps.

An ordinary punch and riveting hand tool allows inserting wide-head nonbioabsorbable biocompatible, such as tantalum, rivets to anchor suture end-tether suture ties, articulated stent-jacket connecting wires (below), or both to the outer surface of any stent-jacket base-tube. One end-tether suture tie is secured flush to the outer surface facing the operator near to either end of the base-tube by a wide-head rivet as are the connecting wires used with articulated stent-jackets. The end-tether straps must not be so narrow that too few hooks and loops are available to secure the straps in flat down or flush relation to the surface of the ductus or so that the edges of the straps could cut into the adventitia.

An articulated chain of stent-jackets positioned along a flexing ductus can be tethered at its end stent-jackets, in which case the same wide-head rivets secure both the end-tether ties and interjacket connecting wires. To prevent premature hook and loop connection during placement, a pressure-sensitive strip is peeled away from either the hooks or loops before the ductus is strapped about. As is the stent-jacket, the straps are fitted to the ductus during smooth muscle inaction for expansion with the pulse or peristalsis.

### Expansion Inserts for Time-Discrete Incremental Contraction of Stent Jackets

The object in temporarily expanding the stent-jacket is to continuously accommodate a substrate ductus which, anti-inflammatory medication notwithstanding, will subside from an initially enlarged condition over an extended period. Whether the substrate ductus or segment thereof is enlarged or ectatic due to temporary swelling or to inflammation resulting from chronic disease, injury associated with angioplasty, the direct result of injury associated with ballistic implantation, or some combination of these, a stent-jacket with expansion insert is devised to contract in circumference substantially in step with subsidence in the temporary swelling.

Since the insert acts against the restorative force of the base-tube, the capacity of the base-tube for further expansion in compliance with muscle action in the ductus in its swollen condition is reduced; that is, the use of an expansion insert partially uses and deducts from the overall expandability of the base-tube. Expansion inserts are bisected lengthwise as extend the base-tube in circumference while maintaining a side-slit or side-slot. The free edges of this extended side-slit or side-slot must be hard and smooth to allow the stent-jacket insertion tool (below) to be slid along the edges upon insertion. Any cut-outs in the stent-jacket that would be needed to clear anatomical side branches of the target ductus must be incorporated into the expansion insert.

Ideally, when an insert is layered to collapse decrementally, the time of dissolution of the adhesive used to bond each successive layer exhibits an absorption time reasonably synchronous with the material of its respective layer. Hyperplasia generally recommends treatment by means other than described herein; however, enlargement sequelary to such treatment may be susceptible to treatment by means of an external stent that is adaptable in diameter. Initial enlargement in a ductus can result from coexisting (comorbid, compounded, overlapping) conditions of which the period for the subsidence of each is separate in time so that the ductus recedes in steps. When the time for these successive regressions is reasonably predictable, the object is to time the contraction of the stent to the reduction schedule anticipated.

While to make the base-tube itself of a material that shrinks at a desired average rate would sustain superior circumvascular circularity during subsidence, the material would at the same time have to present the correct combination of flexibility, resilience or restorative force and shape memory, permanence, implantability, and certainty as to diameter when fully contracted, or ending diameter. The use of a temporary expansion insert based upon an absorbable material or layers of absorbable materials is indicated when 1. The condition is familiar affording a rate in subsidence that is substantially predictable, 2. Subsidence will occur over a relatively short interval, 3. The reduction in size to be expected is relatively small, and 4. The potential consequences of miscalculation would prove nugatory.

The dependability of the time of stent contraction (dissolution of the expansion insert, whether in stages), is increased by incorporating the agents of dissolution respective of each of its absorbable layers into the insert and when necessary, by relying upon polyester-based materials that are more predictable than gut as to time of absorption. Similarly, the absorbable adhesives used to bond expansion inserts to the free ends and outer surface of the stent-jacket side-slit and to bond different segments within the insert when present are better predictable as to dissolution time as well as tissue compatibility when consisting of a polyester-based synthetic such as specified below rather than a natural material, such as collagen, gelatin-resorcinol pentanedial, or gelatin-resorcinol ethanedial.

The expansion insert is bonded along the facing free edges of the base-tube side-slit with sufficient overlap onto the internal or external surface to assure adhesion. In manufacture, the insert is bonded at either end to the free facing edges and a portion of the outer surface of the stent-jacket base-tube to bridge the gap of the side-slit that it is used to expand. The base-tube-insert interface requires bond strength proportional to the length of the insert and restorative force of the base-tube material. Once the adhesive joining the insert at either end has cured, the insert is bisected. The length of the insert and thus the extent of expandability of the stein-jacket varies inversely as the flexibility of the base-tube. Since this flexibility results from the intrinsic resilience of the base-tube material, its thickness, and any perforations, for a given material, the thickness must be increased and any perforations reduced in area as the extent of expansion is increased. However, the flexibility of the base-tube must achieve good compliance with the smooth muscle function of the ductus.

Whether the insert is absorbable or destructible, overlap onto the internal surface must not be abruptly terminated as to present a edge or nosing that irritates by protruding into the substrate ductus. Neither should the overlap protrude centrifugally as to encroach upon neighboring tissue or extend onto the external surface as to displace magnets required upon reduction to the target circumference, which will compensate for a loss in shape memory in the base-tube while expanded when the material of which it is made is less resilient. When exposure to the surrounding agents of dissolution, for the most part water and proteolytic enzymes, is sufficient, absorbable materials such as dried sugars, syrups, absorbable suture polymers, or less often, collagen (gut), can be used, the persistence of gut increased as with chromic catgut through treatment with aldehyde solution and chromic salts (chromium trioxide), as is conventional with suture. Gut is restricted to conditions more tolerant to relative variability and less predictability in dissolution time as compared to synthetic materials.

The chemical environment of the expansion insert is, however, quite different than and more widely variable than that of suture, which unlike the expansion insert, remains in intimate contact with the surrounding tissue over its entire proportionately large surface area. The mechanical factors pertinent to suture also differ in that the edges of the tissue to be united remain coapted or are held in apposition despite swelling, and in that tensile strength, amenability to knotting, and capillarity that would permit entry by bacteria are important. The substrate layers for dissolution and any dissolution agents included with these are chosen on the basis of the environment of the side-slit. Water and native enzymes peculiar to the environment break down their respective substrate insert layer or layers. As uniform exposure of the different sides of given insert layer to the chemical environment cannot be depended upon, it will usually be preferable to incorporate agents of dissolution adjacent to the respective substrate layers. Agents of dissolution for absorbable layers include water and enzymes. Nonabsorbable layers are used only when the expansion period desired exceeds that dependably obtained with absorbable materials and justifies a second procedure to destroy the insert.

However, contact of the insert with the dissolution agents respective of each of its layers may not be present in the environment or not in close contact with the insert. Thus, while the rate in subsidence of the ductus may be predictable, that of the dissolution in the expansion insert, if predictable at all, will be less so, and will not often be amenable to accommodation using an expansion insert that consists of absorbable materials. For this reason, the agent or agents of dissolution are incorporated into the expansion insert for activation at the time of placement or at a later date.

When the necessary chemical environment to assure dissolution is not present and the period for expansion falls within the range that can be provided by either natural or synthetic absorbable materials, the agent or agents of dissolution are incorporated into the expansion insert itself. Except when a thermal angioplasty barrel-assembly is used to accelerate dissolution, this avoids the need for reentry at a later date, as would intracavitary (intraperitoneal, intrapericardial) fluid infusion such as used in ultrasonography with saline solution, where the fluid (water) would instead contain the solvent or enzyme for dissolution. When the period for subsidence exceeds that dependably available using absorbable materials, nonabsorbable materials must be used. Such requires a follow-up lithotripsy, and unless tissue continuity relates the source of excitation to the insert as target, intracavitary infusion is necessary for the lithotriptor to pulverize the stone insert or final layer thereof. Pulsed laser lithotripsy via laparoscopic entry should not be necessary.

When absorbable layers or linear segments having different dissolution times are required, the dissolution agent respective of each layer is applied in adjacent relation to its respective substrate layer or segment. Such dissolution agents generally consist of acid hydrolytic and proteolytic (collagenolytic) enzymes for gut (catgut, collagen) and water to hydrolyze ester bond based polymers. Physiologically exposed polyglycolide is also broken down by enzymes that exhibit esterase activity. With either gut or polymers, a hydrogel may be used for the release of water to activate chemically constituent or mechanically included (enclosed, embedded) proteolytic enzymes or to nonenzymatically hydrolyze the ester bonds of alpha-polyester polymers.

A water releasing hydrogel adhesive or layers thereof interleaved among absorbable layers may be used to bond layers of water-degraded materials where each moving toward the surface of the stent-jacket has a progressively longer degradation time. The adhesive hydrogel may additionally release medication (Roorda, W. E., Bodde, H. E., de Boer, A. G., Bouwstra, J. A., and Junginger, H. E. 1986. “Synthetic Hydrogels as Drug Delivery Systems,” Pharmacy World and Science [Pharmaceutisch Weekblad. Scientific Edition] 8(3):165-189). By embeddment or interleaving within collagen or absorbable polymers, medication can also be released upon dissolution of the absorbable materials.

FIGS. 6 and 7-8 show different expansion inserts, respectively short and long, where both are equally divided between the opposing edges of a stent-jacket side-slit. Illustrative, more materials and layers are shown than is likely to be necessary in most real expansion inserts, which would have to clear postoperative inflammation and then contract in a single step. Thus, the longer expansion insert of FIG. 8 would typically consist of but a single polyester such as polylactic acid interleaved with hydrogel water-releasing segments, bisected at the midline, and fastened to the free edges of the side-slit with absorbable ethyl 2-cyanoacrylate cement (above). The smaller expansion insert shown in FIG. 6 is intended for finer control over a longer period.

The materials comprising either side of an expansion insert can consist of different materials making the insert asymmetrical and extending its overall dissolution time. In FIG. 6, the smaller insert is bilaterially symmetrical, free-edge segments X crystalline sucrose or rock candy inclined so as to use uneven distribution of the compressive force exerted by the bare-tube to accelerate break-down, layers X a water-releasing absorbable hydrogel adhesive, segments X polyglycolic acid, layers X a water-releasing absorbable hydrogel adhesive, segments X polylactic acid, of which the ester bonds hydrolyze somewhat more slowly than polyglycolic acid, segments X a water-releasing absorbable hydrogel adhesive, segments X polycaprolactone, slower still, and layers X a water and proteolytic enzyme releasing hydrogel adhesive.

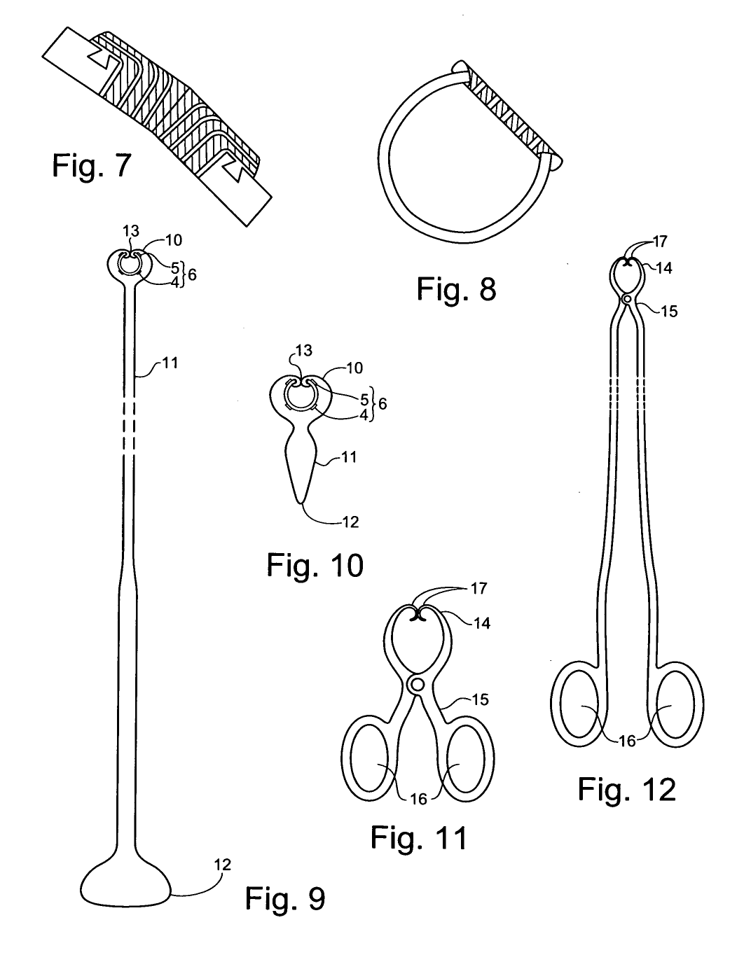

For better adhesion and positional stability, the layers are lapped around from the cut ends of the side-slit and onto the external surface of the stent-jacket. Lapping onto the internal surface is avoided to preclude abrasive irritation to the substrate ductus. In FIGS. 7 and 8, the longer insert is bilaterally symmetrical with free-end segments X of polyglycolic acid, backed by layers X of hydrogel adhesive, then segments X of glycolic-lactide copolymer, layers of hydrogel adhesive, polycaprolactone, and layers of hydrogel adhesive.

Expansion inserts consisting of absorbable materials are used in the expectation that dissolution will be spontaneous and thus eliminate the need for a second procedure at a later date to effect contraction of the stent-jacket. When the degree of unpredictability in the rate of insert dissolution to be expected is unacceptable, and especially when unanticipatable retardation in dissolution would positively interfere with subsidence and healing, acceleration in the absorption of an expansion insert consisting of absorbable materials is accomplished through exposure of the expansion insert to a dissolution-accelerating solution or ultrasonic cavitation as active means for exercising greater control over the time of stent-jacket contraction. The procedure and materials required for both of these follow-up procedures are described below.

When the insert must reliably last longer than were it absorbable, materials such as crystalline calcium oxalate monohydrate or dihydrate, calcium phosphate, and ammonium magnesium phosphate salts synthesized for minimal overlap in resistance to disintegration by lithotriptor-generated shock waves is used (for such distinctions in lithotriptor dosage essential to achieve the breaking up of different stones, see for example, Bouropoulos, N., Mouzakis, D. E., Bithelis, G., and Liatsikos, E. 2006. “Vickers Hardness Studies of Calcium Oxalate Monohydrate and Brushite Urinary Stones,” Journal of Endourology 20(1):59-63, and Johrde, L. G. and Cocks, F. H. 1985. “Fracture Strength Studies of Renal Calculi,” Journal of Materials Science Letters 4(10):1264-1265) are used to make the insert, and shock wave lithotripsy is used to break up the stone layers or segments of the insert. To constitute an expansion insert by superimposing layers of absorbable upon lithotriptor-destructible materials is considered obvious.

When nonabsorbable, the layers of the insert are prepared for selective destructability and when stone, need not chemically duplicate stones of endogenous origin. Differential resistance to ultrasonic disintegration is obtained by chemistry, dimensioning, and extent of gas entrapment Vakil, N. and Everbach, E. C. 1991. “Gas in Gallstones: Quantitative Determinations and Possible Effects on Fragmentation by Shock Waves,” Gastroenterology 101(6):1628-1634). To facilitate pulverization (disintegration, fragmentation), stone inserts or the layers thereof can be prebored or pre-lithotresed.

Lithotripsy used to initiate the dissolution of shorter term absorbable or to pulverize longer-term stony temporary materials in order to reduce stays or stent-jacket expansion inserts with subsidence in swelling of the substrate ductus and/or to initiate the release or time-release of medication from these or from medicated miniballs, cannot span an air gap and requires continuity of medium for transmission from the shockwave generator or source of excitation to the target; unless fluid-filled, the intervention of a body cavity will truncate transmission of the waves, which must ensonify the target. A post-implantation follow-up procedure is not essential for shorter-term absorbable materials, which can incorporate the means of their own dissolution when necessary, but ultrasonic, and possibly pulse laser lithotripsy as described below may be necessary.

The insert material must directly adhere or be bondable to the base-tube. If layered with dissolution time distinct materials, then each ply must be bondable to that subjacent to it. When to achieve the adhesion of an insert material to its respective substrate requires the use of an absorbable adhesive, the dissolution time of the adhesive is important according to whether it fully encloses the substrate layer from the environment. If the underlying material is inaccessible until the superjacent adhesive disintegrates, then not is no less important than that of the primary insert materials.

Contraction of the stent-jacket base-tube is due to the gradual erosion and loss in compressive strength of the insert material, which results in its breakdown and collapse. Depending upon the chemical environment, which can be significantly altered in disease, absorbable materials vary in rate of dissolution. When an extended period necessitates the use of an insert material or materials that ultrasonic agitation would not disintegrate, laser lithotripsy is used. Whether passive-absorbable or actively destroyed, the insert can be layered to allow contraction on a gradual or incremental basis.

Stent-jacket expansion inserts may be simple in consisting of a single layer or compound in consisting of several layers of different materials. Depending upon the chemistry and configuration of the material or materials of the insert and the medical condition of the patient, the rate in breakdown or erosion will vary whether attributable to absorption by enzymatic proteolysis (gut, collagen), other chemical alteration, liquid infiltration, and hydrolysis (synthetics). While absorbable suture must possess tensile rather than compressive strength, experience with suture indicates the variability in persistence in different thicknesses of absorbed implantable materials under various medical conditions.

In most instances, one ply or single layer Type A (plain, nonchromic) catgut material in the suitable thickness for any perforations contained will achieve the breakdown time required, with mild or light chromic catgut (Type B), medium chromic catgut (Type C), or extra or heavy chromic (Type D) of like conformation extending the breakdown period by about seven to ten additional days each in the order stated. When layers of different materials are superimposed, the vertical alignment as superimposed or offset between the holes of the different layers, also affects the overall rate of dissolution for the layered expansion insert as a whole. That is, vertical alignment of the perforations in the different layers allows access along the inner edges of the perforations of the deeper layers to the environment, whereas staggering the perforations denies such access until the superjacent layer breaks down. The perforations can vary in size and shape.

Base-tube expandability is limited by the loss in roundness as expanded and circumference relative to that of the substrate ductus after contraction. When the rate of breakdown requires the interposition between the free facing edges of the base-tube of more than a single layer of absorbable material, layers of different absorbable materials are plied or bonded face to face with adhesives likewise related by differences in absorption time. The dissolution time of each layer is governed by its thickness and the size, number, and shape of any perforations.

As seen in FIG. 7, for adequate adhesion to the facing ends of the stent-jacket, the layers can be lapped over and additionally bonded onto the outer surface of the stent-jacket, not to cover an arcuate segment of the outer surface of the base-tube large enough to displace or require the elimination of a magnet or ferromagnetic attractant unless the latter is accommodated by a cut-out so that the expansion insert encircles the magnet. The percent expansion is limited not only by the fact that the open circumference of the stent-jacket proper must sufficiently conform to the ductus once subsided but by the fact that the magnets must not be extended to the insert.

With an enlarged ductus, as well as with a large elastic artery diagnosed as incipiently aneurysmal (for which a stent-jacket with hook and loop spandex straps without magnets can be used), the ductus should revert to the normal or more nearly normal diameter with healing over time. To not seal off the outer surface of the ductus from the surrounding cavity, the base-tube for such application is perforated and the inner surface of the stent-jacket textured. When the extent of swelling to result from implantation cannot be predicted with confidence, implantation must precede placement of the stent-jacket, which is the normal sequence.

Referring now FIG. 7, to create a self-contracting stent-jacket base-tube, layers of materials and mixtures of materials with known independent and intermingled dissolution times as solid are bonded in sequential layers according to relative persistence, that first to dissolve placed along one or both free edges of the side-slit. The base-tube then closes with the break down of each layer. Especially when high density implantation is used to uniformly and more widely distribute the magnetic traction in order to reduce the risk of pull-through or perforation of the ductus wall, which may lack normal hardness, swelling can result in occlusion, necessitating wider retraction. If the ductus is so malacic that expansion cannot be risked, then the use of stays (below) should be considered before replacement or bypass grafting.

A self-contracting extraluminal stent-jacket includes an insert along one or both free edges of the base-tube side-slit. The object is to so choose materials that will disintegrate or break down and allow the stent-jacket base-tube to contract at the same rate that the ductus heals and subsides. The materials of the expansion insert may be chosen to dissolve over a short period, or to be absorbed over a longer period, or to dissolve and be absorbed according to an anticipated schedule for healing that to accomplish the pacing or synchrony desired necessitates the interleaving of short and long term materials.

For quick contraction, substances that dissolve in serous fluid are used, whereas to contract over a longer period to allow for the slower regression in enlargement, aneurysmal distension, or healing, materials conventionally used to make absorbable suture are used. The dissolution times of both kinds of material are reduced by exposing more surface area or increased by use in a solid (continuous) block of greater thickness. The different materials and shapes can be sequenced and mixed to control the rate of stent-jacket contraction in accordance with the reduction in diameter anticipated.

Following treatment, vessels swollen by disease or as the result of reperfusion, or which were incipiently aneurysmal spontaneously resolve, or revert to, substantially normal dimensions over time. Moreover, all stent-jackets are elastic and thus self-adjust to reductions in ductus cross-sectional area or caliber over a limited range. This elimination of criticality is a significant advantage of avoiding the use of less flexible if inflexible endoluminal stents. However, based upon the absolute diameter, the range of such self-adjustment is sufficient for the normal expansion and contraction of the vessel or duct and deviation at the extremes of not to exceed about 20 percent. Beyond this deviation, had a stent-jacket to be chosen that was incapable of adapting in size as the enlargement receded, the likelihood is that an oversized one would be chosen.

That is, rather than to choose a stent jacket that would eventually be the correct size but which was initially too small and applied excessive constrictive force likely to interfere with healing and create complications, a stent-jacket would be chosen that is too large for the ductus once it had spontaneously resolved. Accordingly, stent-jackets must be provided that will progressively conform to the gauge (outer diameter) of the substrate structure on a parallel time frame. A significant factor in selecting short-term dissolution materials for incorporation into the temporary expansion absorbable insert is that the free water and enzymatic composition of serous fluid differs in disease (see, for example, Ben-Horin, S., Shinfeld, A., Kachel, E., Chemit, A., and Livneh, A. 2005. “The Composition of Normal Pericardial Fluid and Its Implications for Diagnosing Pericardial Effusions,” American Journal of Medicine 118(6):636-640).

Materials ordinarily used in absorbable suture vary in rate of dissolution among individuals, and more so in patients with disease. Adding to the unpredictability as to timing of subsidence, natural materials (collagen, usually bovine or sheep gut, whether treated with aldehyde solution and chromium trioxide to extend absorption time as ‘chromic catgut’), degrade by proteolytic enzyme breakdown and vary more in absorption time than do polyester based synthetics, which degrade by nonenzymatic hydrolysis of ester bonds. Synthetics are thus preferred as reducing overall unpredictability as to the mean dissolution time of the materials to constitute the stent-jacket expansion insert.

### Intracavitary Infusion of Fluid for Lithotriptor Dissolution of Long-Term Controlled Destruction-Time Controlled Expansion Inserts or a Final Stone Base-Tube Bonded Layer in Multilayered Expansion Inserts

The use of stone to allow the use of lithotripsy to exactly control the time for long-term stent-jacket contraction requires a followup procedure, demands assurance that irretrievable degradation fragments will be innocuous, and is undertaken only when necessary. Long-term temporary stays used without a stent-jacket are never made of stone. The use of electrohydraulic probes to accomplish the lithotripsy is inadmissible. When not interposed by a body cavity (potential space), lithotripsy coupling is conventional (percutaneous, extracorporeal). This will rarely be the case, vasa treated by the means herein described running just beneath visceral serosae, and thus requiring cavitary infusion of a shock wave propagation medium to achieve coupling.

Cavitary infusion is achieved either by injecting and afterwards aspirating away the coupling medium with water or by inserting a tightly rolled empty silicone cushion membrane through a local laparoscopic entry wound, injecting the intracavitary membrane with the medium, when so contained, preferably ultrasonography jelly (Cartledge, J. J., Cross, W. R., Lloyd, S. N., and Joyce, A. D. 2001. “The Efficacy of a Range of Contact Media as Coupling Agents in Extracorporeal Shockwave Lithotripsy,” British Journal of Urology International 88(4):321-324). Postlithotripsy, the medium is suctioned (aspirated) away and the cushion membrane if any retracted. Due to the risk of injury to the subjacent adventitia of shock wave destruction, stone inserts are not lapped to the internal surface of the stent-jacket base-tube in order to increase the surface area for adhesion.

Stent-Jacket Expansion-Insert Materials with Relatively Short Breakdown Times

Shorter-term materials in order of increased time for dissolution or loss of compressive strength include: 1. Glucose, dextran-40 and disodium (1-4)-2-deoxy-2-sulfoamino-β-D-glucopyranuronan (S-chitosan); 2. Poly(ethylene glycol), (PEG) and sugar (Wang, X., Yan, Y., Zhang, R., Fan, Y. W., Cui, F. Z., Feng, Q. L., and Liang, X. D. 2004. “Anastomosis of Small Arteries Using a Soluble Stent and Bioglue,” Journal of Bioactive and Compatible Polymers 19(5):409-419); and in order of increasing degradation 3. Polyglycolic acid; 4. Polylactic acid, and 5. Polycaprolactone (Benicewicz, B. C. and Hopper, P. K. 1990. “Polymers for Absorbable Surgical Sutures—Part I,” Journal of Bioactive and Compatible Polymers 1(5):453-472; W. J. Ciccone II, C. Motz, C. Bentley, and J. P. Tasto 2001. “Bioabsorbable Implants in Orthopaedics: New Developments and Clinical Applications,” Journal of the American Academy of Orthopedic Surgery 9(5): 280-288). Gut is not preferred as less predictable in its breakdown time.

Other stent-jacket expansion-insert materials based upon materials used in bioabsorbable suture, staples, endoluminal stents, and tissue engineering scaffolds include: 1. Polyglactin 910, which can be treated for more rapid breakdown; 2. Polydioxanone; 3. Poliglecaprone 25 (copolymer of glycolide and E-caprolactone); and 4. Woven and various blends of polyglycolic acid. Others such materials include Poly(lactide-co-glycolide) and Poly(glycolide/L-lactide) (see also Jeong, S. I.; Kim, S. H.; Kim, Y. H.; Jung, Y.; Kwon, J. H.; et al. 2004. “Biodegradable PLCL Scaffolds for Mechano-active Vascular Tissue Engineering,” Journal of Biomaterials Science-Polymer Edition 15(5):645-660; Grayson, A. C. R., Voskerician, G., Lynn. A., Anderson, J. M., Cima, M. J., et al. 2004. “Differential Degradation Rates in Vivo and in Vitro of Biocompatible Poly(lactic acid) and Poly(glycolic acid) Homo- and Co-polymers for a Polymeric Drug-delivery Microchip,” Journal of Biomaterials Science-Polymer Edition 15 (10): 1281-1304; and Lee, S. J., Lee, I. W., Lee, Y. M., Lee, H. B., and Khang, G. 2004. “Macroporous Biodegradable Natural/Synthetic Hybrid Scaffolds as Small Intestine Submucosa Impregnated Poly(D,L-lactide-co-glycolide) for Tissue-engineered Bone,” Journal of Biomaterials Science-Polymer Edition 15(8):1003-1017); polyhydroxybutarate valerate; polyorthoester; and polyethylenoxide/polybutylene terephthalate.

### Lithotriptor-Destructible Stone Stent Jacket Expansion-Inserts and Differentially Destructible Expansion-Insert Layers

Attaining dimensions such that individual stones can serve as a source of material for the machining of many stent-jacket expansion-inserts for eventual extracorporeal shock wave lithotripsy, gallstones (choleliths), which consist primarily either of cholesterol, or of bilirubin and calcium salts, and urinary tract (kidney, ureteric, and bladder) stones, which consist of calcium oxalate monohydrate (whewellite) or dihydrate (weddellite); magnesium ammonium phosphate hexahydrate (struvite), struvite-carbonate apatite, uric acid, calcium phosphate, or cystine, are routinely harvested by slaughter houses. Large urinary calculi such as staghorn calculi and large gallstones are also more economically and readily harvested at lithotomy than synthesized.

To allow differential lithotripsy and thus controlled reduction in stent-jacket expansion at intervals determined on the basis of diagnostic imaging of the residual enlargement of the ductus, harvested and synthesized calculi for use in multi-stone layered stent-jacket expansion-inserts, wherein the layers have different shock wave exposure breakdown times, can be chosen based upon composition (for differential breakdown times, see, for example, Pelander, W. M. and Kaufman, J. M. 1980. “Complications of Electrohydraulic Lithotresis,” Urology 16(2):155-157).

The greater resistance to lithotripsy of calcium oxalate monohydrate compared to dihydrate, for example, has long been recognized. Naturally formed calculi can be modified in mechanical properties through chemical treatment (see, for example, Johrde, L. G. and Cocks, F. H. 1986. “Effect of pH on the Microhardness of Renal Calculi,” Journal of Biomedical Materials Research 20(7):945-950). The persistence of a change in hardness following implantation of a calculus obtained from nature as the result of having been chemically treated warrants investigation.

The extracorporeal preparation (synthesis) of calculi (see, for example, Grases, F., Millan, A., and Conte, A. 1990. “Production of Calcium Oxalate Monohydrate, Dihydrate or Trihydrate,” Urological Research 18(1):17-20; Lepage, L. and Tawashi, R. 1982. “Growth and Characterization of Calcium Oxalate Dihydrate Crystals (Weddellite),” Journal of Pharmaceutical Sciences 71(9):1059-1062) can emulate the physiological conditions under which these are produced in the body (see, for example, Balaji, K. C. and Menon, M. 1997. “Mechanism of Stone Formation,” Urologic Clinics of North America 4(1):1-11; Mandel, N. 1996. “Mechanism of Stone Formation,” Seminars in Nephrology 16(5):364-374) or can introduce extracorporeal innovations such as, biocompatibility allowing, exercising control over the morphological development of calcium oxalate dihydrate crystals (see, for example, Zhang, D., Qi, L., Ma, J., and Cheng, H. 2002. “Morphological Control of Calcium Oxalate Dihydrate by a Double-Hydrophilic Block Copolymer,” Chemistry of Materials 14 (6):2450-2457).

### Intrinsic Shorter-Term Insert-to-Base-Tube and Layer-to-Layer Bonding Agents (Adhesives)

Polylactic acid (see, for example, Liu, L. M., Song, Y. M., Duan, H., Ding, Y. L., and Lu, B. 2006. “Effect of Polylactic Acid Glue in Preventing Epidural Scar Adhesion after Laminectomy in Rabbits,” Chinese Journal of Traumatology 9(3):146-151), polycaprolactone (see, for example, Jeans, L., Gilchrist, T., and Healy, D (2006. “An Evaluation of a Controlled Release Glass Wrap in the Repair of Peripheral Nerves,” Journal of Hand Surgery 31(1):47), and at certain temperatures and the use of suitable catalysts, these and other absorbable materials as specified above exhibit tackiness that makes these materials suitable as hot-melt adhesives, in some cases negating the need for an extrinsic glue (Stolt, M., Viljanmaa, M., Södergård, A., and Törmälä, P 2003. “Blends of Poly(-caprolactone-b-lactic acid) and Poly(lactic acid) for Hot-melt Applications,” Journal of Applied Polymer Science 91(1): 196-204; Leadbetter, K. J. and Shalaby, S. W. 1993. “Study of Interfacial Bonding in Fiber Reinforced Absorbable Composites,” Journal of Bioactive and Compatible Polymers 8(2): 132-141).

### Longer-Term Expansion Insert-to-Base-Tube and Layer-to-Layer Bonding Agents

Longer-term insert-to-base-tube and layer-to-layer bonding agents (adhesives, glues) to bond expansion inserts to persist over a longer period must break down into harmless degradation products. The immediate chemical environment of the material used to bond the expansion insert to the base-tube will be the primary determinant of the dissolution time, so that expansion inserts in less intimate contact with the environment are more likely to necessitate interventional measures due to persistence beyond the period desired. Long-term bonding agents are thus more suited to the bonding of stone inserts to be actively destroyed whenever desired. Persistence of adhesion in stone to base-tube polymer bonds is thus pertinent.

### Extrinsic Shorter-Term (Absorbable) to Longer-Term (Stone) Layer Bonding Agents (Adhesives)

Long carbon chain cyanoacrylate adhesives as short as butyl 2-cyanoacrylate (B2-CA) but not isobutyl-2-cyanoacrylate (bucrylate), which has been identified as a potential carcinogen (Vinters, H. V. Balil, K. A., Lundie, M. J. and Kaufmann, J. C. 1985. “The Histotoxicity of Cyanoacrylates,” Neuroradiology 27(4):279-291; Vinters, H. V., Debrun, G., Kaufmann, J. C., and Drake C. G. 1981. “Pathology of Arteriovenous Malformations Embolized with Isobutyl-2-cyanoacrylate (Bucrylate). Report of Two Cases,” Journal of Neurosurgery 55(5):819-825) can be used to bond one absorbable layer to another, whether in the expansion insert or at the insert to stent-jacket base-tube interface. Depending upon the specific materials, contact surface area and conformation, this kind of adhesive can also be used to bond an absorbable layer to a stone layer, a stone layer to another stone layer, or a stone layer to a stent-jacket base-tube. In addition to the bonding of an absorbable polymer to stone, which is used when the stent-jacket, and more particularly, a magnetless stent jacket, is to remain for a limited time, with an adhesive such as a long carbon chain cyanoacrylate adhesive, in certain cases, a layer of stone can be deposited onto such a polymer (see, for example, Yokoyama, Y., Oyane, A., and Ito, A. 2007. “Biomimetic Coating of an Apatite Layer on Poly(L-lactic Acid); Improvement of Adhesive Strength of the Coating,” Journal of Materials Science: Materials in Medicine 18(9): 1727-1734).

### Retardation in the Dissolution of Absorbable Materials

Whether used to make stent-jackets, stent-jacket expansion inserts, or stays, absorbable materials suitable for implantation can be modified to retard dissolution (see, for example, Maquet, V., Boccaccini, A. R., Pravata, L., Notingher, I., Jérôme, R. 2004. “Porous Poly(alpha-hydroxyacid)/Bioglass Composite Scaffolds for Bone Tissue Engineering. I: Preparation and In Vitro Characterisation,” Biomaterials 25(18):4185-4194; Maquet, V., Boccaccini, A. R., Pravata, L., Notingher, I., and Jérôme, R. 2003. “Preparation, Characterization, and In Vitro Degradation of Bioresorbable and Bioactive Composites Based on Bioglass-filled Polylactide Foams,” Journal of Biomedical Materials Research. Part A 66(2):335-346; Slivka, M. A. and Chu, C. C. 1997. “Fiber-matrix Interface Studies on Bioabsorbable Composite Materials for Internal Fixation of Bone Fractures. II. A New Method Using Laser Scanning Confocal Microscopy,” Journal of Biomedical Materials Research 37(3):353-362; Ibnabddjalil, M., Loh, I. H., Chu, C. C., Blumenthal, N., Alexander, H., and Turner, D. 1994. “Effect of Surface Plasma Treatment on the Chemical, Physical, Morphological, and Mechanical Properties of Totally Absorbable Bone Internal Fixation Devices,” Journal of Biomedical Materials Research 28(3):289-301; Assimos, D. G., Smith, C., Schaeffer, A. J., Carone, F. A., and Grayhack, J. T. 1984. “Efficacy of Polyglycolic Acid (PGA) Tubing Stents in Ureteroureterostomies,” Urological Research 12(6):291-293, and this in conjunction with the timed release of medication (see, for example, Tarcha, P. J. 1999. Polymers for Controlled Drug Delivery, Boca Raton, Fla.: Chemical Rubber Company Press division, Taylor & Francis. The immediate chemical environment of the material as used in stays or expansion inserts, for example, will be the primary determinant of the dissolution time. Expansion inserts, for example, in less intimate contact with the environment, are more likely to necessitate interventional measures due to persistence beyond the period desired.

### Articulated (Jointed) Stent-Jackets

When the segment of the ductus requiring treatment must flex so that the otherwise countermigratory clutching of separate stent-jackets would be overcome or would injure the substrate ductus, stent-jackets are linked end to end along its length to form an articulated chain. To resist proximating as well as distancing displacement among the component stent-jackets, articulation or connection is with nonmagnetic stainless steel wire in the manner of a jointed Palmaz-Schatz stent, except that here the wires are extravascular. The distance from the ends of adjacent base-tubes that a securing wide-head rivet is passed through the base-tube on either side depends upon the resilience and durability of the base-tube material. The ends of the wires are wrapped around the rivets of adjacent base-tube ends on the outside only. Whether longitudinally or circumferentially, the rivets for the articulation connection wires must be offset from the area of attachment of any expansion insert.

### Stent-Jacket Placement Tools (Stent-Jacket Applicators, Base-Tube Retractors, Side-Slit Retractors, Side-Slot Retractors, Side-Slit Expanders, Etc.)

Special tools such as those shown in FIGS. 9-12 are made of nonmagnetic metal or plastic and provided in a range of sizes to expand stent-jackets of different diameters and thicknesses for placement about (in surrounding relation to) vessels or ducts at different depths. The terms ‘tube retractor’ and ‘tube expander’ already in use for unrelated devices, terms such as ‘base-tube (slit) expander’ or ‘base-tube slit-edge retractor’ are necessary to distinguish such a stent-jacket base-tube expansion and placement device. Stent-jacket insertion tools must be nonmagnetic and made, for example, of austenitic stainless steel, such as 18-8, 304, or 316 amenable of hardening in smaller thicknesses.

Such tools are made to conform to the many dimensional requirements of base-tubes having different thicknesses and the need for handles of a length adequate to access deeper locations within the body. To allow passage through as small an incision as possible, the tools are long-handled and narrow but with burnished edges and in a gauge unlikely to injure anatomy that must be moved aside to reach to target ductus. While any number of complex linkages, ratchets, or pulleys could be incorporated into such a tube slit expansion device, any of which could further be made adjustable for use with stent-jackets over a range of sizes, for the least expense and greatest dependability, the use of simple tools is preferred. That stent-jacket insertion tools can be provided with handles suitable for use with a robot is considered obvious.

That shown in FIG. 10 is essentially a small spring-tongs or Finsen-type retractor for shallow placement, but with continuously flat and curved spatula-shaped blades 10 as in a Deaver retractor, which have been reversed or everted so that the major convex surfaces face outwards and with the distal ends of the scoop-shaped blades bent around to appear hooks 13 in profile for engaging the edges of the slit in the base-tube 5 with the minor convex surfaces of the hooking ends or tips 13 facing inwards towards one another or medially and thus reversely curved relative to the major portions of the blades 10. That shown in FIG. 9 is for deeper lying ducti. The tools are made of heavy gauge spring steel, which may be plated for corrosion resistance. The stent-jacket insertion tool shown in FIGS. 9 and 10 are not shown in an edge-on view because it is uniform in width proportional to its overall size, but always close to five millimeters.

To avoid being caught along when slid over the edges of the stent-jacket side-slit during application to the ductus, the edges of the insertion tool hooking ends are burnished round, and the hooking ends of smaller tools are coated with polytetrafluoroethylene. To prevent the snagging of neighboring anatomical structures, the outer edges are rounded as well. However, the incorporation of rollers to line the hook is not preferred as unnecessarily difficult to manufacture and costly. The conformation of hooking tips 13 must allow free disengagement from the free edges of the stent-jacket side-slit by a quick forward movement.

Nonmagnetic polytetrafluoroethyene coated hooking tips 13 retain the width of the blades 10 proximal or leading up to these. The handles or arms 11 are configured and united below at a junction 12, and since the tools would normally be made of one continuous piece of metal, this would usually be at least one but here shown as two sharp bends where the handles of the two sides join to enhance the outward springiness or restorative force that acts to expand the base-tube slit when not forcibly closed by squeezing the sides together. Finger rests, or widened portions along handles 11 for the thumb and index finger to pinch the arms together could be added, but have not, because at uniformly five millimeters in width for a tool of average length, the lever handles are wide enough that the tool will not rotate between the fingers, especially when covered with gloves of neoprene, synthetic polyisoprene latex, or latex.

To incorporate sliding finger rests that would allow taking advantage of the optimal moments of force in the surgical layout involved would be more a hindrance than aid, the operator intuitively moving to the position affording the best leverage. The working or blade portions distal to the crook in the handles can be joined to the straight portions of the handles by a rivet to create a swivel joint (not shown) that allows the angle between the straight portions and the working ends to be varied. The rivets are tightened enough to prevent the ends from unintentional rotation.

The stent-jacket placement tool shown in FIG. 11 is a scissors-tongs rather than a spring-tongs or tweezer-type configured embodiment for shallow placement that is similar to a Weitlaner type retractor just as the tweezers type is similar to a Finsen-type retractor, except that it is designed to pull in only two rather than three directions. The continuous flattened spoon or scoop-shaped retractor blades 14 are the same as those of the tongs-type retraction tool described immediately above. The handles 15, made as single parts, are reversely bent so that closing the finger holes 16 opens rather than closes the end hooks 17. The scissors-tongs can be urged either open or closed by means of torsion or leaf springs, as is well known in the scissors-making industry. The stent-jacket insertion tool shown in FIGS. 11 and 12 are not shown in an edge-on view because these are uniform in width proportional to overall size, but always close to five millimeters.

As with the tweezers type tool of FIGS. 9 and 10, to minimize the length of the incision necessary to access the vessel or duct from the outside for placement of the stent-jacket, the working ends distal to the handles can be joined to the handles by rivets to create swivel joints (not shown) that allow the angle between the handles and the hook-like tips 17 for engaging the free edges of the base-tube side-slit or slot to be varied when a probe is used to prod the base-tube to the angle required. The rivets are tightened enough to prevent the ends from other than intentional rotation. An embodiment configured as shown in FIG. 11 or as a pistol grip, for example, can be made with a ring that allows use of the index finger to adjust the angle of the working ends (not shown). The finger-ring retracts or pushes forward a rod connected to lever arms joined to the working ends distal to the swivel joints to change the working angle. The design of surgical retractors make such configurations familiar.

### Use of the Placement (Insertion) Hand Tool Applicator to Place the Stent-Jacket

In use, the stent-jacket is placed on the insertion tool so that the free edges of the stent-jacket are held within the recurved distal working hook tips XX of the tool and the side-slit is directed forward. In preliminary testing prior to application, depending upon its pliancy and length, the stent-jacket when pulled open at its midpoint will open sufficiently for placement along its entire length or only over the segment proximal to the hooking ends of the insertion tool. If expansion is limited in length, then the ease with which the hooking tips slide along the cut edges of the stent-jacket side-slit or side-slot with one end of the stent-jacket fixed in position is checked prior to insertion, and if, polytetrafluororoethylene coating notwithstanding, the tool resists being slid along the pulled apart edges of the side-slit, the working ends of the insertion tool are wetted with a lubricant (antiadherent, glidant) such as ACS Microslide®, Medtronic Enhance®, Bard Pro/Pel® or Hydro/Pel®, or Cordis SLX® just prior to entry.

The stent-jacket can be expanded (pulled open) for placement either prior to entry or upon reaching the target ductus. Depending upon the angle of approach, length, the need to clear neighboring structures, and resilience of the stent-jacket, the end-hooks of the insertion tool are used to pull open the stent-jacket at its center or close to one or the other of its ends, ideally, prior to introduction through the entry incision. The stent-jacket is introduced through a small incision (laparoscopic microincision) close to the location for placement. The width of the entry wound required depends upon the size of the stent-jacket, whether it is pulled open prior to entry, and whether it can be inserted parallel to the handles of the insertion tool.

Entry with the stent-jacket pulled open and parallel to the handles of the insertion tool allows passage through a smaller incision. Whether the stent-jacket has been expanded prior to insertion, entry can be parallel to surface of the body, with one side of the stent-jacket placed through the entry incision at a time. Since the insertion tool hook-tips are rotatable, a sterile wooden stick probe can be used along the path to the target ductus to angle the stent-jacket as necessary to avoid neighboring structures. When the side-slit is proximal, facing, parallel to the target ductus, and the stent-jacket can be sufficiently expanded to apply it without further effort, it is pushed forward to encircle the substrate ductus.

When the stent-jacket is longer or more pliant, use of the insertion tool can be made difficult by the tendency of the base-tube to contract over too short a length to the sides of the tool working tips. More often a probe held against the closed end or one of the bar magnets will be needed to stabilize or push the stent-jacket so that the insertion tool hook ends slide along the free edges of the base-tube side-slit to progressively pull the stent-jacket open along its entire length for encirclement of the target ductus. That is, the insertion tool is slid along the free edges of the stent-jacket side-slit, or the free edges of the stent-jacket side-slit are slid through the hook-ends of the insertion tool, or these movements are used in combination as the anatomy and forces involved dictate.

### Jacketing of a Ductus to Exert Tractive Force Upon a Neighboring Structure by Means of a Magnet-Wrap (Magnet Wrap-Surround, Magnet-Bandage, Magnet-Cuff)

A magnet-wrap is used to encircle a neighboring ductus to exert retractive force upon another diseased ductus which has been implanted with miniballs or stays or encircled by a clasp-jacket, which is described in the section to follow. If not sufficiently immobile, the structures paired thus are stabilized with suture to adjacent tissue. A magnet-wrap looks like a small wrist cuff, wrap, or bandage and is secured by hook and loop flaps to a neighboring structure when the use of a stent-jacket or subcutaneous or suprapleural magnets are less suitable and eccentricity of the pathology negates a need for the fully circumferential retraction of the lumen wall. With vasotonic and pulsatile or peristaltic, magnetic, bending, and gravitational forces involved, the use of a stent-jacket is normally preferred as affording the most compliant and least disruptive retraction of the substrate lumen wall radially outwards by the attraction of the miniballs to the magnets mounted to the outer surface of the stent-jacket.

The stent-jacket can retract any eccentric arc of a ductus wall or draw it outwards circumferentially, and concentric, is closest, allowing use of the smallest possible ferromagnetic elements. However, in some situations, alternative means for the support of the magnets will be necessary. Attachment that necessitates excessive dissection such as a substrate ductus that is unapproachable from the side without much cutting, and when traction that entirely surrounds the vessel or duct to be treated is not necessary, a magnet-wrap can be wrapped around a neighboring vessel or duct to attract the ferromagnetic stays implanted beneath the adventitia or magnets held to the external surface of the vessel or duct to be treated by means of a magnet-wrap. A magnet-wrap situates magnets to exert eccentric traction on a collapsed or lumen intruded arc of a neighboring ductus wall.

A collapsed ductus secured by connective tissue along one side as not to require retraction in that direction requires tractive force that is directed rather than circumvascular. For example, having longitudinal muscle and diffuse connective tissue peripherally rather than a harder outer layer, the esophagus, as can be true of any diseased malacic vessel, does not lend itself to implantation. Nevertheless, A magnet-wrap can be used, for example, to suspend miniballs implanted along dorsolateral lines of the collapsed trachea in a toy breed dog from ventrolateral magnets along complementary parallel lines of magnets held within a magnet jacket or jackets wrapped around the esophagus. Eccentric lesions in the vasculature are generally not so anchored along one side and would be tugged out of the normal position if eccentrically pulled at thus.

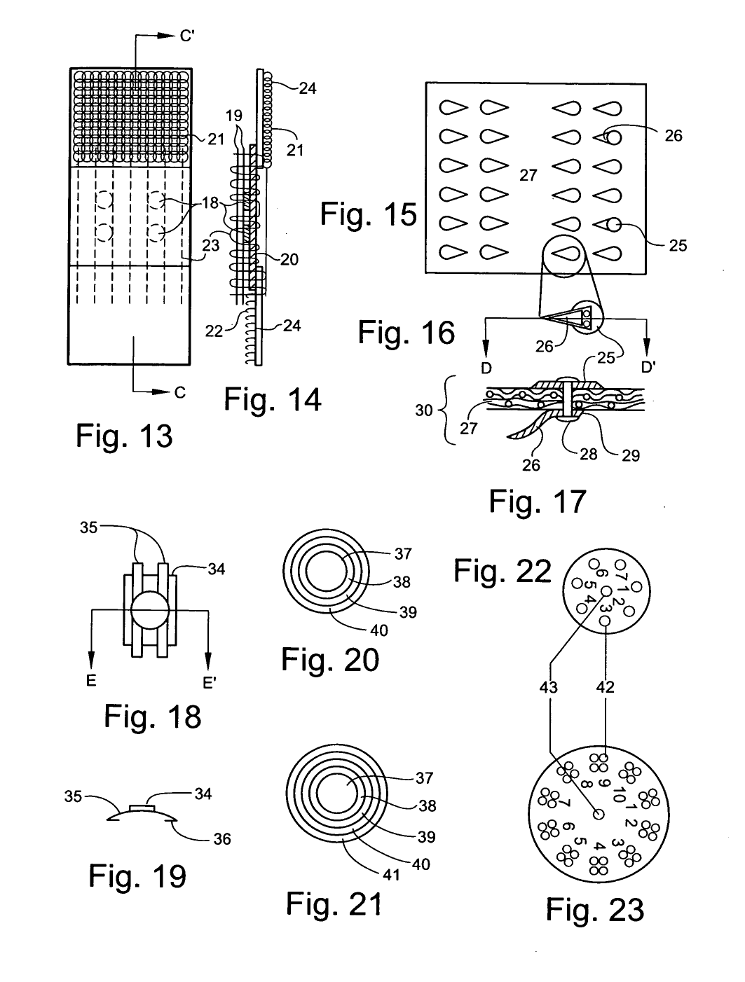

In this, a primary consideration is not to interfere with esophageal motility as might induce discomfort or dysphagia, or with an alternative ductus, not to interfere with its smooth muscle action whether peristaltic, pulsatile, or vasotonic. To this end, the backing bandage of the magnet-wrap must be sufficiently elastic to move with the outer surface of the substrate ductus without loosening, bunching or migrating. As shown in FIGS. 13 and 14, a magnet-wrap mounts magnets 18 that for minimum size, hence, least obtrusiveness or encroachment upon neighboring structures, are preferably made of high energy product sintered neodymium iron boron which have been encapsulated for biocompatibility as described below in the section on miniballs.

Four magnets 18 are shown only in an exemplary sense, the size, number per unit area, and shape of the magnets of sintered neodymium iron boron core for the maximum tractive force-to-magnet mass and size ratios all varying according to the application. Regardless of shape, magnets 18 are fixed in position by the same running stitch 23 which is reversed to fill in gaps of the first run for added strength, thus binding together the four plies of the magnet-wrap, two behind and two in front of the magnets. Stitching 23 preferably consists of nonabsorbable synthetic, strength retaining, nonallergic thread or suture, single strand, or monofilament, to discourage colonization, preferably made of polypropylene (Prolene®) or nylon (Ethilon®) suture. The two layers or plies to the rear (facing inside; toward the outer surface of the substrate support ductus) consisting of soft and elastic nonabsorbable, nonallergenic, and colonization-resistant fiber-based gossamer grade woven surgical gauze 19, preferably made of polypropylene.

The inner of the two outer layers consists of a loosely braided multifilament spandex fabric layer 20 that is specially woven for breathability. The outer layer consists of a breathable biocompatible fabric, such as an open nylon webbing. The inner surface of the outer layer, which serves to wrap around the ductus and secure the magnet-wrap on the side opposite the magnets, is divided to omit the portion of the underlying spandex layer 20 that overlies magnets 18. The portions of the outer layer divided thus are stitched against the outer surface of and toward the ends of spandex (elastane, Lycra®, Linel®, Elaspan® and Dorlastan®) layer 20. Small loops 21 on the outside (facing away from the outer surface of the substrate support ductus) and hooks 22 mounted on backing 24, usually made of nylon (polyamide, Zytel®) on the inside (facing toward the outer surface of the substrate support ductus) allow the end-straps or the two portions of the outer layer to be wrapped entirely around and securely fastened behind the substrate ductus. A silicone or wax coated paper release strip (not shown) is applied by means of a thin layer of an adhesive with low bond strength, such as corn starch, to either the hooks or loops to prevent unintended fastening during placement.

### Use of a Magnet-Wrap

The dimensions of magnet-wraps vary with the size of the ductus to be encircled. For most blood vessels, magnet-wraps are fifteen millimeters in wrap around length and provided in two millimeter increments and generally mount bar magnets that exert a tractive force proportional to their dimensions. The width of the magnet-wrap is determined by the length of the segment of the substrate ductus to be encircled. The magnet-wrap is selected according to the circumference and length of the substrate support ductus to exert the minimum tractive force sufficient to attract the miniballs over the gap present. The tightly rolled magnet-wrap is inserted through a local incision or trocar portal. It is then unrolled and wrapped about the substrate structure only so taut that the elastic webbed surgical gauze 19, preferably made of polypropylene (Prolene®) is not impeded from compliance to the smooth muscle movements of the substrate vessel or duct and so that the magnets are directed toward the target implants.

Finally, the paper release strip is stripped away from the hooks or loops, and end-flaps 20 are wrapped around the underlying substrate structure securing the magnet-wrap in place, the webbed texture of the gauze also assisting to reduce the risk of migration. When necessary, adding end-tethers or end-ties of hook and loop backed by loosely braided multifilament spandex fabric specially woven for breathability elastomer strapping, or as described in the section on stent-jackets above, Durasil® suture (not shown), affords added resistance to migration. It is considered obvious that the attracted and attracting parts, herein magnets and ferromagnetic pieces, such as in any wrap device described herein, could be reversed in position to obtain a similar if not identical result.

Creation of an Artificial Tough Outer Layer or Synthetic Adventitia about a Ductus by Means of a Clasp-Wrap (Clasp Wrap-Surround, Clasp-Jacket, Clasp-Bandage)

Clasp-wraps are bandages or wrap-surrounds that are used to wrap around a collapsed or stenotic ductus for use with a circumvascular stent-jacket. An acceptable clasp-wrap must not arouse adverse tissue reactions or disintegrate, and must not interfere with autonomic function. Such requires that it be sufficiently elastic to move with the surface during intrinsic and gross movement, and remain permeable to gas and moisture. The use of clasp-wraps and magnet-wraps is subject to the results of the adventitia-media delamination test described below under the section entitled Endoluminal Pretest for Adventitia or Media Delamination. As is true of stent-jackets and stays, a clasp-wrap has as one basic object compliance with the intrinsic movement in the wall of the vessel or duct. A clasp-wrap can be used with a stent-jacket to exert tractive force either eccentrically or entirely around the ductus with equal or different tractive forces over different arcs.

Retracted by means of a distant rather than encircling source of magnetic attraction, such as a magnet-jacket or a subcutaneous or suprapleural clasp magnet or by a plurality or some combination of these, the attractive field is angularly limited and therefore eccentric. When the ductus is malacic (soft, weak), miniballs and stays with too little contact area to adequately distribute or divide the force of attraction are likely to pull through. When the malacia or delamination is superficial (peripheral, adventitial), the use of a conventional (endoluminal) stent (because it is medial or deep to the diseased outer layers) is indicated. When the diseased tissue is more medial (deeper in relation to the outside surface), an endoluminal stent should be avoided. A diseased ductus that is not implantable without perforation, pull-through, or delamination using less densely spaced apart implants can sometimes be grasped about through use of a clasp-wrap, which more evenly distributes the tractive force and thus is better able to resist detachment.

Such use is especially appropriate where the ductus wall will remain in need of support after healing with the weakened condition having subsided, where the use of a wrap-around graft of stronger tissue would demand much time to heal, and an anastomotic graft would pose a risk of leakage as well as take time to heal. More specifically, to avoid a lengthy preliminary interval for healing before a second procedure to implant miniballs or stays can be performed that the urgency of the condition may not allow, such a synthetic exogenous patch material is preferable to even an autogenous graft. Furthermore, any donor tissue would have to exhibit the properties of strength and elasticity substantially unique to the type tissue of the ductus. Made entirely of nonallergenic synthetics, a stent-jacket likewise requires no antecedent procedure and poses no risk of rejection. Without a second operator to harvest the donor tissue, the use of synthetic materials saves even more operating time as well as avoids specifically harvesting trauma.

Approaches that would so strengthen the outer layers of a diseased ductus that miniball or stay implants could be used without the need for a clasp-jacket require further development. These include a bioinert penetrating and hardening resin for wetting the outer surface of the ductus that would have sufficient pliancy upon curing as not to interfere with autonomic motility, and the use of radio frequency treated collagen (see, for example, Shields, C. A., Schechter, D. A., Tetzlaff, P., Baily, A. L., Dycus, S., and Cosgriff, N. 2004. “Method for Creating Ideal Tissue Fusion in Soft-tissue Structures Using Radio Frequency (RF) Energy,” Surgical Technology International 13:49-55; Mohr, L. G. Jr. and Edwards, S. D. 1999. “Treating Aneurysms by Applying Hardening/Softening Agents to Hardenable/Softenable Substances,” U.S. Pat. No. 5,921,954 (expired Jul. 16, 2003 for nonpayment of maintenance fees). The many tissue fixatives developed for microscopy do not exhibit both the pliancy and tissue compatibility essential for the present purpose.

Provided the ductus is not too malacic, a clasp-wrap with a sufficient number of ferromagnetic clasps per unit area to undercut the outer layers of the ductus, especially when bonded to the surface of the ductus with a stretchable tissue adhesive and devised to allow tissue infiltration to compensate for the inevitable breakdown in the adhesive over time, can adhere longer than is allowed through the use of an adhesive alone. An adhesive that exhibits elasticity upon curing can achieve adequate broadness of adhesion, but with bonding limited to the surface, loses its grip as soon as the tissue to which it is applied is replaced through normal turnover and in the adverse chemical milieu inside the body, is likely to break down over time.

As shown in FIG. 15, which shows the internal surface of a clasp-wrap, FIG. 16, which shows a clasp in detail, and FIG. 17, which shows a clasp in section, mechanical fastening is accomplished through the use of clasps 30, each with a single very sharp prong tip 26 directed downward at an angle for undercutting the more superficial layers of the substrate ductus. In overall conformation, the prong is similar to those of an athletic bandage clasp but narrower. The clasps alone are used to retain the clasp-wrap against the outer surface of the ductus; alternatively, the clasp-wrap, to include the prongs, can be coated entirely with an adhesive, preferably one that exhibits elasticity upon curing, or partially with an adhesive and partially with phosphorylcholine to reduce tissue reaction, or provided with hook and loop extension at either side to assist in retention. Clasp-wraps generally do not require supplementary stabilization by means of side-tethers as might stent-jackets.

Synthetic and cleaned of any polymerization or environmental contaminants, spandex elastomer, which is a block copolymer of polyurethane segments alternating with segments of polyethylene glycol, is non-sensitizing and nonallergenic. As shown in FIGS. 15-17, the clasp-jacket is made of layers of spandex or elastane 24. Spandex is sold under the trade names of Lycra®, Linel®, Elaspan® and Dorlastan®. Such material is less susceptible to microfracture and brittleness over time than is natural rubber, and is thus able to resist the chemical insults associated with circumvascular placement for a long time. Spandex effectively does not disintegrate in circumvascular placement. It is considered obvious that the attracted and attracting parts, herein magnets and ferromagnetic pieces, such as in any wrap device described herein, could be reversed in position to obtain a similar if not the same result.

As shown in FIG. 15, formations of individual clasps are mounted on a woven stretchable backing 27 of spandex, which allows air to reach the surface of the substrate ductus. Backing 27 can also be made of plies of spandex alternating with plies of surgical gauze. Alternatively, the inner surface of the miniball-jacket can be coated with a highly tacky pressure-sensitive hypoallergenic adhesive consisting of a mixture of synthetic rubber, which has high initial bond strength that tends to degrade over time, and pressure sensitive acrylic emulsion polymer adhesive 30, which tends to increase in bond strength over time.

Sufficient breathability may necessitate introducing small perforations through this backing. For such an adhesive to bond well may require that the outer surface of the ductus be dried with a blow dryer or by swabbing with an absorbent cloth. Clasps with mutually facing prongs can be arranged in different formations, to include placing all facing in one direction as a group on one side of the internal surface with those facing in the opposite direction on the opposite side of the internal surface, or in rows wherein the prongs of adjacent clasps face one another in pairs. As shown in FIGS. 16 and 17, an individual ferromagnetic clasp consists of a single prong 26 bent when die-cut to be continuous with a small stoop-like tab containing a hole.

The dimensions of the stoop-tab are determined by the properties of the spandex (elastane) or other stretchable and breathable clasp backing material used 27, being made large the softer, more pliant so that the clasp is overly inclined under the tractive force of the magnets, and more subject to being torn is the intervening material. Rivet 28 is passed through this hole, through the intervening spandex sheeting and a flange cover-plate on the outer surface. Tab-stoop of prong 26, flange cover-plate 25, and rivet 28 thus cooperate to clamp spandex layer 27 and fix prong 26 in position.

The prongs, which depending upon the condition of the ductus are chosen in length to penetrate the adventitia or media (but not perforate into the lumen), are textured and fenestrated or punched to encourage tissue infiltration and are encapsulated for bioinertness as specified in the sections on miniballs and stays. Flange cover-plate 25, to include fenestrae and bends, and prongs 26 are die-cut, the angle of the bends varying with the overall dimensions according to the type substrate ductus and depth of penetration desired. Whereas a magnet-wrap need not firmly adhere to the outer surface of the neighboring ductus it mantles about, a clasp-wrap must adhere to the outer surface of its substrate ductus immediately upon placement despite magnetic traction on the clasps. To prevent irritation to the substrate ductus, all surfaces of the clasps facing inward, and to prevent irritation to neighboring tissue, all surfaces of the clasps facing outward, must be flat and smooth.

### Use of a Clasp-Jacket

The clasp-wrap is inserted through a small incision in the body wall while still tightly rolled, and an adhesive if any lightly painted onto the fascia cleaned surface of the ductus. The clasp-wrap is then positioned perpendicularly to the ductus, stretched only enough to allow the prongs to fully engage the outer layers of the ductus without perforating into the lumen or restraining the intrinsic motility, then pushed down gently with a fingertip to assure good contact and adhesion. Provided the ductus is motile upon application, the use following the clearing away of diffuse fascia of a long-chain methacrylate cement in a light film to bond the clasp-jacket to the outer surface of the ductus need little impede stretching of backing 27 nor smooth muscle motility; the stretching of backing 27 notwithstanding, adhesion generally succeeds at a sufficient number of points to maintain the bond until such time as tissue integration of the prongs takes over adhesion when this is eventually lost due to tissue replacement. In some instances, the methacrylate is applied with a multi-dot applicator for improved spandex stretch compliance.

### Alternative Methods for Achieving Adhesion to the Outer Surface of the Ductus

A malacic ductus may be unsuited to treatment by ballistic means, and a severely malacic or otherwise weakened ductus will be untreatable by any of the means and methods described herein with the possible exception of widely configured unretracted versions of the nonferromagnetic stays described below, all of the other means necessitating some resistance to tractive force applied from without. If endoluminal stenting must also be discounted, then a bypass graft is indicated. When malacia would not allow ballistic implantation but is not so severe or expected to progress to the degree that the wall of the ductus must not be grasped and retained under tension sufficient to maintain patency, then various nonballistic or stay methods and means to be described can be applied.

### Injectable Magnetic Fluid (Ferrofluid)

Existing injectable magnetic fluids are meant for temporary use to assist in manipulation during delicate ophthalmic procedures (Dailey, J. P. Phillips, J. P., Li, C.; and Riffle, J. S. 1999. “Synthesis of Silicone Magnetic Fluid for Use in Eye Surgery, Journal of Magnetism and Magnetic Materials 194(1):140-148(9); and U.S. Pat. Nos. 6,464,968, 654,636, and 6,612,311). Quickly dissipated, such means are inapplicable for the present purposes. At the time of filing, a bioinert and therefore permanent injectable magnetic fluid was not yet available.

Arcuate Stent-Stays (Stays, Stent-Ribs, Ribs) for Use with Stent-Jackets

### Structure

Arcuate stays are nonballistic implants for insertion into the walls of a collapsed or stenotic ductus some of which are used with a circumvascular stent-jacket or more distant magnet-wrap, subcutaneous, or suprapleural clasp magnets, and some of which are nonmagnetic. Some eccentric or radially asymmetrical conditions are correctible ferromagnetically whether implanted ballistically or applied nonballistically with clasp-wraps or stays while others do not warrant ballistic implantation or magnetic traction. The latter can be corrected with stays made of plain plastic, nonmagnetic use being peculiar to nonmagnetic stays. As is true with magnet and clasp-wraps, when the ductus to be treated is extensively attached by connective tissue, circumferential implantation may have to be intraluminal or ballistic. Stays are usually textured and/or perforated for added stabilization by tissue infiltration.

Stays are mildly curved (bowed, cambered) strips or bands made of a nonmagnetic material, such as thin and flexible stainless steel or polyester, that is hard enough to allow a leading edge to incise through the external surface of the ductus. When ferromagnetic, a soft iron disk is chemically isolated as embedded at the center of the stay and so that the force exerted on the disk will not cause the stay to rotate injuring the lumen wall. As can miniballs, and clasp-wraps, ferromagnetic stays can be used with stent-jackets or magnet-wraps. Whether implantation is of spherules ballistically or stays manually, the stent-jacket employed is unaffected, the content herein unified in this regard. Stays can be coated as described for miniballs, either of which may also be surface coated with an antibiotic polymer such as produced by Covalon Technologies, Mississauga, Ontario. in some instances, stays are applicable without a stent-jacket, hence, the need to include ferromagnetic material.

Once an arcuate stay-shaped core has been produced such as by pouring the liquid medication into a mold to dry, stays that consist entirely of medication or medication in successive investment of subjacent layers of medication can be produced by the same methods stated for the making of medication miniballs below, which include pan tumble coating, centrifugal extrusion, vibrational nozzle technique, and spray-drying. Similarly, a stent-stay may consist of a radiation source seed core and investing medication. Thus, the core of a stent-stay can be an absorbable or nonabsorbable polymer, a drug, or a radiation seed.

To minimize the risk of penetrating the lumen, stays are never to be used except 1. In sufficiently thick-walled ducti, 2. Unless insertion is constantly monitored for endothelial puncture with endoluminal endoscopy or endoluminal ultrasonography (intravascular untrasonography (IVUS), ultrasound probe sonography; ultrasound catheter probe-assisted endosonography; catheter probe assisted endoluminal ultrasonography), and 3. The stays are highly visible, whether with the aid of orally administered pronase (Sakai, N., Tatsuta, M. Iishi, H. and Nakaizumi, A. 2003. “Pre-medication with Pronase Reduces Artefacts During Endoscopic Ultrasonography,” Alimentary Pharmacology & Therapeutics 18 (3) 327-332). In some instances, plastic stays, coated with tantalum for high radiopacity and usually phosphorylcholine to reduce tissue reaction can maintain the patency of a ductus without the need for a stent-jacket.

With conditions unpredictable as to the eventuality much less the time of subsidence, the stays employed can still contain a ferromagnetic disk to allow the application of a stent-jacket at a later date if necessary; otherwise, both magnetic disk and stent-jacket are optional; however, stays should always be radiopaque, and the inclusion of ferromagnetic material is preferred as expediting recovery if necessary. With predictable subsidence, stays can be absorbable, partially absorbable, or self-shrinking, and can release medication associated with the process of dissolution, as described below. The applications of stays can be completely unrelated to the use of magnet traction to retract a ductus wall outwards towards a stent-jacket or magnet-jacket. The stay and stay insertion tool can also be used to introduce stays that consist entirely of medication or radiation seeds in the form of stays into the wall of a ductus. Stays used to localize medication or radiation within the lumen wall can be absorbable, in which case, the same materials used in absorbable suture and most tissue engineering scaffolding are used, as described below.

The stays are curved for concentricity to the resting circumference of the ductus and, depending upon the condition to be treated, variable in proportional length to the diameter of the ductus to be implanted, but seldom longer than the radius of the ductus from the lumen center to the surface, or half of the outer diameter. Once an antecedent angioplasty, if any, has been completed, stay insertion is without transluminal component, access accomplished by local exposure. The external surface of the adventitia in contact with the internal surface of the stent-jacket, and the implanted stay closely subjacent thereto, the stay is unable to move in a manner as would cut the media. The camber of the stay is such that upon insertion along a tangent normal or perpendicular to the axis of the ductus with the convex side directed outwardly (radially), the stay remains substantially concentric to the ductus, that is, parallel to the ductus longitudinally and concentric to it perpendicularly, with bowing contrary to such bodily displacement slight at most. Substantial concentricity with the circumference of the ductus causes the stay to move with the rest of the lumen wall preventing its ends from incising the wall medially toward the lumen.

Once implanted, the risk for escape into the surrounding body cavity or tissue is small, and once the stent-jacket has been applied, stays are prevented by magnetic attraction from escaping into the lumen and by physical obstruction by the stent-jacket from perforating outward. While the risk for a stay to drop away from the ductus, much less become lost to view in the usually moist environment strongly adherent for it is slight to nonexistent, the stent-stay insertion tools to be described also serve as hand-held electromagnets that would allow a dropped stay to be recovered. Situated outside the lumen, no implant described herein should ever be heated by an alternating current-powered hand-held electromagnet as a noninvasive means for accomplishing followup thermal angioplasty to treat restenosis. To do so would burn extraluminal tissue. Shorter stays are meant to achieve lumen patency by attraction to the surrounding stent-jacket, while, depending upon the condition to be treated, longer stays without an embedded ferromagnetic disk at the center can be used to exert a patenting effect without the need for a circumvascular stent-jacket. To the extent that it does not result in a degree of flexibility that allows deflective bending during entry incision, the stays are made thinner toward the ends to be flexible for compliance with the contractile action of the ductus.

The stays are inserted through the external surface of the ductus to undercut the adventitia with a special insertion tool to be described and are cold process physical vapor deposition or sputter-coated with tantalum for increased radiopacity. For improved tissue acceptance, a coating of phosphorylcholine or a polymeric blend thereof (see Lewis, A. L., Vick, T. A., Collias, A. C. M., Hughes, L. G., Palmer, R. R., Leppard, S. W., Furze, J. D., Taylor, A. S., and Stratford, P. W. 2001. “Phosphorylcholine-based Polymer Coatings for Stent Drug Delivery,” Journal of Materials Science: Materials in Medicine 12(10-12):865-870(6); Jones, S. A., Stratford, P. W., and Rimmer, S., assignors to Biocompatibles Limited, Uxbridge, England 2000. “Polymeric Blends with Zwitterionic Groups,” U.S. Pat. No. 6,150,432) is then applied to the tantalum and can also be added to the concentrated sugar solution used to bond and position the stays together in strips. The use of a specific formulation of phosphorylcholine is secondary to the requirement for noninterference with the tackiness essential to seal the adventitia entry slit or for the cohesion of the stays within a strip for insertion in the stay insertion tool (below).

Polyester affords arcuate shape-holding ability consistent with flexibility, a low coefficient of friction, implantability without concern for substituent toxicity in the event of degradation, and tantalum coatability. Other polymers usable are polyethylene terephtalate with or without glass fiber and polystyrene. If, as determined by the empirical probe-rod test described below, the susceptibility of the adventitia to delaminate over time is ascertained to occur at a tractive force that is less than that to be exerted by the stent-jacket, then the stay is inserted to a greater depth, that is, into the media, which is accomplished by applying slightly greater downward force upon the insertion tool, the action more clearly viewable with the aid of an attached endoscope, for example, as described below.

Due to the continuous replacement of connective tissue, the adventitial insertion incision adhesive-sealant automatically ejected with each stay by the insertion tool (of which little reaches the underside of the stay in any event) will not afford long term adhesion as would prevent adventitial or external elastic laminal delamination. Since the stent-jacket is chosen for the minimum effective attractive force, the choice of a stent-jacket having magnets that are so weak as to avert delamination will have been precluded. As shown in FIG. 70, while stays are a little wider at the center for increased surface area and thus more forceful retraction with reduced tendency for becoming displaced, these are still sufficiently straight-sided to assure linear feed through the insertion tool ejection slot and typically measure 2-3 millimeters in width by 4-5 millimeters in length, with rounded or blunted corners and honed edges at the ends. Symmetrical, the rear edge is configured for engagement by the v-notch at the driving forward edge of the highly flexible spring steel or polyester-ferromagnetic steel laminate insertion tool plunger-blade (below).

Molding soft iron disk XX in the center of the stay chemically isolates the disk. The stays flexible except at the center but incapable of rotation as would lacerate the tissue surrounding the edges and tips, surface texture or embossing can be molded into the stay to promote tissue infiltration for stronger positional fixedness once implanted, it. Texturing the surface also allows better temporary adhesion of the sugar used as a bonding agent between adjacent stays to fasten these together into a strip in which the position of each is stable for ejection. As shown in FIG. 68, stays are separably connected into strips for sequential ejection similarly to staples. The floor of the ejection slot and surrounding walls of the stay magazine serving to substantially position the stays for sequential ejection, the stays are weakly tacked face to face to allow insertion in the magazine as a clip of several at a time and to more exactly align and separate the stays XX, which bowed and thinner toward the ends than at the center must nevertheless be stacked at the correct angle for sequential concentric implantation and engagement by the plunger-blade XX along the rear edge.

The stays are tacked together into a unified strip by insertion into a mock magazine. A sterile concentrated solution of sugar is brushed along either side of the stays stacked in the magazine so that capillarity draws the solution into the recesses between adjacent stays formed by the thinner end portions. On drying, the strips are sealed in sterile packages with inflexible sides as prevent breaking the strips. Once conveniently placed within the insertion tool magazine in a strip rather than individually, the stays are correctly positioned, breaking of the sugar bonds by the downward compressive force of the magazine spring no longer mattering. For the number of stays in a strip, the residue that accumulates at the bottom of the stay insertion tool magazine is not sufficient to jam the tool. Almost all of the sugar that accumulates at the bottom of the stay insertion tool magazine is pushed out of the ejection slot by the next stay upon ejection, is swept to either side of the ductus entry incision, and falls away into the body cavity wherein it is harmless, trace amounts entering the ductus being likewise harmless.

The nuisance of inserting each individually into the insertion tool magazine notwithstanding, unused stays are best sterilized with ethylene oxide gas, peroxide plasma, or gamma radiation. It is obvious that the attracter-attractant relation could be reversed so that the soft iron or other ferromagnetic disks within the stays could be magnetized axially, in which case encapsulated soft iron or other nonmagnetized ferromagnetic material would be mounted in place of small bar magnets about the surface of the stent-jacket.

### Partially and Completely Absorbed Stent-Stays

Stent-stays that shrink without change in conformation are discounted as demanding constancy in mechanical properties while decreasing in size as would contracting stent-jacket base-tubes. Substantially the same alterations in conformation can be obtained through the use of partially absorbed stays. Stays can interleave material used for absorbable suture or staples (see Ravo, B., Rosales, C., Serino, F., and Castagneto, M. 1991. “The Use of Absorbable Staples for Construction of a Bladder Tube,” Surgery, Gynecology, and Obstetrics 173(1):29-32) with the agents of their dissolution, so that catgut (collagen) is interleaved with a proteolytic enzyme or a synthetic with a water releasing hydrogel, and completely or partially absorbed stays for maintaining patency during the course of a temporary or subsiding condition can embed or interleave medication that is liberated by absorption. The cyanoacrylate cement, such as ethyl 2-cyanoacrylate tissue adhesive or hydrogel (above), used to bond plies of an absorbable or partially absorbable stay can be absorbable, release medication, or both.

Anticlotting medication administered, it is feasible to initiate the absorption of a stay (or miniball, but not a stent-jacket expansion insert, which lies out of effective reach), its release of medication, or both in the bloodstream by thermal angioplasty with a barrel-assembly or thermal balloon catheter. Absorbable inserts implantable by means of the stay insertion tool can consist purely of medication compacted with or without an excipient base, medication applied to an absorbable base-stay, or absorbable material and medication particulates intermixed and compacted into a tablet of insertable shape. When not collaterally serving a lumen patenting or stenting function, such implants can be shorter than the shape of a patenting stay as described above. When the material of the tablet otherwise lacks sufficient hardness to provide a honed leading edge of penetration strength, a rim surround of absorbable material is used to provide this strength. Medicating and/or irradiating stays can be produced with the medication coated onto the stay, which is made of the same materials as specified in the section above entitled Stent jacket expansion-insert materials with relatively short breakdown times that are used to make absorbable suture for more immediate release whether timed, or the medication can be interleaved with or interspersed through the absorbable material for more extensive timed release.

### Conditions Recommending the Application of Stent-Stays

The use of stays is indicated when, whether due to injury by the antecedent angioplasty or pathology, the intima must or would best be avoided, or where the limited time predicted for continued adhesion of a clasp-wrap to the adventitia would fail to provide extended effectiveness as required. Stays can be used in combination with ballistic implantation for segments of the ductus over which ballistic implantation is contraindicated. Under these circumstances, stays afford a suitable expedient when only the accessible side requires treatment or when the ductus is accessible for encirclement without the need for excessive dissection or torsion to allow access to sides in abutment with adjacent tissue.

Stays are most useful when only the facing side of the ductus requires support. In this situation, stays will sometimes serve to support the ductus without the application of a stent jacket. With or without a stent-jacket, stays should be avoided where placement is near to the body surface without sufficient surrounding soft tissue to absorb an impact from without as would avert incision by the edges of the stay. However, endoluminal stents pose a danger with a direct blow. As with ballistically implanted miniballs, while neodymium ferrite magnets afford considerable energy products, retention of a stent-jacket must not depend upon magnetic attraction alone but be afforded sufficient circumference to reliably engage the ductus mechanically through the resilience of the tube base material with or without the aid of suture or hook and loop spandex elastomer strapping end-ties. Stays can be medicated or irradiative as discussed under the section on miniball implants above.

### Stent-Stay Insertion (Injection) Tools

### Structure

As shown in FIG. 64, with the exception of sealant (adhesive) air pump piston XX, which is connected to and moves with thumb plunger shaft XX, the parts of the stent-stay tool to the right of thumb plunger shaft XX remain stationary, whereas the parts of the tool to the left move up and down. To use the stay insertion tool, the ductus is accessed through a small incision, which can be held open by retractors or a trocar cannula. The size of the insertion tool necessarily gauged to the diameter of the ductus to be treated, the distal end of the insertion tool is typically 5 millimeters wide and 8 millimeters from front to back.

The insertion tool must minimally interfere with imaging equipment needed to confirm the concentricity of insertion. In the pusher-type embodiment described first, the adhesive delivery mechanism is in line with the stay magazine and the stay recall or retraction and recovery electromagnet is situated behind the tool. Placing both beneath the wrist of the operator resulting in minimal obstruction to vision and manipulation. Provided insertion results in substantial concentricity, even a suddenness and amplitude of pulse or peristaltic action that exerts considerable outward compressive force upon a lumen wall reduced in elasticity by disease will not cause the stay to incise toward the lumen. The insertion tool must therefore introduce the stays to be concentric to the ductus.

While a growing resistance posed by adhesive buildup will become apparent tactually, and the application of adhesive to each stay and proper sealing of each adventitia entry incision can be seen with the binocular telescopes and head-lamp when the tool is lifted aside from each stay insertion site, an endoscope allows proper operation of the tool to be confirmed without the need for removal. The material must also be strong enough that at typically 5 millimeter wide and 8 millimeter from front to back with sides 1.5 millimeter thick, the working end will not fracture or fall inside the body. In addition to providing transparency, the plastic body also serves to prevent interference with the onboard stay retraction or recall device to be described.

For these reasons, the tool is preferably made 18 centimeters or more in length and of transparent polyethylene terephthalate, polystyrene, high-density polyethylene, or acrylonitrile butadiene styrene (but not methyl methacrylate (acrylic), which is too brittle to preclude fracture at the small working or distal end), so that the parts will minimally interfere with the views from different angles of the work area. Two embodiments are provided, one, shown in FIG. 64, a pusher type or passive inserter that allows the force of insertion to be set by the restorative force of the plunger or plunger-slide return spring but force added by the operator if necessary, and a puller type or active inserter shown in FIG. 66, that allows the operator to control the force of insertion. Thumb, index, and middle finger rings allows the plunger in the pusher type to be pulled up as well as depressed.

Since the stay magazine must queue the stays for contact with the ductus at the same time that clearance must be allowed for the ductus itself, a rearrangement of the parts as would give better access to the bottom of the far side of the ductus is ruled out. Using the designs shown, rotation of the insertion tool is limited by the length of the cut-down incision normal to the ductus and the attachment of the far side. The stay insertion tool is applied to the ductus at bottom arcuate butt or sole XX, which ending in the back at butt or heel XX and the front at toe XX must be matched in diameter to the ductus. The surface of insertion tool butt XX is serrated from toe to heel or is covered with small dentate or round pillbox projections to stabilize the ductus and nonslidably engage the stay insertion tool to the surface of the ductus XX.

Fully circumferential access requires that the ductus be detachable over a sufficient segment and sufficiently torsional to allow otherwise inaccessible arcs to be implanted. However, attachment at the far side may serve to retract the rear wall of the lumen with only the proximate side requiring retraction by means of a partial stent-jacket. If not and far-side implantation is necessary, the far side will usually be implantable endoluminally by means of a barrel-assembly.

Referring now to FIG. 64, for maximum comfort, index and middle finger stops XX of the pusher-type insertion tool are mounted on a rotatable collar that can be angled or cocked 45 degrees clockwise by a right-handed operator or counterclockwise by a left-handed operator, and the adhesive-sealant XX and stay retraction electromagnet XX components are placed far enough down the central spring loaded plunger XX to afford hand clearance. For minimal interference with viewability, stay retraction electromagnet XX and its probe extension are placed at the back of the tool. Placing the stay retraction electromagnet XX high on the tool tends to keep it extracorporeal, reducing the chances that a longer incision will be needed for entry.

Placing the index and middle fingers under finger stops XX and using the thumb to depress thumb rest XX causes expansion spring-returned plunger XX, which slides through plunger sleeve XX as in a hypodermic syringe, to retract plunger-blade XX to a point behind the queue to clear the way for the compression spring to seat the next stay from the queue but with the forward edge of the plunger-blade XX remaining inserted within the rearward extension of ejection slot liner XX, which extends the roof XX, sides XX, and floor XX of ejection slot XX rearward. Both stays and plunger-blade are flexible to allow the stays to be bent during ejection to allow a better entry angle to achieve substantially concentric insertion.

Plunger-blade XX is made of flexible spring steel, or of polyester coated with flexible ferromagnetic metal, or of polyester interleaved with ferromagnetic bands or laminations of flexible ferromagnetic metal to participate in the stay recall magnetic circuit and is fastened at its distal end to the back wall XX rising from heel XX of tool butt XX by ferromagnetic rivet XX. It is obvious that by introducing angles into slide XX that pulls plunger-blade XX through ejection slot XX that the angle in relation to the axis of the tool of the flexible plunger-blade XX could be changed to apply in different conditions of encroachment by the surrounding tissue, and that the same may be said for the stays XX in magazine XX.

To prevent undesired incisions as could result from involuntary deflection of the tool sideways during insertion, the front corners of plunger-blade XX are blunted or rounded. So that the front edge of the plunger-blade (below) engages rather than just abuts upon the back edge of the stay so that separation of the two would leave the stay mispositioned or loose, the plunger-blade is thicker than the stays and v-notched along its front edge to span and encompass the stays. To accommodate this distinction in thickness, the stays are coated with freeze-dried sugar that is absorbed and metabolized shortly after implantation, which process is not significantly impeded by the cement used to seal the entry incision.

This retention within the rear portion of the ejection slot when the plunger-blade is retracted prevents the plunger-blade from becoming disengaged and misdirected from the ejection slot. The front, back, and sides along the path followed by the stays through the magazine and ejection slot fit flush to the sides of the stays. In order to countersink the near edge of the stay once implanted so that it will come to lie beyond the entry incision through the surface of the ductus sufficient to prevent backup through the same path and allow placement concentric as possible, plunger-blade XX extends sufficiently down the side of the ductus and beyond ejection slot liner extension XX.

Plunger-blade shield or guard XX encloses the exposed portion of the plunger-blade from and thus prevents displacement or pinching of the ductus XX. Plunger-blade shield or guard XX is continuous with floor XX of ejection slot XX, which is fastened at the bottom to the sides of stay cartridge XX by ethyl cyanoacrylate, 2-octylcyanoacrylate, or n-butyl cyanoacrylate cement and thus remains stationary as plunger-blade XX moves up and down behind it. Withdrawing plunger-blade XX allows compression magazine spring XX to expand inserting the next stay from the magazine load queue to be seated on the floor of ejection slot liner XX.

Releasing plunger XX then causes compression spring XX to pull plunger-blade XX back up through ejection slot liner XX ejecting stay XX out the front end or exit XX of the ejection slot liner. Forward extension XX of ejection slot liner XX beyond the outer surface of the stay magazine XX omits the floor of the ejection slot but preserves the sides and roof. The side walls and roof of the forward extension of the ejection slot liner angle downwards to remain flush to the surface of the ductus. The honed leading edge of the stay thus emerges from the liner in contact with the surface of the ductus, and the stay is prevented from veering aside or upwards before the honed front edge of the stay penetrates ductus XX.

The puller type insertion tool shown in FIG. 66 reverses the action of the insertion tool shown in FIG. 64 by using an compression spring XX to return trigger XX to its forward position, which pulls plunger-blade XX up into ejection slot liner XX to eject stay XX. Plunger-slide XX is slidably engaged with stationary overlying magazine XX by means of side tracks or rails XX. Pulling back trigger XX then draws plunger-blade XX past the entry extension of ejection slot liner XX forcing the stay out ejection slot exit XX. Except for placement of the battery in the pistol grip portion and the electromagnet, of which the probe tip must remain in contact with the heel rivet of the butt during movement, the stay insertion mechanism, to include ejection slot entry XX and exit extensions XX, and stay adhesive applicator pump XX feed line XX and end tip XX is the same as that described for the pusher type insertion tool shown in FIG. 64.

### Stay Insertion Tool Inmate Ductus Entry Incision Wound Sealing Mechanism (Slit-Sealer; Glue Applicator; Adhesive Applicator)

### Sealant Cartridges and Sealants (Adhesives)

As shown in FIGS. 64 and 68, the disposable refill cartridges or capsules XX incorporate features of disposable hypodermic syringes, the caulk or grease refill tubes used in caulking and greasing guns, and airgun CO, canisters (cartridges, ‘pistolets,’ ‘powerlets’). The cartridges XX are essentially shortened and miniaturized caulk tubes that are punctured at the outflow end by means of hollow hypodermic type needle type puncture pin XX fixed in position at the bottom or distal end of the adhesive refill chamber. FIG. 64 shows a single glue column for a single-component adhesive, which pending the availability of fully absorbed cyanoacrylate cements, is preferably octyl-cyanoacrylate or N-butyl-2-cyanoacrylate cement if not a longer chain acrylate.

As commonly seen in epoxy injectors for use in the home, when as pertains to the adhesives preferred for incision closure, the adhesive employed to seal ductus stay entry incisions is two-component, such as gelatin-dialdehyde (Geister Gluetiss®) or hydrogels, the cartridge contains two vials, one each for each of the components. Until single component fibrin sealants become available, this form of fibrin sealant is likely to remain preferable. For the present application, two component fibrin sealants supplied in four separate vials, especially when requiring temperature or other different preparation for each of the four constituents, such as with Baxter Tisseel VH®, are considered too complicated.

While preferred for fabricating some of the nonimplanted apparatus described herein, butyl 2-cyanoacrylate (B2-CA) cement is not preferred for sealing a wound as non-bioabsorbable, possibly bioincompatible, and in some cases, potentially carcinogenic. The use of a lower viscosity much less fluid cement, allows the complexity, precision, and expense of the advancement mechanisms seen in caulk and grease guns to be avoided, but requires the prevention of backup seepage about the edges of the plunger-piston used to eject the adhesive.

For this reason, plunger-piston XX is of the multiple elastomeric flange kind used in syringes. The plunger-piston is intermittently driven forward under air pressure developed through the reciprocal action of the stay insertion tool, which as explained below, is adapted to provide an integral air pump. To reduce off-axis deflection and jamming of the plunger-piston in its channel, the upper surface of the plunger-piston is dished or hollowed out to concentrate the air pressure at the center. The cartridges are individually sealed in sterile envelope packages and discarded following use.

### Insertion Tool Onboard Sealant Delivery Mechanism

An inmate sealant (adhesive) application mechanism for sealing the incision through the adventitia produced by insertion of the stay eliminates the need for the alternate insertion and removal of a separate device through the access incision or cannula. With an inmate gluing mechanism, the seal can be accomplished as part of the insertion cycle without the need to relocate each incision. To this end, the reciprocating configuration of these tools lends themselves to the operation of an air pump, which allows the elimination of numerous mechanical parts.

The ductus entry incision sealing mechanism consists of chamber XX which accepts disposable cartridge XX containing a surgical adhesive-sealant, one-way or unidirectional air valve XX, reciprocating air pump-piston XX, which is connected to the trigger or plunger slide through a longitudinal slot, and adhesive feeding tube XX, which extends from cartridge puncture pin XX to forward extension XX of ejection slot liner XX. Sealing of the incision is completed by lightly pressing down on the insertion tool to tamp down the incision. Since the stays are significantly countersunk by the plunger-blade, which is longer, this may require cocking or inclining the tool forward or axially rotating it full circle to the opposite side of the ductus.

The substantially constant temperature and humidity in the catheter laboratory obviate the need for compensation in the air pumping mechanism. One-way air valve XX admits a volume of air into cartridge chamber XX, the pressure pushing down against plunger-piston XX incrementally forcing the plunger downward with each additional incremental volume of air. This causes adhesive-sealant refill cartridge plunger-piston XX to propel adhesive-sealant XX down delivery tube XX to ejection slot forward extension XX causing each stay to be swathed with adhesive upon ejection. Incorporation into the stay insertion tool of an electrical cautery or harmonic scalpel is discounted as needlessly complex and expensive.

Unnecessary complexity is eliminated by allowing the direction of the air pump piston XX to move integrally with the insertion tool plunger rod rather than to be reversed by means of gears, rack, ratchet; or levers. This causes adhesive XX to discharge during the stay seating rather than the stay ejection half of the tool reciprocating action cycle. Any excess adhesive applied to the stays is then skimmed or squeegeed away by the upper lip of the ductus entry incision where it is easily wiped away if thought consequential. Backward displacement of air pump piston XX during the stay insertion portion of the cycle exhausts air behind the piston through pressure equalization or exhaust aperture XX while drawing air through one way air valve XX.

As shown in FIGS. 64 and 66, the insertion tool inmate adhesive delivery mechanism consists of an integral air pump, camber for the insertion of adhesive cartridges as described, and a path for the delivery to the stays upon implantation of adhesive. Adhesive XX passes through puncture pin XX and down tube XX to the front lip XX of forward ejection slot extension XX. The air pump consists of air pressure equalization hole and air pump-piston XX, which is connected to stay insertion plunger XX by means of a tab that slides along a slot in stay insertion plunger sleeve XX.

The delivery tube XX is made of any suitable polymer tubing and continues from adhesive puncture pin XX through the roof of the magazine XX, down through the center of the turns of magazine compression spring XX until just short of roof XX of the stay magazine clip chamber XX, at which point it emerges out and spirals around to continue downward along the front face of the insertion tool. Delivery tube XX then courses down the center of the front of the stay insertion tool and over the roof of the front extension of the ejection slot XX being affixed along the backside of the tube by means of an adhesive.

As shown in detail in FIGS. 67 and 68, the distal tip or adhesive emitting end of adhesive delivery tube XX is co-shaped with the front edge of forward ejection slot extension XX front lip XX to swath the upper surface of each stay with adhesive as these brush against the front extension roof XX upon ejection. Forward displacement of air pump piston XX in the stay seating portion of the cycle then forces the air trapped between the front of air pump piston XX and the surface of adhesive plunger-piston XX against adhesive plunger XX driving plunger-piston XX farther into adhesive cartridge XX and therewith an equivalent volume of adhesive XX through adhesive delivery tube XX through outlet XX.

Each time air pump piston XX is retracted, an additional volume amount of air is introduced through one way air valve XX into air pump chamber XX. Thus, plunger-piston XX is incremently driven forward by an equivalent distance for each volume of air added to the air column trapped in air pump chamber XX. Access to the battery, adhesive-sealant cartridge, and stay refill chambers, of which the interiors are contoured to conform to and thus secure the refills, is through side entry snap covers of the kind used to cover the compartment used to contain the replaceable battery in the back of a pocket calculator.

### Stent-Stay Insertion Tool Endoscope Mounting Clips

Examination of the insertion tool when not functioning smoothly is generally determined and any build up of adhesive accomplished by withdrawal of the tool from the work area for direct viewing, which the transparency of the materials used allows. In most instances, the overhead lamps and head lamp should provide adequate illumination down through the entry incision, and binocular telescopes should afford sufficient magnification. A small downward directed lamp that draws power from the internal battery can be mounted on the side of the instrument. An endoscope can, however, provide a more detailed view of the work area. To allow a closer view, a conventional flexible or fiber optic endoscope with light delivery system can be affixed alongside the tool.

To attach the endoscope, clips XX are positioned at intervals down the sides of the stay insertion tool. The endoscope can target the ductus to receive the stay implants or to discover that adhesive is not properly applied before this becomes apparent through clogging sensed tactually, the front edge of the ejection slot roof. Roof XX of ejection slot XX and the sides of the insertion tool are transparent, allowing the reflective liquid adhesive to be distinguished from the flat tantalum coating of the stays.

### Stent-Stay Insertion Tool Vacuum (Aspiration, Suction) Line Mounting Clips

Clips on the side of the stent-stay insertion tool opposite those for the attachment of an endoscope allow a vacuum (aspiration, suction) line to be fastened alongside the tool. To distribute the force of suction on the outer surface of the ductus to be treated, the distal soft tip of the suction tube may be flared outward towards the sides as aligned to the long axis of the ductus. A collapsed or collapsing ductus can then be drawn up toward the butt of the tool to allow the stays to be inserted to the depth sought. The disposal of used vacuum tubes and control of the vacuum level as, for example, by means of a Hall effect flow meter, lies outside the present scope.

### Stay Insertion Tool Inmate Stay Recall (Retraction) and Recovery Electromagnet

Because the insertion tool is devised to securely hold and move the stay during the ejection process, mispositioning will more often be due to operator error in choosing the insertion site than to malfunctioning of the tool. To allow a mispositioning stay to be recalled or returned into ejection slot XX at any point during insertion prior to withdrawal or the insertion tool, the insertion tool is provided with inmate electromagnet XX. A similar but larger (6 7/16 inches in length) and less specialized battery-powered electromagnetic probe was described by Crawford, W. A. 1976. “Hand-held Electromagnet-probe,” American Mineralogist 61(1-2):173, available at http://www.minsocam.org/ammin/AM61/AM61_{—}173.pdf.

Battery XX, electromagnet XX, and soft iron core XX and probe XX are connected to and move with plunger XX. Electromagnet XX is wound with American Gauge number XX copper magnet wire. Core XX extends downward (distad) as probe XX, which is connected by means of the same ferromagnetic (magnetoconductive) rivet XX that fastens distal end XX of plunger-blade XX to the back wall rising from heel or back XX of the tool working end. The term recall denoting retraction of the stay prior to withdrawing the tool and recovery the retrieval of a loose stay, control knob XX allows adjusting the current supplied by battery XX through a variable resistor, hence, the magnetomotive force generated, from a recall to a recovery level.

Electromagnet XX and battery XX generate sufficient field strength for recovery without the need for a power supply and socket for connection of the same as an alternative power source. Adjusted to the center position, three position switch control knob XX turns the electromagnet off, whereas the positions to either side turn the electromagnet on and set its polarity. To demagnetize the probe, the variable resistor current control is turned all the way down and the polarity momentarily reversed. The stay insertion tool can be used as a hand-held tractive electromagnet to retrieve or portative electromagnet to move any small ferromagnetic object in or out of the body.

Once implantation is complete, even if the insertion tool has not yet been removed, extraction is least injurious by incision and closure with a suitable adhesive, such as butyl 2-cyanoacrylate or fibrin sealant, the use of an harmonic scalpel avoided as thrombogenic. Further to allow plunger-blade XX to be incorporated into a magnetic circuit that allows stays which have not inserted concentrically (misinserted, mispositioned) to be withdrawn, the parts about the plunger-blade XX are formed of nonferromagnetic material, such as the plastic resins specified above.

### Use of Stent-Stay Insertion (Injection) Tool

The battery, electromagnet, and magnet control are tested on a staple or pin. A strip of stays is inserted into stay chamber XX. A cement cartridge is placed in the chamber, the narrow distal end punctured by pressing down on hollow outlet needle XX, and the plunger XX or trigger handle XX cocked until cement begins to emerge at the end of cement applicator tube XX. Two or more stays are ejected to test the tool before entry through the entry incision. If the tool does not perform correctly, the transparent tool allows the cause therefor to be directly observed. The stay clip is removed and a hypodermic needle used to inject acetone into adhesive cartridge puncture pin XX and through the delivery tube to flush it of adhesive wet or caked. A dental probe-hook is used to evaluate the pliancy of the ductus.

The stay insertion tool is passed through the entry incision and positioned on the ductus with toe XX and arcuate bottom of the working end flush. The depth of implantation is set by adjusting the downward force on the ductus. When properly employed on a ductus of the prescribed diameter for the specific tool used, setting positioning butt XX with no more downward force than is necessary to keep the tool from shifting will achieve subadventitial placement. Applying somewhat more force will cause the stays to enter more deeply into the media as is unavoidable should the adventitia delaminate from the subjacent tunic.

The amount of downward force might be quantified with a built in scale; however, clinical experience is preferable, the recommendation of specific forces for variable conditions ill advised. While improbable, a ductus that slides or rolls aside despite the indented butt or sole of the tool is stabilized with the aid of a probe. The stay is inserted. To tamp down and seal the incision, the tool is moved slightly forward or reversed, and a slight downward force applied. If implantation is suspected to be mispositioned prior to ejection, the recall magnet is energized to withdraw the stay and the tool tested outside the body. The operator confirms the successful sealing of each before proceeding to the next. If the ductus stay entry incision is not sealed, the tool is removed and a long chain methacrylate adhesive introduced into the ductus stay entry incision at the end of a dental pick. The stay insertion tool is tested outside the body.

Upon completion of the procedure, the adhesive cartridge is removed and discarded and the delivery tube flushed with a commercial long chain methacrylate glue remover or solvent such as Duro® Super Glue Remover, acetone, or acetone in the form of nail polish remover before sterilizing as described below. The solvent is passed through inmate delivery tube XX through a length of tubing used to connect puncture needle XX to the outlet of the syringe that is used to inject the flushing solvent. Proper functioning of the tool is confirmed by direct visual examination of the tool outside the body, which is made easier by the incorporation of materials that afford transparency with good optical clarity.

In situ, transparency serves not only to improve the viewability of the work area from different angles to confirm proper contact and circumferential relation of the tool butt to the ductus surface, but with the aid of an optionally attachable endoscope, allows the stays to be observed as these pass through the ejection slot XX. Should the ductus to be stay-implanted be collapsed or collapse under the stay insertion tool or waver due to smooth muscle action, a vacuum (aspiration, suction) line fastened to the side of the tool opposite the endoscope is used to better stabilize and achieve the tool-ductus relation required. In some instances, a muscular artery may require to be immobilized with a forceps or hemostat.

### Subcutaneous and Suprapleural Clasp-Magnets (Patch-Magnets)

While muscle fascia is ill-defined from the subjacent epimycial fascia, so that pinching skeletal muscle fascia does not, for example, lift the fascia away from the underlying muscle, the integument is substantially free to move in relation to underlying skeletal muscle. That is, the integument is loosely attached as to easily slide over the underlying muscle, which is stable in average position relative to the skeleton. Therefore, provided it presents smooth and curved profiles at the top and sides, a thin magnet attached to the muscle fascia will move with the muscle, freely sliding along the internal surface of the integument without abrasion. To preclude placing the esophagus under any significant compression, the strength of the magnetic pull should be no greater than that required to prevent the dorsal membrane from being drawn down by the tidal flow of respiration.

In dogs, the loose attachment of the integument is conspicuous, and the support of collapsed bronchi when alternative measures (endoluminal stents) are contraindicated, for example, may represent the only option. The magnets must be positioned so that the esophagus limits the distance that the tracheal ceiling can be drawn, and lung tissue limits the distance that the bronchi can be drawn and selected for a pulling force that effects suspension without imposing unnecessary force on these limiting tissues. In such application, the magnets are attached to the outside of the skeletal muscle or to pleura above the bronchi to run parallel with the underlying miniball implants. Since the subcutaneous implants are more readily interchanged to ascertain the best strength magnet to use at points superjacent to the miniballs, the internal implants are generally placed first and the magnets placed thereafter. Since the miniballs are implanted with the patient supine, and have little mass, collapse is not aggravated during the interval until the magnets have been placed.

Unless there has been asphyxiation, it should be possible to perform the implantation in one an initial procedure, and placement of the magnets in another. By pressing the magnets down to the level these would occupy once attached, this can be accomplished before incision. Just before closing, the area surrounding the prongs is treated with a long-effect local anesthetic, such as lidocaine (lignocaine; Xylocalne®). Because it immediately surrounds the affected ductus and inherently but flexibly limits wall excursion, when not otherwise contraindicated, the use of subadventitially implanted miniballs with a stent-jacket is preferred to alternative methods such as the use of a miniball jacket with stent-jacket, minimagnet with a miniball jacket or with implanted miniballs, or subcutaneous or superpleural magnets. Where a stent-jacket cannot be used, such placement can afford a suitable location for peripheral magnets to act upon more deeply implanted miniballs. Subcutaneous placement also allows retrieval with relatively little difficulty, but may require preventing the patient from lying beside some objects made of ferrous metal, such as a filing cabinet.

Referring now to FIGS. 18 and 19, shown is a patch-magnet or clasp-magnet that incorporates disk magnet 34 for attachment to the muscle fascia or pleura. The magnet 34 is bonded to subjacent strips 35, preferably made of an austenitic stainless steel such as 18-8, 304, or 316, cold-worked to full hardness to induce shape-memory or restorative force so as to act as a small nonmagnetic leaf-spring. These strips 35 run beneath and in tangential relation to the magnet 34 along its undersides and are cambered, with the magnet surmounting the center convexity on the upper surfaces.

In manufacture, the die cut includes perforations in parts to contact tissue, especially the clasps, which typically penetrate the substrate tissue to a depth of 3-4 millimeters, which is intended to allow tissue infiltration and integration for increased retentive tenacity. Since these elements will be completely encapsulated and isolated within a bioinert plastic jacket, the adhesive used to bond these elements pending encapsulation is not significant, but can be, for example, cyanoacrylate cement or Loctite Hysol Cool Melt®. The encapsulated magnets are consistently mounted to the strips with south poles directed downwards.

As is seen in FIG. 19, the strips 35 end in recurved tines or prongs 36 with pointed sharp ends sized and configured for fastening the patch-magnet to the surface of the overlying fascial sheet by no deeper than to undercut and catch hold of or clasp the superficial muscle fascia, innervation avoided, causing the patient the least discomfort. In use, an incision is cut into the integument only so large as necessary to introduce the magnet, which is then positioned, and pushed down, depressing the leaf spring supports. Upon releasing the downward force on the magnet, the leaf-spring seeks to recover to the curved form, engaging the end-prongs under the fascia.

Some force on the prongs and possibly pinching of the fascia when the magnet is set in place are necessary to fully engage the prongs. The prongs stimulate the generation of cicatricial (scar) tissue that uninnervated, is numb, preventing further incision and discomfort. However, if following a suitable interval for healing, irritation due to the prongs persists, then the subcutaneous or suprapleural magnets are removed and the determination made whether to suture these in place would remedy the problem. If not, then esophageal tacking to a magnet-wrap as described above is accomplished.

The bonded magnet 34 and strips 35 unit is then encapsulated by dip-coating in biocompatible plastic, such as polyvinyl chloride, which depending upon finer chemical details, has a melting point that ranges from 175 to 212 degrees Fahrenheit, or highly polymerized (high molecular weight) polypropylene with a melting point of 320 degrees Fahrenheit. These are sub-demagnetizing melting points for the neodymium iron boron magnets, which depending upon the exact ferrite material, have a Curie temperature of around 590 degrees Fahrenheit. The higher melting point of polypropylene has the additional advantage of allowing the maker to sterilize the magnet-patch by steam autoclave before placing it in the sterile package.

The previously presumed nonbiocompatibility of the plasticizers used to make phthalate esters and polyvinyl chloride pliable has since been discredited by the American Council on Science and Health 1999 and the European Commission on Health and Consumer Protection Directorate-General 2002. In manufacture, once cured, the incisive ends of the prongs are exposed (denuded) by stripping away this coating. To gold anodize more than the exposed prongs of the unitized magnet with clip mounting prior to dip-coating is considered redundant for use in this subcutaneous environment, which in a patient free of subcutaneous disease, is substantially noncorrosive, nondegradative, and pathogen free.

### Chemical Isolation of Implanted Components

Encapsulation to impart bioinertness to the miniballs, stent-jackets, miniball-magnets (magnetized miniballs; magnetized spherules) and subcutaneously or suprapleurally implanted disk magnets described in connection with extraluminal stenting is accomplished by overlayment with gold, tantalum, titanium, or a bioinert plastic polymer resin applied by dip-coating or lamination between sheets. The radiopacity of the miniballs notwithstanding, the tiny size of these encourages improvement through the addition of an outer coating of tantalum. The further encapsulation of the tantalum layer with a medium, such as starch or sugar-based, for delivering medication upon dissolution does not detract from radiopacity.

Of the various methods for applying a coating of gold to these components, conventional electrolytic barrel plating may produce a microporous surface not entirely free of plating bath solution chemicals (see Sahagian, R. 1999. “Critical Insight: Marking Devices with Radiopaque Coatings,” Medical Device and Diagnostic Industry Magazine, Canon Communications, May 1999), in which case the coating fails to provide the biocompatibility that was its object. The materials and chemical isolation of the various components described herein are further addressed under the section devoted to each.

### Miniballs (Miniature Balls, Spherules, Minispheres)

Miniballs for use in simple pipe-type barrel-assemblies generally range in diameter from 0.7 to 2.4 millimeters, while those for use in radial discharge barrel assemblies for use other than in the airway range between 014 and 2.4 millimeters. With ferromagnetic miniballs, sphericity presents a poor gap for magnetic flux, recommending the use to attract these of neodymium magnets of high energy product for small size with low mass, which minimizes if not completely eliminates encroachment, upon neighboring structures and causes the least discomfort to the patient. At the same time, ballistic implantation, as described herein, is as clean, i.e., as bloodless and atraumatic as possible, while readily lending itself to aseptic delivery.

Producing a trajectory or path of insertion little wider than the miniball itself, implantation by such means is least disruptive to surrounding tissue. Cells along the trajectory and its terminus are crushed and release fluid contents; however, this injury is small in extent, highly localized, and the resulting inflammation is medically manageable. Furthermore, the fluid released lubricates the spontaneous rolling around into optimal polar orientation of magnetized miniballs having a core of neodymium ferrite and expedites the delivery of medication from miniballs with an outer coating that contains a drug or drugs. However, implantation by such means requires projectiles that are spherical.

As shown in cross section in FIG. 20, a miniball consists of an iron or steel core 37. When tissue compatible, core 37 represents the sum total of the miniball; if not, the miniball is plated with gold, heated, and sputtered to remove contaminants (see Edelman, E. R., Seifert, P., Groothuis, A., Morss, A., Bornstein, D., and Rogers, C. 2001. “Gold-coated NIR Stents in Porcine Coronary Arteries,” Circulation. 103(3):429-434). Alternatively, stainless steel tending to afford relatively low radiopacity, the miniball is coated with tantalum, such as with Danfoss Tantalum Technologies Danfoss Coating®, in which case 38 represents the outermost coating, the additional layers in the figure not required. When patient life expectancy justifies the additional expense, the implants are encapsulated in gold, platinum, or tantalum, which are radiopaque.

Other than for those having a neodymium ferrite core, the encapsulation of implants for bioinertness is precautionary. When the patient is expected to survive for years and the chemical breakdown of core 37 could release a nocuous constituent over time, then the core 37 is encapsulated for bioinertness by overlayment with gold, tantalum, titanium, all of which additionally contribute high radiopacity, or a bioinert plastic polymer resin shown as layer 38 surrounding the core.

Should core 37 be enhanced in radiopacity with a coating of tantalum before encapsulation in a resin for even greater isolation, the tantalum coating is represented by 38 and the resin by 39. If not itself of tantalum, which is bioinert and affords good radiopacity, layer 38 for chemically isolating the core can be coated with an additional layer 39 of tantalum. Still referring to FIG. 20, a steel-core 37 gold plated (layer 38), and microfused (layer 39) miniball may be further coated with a layer of tantalum 40.

For magnetic optimization, core 37 is preferably made of a corrosion-resistant ferromagnetic stainless steel, and proportionally large as possible within the thickness constraints of the outer coating materials when present. Ferromagnetic stainless steels include those of 400 series and heavily cold worked 8 percent ferrite 316 in CF8M alloy. Ferritic, martensitic, and to an extent, less austenitic stainless steels can also be used as cores.

Since gold plate can present microfractures, unless an outer coating of tantalum already applied for improved radiopacity also fully seals the exterior, a process whereby additional gold is applied to completely seal the core, microfusion, is applied, or otherwise, replating, in which case layer 39 represents this layer. In addition to close inspection under a stereomicroscope, microfractures of the electroplated surface can be detected by standardized corrosion protection tests, such as the salt spray or salt fog test (American Society for Testing and Materials Standard B 117) and Kesternich (Deutsches Institut für Normung Standard 50018 or International Organization for Standardization Standard 3231) tests.

Thus, if gold-electroplated, surface contaminants must be eliminated and any voids filled by microfusion or vacuum deposition plating, which involve temperatures, typically 70-120 degrees Fahrenheit, well below the 590 degree Fahrenheit Curie or de-magnetizing temperature of neodymium iron boron ferrite magnets. An outer coating that consists of a polymer rather noble metal is applied by means of in-situ or matrix polymerization, which methods are widely used in the manufacturing of pharmaceuticals.

Gold is of high specific gravity, but not such as would result in too heavy an intravascular component of an extraluminal stent. For miniballs and miniball-magnets, however, adjustment in plating thickness allows precision in achieving a certain mass. Combined with variability in the material used to encapsulate these components, some variability in core and wide variability in plating thickness may be applied to achieve considerable exactitude in mass and aeroballistic performance. When the combination of concentric layers as depicted in FIG. 20 or 21 results in a caliber too large for the barrel-tubes, consideration should be given to use rotary magazine clips loaded to provide alternative irradiated and/or medication and ferromagnetic miniballs or miniballs that variously combine layers to provide adequate coverage over the area to be treated.

### Radiation-Emitting, Medication, Drug-Eluting Magnetized, and Magnetized Miniballs

### Radiation-Emitting (Brachytherapeutic, Endocurietherapeutic, Sealed Source Radiotherapeutic, Internal Radiation Therapy) Miniballs

The ability to transluminally approach a level of a ductus that is a site of disease and infix within the lumen wall a discrete implant that emits radiation and/or medication affords advantages over the prior art, in that to accomplish a similar result, one previously had to introduce an endoluminal stent that albeit absorbable, brought with it the several problems discussed above. Miniball implants can consist entirely of medication, which by concentric layering can be multiple, can include a core that is an irradiating seed, and miniballs implanted in adjacent relation can combine, separate, and alternate in medication and radiation. Tantalum or comparably vivid markings at the muzzle-port or ports is discussed elsewhere.

The placement of radiation-emitting seed miniballs by the means described herein is not intended for transluminal implantation of the prostate gland, which if introduced through the urethra would injure the epithelium exposed to urine and could injure the urethral verumontanum. Neither does ballistic implantation allow the suturing together of seeds for positional stability as does conventional needle insertion. When the muzzle-head can be brought beyond the distal extremities of the bronchi so that little more than manageable hemorrhaging would result, use of this method may be given consideration.

In most instances, even though the patient is inconvenienced, high-dose-rate radiation administered with an automated afterloader machine is likely to be preferred. Provided the seeds are spherical, any barrel-assembly of matching gauge or caliber can be used to implant low-dose-rate ceramic-titanium encapsulated radioisotopes of cesium-131, iridium-192, bismuth-212, lead-212, iodine-125, gold-198, phosphorus-32, ytterbium-169, yttrium-90, or palladium-103 within the wall of a ductus. No endoluminal stent present, radiation seeding pertains to primary stenotic conditions best treated by highly localized sources rather than to reducing the risk of in-stent restenosis. The interstitial brachytherapeutic seeds required differ from those currently made by Implant Sciences Corporation, Wakefield, Mass., IsoRay Medical Incorporated, Richland, Wash., North American Scientific, Chatsworth, Calif., or C. R. Bard, Incorporated, Covington, Ga. only in being spherical.

The forces generated by airgun expulsion in medical use do not attain values as would jeopardize seed integrity as conventionally manufactured, so that no additional precautionary structural modifications to the seeds is required. Furthermore, whereas low-dose-rate seeds have been considered unrecoverable, by enclosing ferrous metal within the seeds, the recovery electromagnets, which just in front of (distal to) the muzzle-ports are immediately present to the implantation site, are present to retrieve any seeds that should become mispositioned. Depending upon the maximum diameter of the miniballs that can be used, the spherical seeds may be further encapsulated within layers of medication or sequentially time-released medication. In this connection, and with respect to the pure medication miniballs, a medical layer can contain a gamma-emitting isotope such as indium-111 or molybdenum-99.

Conventional intravascular brachytherapy is compatible with the use of a barrel-assembly. The intravascular brachytherapy catheter is passed down the barrel-assembly (as is a cooling catheter, below). The same applies to a high-dose-rate source delivery catheter as controlled by an automated afterloader. When performed with another catheter-based procedure, radiation seeding by this means differs from intravascular brachytherapy in not requiring the withdrawal of one catheter and the introduction of another; only the extracorporeal rotary magazine clip in the airgun requiring to be changed.

### Medication Miniballs

Spherules consisting of a core and concentric shells for ballistic implantation in the walls of diseased arteries, for example, can be produced using conventional methods in the pharmaceutical industry to include pan tumble coating, centrifugal extrusion, and spray-drying. For most formulations, the vibrational nozzle technique affords superior exactitude of sphericity, which is important for maintaining accurate control over the discharge velocity from the barrel-assembly in proportion to the distance the miniball must transit from the chamber of the airgun to the exit port in the muzzle-head. In every case, medication or tablet miniballs, with or without an encapsulated ferromagnetic or radiation source seed core, must be given an outer coating that will withstand the tangential shear stresses encountered in transmitting the barrel-tube.

In radial discharge barrel-assemblies, the barrel-tubes consist of a fluororo or another low friction polymer, or are lined with a fluororopolymer produced by coextrusion. The crushing-strength, disintegration time, porosity, and friability of tablets relating consistently (see, for example, de Jong, J. A. H. 2005. “Relations Between Tablet Properties,” Pharmacy World and Science 9(1):24-28), resistance to the smaller impact forces of ordinary handling varies proportionally to the strength required to prevent miniball tablet fracture upon discharge.

Discharge and travel through the barrel-tube subject miniball tablets to tangential shear forces that exceed those encountered by tablets in ordinary handling. To prevent surface fractures, much less overt breakage during discharge, which could not only affect discharge but result in the deposition of debris along the barrel-tubes that might lead to jamming, miniball tablets are strongly compressed as true spheres and may be additionally covered with a fracture-resistant coating, such as an ester bond based bioabsorbable polymer, typically polyglycolic acid, polyester, or poly (p-dioxanone), which to present a low coefficient of friction is further coated with N-laurin and L-lysine.

Overlying the core-encapsulating layer that imparts bioinertness in a miniball with a ferrous or ferrite core, such as produced by means of gold microfusion, miniballs for intravascular stenting can be further encapsulated with medication or contain an irradiating seed as a core. Irradiating core miniballs are usually conventional seeds with titanium jackets typically lined with a ceramic but in spherical form, that are used apart from stenting function, which would necessitate the incorporation of ferrous material. Some stenosed conditions of a ductus may recommend seeds containing ferrous metal to alleviate the stenosis by means of encircling the ductus with a stent-jacket and to pinpoint the sources of radiation with the same material.

A drug-releasing external layer can consist of polylactic acid (Dev, V., Eigler, N., Fishbein, M. C., Tian, Y., Hickey, A., Rechavia, E., Forrester, J. S., and Litvack, F. 2997. “Sustained Local Drug Delivery to the Arterial Wall via Biodegradable Microspheres,” Catheterization and Cardiovascular Diagnosis 41(3):324-332), a sugar, starch, or syrup coating tumbled to assure sphericity while heat-blown or freeze-dried (lyophilized). Neither a medicated coating that encapsulates and is meant to remain with the miniball until implanted nor the fixative cornstarch, rice starch, other syrup, molasses, acacia, methyl cellulose, povidone (polyvidone, polyvinyl pyrrolidone, PVP), or gelatin used to position the miniball in the ring-hole may be friable as to powder and leave a particulate residue along the barrel to any significant degree.

Referring now to FIG. 21, some miniballs include all of the layers represented in FIG. 20, to include a core 37 and additional layers 38 thru 40, to which is now further added an outermost coating 41 representing a drug-delivering, drug-eluting, or radiation-emitting medium. When subjacent layer 40 is a coating of tantalum for enhanced radiopacity, adding a medicated or irradiative outermost coating 41 does not annul this property.

Within the overall range for its diameter, adjusting the relative thickness, or varying the relative diameter, of the five layers allows a miniball of specified diameter to be given a specified mass or magnetic susceptibility. Miniballs implanted in arteries can be medicated with, for example, antithrombogenic, anti-inflammatory, or antiseptic medication to reduce the risk and the severity of abrupt closure by spasmodic reflex to ballistic implantation. The addition in manufacture to an irradiating seed with a metallic, such as titalnium, outer jacket of concentric shells containing medication by means of air-suspension is limited by the mass of the miniball.

Serious complications have been associated with drug-eluting intravascular stents. Often introduced as secondary endoluminal stents to reduce the reocclusion of a stent placed earlier, time-release drug-eluting stents have been implicated in clotting and restenosis requiring surgery and may lead to death. However, such complications as pain, rash, hives, itching, fever and changes in respiration or blood pressure are likely the result of allergic hypersensitivity to the specific drugs used. Such reactions are maximized when the stent is in the bloodstream, and when intended for local absorption rather than entry into the circulation, the blood level of the drug obtained from an extraluminal stent can be reduced to subclinical.

A magnetized miniball, or miniball-magnet, differs from a miniball in having a core made of magnetized material, usually sintered neodymium iron boron rather than iron, steel, alnico, or alternative ceramics, and owing to function and poor magnetic efficiency of a spherical contour, is often larger. Unlike alternative magnets, which are mounted to an organ or vessel surface, a miniball-magnet like any miniball, can be implanted in deep tissue as well as coated with additional layers for the immediate or time-delayed delivery of medication.

Such medication can be analgesic, antipyretic, anti-inflammatory; antibiotic; an anticoagulant, such as heparin; can be radiation particle-emitting, or some combination of these. There is some evidence to indicate that an outer coating of polylactide-co-sigma-caprolactone copolymer eluting paclitaxel would make possible sustained paclitaxel delivery to suppress neointimal hyperplasia for months after implantation and beyond the time for delivery of the drug to have been completed and the polymer to have dissipated (Drachman, D. E., Edelman, ER., Seifert, P., Groothuis, A. R., Bornstein, D. A., Kamath, K. R., Palasis, M., Yang, D., Nott, S. H., and Rogers, C. 2000. “Neointimal Thickening after Stent Delivery of Paclitaxel: Change in Composition and Arrest of Growth over Six Months,” Journal of the American College of Cardiology 36(7):2325-2332.

The crushing of cells along the trajectory of the miniball liberates the aqueous protoplasmic contents to dissolve this outer coating releasing the medication, which may be an antibiotic, anti-inflammatory, or contain a beta or gamma radioactive isotope, into the surrounding tissues and bloodstream. The fluid state at the trajectory terminus also allows magnetized miniballs to roll around into tractive orientation. For practical application, the chemistry and physics of layers added to that bioinert, to include encapsulation with medication or a radiated layer, the friability and rolling resistance of such an added layer under a given force of expulsion from the airgun, the rolling resistance due to friction and any bends in the barrel-assembly structure or as dictated by the anatomy, the tolerance of the miniball outer layers to withstand these forces, and the need to further coat such an added layer in order to alter these characteristics must be considered.

A preferred method for incorporating medication for timed-release in the sugar-based outer coating of medicated miniballs is by liquid feed micro-encapsulation as may be produced using apparatus available from the Sono-Tek Corporation, Milton, N.Y. Depending upon the application, miniballs may be coated, as with ion exchange resins, for example, not just to deliver medication or radiation but to minimize the embologenicity of the metal surface. In order not to affect the penetration characteristics of the miniball in any significant way, such added coats must be hard.

Improved acceptance by the ductus of the miniball and stay implants described herein can be obtained by phosphorylcholine coating (Chen, C., Lumsden, A. B., Ofenloch, J. C., Noe, B., Campbell, E. J., Stratford, P. W., Yianni, Y. P., Taylor, A. S., and Hanson, S. R. 1997. “Phosphorylcholine Coating of ePTFE Grafts Reduces Neointimal Hyperplasia in Canine Model,” Annals of Vascular Surgery 11(1):74-79; Whelan, D. M., van der Giessen, W. J., Krabbendam, S. C., van Vliet, E. A. Verdouw, P. D., Serruys, P. W., and van Beusekom, H. M. M. 2000. “Biocompatibility of Phosphorylcholine Coated Stents in Normal Porcine Coronary Arteries,” Heart 83:338-345), and hydrogel polymers incorporating phosphorylcholine can be used as a bioinert medium for delivering medication (Lewis, A. L. 2006. “PC [Phosphorylcholine] Technology as a Platform for Drug Delivery: From Combination to Conjugation,” Expert Opinion on Drug Delivery 3(2):289-98.

### Rotary Magazine Clips

FIG. 22 shows a seven-shot rotary magazine clip for use in a single barrel radial discharge barrel-assembly, and FIG. 23 a 10-shot rotary magazine clip for use in a four barrel or four-way radial discharge barrel-assembly. The single-shot rotary magazine clip can be adapted for use in a four barrel or four-way radial discharge barrel-assembly where only one barrel-tube is used, but the reverse. The miniball-holes 42 in each clip have been numbered for reference and do not appear on clips.

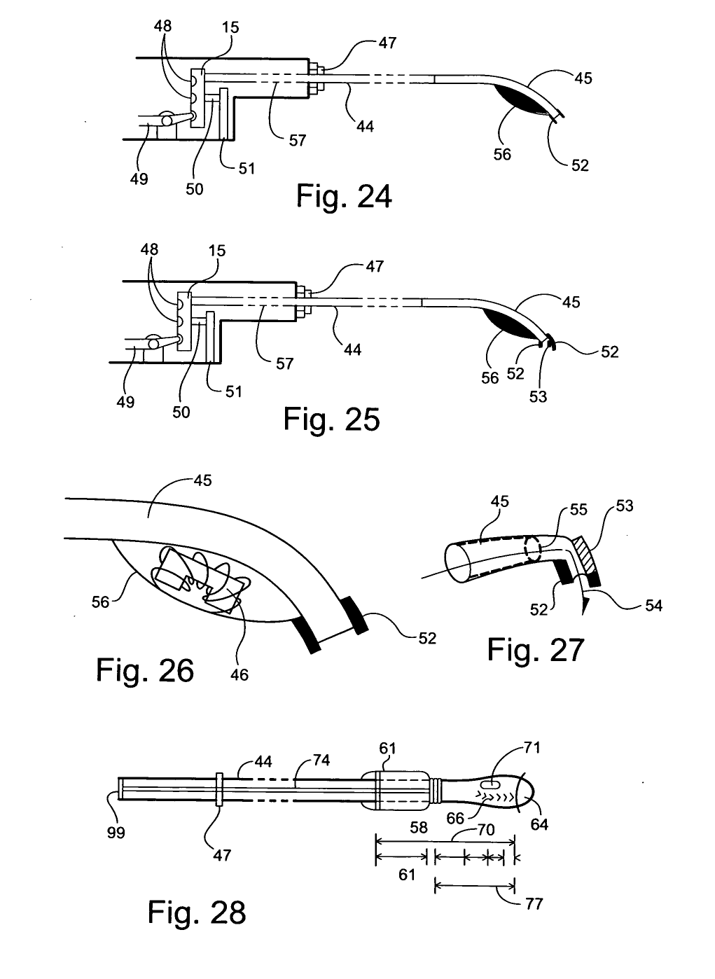

Rotary magazine clips are separately sealed in a sterile airtight package and disposed of after use. As a long practiced mechanism, rotary magazine clips are mounted by means of an axial hole at their center 43 to a hub or axle in the airgun and are rotated by the indexing action of an arm or pawl that rises to engage the circularly successive notches molded into the clip about its periphery or outer edge and pushes against each notch seen in FIGS. 24 and 25 but on the reverse or back side of the views provided and thus not seen in FIGS. 22 and 23) to obtain rotation as the sum of these rotational increments. Reloading the airgun is accomplished quickly, one rotary magazine clip pulled off the axle in the airgun and another inserted in its place.

The rotary magazine clips used in the airguns to be described are conventional in overall dimensions but situate one or more, usually up to four, miniballs for discharge at a single time. Barrel-assemblies that radially discharge eccentrically to treat eccentric lesions are fed from the same kind of rotary magazine clips with the changes in direction obtained by the course taken within the barrel-tubes. To more axially direct the propulsive force when the chamber is not airtight behind the barrel, and thus reduce diagonal vectors from the gas entry portal at the back of the chamber directed toward the rear of the slightly off-axis miniballs that are otherwise concentric thereto, the back hole can be drilled out to a larger diameter.

Incorporation into the rotary clip holes of a slight circumferential ridge midway from the front to the back face prevents the miniballs from dropping out of the rotary magazine into the barrel before discharge. The addition of this ridge allows the Model 617X airgun made by Maruzen Kabushiki Kaisha according to the specification of and sold by the Daisy Outdoor Products Company to shoot either miniballs or pellets, this model differing from the Model 622X of the same maker only in caliber and the ability to shoot miniballs as well as pellets. The rotary clips to be provided for use in an interventional airgun are unique in the caliber of the miniballs to be loaded. To spare the cost of preparing original molds to make the rotary clips, those in manufacture can be adapted by inserting a disk into the existing holes which contain the holes for the miniballs. When all the miniballs to be discharged at the same time are alike in mass, a slight midcircumferential ridge along the internal surface of each hole may be used.

Another way to retain the miniballs in the holes until discharge is to run povidone, cornstarch, syrup, molasses, acacia, methyl cellulose, or gelatin into the groove formed between the circumference of the miniball and the edge of the hole, allowing the syrup to form a uniform layer by surface tension, which is then freeze or quick dried by being passed under a heat lamp. Varying the formulation of the syrup in concentration and ingredients, such as sugars, starches, and the others just mentioned allows different degrees of adhesion that allow the loading of miniballs that differ in mass on the same rotary clip. Varying retentive adhesion inversely as the mass of the miniballs to be discharged together allows treatment of an eccentric distribution of lesions where some areas pose greater resistance to penetration or may require different medication in the form of an outer layer applied to the miniballs.

### Airgun and Electrical Connections and Controls of Barrel-Assemblies by Functional Type

### Ablation and Angioplasty-Incapable Barrel-Assemblies

An ablation and angioplasty-incapable barrel-assembly is a catheter extension of an interventional airgun barrel that is used only for stent implantation while engaged in an airgun. Primarily meant for use in ducti other than arterial, an ablation and angioplasty-incapable barrel-assembly is for use in the arterial system only following an angioplasty or an atherectomy that is depended upon to have minimized the risks for plaque rupture and the release of embolizing debris by contact with the muzzle-head. Since the use of an ablation and angioplasty-capable barrel-assembly allows angioplasty and stenting with a single entry and withdrawal, an angioplasty or atherectomy preceding the use of an ablation and angioplasty-incapable barrel-assembly would have been performed using prior art (conventional) means.

Intended for use mostly in ducti other than vascular, the use of an ablation and angioplasty-incapable barrel-assembly in the arterial tree to stent without an antecedent angioplasty, even with the addition of a distal embolic protective filter, is specifically renounced as risking the release of embolizing debris. Ablation and angioplasty-incapable barrel-assemblies include simple pipes and radial discharge mono- and multibarrel radial discharge barrel-assemblies. No independent (intrinsic, inmate) thermal ablative or angioplasty means are incorporated to allow use as separate from the airgun for freedom of movement, and no on-board electrical components or connections for independent power or control are installed. However, as the turret-motor is required positionally and the recovery electromagnets are required to retrieve dropped or to extract misplaced miniballs during implantation discharge, ablation and angioplasty-incapable barrel-assemblies require electrical connection to the airgun power supply, and this is accomplished through the types of contacts shown in FIGS. 49 and 52.

The incorporation of side-sweepers into the muzzle-head of an ablation and angioplasty-incapable barrel-assembly is to impart a radial nudging capability as a part of positional control rather than an ablative or angioplastic function. Because an ablation and angioplasty-incapable barrel-assembly may include side-sweepers but lacks an on-board ablation and angioplasty control panel, a control for deployment of side-sweepers is always included in the positioning and discharge control panel mounted to the cabinet of the airgun. For this reason, when an ablation and angioplasty-capable barrel-assembly is used, the side-sweepers can be deployed from either the ablation-angioplasty control panel on-board the barrel-assembly or the positional and discharge control panel mounted to the cabinet of the airgun.

Conversely, because insertion in the airgun prevents free and independent movement unless the electrical connections are as shown in FIG. 52 and there is sufficient slack (and not as shown in FIG. 49), an ablation and angioplasty-capable barrel-assembly is used for ablation or angioplasty before engagement in the airgun. Therefore, the positional controls mounted to the airgun would not usually be those used to rotate an eccentric turret-motor slot or slit heat-window or to eccentrically deploy a side-sweeper during ablation or atherectomy. The controls for these are on-board the free-standing ablation and angioplasty-capable barrel-assembly.

### Minimally Thermal Ablation and Angioplasty- (Lumen Wall Priming-Searing-) Capable Barrel-Assemblies

There are essentially two kinds of barrel-assembly: ablation and angioplasty-incapable, which are used only while engaged in the airgun, and ablation and angioplasty-capable, which are used independently of the airgun until implantation discharge is performed. A position and discharge control panel, which functions go to the airgun, is mounted on the cabinet of, the airgun, and is described below under the section entitled Barrel-assembly onboard angioplasty control panel, while a control panel for a barrel-assembly of sufficient angioplasty capability for use while separated from the airgun, which goes to the barrel-assembly as a free-standing apparatus until engaged in the airgun to initiate implantation discharge, is mounted on the barrel-assembly, and is described below under the section entitled Airgun (stewing) control panel. The potential of thermal or cryogenic angioplasty, of a bonding agent, or of these in combination to repair a delamination between layers or the tunics within the wall of a ductus warrants study. No laser-based apparatus similar to those used to reattach a retina appears available. When the intima or internal layer is the source of the stenosis, the apparatus and equipment described herein can be used only if it can be reattached to the media. Then any stent used must be endoluminal or conventional.

To allow the cost to be reduced, certain components that would be incorporated into a fully capable barrel-assembly but which are considered unnecessary for a specific procedure are omitted. Intermediate forms for use while engaged in the airgun, which precludes the attachment at the back of the barrel-assembly of a vortex tube or CO₂ or NO₂ cartridge for thermal angioplasty and denotes electrical connection through the airgun to the power supply either as shown in FIG. 49 or FIG. 52, allow omission of an inmate power source (battery-pack), side-sweepers, and the inclusion of fewer controls on the on-board control panel, but are limited to light and simple implantation discharge preparatory angioplasty or ablation. The positional control connections for the turret-motor and miniball recovery function of the two tractive electromagnets in the nose of the muzzle-head are always either of those depicted in FIG. 49 or FIG. 52, and when an ablation and angioplasty-capable barrel-assembly is connected thus, the duplicate muzzle-head rotatory control onboard the barrel-assembly is not disabled.

To stent without an antecedent angioplasty or where it is suspected that a previous angioplasty left rupturable plaque in place requires the addition of minimal angioplasty means to minimize the risk of releasing embolizing debris by contact with the muzzle-head. Accordingly, any time that a barrel-assembly is introduced into an atheromatous artery, especially one that has not been angioplastied or that a preceding angioplasty notwithstanding, is believed could retain rupturable plaque, thermal or cryogenic angioplasty is performed. The application of heat to the lumen wall is for precluding to the extent possible, the rupture of plaque and not for altering the mechanical properties of the lumen wall in preparation for implantation, which requires only routine adjustments in ejection force or exit velocity. The means for achieving good thermal conductivity and focus are described below in the section entitled Thermal Conduction Windows (Heat-windows) and Insulation of the Muzzle-head Body in thermal ablation or thermal angioplasty-capable barrel-assemblies. A minimal angioplasty capability is attained by making the windings already present in the implant spherule recovery electromagnets heatable to achieve a minimal thermal angioplasty capability.

While barrel-assemblies intended for use in both diseased arteries and stenosed ducti of other types provide temperature settings from 50 to 100 degrees centigrade in ten increments of five degrees each, barrel-assemblies for use limited to atheromatous arteries are set to 90 degrees centigrade. Such a precautionary angioplasty would best be accomplished passively as an ancillary or incidental function attendant upon, rather than as a separate procedure preliminary to, implantation. However, a. The recovery electromagnets toward the front of the muzzle-head cannot be heated and used tractively at one and the same time, b. The use of one recovery electromagnet to heat while the other is used to recover would represent a circumferential insufficiency on both scores, c. For both (1) Freedom of movement and (2) Access to the free end for insertion of a cooling catheter, the barrel-assembly, even when used for minimal thermal angioplasty- (lumen wall priming-searing capability, is best completely separate from and independent of the airgun.

Thus, even though such a precautionary angioplasty is not discretionary as is an angioplasty that targets atheromatous tissue, a minimal thermal angioplasty- (lumen wall priming-searing-) capable barrel-assembly is still used independently of an airgun and provided with an on-board hand-grip that contains a battery pack and mounts a control panel. The minimal capability angioplasty barrel-assembly is thus an abbreviated version of which the capabilities fall within the scope of those included in an angioplasty-capable barrel-assembly. However, lacking a heatable turret-motor and side-sweepers and therefore the means for performing a proper angioplasty, such an intermediate level barrel-assembly can be produced at lower cost.

In such a barrel-assembly, there need be only a nose-cap or nose-envelope heat-window at the front of the muzzle-head where contact is first made with the lumen wall and not a turret-motor heat-window, which is generally directional (circumferentially oriented and longitudinally delimited). Once an angioplasty-capable barrel-assembly is inserted into the airgun, the electrical connection used to energize the electromagnets and turret-motor, now used positionally and not as heating elements, is either made automatically by engagement of the barrel-assembly in the airgun chamber as shown in FIG. 49 or by plug connection on the outside of the barrel-assembly shortly to the fore of the airgun muzzle as shown in FIG. 52.

### Thermal Ablation and Angioplasty- (Lumen Wall Priming-Searing-) Capable Barrel-Assemblies

The trend toward stenting without first performing an angioplasty makes the measures taken to avoid disrupting vulnerable plaque all the more significant. Initial contact by a muzzle-head with the wall of an atheromatous artery occurs both with an ablation and angioplasty-incapable barrel-assembly and an ablation and angioplasty-capable barrel-assembly. Barrel-assemblies are usually selected to flush-fit the segment of the lumen to be treated and are thus frequently in contact with the lumen wall. Furthermore, though blood-grooves and other passages ameliorate the problem, the muzzle-head substantially obstructs the lumen, promoting means and techniques to hasten completion of the procedure, such as the providing multiple barrel tubes and automated discharge at preselectable specific intervals. The combination of contact with the lumen wall and haste would risk ruptures and the release of debris were not certain additional countermeasures incorporated.

These include providing the muzzle-head with a nose-cap heat-window that is heated by running current through the windings of both recovery electromagnets. Since a distal embolic protective trap-filter would also be capable of disrupting plaque, none is deployed when the presence of vulnerable plaque is suspected if not confirmed. A nose-cap heat-window is thus necessary in all but ablation and angioplasty-incapable barrel-assemblies that are used only to stent implant where plaque is not present. In addition to a nose heat-window, a fully angioplasty-capable barrel-assembly usually has heat-windows that conduct heat produced by passing current through the windings of the turret-motor.

Whereas the recovery electromagnet nose heat-window must deliver heat round and about, those over the turret-motor in an angioplasty-capable barrel-assembly are in the form of circumferential or arcuate slits, slots, or rectangles that can be directed toward eccentric plaque or other type lesions. Because temperatures other than 90 degrees centigrade tend to be thrombogenic, the insulation surrounding heat-windows whether omnidirectional or directional (eccentric) must minimize the generation of thrombogenic heat in the gradient areas bounding the heat-windows.

Because of the severe restriction in thickness, perfect insulation cannot be achieved; however, because the 90 degree focal area continuously passes over the lumen wall, areas within the cooling gradient bounding the heat-window within the segment of the artery to be treated are instantly exposed to the target temperature. Thus, the insulation is made as effective as possible but not perfect. There will always be end of treatment areas where the muzzle-head will not pass, and thromobolytic medication, which is always to be administered in the smallest dose effective, must be relied upon to protect against thromboembolisms.

The same applies when only one recovery electromagnet or the turret-motor are used directionally to treat eccentric lesions in a blood vessel. A probability if not the confirmation of vulnerable plaque demands additional protection against the release of debris. This is attained by incorporating a distal embolic protective filter. Although not fully angioplasty-capable, such a barrel-assembly can and therefore is made capable of performing a thermal angioplasty independently of an airgun. Since it follows the nose-cap thermal window at an interval, making the turret-motor heatable is subsidiary in the preventing ruptures.

To function as a thermal angioplasty- (lumen wall priming-searing-) capable barrel-assembly requires connection either a. To the power supply using the plug and socket arrangement shown in FIG. 52 with a power cord of sufficient length to allow freedom of movement, or that b. The barrel-assembly be made completely separate and independent of an airgun with on-board power and controls to perform a thermal angioplasty. Both configurations allow access to a service channel whether the central canal or a spare barrel-tube to introduce a catheter down to a muzzle-port in order to deliver medication or a lubricant. Whether power is derived through connection to the airgun power supply or from an on-board battery pack, an on-board thermal angioplasty control panel is provided. In barrel-assemblies for use not limited to atheromatous arteries, thermal ablative temperatures other than 90 degrees centigrade are provided.

Once completed, the proximal end of the barrel-assembly is engaged in the airgun and implantation initiated. The equal applicability of semi-tethered and nontethered means for drawing power reflects the transitional status of such a barrel-assembly as between angioplasty functionality that is slavish or is independent, the choice in componentry depending upon whether the barrel-assembly is to be usable independently.

### Ablation and Angioplasty-Capable Barrel-Assemblies

The addition of side-sweepers to a thermal angioplasty-capable barrel-assembly yields a barrel-assembly that can be used to perform a thermal ablation (thermbablation) in a ductus other than vascular or an angioplasty as an independent procedure, regardless of whether angioplasty is followed by conventional stenting or insertion of the angioplasty-capable barrel-assembly into an interventional airgun for stenting implantation without the need to withdraw from and reenter through the introducer sheath and irritation to the entry wound. For use independently of an airgun, the barrel-assembly must be fully self-contained with distal embolic protective filter, on-board power pack, and angioplasty control panel.

Such a barrel-assembly requires insertion into an interventional airgun if and only if to be used for stenting implantation. For thermal ablation of ducti other than vascular, temperatures other than 90 degrees centrigrade must be provided. Combination-form fully angioplasty-capable barrel-assemblies additionally incorporate an atherectomy burr to cut through calcified plaque if necessary or a laser to perform an atherectomy in any type ductus. All fully angioplasty-capable barrel-assemblies incorporate a distal embolic protective trap-filter in the nose that can be remotely deployed or retracted.

### Barrel-Catheters, Barrel-Tubes, and Barrel-Assemblies

The barrel-catheters of simple pipes, comprising a single barrel intended for use primarily in the airway are one and the same tube. Single (monobarrel) and multiple barrel (multibarrel) radial discharge barrel-assemblies are designed for use in the bloodstream where air embolism and fouling of the mechanism by the inflow of blood must be averted even were discharge inadvertent with no miniball ahead of the expulsive gas. The barrel-tubes in radial discharge barrel-assemblies must therefore be enclosed within a jacket, or barrel-catheter, that allows the internal equalization of differences in pressure.

Except at the muzzle-hole, a radial discharge barrel-catheter is otherwise substantially airtight. The introduction of gas into the bloodstream is averted by providing a return path of less resistance than is posed by the blood even in antegrade (downstream) flow. This is achieved by perforating the barrel-tube or tubes so that gas may circulate within the enclosed barrel-assembly. The use of various tubing materials to include coextruded or compound tubing makes possible a wide range of pliancy and diameter in the unitary catheter, and this is significantly augmented in multiple barrel barrel-assemblies where the barrel-tubes may be made of different materials and adjusted in distance from the central axis.

[fig nr. Update Ended at this Point]

As represented in FIGS. 27 thru 32 described below, the pliancy of multiple barrel barrel-assemblies is also affected by the distance from the longitudinal central axis of the barrel-catheter of the barrel-tubes 74 through the holes 91 in and intervals along the barrel-catheter of the centering devices 95 used to position the barrel-tubes 74, and the incorporation, materials, and angles of blood-tunnels 96 seen in FIGS. 27, 31 and 32. When the diameter is too small to incorporate tunnels as buttresses, these are made solid or omitted. For increased flexibility or trackability and/or to absorb the shock of recoil, the barrel-catheter can include convoluted segments at intervals variable in length, interval, and number. By discontinuously applying the convolution impressing mold to the straight-walled tubing as originally extruded, the tube manufacturer can make tubing with any pattern of alternately convoluted and straight-walled segments. The barrel-catheter also includes a convoluted segment for flexion and recoil absorption.

The torque ratio or resistance to twisting and bending deformation of the barrel-catheter depends upon several variables, to include 1. The intrinsic pliancy of the material or if coextruded materials, 2. The wall thickness, and 3. Diameter of the barrel-catheter, 4. The intrinsic pliancy of the material of which each barrel-tube is made, 5. The radial distance set by the centering devices of the barrel-tubes from the longitudinal axis, 6. The longitudinal interval separating adjacent centering devices, 7. Whether the barrel-catheter incorporates blood-tunnels, 8. Whether the operator chooses to preinsert a catheter or rod of widely variable pliancy down an available barrel-tube, 9. The incorporation into the barrel-catheter of convoluted segments, the lengths of and intervals separating any convoluted segments, 10. Whether the centering devices are left free to rotate at the edges or have been bonded to the barrel-tubes and the internal surface of the barrel-catheter, and 11. The resistance to twisting of any other lines running through the barrel-catheter, such as the wires to the turret-motor and the electromagnets and the optical fibers when a laser, for example, has been incorporated, and so on.

The use of materials and incorporation of internal structural features to be described and the selection of barrel-assemblies to avoid nonessential length allow approximating a 1:1 torque ratio, and the muzzle-head bears tantalum markings for high radiopacity. Anatomy permitting, this allows even an exceptional muzzle-head that lacked a turret-motor to be accurately rotated into the desired position manually. The resistance to bending of the extracorporeal portion of the barrel-assembly is more significant in angioplasty-capable barrel-assemblies, where excessive pliancy or droop under the weight of the battery-pack and electronics with control panel would prove a source of annoyance.

The use of a cooling catheter to return the temperature of the turret-motor and/or recovery tractive electromagnets when sent heating current for thermal angioplasty, as will be described below, is more effective when the diameter or gauge of the cooling catheter can be larger as passable down the central canal of a center-discharge barrel-assembly rather than down a barrel-tube as is necessary when using a combination-form barrel-assembly that incorporates an atherectomy burr or laser cable at the center. The general concept of a cooling catheter is not new.

Considered in cross-section, the heat conduction path from within the otherwise insulative barrel-tube to the turret-motor and electromagnets is asymmetrical (off-center, eccentric); however, for equalizing the pressure of discharge to avoid arterial or venous gas embolism (air embolism) (see, for example, Mendenhall, M. L and Spain, D. A. 2007. “Venous Air Embolism and Pressure Infusion Devices,” Journal of Trauma 63(1):246; Wittenberg, H. G. and Allison, J. R. 2006. “Venous Air Embolism,” eMedicine, available at http://www.emedicine.com/emerg/topic787.htm) the barrel-tube has been perforated over the distal segment aligned to these heated elements, which perforations pass the heat. The exit velocity of the barrel-tubes within a barrel-assembly are not equalized as necessitates that all be perforated exactly alike.

### Types of Barrel-Assemblies

Barrel-assemblies in themselves are limited to delivering implants. Barrel-assemblies for use in the circulatory system must incorporate the features described herein for internally dissipating the pressure of discharge and not inject gas into the bloodstream. Included are barrel-assemblies of the simple pipe and radial discharge only kinds. Angioplasty barrel-assemblies can be used to perform an angioplasty and include radial discharge barrel-assemblies with side-sweepers and a turret-motor that as a positional control second, nonconcurrent, and unrelated function can be heated to serve as a temperature controlled heating element that warms the muzzle-head shell for thermal angioplasty. Combination-form angioplasty barrel-assemblies additionally include a rotational atherectomy burr or laser. The burr or laser cable is contained along the central axis requiring the displacement of the barrel-tubes radially outward, which configuration is referred to as edge-discharging, a barrel-assembly with the center free referred to as center-discharging.

Neither type cable prevents flexion of a convoluted segment joining proximal and distal portions of a flexible muzzle-head. Angioplasty barrel-assemblies require a muzzle-head body or shell with heat conductive and insulative properties to permit the heat generated within the body to be radiated toward the lesions, most often atheromatous, which are usually delimited longitudinally and radially asymmetrical, in a directed manner as described below. Polytetrafluoroethylene-coated nonferromagnetic stainless steel affords the surface slippage to avert endothelial clinging.

Whenever the turret-motor is joined to the more distal elements of the muzzle-head (ejection head, recovery electromagnet assembly) by a segment or joint of flexible convoluted tubing, the muzzle-head body shell must be divided between the portions of the muzzle-head distal and proximal to the flexible joint. While heat-windows, slits, or slots for thermal angioplasty (below) heated by sending heating current to both the turret-motor and the electromagnet assembly in a center-discharge muzzle-head must be divided between the shells proximal and distal to the flexible joint, the path through the barrel-assembly for passing a capillary catheter for rapid cooling (below) of the heated elements to body temperature through the central canal of the peribarrel space and up through the capillary catheter channel in the ejection head in only this type of barrel-assembly is continuous.

In a combination-form barrel-assembly, the center is taken up by an atherectomy burr or laser cable and is thus unavailable for insertion of a cooling catheter. With this type of barrel-assembly, which requires an edge-discharge type muzzle-head, the cooling catheter must be passed down to the muzzle-head through an available barrel-tube. If in order to use miniballs of a certain diameter this necessitates the use of a barrel-assembly of larger diameter, use of a cooling catheter will have been preempted and therewith the use of combination-form barrel-assembly, which uses an edge-discharge muzzle-head. Because the internal diameter (caliber, gauge) of the barrel-tube can be on the order of 0.4 millimeters, the cooling catheter must be thin. To allow the cooling catheter to be fed down to the muzzle-head thus requires that it be made of a relatively rigid polymer, seldom other than polytetratluoroethylene.

In barrel-assemblies designed to use the turret-motor or tractive electromagnets as heating elements for thermal angioplasty, these same materials afford the effectively nonmagnetic properties and low thermal conductivities or heat transfer coefficients, so that by placing a pane of sheet silver or copper, which are high in thermal conductivity, in the muzzle-head body, allows heat regulated in temperature by controlling the current to the windings, to be directed, in effect beamed, from the body toward the lumen wall over a defined area.

Unlike simple pipe barrel-assemblies, which are intended for use primarily in the airway, radial discharge barrel-assemblies are intended to be usable in the vascular system as well as small and structurally undifferentiated ducts. The latter factor and need for operative speed account for embodiments that are able to deliver multiple implants with each discharge. As opposed to use in a body cavity, the use of a simple pipe barrel-assembly in a ductus or vas in a human is essentially limited to the airway. Ablation and angioplasty-incapable (plain discharge, limited-purpose) radial discharge barrel-assemblies as shown in FIGS. 24, 25, 27, and 38 are limited to use with an airgun for implantation and are never used independently of an airgun.

By contrast, angioplasty-capable barrel-assemblies (angioplasty barrel-assemblies, thermal angioplasty barrel-assemblies) might be used solely to perform an angioplasty even when not planned to be followed by stenting, or might first be used independently of an airgun for angioplasty, and thereafter, without withdrawal from the patient, inserted at the free or proximal end into the barrel of an airgun to initiate stent implantation. Unless it incorporates a rotatory atherectomy burr or laser catheter, an angioplasty barrel-catheter powered by a hand-grip shaped lithium-polymer or silver-zinc battery pack need not be tethered, or connected by hard wiring to a power supply whether inmate in the airgun or another.

With either a rotational cutting burr or laser incorporated, local controls are included in the onboard barrel-assembly control panel, but the burr pneumatic drive and laser photoactivation components within the consoles of these cannot be miniaturized for incorporation into a barrel-assembly, which accordingly requires a pneumatic or fiberoptic cable connection. However, stenting always follows angioplasty, and the connection to either such drive, which is located at the proximal end of the angioplasty barrel-assembly, is designed to allow immediate disconnection from the console and reconnection to the airgun by means of the same connector fitting.

The barrel-assembly should be devised and chosen to avoid unnecessary length. Bends are eliminated from the initial period of discharge by providing the airgun with a barrel of some length, into which the proximal length of the barrel-assembly is inserted. In an angioplasty barrel-assembly, this would result in the length of the barrel-assembly to be inserted into the barrel of the airgun extending proximally past the hand grip shaped battery pack with control panel mounted, thus denying use of this length when or while the barrel-assembly was used independently of the airgun for angioplasty.

Rather than to allow the proximal segment of the barrel-assembly, or barrel insertion segment, to be denied for intraductal insertion, the hand grip is slid backward or toward the proximal end of the barrel-assembly when the barrel-assembly is used independently of the airgun for angioplasty. Angioplasty preceding stenting, the hand grip is initially in the proximal position where it is in electrical contact with the terminals to each side-sweeping brush-lifting thermal expansion wire (below), and the turret-motor and recovery electromagnets while used as heating elements for thermal angioplasty.

When the barrel-assembly is to be inserted into the barrel of the airgun, the grip with forward drive stabilizer if present is slid forward or distally up to a detent or stop marking off the length of the airgun barrel and disconnecting the onboard battery pack from electrical contact with these terminals, which then can make contact with the terminals within the chamber of the airgun or attached to the hand-grip. In use for implantation, the barrel-assembly is generally a passive component of the interventional airgun.

### Simple Pipe Type Barrel-Assemblies

When the anatomy within the lumen, such as in the trachea, is structurally differentiated necessitating the discriminatory placement of each implant and the distances separating successive discharges are large enough for manual placement, automatic transluminal movement and triggering in uniform measured increments as is appropriate in a structurally undifferentiated lumen of small diameter is unsuitable. The simple pipe type barrel-assembly is intended for targeted implantation in a surgically entered body cavity or in a ductus or vas that is open to the exterior and large enough to allow the muzzle-head to be maneuvered without the need for repeated withdrawal and reentry. Directly manipulated rather than remotely controlled by a positional control system, the simple pipe barrel-assembly is suitable for use in larger ducti that can be implanted with larger and more sparsely spaced miniballs with little risk of pull-through. Since the tortional stiffness or ratio of applied torsion moment to angle of twist or twistability of the simple-pipe barrel-assembly is likely to restrict free rotation at the muzzle-head when the extracorporeal portion of the barrel-catheter is short, the muzzle-head is generally formed as a separate handpiece that is connected to the barrel-catheter by a rotary joint at a level along the length of the barrel-catheter proximal to that for intracorporeal intermission.

The operator grasps the portion distal to this rotary joint, the muzzle-head; by a sliding spring loaded pinch clamp grip XX that is lined with a nonallergenic synthetic substitute for latex having a ribbed or textured internal surface to cushion the compression against as not to distort the barrel-catheter and to prevent unintended displacement. Since a simple pipe type barrel-assembly requires that the operator not let go of the muzzle-head pinch clamp grip XX to discharge, the aid of an assistant is required. When the simple pipe is connected to a modified air pistol (hand airgun, air handgun) as described under the section below entitled Modification of Marketed Airguns, the gun, whether slid along a table top adjusted to the height of patient entry or held by an assistant, is allowed to move freely, and when the operator indicates that he is about to depress the trigger, the assistant is given a brief interval during which to make certain that the barrel-catheter is straight.

The barrel-catheter is not pulled straight as would displace the aimed muzzle-port but rather held level at the midpoint where it sags, the gun retracted only so much as is necessary to take up the slack. When the simple pipe is connected to an interventional airgun as described under the section below entitiled Dedicated Interventional Airguns, the airgun is mounted on a wheeled tray or small dolly set on a table top adjusted to the height of patient entry, which the assistant allows to roll freely for the operator to indicate that he is about ready to trigger discharge, the assistant moving the table as well if necessary. When the operator indicates the intention to trigger discharge, the assistant assures that the barrel-catheter is straight and level by supporting the sag by hand and retracting the airgun only so much as is necessary to take up the slack. The simple pipe can also be adjusted in sag or lateral curvature intentionally to reduce the exit velocity; however, this must never be done without first testing the airgun as described below with each such bend, and since to do this will require disconnecting the barrel-assembly from the airgun, such use in not preferred to adjustment that uses the sliding valve modification applied to the valve body also described below under the section entitled Modification of Marketed Airguns.

When the veterinary patient with collapsed trachea is so small that the curve cannot be reduced sufficiently to avoid laryngeal injury, a radial discharge barrel-assembly with extracorporeal hand-held electromagnet should be considered. If no barrel-assembly appears usable, then arcuate stays with a stent-jacket should be considered before proceeding with the standard procedure for suturing prosthetic rings about the trachea. For collapse that extends into the bronchi in tiny patients, then implantation by means of a radial discharge barrel-assembly (below) combined with subcutaneous patch magnets (above) is preferable as negligible in level of trauma compared to a thoracotomy. A procedure for correcting and ameliorating the symptoms of tracheal collapse is described below. While demanding more operative time, a simple pipe with modified commercial air pistol is adequate and inexpensive compared to more complex barrel-assemblies and special-purpose interventional airguns.

The combination of barrel-assembly and air pistol is primarily intended for use by veterinarians to repair tracheal collapse in small dogs. The simple pipe can also be used in a normally closed vessel or duct where accidental injury or a primary procedure has given access. Unlike a multibarrel radial discharge barrel-assembly, the simple pipe lends itself to loading from a spring-loaded or gravity-fed linear or queue-type no less than a rotary magazine clip. The limited diameter of most ducti makes a barrel-assembly with a single barrel radial discharge muzzle-head imperative to achieve an outer diameter of two millimeters. Otherwise, such an embodiment is not intended for use in any closed ductus and less still in the vasculature. As may be seen by comparing the simple pipe barrel-assembly shown as engaged within an airgun in FIG. 20 to the single barrel radial discharge barrel-assembly shown in FIGS. 25 and 27, the simple pipe is equivalent and similar in conformation to an individual barrel-tube in a multiple barrel barrel-assembly as described below but provided with a surface surrounding the muzzle-port to protect the lumen wall and trap any loose miniball allowing the barrel-assembly to be withdrawn just enough to direct the magnetic field of the tractive electromagnets toward recovering these. Except in the smallest patients, there is sufficient space in the airway to rotate the tip without the need for a motorized swivel or turret.

In the smallest dogs, it will be necessary to use a single barrel radial discharge barrel-assembly as described in the section on single barrel radial discharge barrel-assemblies below. Since except when necessary to switch to a simple pipe with bounce-plate the barrel-assembly is preferably introduced only once into the airway and the barrel-catheter is not intended for bending mid-procedure as would necessitate withdrawal and reintroduction, the operator must select a tip of suitable angle before entry. The airway and digestive tract do not pose the problems associated with immersion in the bloodsteam as relate to the risks of stretching injury, the possibility of introducing gas into the blood, of inducing ischemia by obstructing the flow of blood, and severely limited working room as limits a single implant discharging device to place implants quickly. The simple pipe thus requires no peripheral blood grooves, tunnels, or means for the equalization of internal pressure as is necessary in radial discharge barrel-assemblies suitable for use in the vascular tree as described below. The treatment of tracheal collapse is discussed in detail below. Multiple barrel barrel-assemblies with radial discharge muzzle-heads suitable for use in the vascular system are discussed thereafter.

Because the airway, unlike the arterial tree, for example, affords sufficient space to rotate the muzzle-port for discharge and recovery without risk of injury, and rather than to be carried off to produce an occlusive problem, loose miniballs stick to the lumen wall, a simple pipe does not require a motorized swivel or more than a single target-directed tractive electromagnet as would increase its cost. Implantation of the tracheal ceiling is accomplished with the patient supine and the muzzle-port directed downwards, generally necessitating rotation of the muzzle-head by no more than 120 degrees to either side. By contrast, to recover a loose miniball in an artery with a single inmate or built in tractive electromagnet would demand immediate rotatability through 180 degrees in either direction. To assure the retrivability of any loose or mispositioned miniball necessitates the incorporation of more than a single tractive electromagnet and rotatability by means of a remotely controllable electrical motor.

Using a simple pipe barrel-assembly in the trachea, whether because a loose miniball must be retrieved, or the progress of collapse is so advanced that the tracheal floor must be implanted as well, rather than to withdraw and re-introduce the barrel-assembly risking injury to the larynx and lumen wall, all implants in the tracheal ceiling are completed first, and thereafter, the patient, not the simple pipe barrel-assembly, which is always to be used with the simple pipe muzzle-head directed downwards, is rolled over from a supine to a prone position under fluoroscopic observation. Nevertheless, should the operator ascertain that a second pass of miniball implants in the reverse direction is needed, the simple pipe is withdrawn and another equipped with a bounce-plate introduced. Referring now to FIG. 20, shown is a simple pipe barrel-assembly engaged in an airgun. A simple pipe barrel-assembly consists of barrel-catheter 44 with curved section 45 toward the distal end to direct the miniballs toward the tissue, usually at an angle of 35 degrees. In simple pipe and single miniball radial discharge barrel-assemblies, the barrel-catheter and barrel are one and the same.

The distal portion of the barrel-assembly is curved to allow a miniball to be implanted, in tracheal application as will be described, at the anterior junction of each successive cartilage ring with the annular ligament, bilaterally, along imaginary lines that would demarcate the lateral edges of the dorsal quadrant of the trachea were it circular. Trap-extraction electromagnet 46 is of the same construction and electrical connection as is specified below for radial discharge muzzle-heads, but is singular and as visualized in cross-section, directed radially toward the tissue rather than as the off-center cords described by a complementary pair of electromagnets. Housing 56 for the trap and extraction tractive electromagnet 46, is fixed in position within the concavity described by the underside of the curve 45. The barrel-assembly comprises the barrel-catheter 44 and electromagnet 46 within electromagnet housing 56 built as a unitized component to be plugged into the barrel 57 of an airgun by means of annular twist-to-lock fitting 47. Making housing 56 of a short-term tissue compatible bendable plastic and providing a small separation between the rear (top side) of the electromagnet and the bottom curve of the pipe handpiece allows the operator to slightly bend the pipe as necessary.

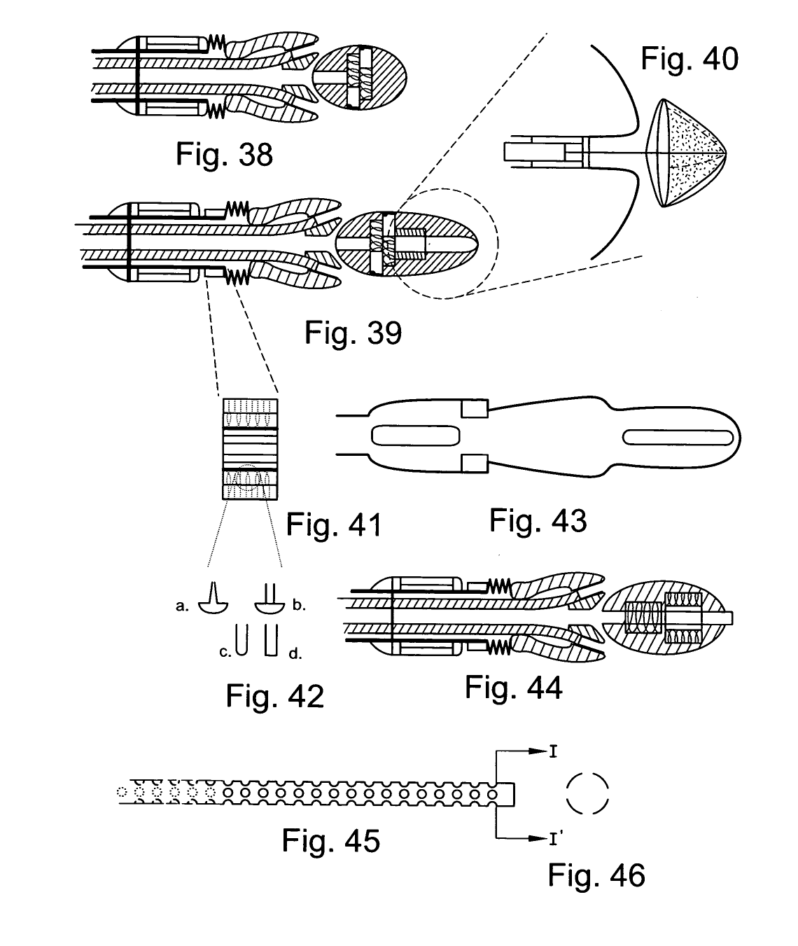

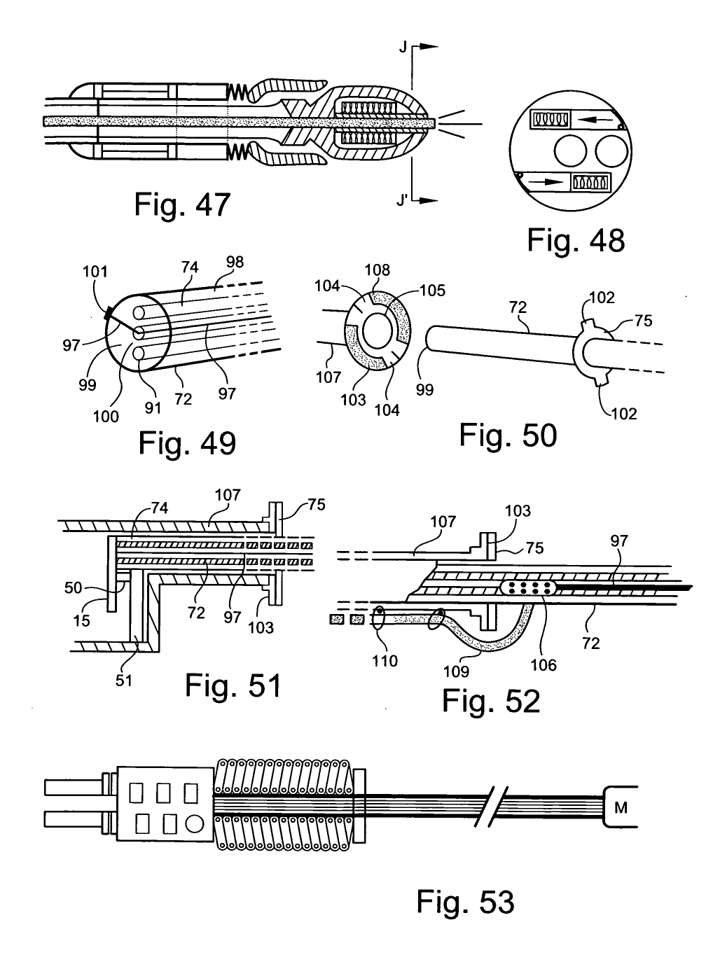

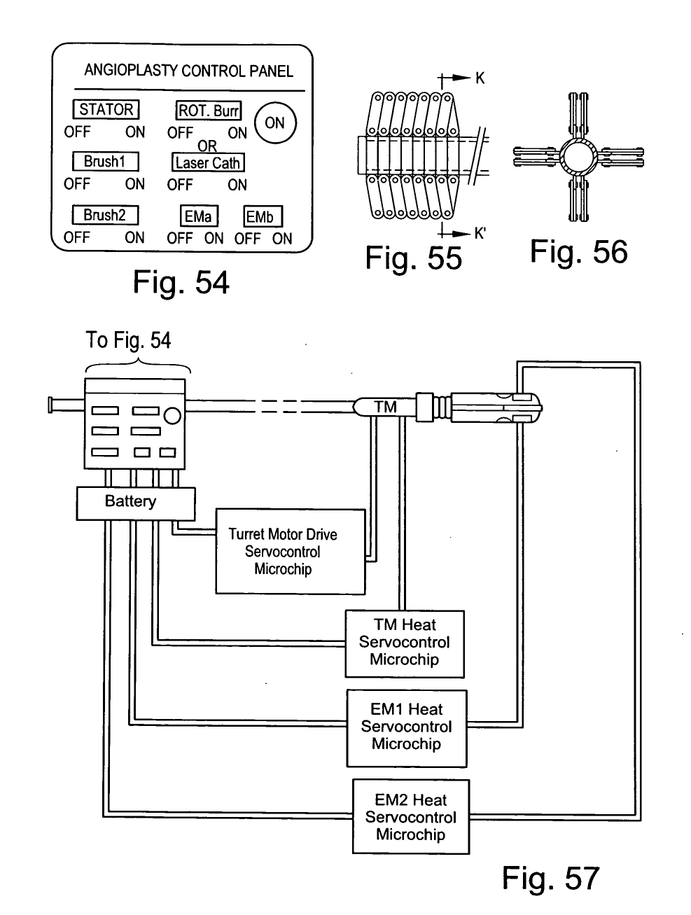

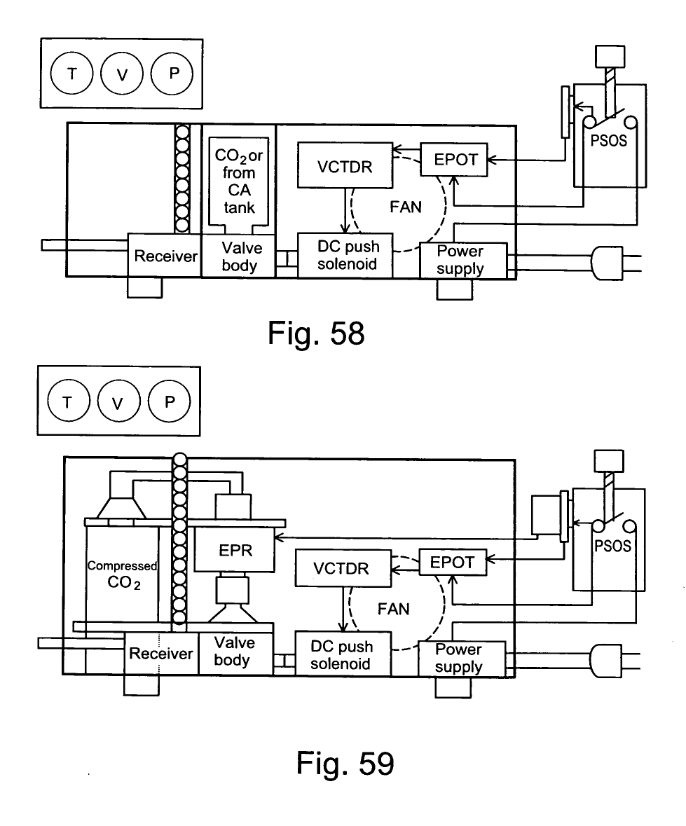

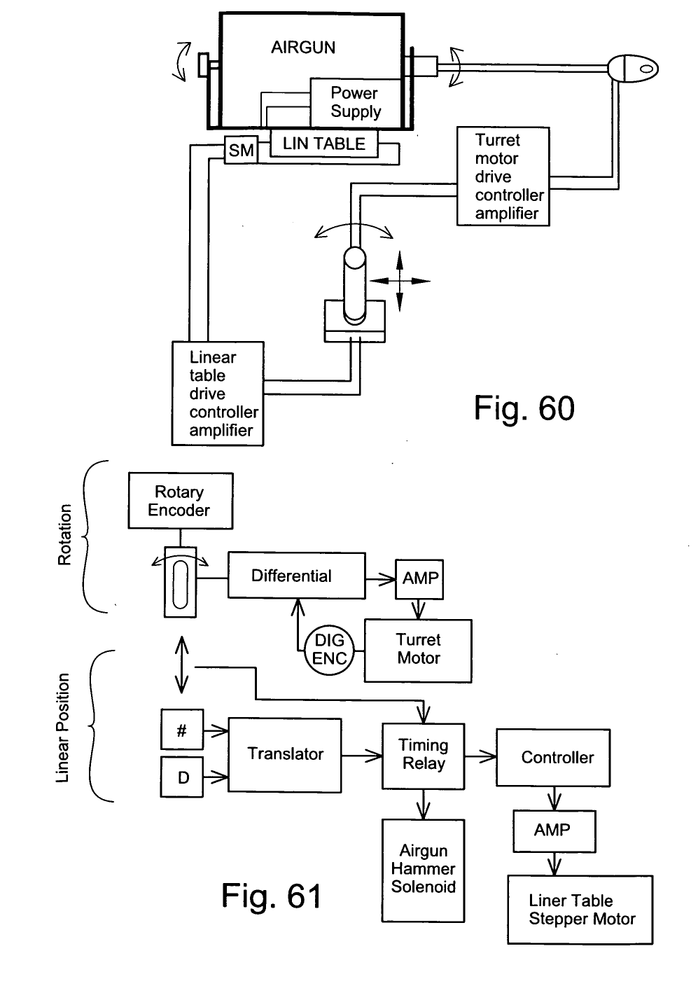

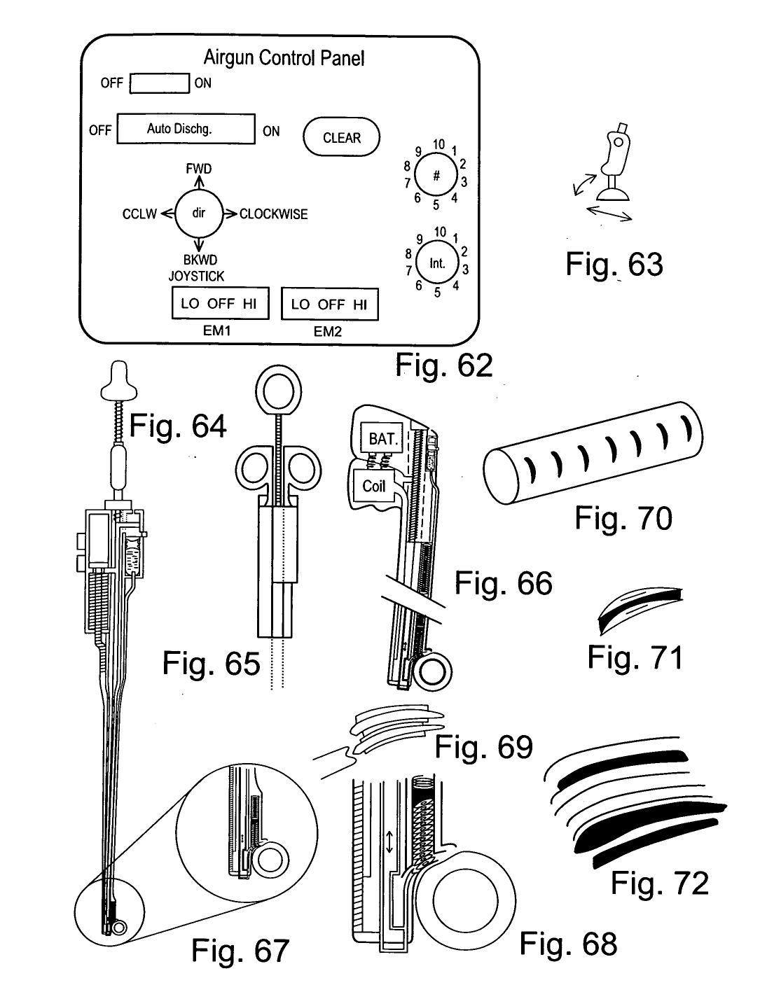

If, for example, the simple pipe is made of a substantially if not perfectly nonmagnetic stainless steel on the outside with an internal coating of polytetrafluoroethylene and the electromagnet housing 56 is made of a pliable polystyrene, then a suitable adhesive is a two part polyurethane, such as Loctite U-05FL, part number 29348, applied with mixer nozzle part number 98454 and dispensing gun part number 98472. Allowing some pliancy makes it possible for the operator to slightly bend the pipe at the curve if necessary. If altered, the pipe must be tested to ascertain the effect upon the exit velocity and consequent need to adjust the controls before use. The simplest and most direct test is that described below in the section below entitled In Situ Tissue Puncture and Penetration Test. This universal connector for engaging a barrel-assembly in the barrel of an airgun, which differs only in diameter for barrel-assemblies that contain multiple barrel-tubes, is described below under the section on the mechanical connection of the barrel-assembly to the airgun, and is shown in FIG. 38.

Simple Pipe Barrel-Assembly with Bounce-Plate

A barrel-assembly for use in the vascular tree must provide quick operation and means for minimizing if not completely eliminating the need for withdrawal and reentry. The substantially undifferentiated structure of the vascular lumen lends itself to multiple radial discharge, which is able to place four if not more miniball implants per discharge. By contrast, the tracheal lumen is structurally differentiated and eccentric, necessitating the discretionary placement of each implant. A bronchoscope lashed beside the barrel-assembly in the airway, a viewable muzzle-head is preferable for aimed ‘shots.’

Because to observe the muzzle-port or ports is unintended and more difficult with a barrel radial discharge barrel-assembly, the simple pipe is preferred for use in the airway. The single barrel radial discharge barrel-assembly is used only in the airway of the smallest dogs where there is not the space to manipulate a simple pipe and in distal segments of the bronchi where these are relatively undifferentiated. The structural differentiation and consequent need to place the implants in a discretionary manner can in some cases recommend the availability of a simple pipe barrel-assembly with bounce-plate (ricochet-plate; rebound-tip; rebound-plate), which allows reversing the direction of the trajectory, that is, directing the miniballs back toward the operator or proximad. This capability can be beneficial, for example, in the trachea to introduce implants into the posterior junction of each successive cartilage ring with the annular ligament, as described below.

However, the avoidance of withdrawal and reentry in the airway is not onerous as it is in the bloodstream, and such a capability is often unnecessary. The single barrel radial discharge barrel-assembly and not the simple pipe is recommended when space is lacking to insert and withdraw the simple pipe without risk of injury to the larynx. In smaller patients, a simple pipe barrel-assembly may be usable for a distance towards the bronchi, down to which the diameter of the lumen becomes so restrictive that it becomes necessary to withdraw and replace the simple pipe with a single barrel radial discharge barrel-assembly. The bounce-plate is thus incorporated into a second simple pipe rather than as an option that would be opened or closed in a single embodiment.

Under such circumstances, withdrawal and reentry is preferable or essential, so that a single embodiment capable of discharge both distad and proximad, which to provide entails additional complexity and cost greater than the sum for separate barrel-assemblies where one does and the other does not have a bounce-plate, is not preferred. Accordingly, a simple pipe barrel-assembly that reverses the direction of the trajectory is provided in a separate barrel-assembly. FIG. 21 shows a simple pipe barrel-assembly with bounce-plate at the distal end of the muzzle-head engaged in an airgun. Except for the addition of bounce-plate 53 and a soft protective annulus 52 adapted for the change in configuration of the muzzle-head that results from the bounce-plate, the simple pipe is the same as that shown in FIG. 20.

A detailed view of the tractive electromagnet 46 mounted in the concavity on the underside of the simple pipe barrel-assembly in the curve 45 approaching its distal end is shown in FIG. 22. The loss of a miniball in the airway being unlikely and posing little risk even were it to occur, an antemagnet chamber as seen in magnet assemblies used in radial discharge barrel-assemblies for use in the bloodstream described below, is not used. Electromagnet 46 is enclosed within electromagnet housing 56 made of any hard plasticizer free resin and bonded in position by means of an adhesive that is pliable after curing as discussed in the preceding section. When the airway is large enough that withdrawal of a nonrebound muzzle-head as shown in FIG. 22 and the introduction of a rebound muzzle-head or muzzle-head with bounce-plate poses minimal risk of injury, the separate embodiments represented by FIGS. 22 and 23 are used.

When the airway is small making entry and withdrawal difficult, a simple pipe with a bounce-plate is used. The bounce-plate is a distal tip cap (crown, ferrule) friction-secured fitting that to attach or replace necessitates removal and reintroduction of the pipe; a deployable and retractable adjustable bounce-plate pipe mechanism that is compatible with guard surround or end-bumper 52 remains to be developed. The bounce-plate is made of nonferrous metal such as copper or aluminum, and as seen in greater detail in FIG. 23, in which the trajectory is represented as 54 and the end of the original muzzle-head 55 to which the bounce-plate has been added, extends proximally along the top and sides of the barrel-catheter to a length sufficient to minimize if not eliminate recoil upon discharge, which length depends upon the material of which the barrel-catheter is made.

A simple pipe made of plastic tubing that having been shaped under heat resists adjustment to the curve toward the muzzle-head as might be desired midprocedurally is requires the use of a slip-over sheath made of a bendable metal such as copper or an extension of the bounce-plate if one is in use can be used to reshape the curve. A shape holding sleeve of copper, aluminum, or plastic can similarly serve this purpose in a simple pipe without a bounce-plate. If a plastic pipe is used at all, the preferability of a straight length of a more pliant tubing with the use of a bendable slip-over sheath is clear. The curve imparting sleeve can be temporary or bonded to the end of the muzzle-head by means of an adhesive. If, for example, the simple pipe is polypropylene on the outside and the nonferrous metal of the bounce-plate is an alloy of aluminum, the adhesive, which must remain pliant after cured, is preferably a two part polyurethane, such as Loctite U-05FL, mentioned above in the preceding section entitled Simple Pipe Barrel-assembly.

The angle of rebound being equal and opposite to the angle at which the miniball strikes the bounce-plate upon exiting the original muzzle-port, seen in FIG. 23 as 55, the angle described between the trajectory upon colliding and rebound off of the bounce-plate is usually 45 degrees. Rebound dissipates the kinetic energy and momentum or propulsive force imparted to the miniball necessitating adjustment of the airgun setting. Since the simple pipe barrel-assembly is intended for use in the trachea and the single-barrel radial discharge barrel-assembly for use in the tracheobronchial tree when the lumen diameter is confining, sections to follow the description of these single barrel barrel-assemblies will be directed to the application of these barrel-assemblies for use in the airway. Multiple discharge barrel-assemblies, which are not used in the airway but rather in vessels and ducts are described later.

Connectors (Couplings) for Quick Release and Reconnection of the Barrel-Assembly to the Airgun with Proper Alignment

Except for size, the mechanical connection of the barrel-assembly to the airgun is substantially the same in embodiments that otherwise differ. This connection must allow the operator to immediately disengage the barrel-assembly for manual transluminal advancement, withdrawal, or rotation and just as quickly reinsert it into the airgun barrel to resume discharge with the assurance that the security of connection and alignment of the barrel-tubes to the holes respective of each in the rotary magazine clip will be exact.

### Twist-to-Stop and Lock Connector (Twist Lock Connector, Keyed Spring Lock Connector)

In use, the barrel-assembly is selected and manually introduced into the lumen through a conventional incision and introducer sheath. There is no guidewire. Once the position along the lumen is reached, the proximal end of the barrel-assembly is engaged within the airgun barrel with the extracorporeal segment straight. It depending upon the pliancy of the specific barrel-assembly, the extracorporeal length results in some downward bowing at the center, then the slack is taken up by backing up the muzzle-assembly with the linear table as described below until the muzzle-head can just be seen to move. If, the muzzle-head must be advanced or withdrawn midprocedure, the linear table is used or the barrel-assembly can be disengaged from the airgun and manually repositioned.

When manually repositioned, upon reconnecting the barrel-assembly, the linear table is used to ‘trim off’ or take up any slack and thus straighten the barrel-assembly. Unless deliberately activated, discharge remains disabled, eliminating the possibility that a bend might affect discharge. Use of the apparatus is described in greater detail following the section on the linear table and in conjunction with a description of the control panel. This is accomplished through the use of a twist-to-lock joint or coupling that incorporates short spring steel tabs with central depressions to receive protrusions on the complementary tabs that are mounted about the barrel-assembly. While a joint of the kind described requires a slight twist to connect or to disconnect, this is preferable to the relative lack of tight connection provided by more costly and complex joints that require no twisting motion, such as the quick disconnect hose couplings used in vacuum cleaners.

In the simple pipe and air pistol, only one barrel-tube need be aligned. However, in a multiple barrel radial discharge barrel-assembly, an end-plate at the proximal end of the barrel-assembly is essential to stabilize the position of the proximal ends of the barrel-tubes, and the rotary distance or throw of the complementary tabs much be such that the stops situate the barrel-assembly in the airgun barrel with the barrel-tubes aligned to their respective holes in the rotary magazine clip. To allow the precise fit (without play) of the barrel-assembly in the airgun barrel without sticking or seizing midprocedure, both the external surface of the portion of the barrel-assembly to be inserted into the airgun barrel and the internal surface of the airgun barrel, which must exactly match in diameter are made of low friction, generally fluoropolymer materials, such as polytetrafluoroethylene.

Mechanical connection of the barrel-assembly within the barrel of the airgun by friction fitting is avoided as risking resistance to removal if unavoidable midprocedure. Regardless of caliber, type, or number of barrels, mechanical connection of the barrel-assembly to the airgun is by means of a push-and-rotate-to-engage keyed flange type connector or coupling, as shown in FIG. 38. Receiving or female part 103 of the flange connector is mounted to the front of airgun muzzle 105 (not to be confused with the muzzle-probe or muzzle-head at the distal end of the barrel-assembly seen as 70 in the radial monobarrel shown in FIG. 22 and as 73 in the radial multibarrel shown in FIG. 24).

The part of the flange connector to be inserted 75, referred to as a stop-and-lock ring, is mounted [ADHESIVE?] around barrel-catheter 72 at the distance from end-plate 99 that barrel-assembly 72 is to enter airgun barrel 107. To engage the barrel-assembly in the airgun barrel, tabs 102 are inserted into slots 104. Rotating the end of the barrel-assembly clockwise causes the tabs to slide beneath the compressive ceiling overlying rotary slideway 108, which closed off at the extremity of rotation stops the tabs at the exact rotational angle at which the barrel-tubes in barrel-catheter 72 are aligned to their respective barrel holes in the rotary magazine clip shown in FIGS. 15 and 16 as 42. Simple pipe and radial discharge monobarrels require no alignment of plural barrel-tubes, but still require that the muzzle-port be directed downwards.

The component of the stop and lock ring component 47 of the twist-to-lock connector fitting mounted about the barrel-catheter has tabs (102 in FIG. 38) that fit into slots (103 in FIG. 38) in the complementary component fitted about the front of the airgun muzzle 48. Twist-to-lock fitting 47 both establishes the limit of intermission of the barrel-assembly into the barrel of the airgun and locks the barrel-assembly in position with its distal end just short of as not to impede the movement of the rotary magazine clip 15 in FIGS. 17 and 18. In accordance with the industry convention for indexing or incrementally rotating clip 15 when the trigger is pulled, notches 48 about the outer edge of rotary magazine clip 15 in FIGS. 20 and 21 are successively engaged by pawl 49. The rotary magazine clips are inserted by placing the center hole 43 over axle 50 mounted to supporting post 51. This action can be accomplished midoperatively in four seconds or less. These notches are on the reverse face of the rotary magazine clips and thus unseen in FIGS. 18 and 19.

Shown in FIGS. 20, 21, 24, and 25 as part 47, and in FIGS. 27, 34, 35, and 36 as 75, the male portion of stop and twist-to-lock connector, has at least two tabs that fit into the slots in a circular archway, or if the archway is divided for each, into the end-openings of the divided circular archway, that is mounted to the front of the airgun muzzle, so that once entered into the slots, twist-to-lock fitting 47 can be slid around through the circular slideway or slideways beneath the ceiling over the archway of the slideway to the slideway ends and so locked in position both angularly and longitudinally. In all barrel-catheters, the proximal portion for entry into the airgun barrel has an outer diameter that precisely fills the airgun bore or inner diameter of the airgun barrel, whether the airgun barrel has been modified with, for example, a polytetrafluoroethylene liner added to cover (blanket over) the rifling as well as to reduce the caliber, or the barrel is original in a dedicated interventional airgun, as later described.

Accordingly, engaged thus, the outer surface of barrel-catheter 44 is flush throughout its airgun-intromitted length against the bore or airgun barrel 57 with the proximal end of the barrel-catheter 44 positioned immediately before the miniball hole or holes 42 in the rotary magazine clip shown in FIGS. 18 and 19. This means for engaging the proximal end of a barrel-assembly in an airgun is common to all barrel-assemblies and thus addressed in a separate section below. Barrel-catheter 44 is made of a single length of tubing with the curve toward the tip 45 maintained by the bond that unites the upper surface of the tractive electromagnet 46 housing 56 to the underside of the barrel-catheter 44. To minimize the risk of injury to the lumen wall or larynx by gouging, the pointed end of the simple pipe barrel-assembly is girdled about with a soft guard (bumper, shield) 52, similar to a dam used in dentistry.

The guard is made of expanded polytetrafluoroethylene (ePTFE) or a pharmacologically active leachant and plasticizer-free engineered nylon polyether block amide resin such as Pebax® 3533 tubing. It is stretched over and fixed in position about the barrel-catheter by its own restorative force. Guard 52 overextends the distal end of the barrel-catheter slightly, typically by 1 millimeter. To accurately rotate the muzzle-head requires that the barrel-assembly resist twisting, or have a torque ratio approaching 1:1. Since radial discharge barrel-assemblies present an outer contour that is rounded and smooth, are provided with a remotely controllable motorized muzzle-head turret, and the muzzle-head is routinely wetted with a lubricant such as ACS Microslide®, Medtronic Enhance®, Bard Pro/Pel® or Hydro/Pel®, or Cordis SLX®, before entry, the risk of gouging, twisting, and stretching injury is avoided. When a single barrel radial discharge barrel-assembly is used in the airway, mucus serves as a lubricant.

When a combination-form muzzle-head includes an excimer laser (below), the ends of the optical fibers can be wetted with lubricant as well, the laser quickly vaporizing this upon activation. The degradation products liberated by such vaporization must, of course, be innocuous in the blood stream. Lubricant can interfere with the cutting action of a rotational atherectomy burr so that lubricant should be used sparingly to avoid the muzzle-head or probe nose. The means of testing for endothelial adhesion and delivery of lubricant to the muzzle-head midprocedurally are described below under the section on testing.

Broadly, whenever the risk of injury due to transluminal, manual, or motor torqueing (rotatory) movement of the barrel-assembly is present, a lubricant is used. Whenever the barrel-catheter of a barrel-assembly used in the vascular tree approaches the diameter of the muzzle-head, the entire barrel-assembly is wetted with a lubricant. Depending upon the materials used to make the barrel-catheter and muzzle-head, the outer surface of both components can be coated with one of the lubricious materials specified below. An outer coating of polytetrafluoroethylene will generally obviate the need for lubrication. Alternatively, silver-based coatings such as is available from Spire Corporation, Bedford, Mass. do not materially detract from lubricity but do appear to reduce the risks of Infection, thrombosis, and stenosis (see Bambauer, R., Mestres, P., Schiel, R., Schneidewind-Muller, J. M., Bambauer, S., and Sioshansi, P. 2001. “Large Bore Catheters with Surface Treatments Versus Untreated Catheters for Blood Access,” Journal of Vascular Access 2(3):97-105).

### Tubing Materials for Barrel-Tubes and Barrel-Catheters

Suitable tubing for the barrel-catheter and the barrel-tubes running through it pose numerous possibilities. The number of barrel-tubes can be from one to four or even more, can be separate or coextruded as a multiple lumen tube of which the luminal tubes are separable for outward flaring toward the distal end. The incorporation of blood-tunnels and spacing apart of the barrel-tubes by the centering devices, described below in the sections on centering devices and blood-tunnels, will affect barrel-assembly bendability. Depending upon the dimensions, rigidity, frictional character, and so on sought in a given type of barrel-assembly, the material, or if coextruded or coated, the materials of the tubes in that type of barrel-assembly are chosen on the basis of empirical testing.

The central canal in a center-discharge barrel-assembly or any available barrel-tube in either a center or edge discharge barrel-assembly can be used to insert a hollow tube or solid rod of a diameter less than that of the path taken made of any polymer free of polymerization residue to alter the flexibility of the barrel-assembly at any time whether before or after entry into the body or before or after reaching the site for treatment, to include a cooling catheter or cooling capillary catheter as described below.

The torque ratio or twisting characteristic of the barrel-catheter can be increased with relatively little effect on flexibility by bonding the centering devices to the inner surface of the barrel-catheter. The tubing in any barrel-assembly should be neither so pliant as to bend with little lateral force and thus change the rolling resistance as to necessitate constant adjustment of the airgun, which invites human error, nor so stiff that the bends encountered with either brachial or femoral entry cause it to kink or injure the lumen wall. For transluminal advancement by the linear positioning table, bending is prevented by a sheathing about the extracorporeal length.

The tubing of the barrel-catheter 44 can be made of any of a number of materials, to include compound (coextruded) tubing to provide, for example, polytetrafluoroethylene within nylon, polytetrafluoroethylene within vinyl, polytetrafluoroethylene or nylon inside with polytetrafluoroethylene or medical grade vinyl outside, or an internal thin coating or thicker layer of polytetrafluoroethylene for ‘bore’ slipperiness and overall stiffness, coextruded with an outer layer of any of numerous materials, such as expanded polytetrafluoroethylene, Pebax®, or Tygon® S-50-HL for pliancy and soft contact with the larynx and tracheal lumen.

Varying the relative thickness of each of these layers allows a continuous wide range of complementary pliancies and torqueabilities in the simple pipe barrel-catheter. Barrel tubing materials having a higher coefficient of friction recommend an inside barrel lining coat of polytetrafluoroethylene. Polytetrafluoroethylene within vinyl, for example, affords relatively greater pliancy, but at the expense of stiff torqueability and greater friction.

Nonessential bends in the barrel-catheter proximal to the patient will increase the rolling resistance for and reduce the exit or muzzle velocity of the miniballs. For this reason, no greater length of the barrel-catheter between the patient and the airgun than is necessary for the operator to freely manipulate the barrel-assembly should be allowed. That is, the barrel-assembly should be as short as practicable. The incorporation into the muzzle-head of a ‘chronometer’ to actuate an alarm when as the result of rolling resistance, miniball velocity drops below a certain value, is discounted due to the lack of space available distal to the front of the electromagnet.

Whereas a radial discharge barrel-assembly will generally be used for high density implantation necessitating leveling of the extracorporeal length for use of the automatic interval increment table, with a simple pipe barrel-assembly, monitoring for bends in the barrel-catheter to prevent reductions in exit velocity can be directly and practicably accomplished by incorporating piezoresistive or optical fiber strain gauges along the barrel-assembly at intervals over the proximal length that remains outside the patient. A threshold excessive output voltage generated within these can be made to actuate an audible alarm. However, vigilance by members of the operating team to any more than slight changes in the conformation of the barrel-assembly obviates the additional cost for a hyperflexed condition detection and alarm system. The ‘bore’ of the barrel-catheter varies with that of the miniballs to be discharged, generally ranging between 1.0 and 2.1 millimeters.

Regardless of the procedure or type barrel-assembly in use, once the apparatus has been positioned and is in use, further movement, much as with a dental hand-piece, is slight and substantially limited to the working end or muzzle-head. The airgun is adjusted for the conformation of the barrel-catheter, and significant deviations from this conformation must be noted and the procedure interrupted to change the adjustment. That once in use movement is limited to a short length of the barrel-assembly toward the distal end does not justify the interposition of a section of tubing that differs in pliancy from the rest.

To assist in orientation and the gauging of distances, radiopaque calibrative markings are applied along the outside of the pipe by etching and applying tantalum. If the outside of the barrel-catheter is made of polytetrafluoroethylene, Acton Technologies, Inc. FluoroEtch®, for example, is used to prepare by scarifying the surface for improved adhesion of the tantalum coating. Further details regarding the mechanical connection, and the electrical connection, of the barrel-assembly to the airgun are described below.

### Forward Drive Stabilizer (Extracorporeal Barrel-Catheter Straightener and Deflection Prevention Stabilizing Linkage)

Any deviation of the barrel-assembly from the longitudinal axis will reduce the airgun exit velocity. Since the testing means to be described is conducted in situ, anatomical bends are accounted for; nevertheless, testing is neither expedient nor advisable for every adjustment in the extracorporeal length of the barrel-catheter. Unless, for example, the operator cancels the action by pressing the ‘cancel’ (clear, abend) key, the airgun in automatic discharge will continue to advance or retract and discharge until the number of discharges directed has been completed making testing for each discharge impracticable. Furthermore, the length of a barrel-assembly and its test rod are constant, and the test rod is flexible so that testing conducted with the maximum extracorporeal length does not give distorted results because the test rod straightened the barrel-catheter.

With an angioplasty-capable barrel-assembly, angioplasty per se with the barrel-assembly removed from an airgun, is usually accomplished manually, the operator grasping the barrel-assembly close enough to the entry wound to prevent the extracorporeal length of the barrel-catheter from bending, thus eliminating the need for a straightening device. The segment of the barrel-assembly to be engaged within the airgun barrel, the stop-and-lock ring, hand-grip (with angioplasty control panel when the barrel-assembly is angioplasty-capable), and forward drive stabilizer are not susceptible to bending.

A forward drive stabilizer can be incorporated into any barrel-catheter, to include combination-forms that incorporate a rotational atherectomy burr or a laser. Since neither is needed once the angioplasty portion of an operation has been completed, the free or proximal end of the barrel-assembly can be disconnected from the console cable and connected to the airgun. This allows the connection to be in the longitudinal axis and end at the proximal end of the barrel-assembly rather than on the side where the cable would interfere with a forward drive stabilizer. To minimize passive sagging due to unsupported length in excess of the self-supporting or bridging stiffness imparted by the inflexibility of the barrel-catheter, the barrel-assembly should be chosen for minimal extracorporeal length and the airgun retracted manually or with the linear table if present as necessary to achieve reasonable tautness and thus minimize this length.

To minimize deviations of the extracorporeal barrel-catheter from the longitudinal axis, whether due to 1. Passive sagging, 2. Increased extracorporeal length and bending should the linear positioning table of a radial discharge barrel-assembly fail to achieve penetration in forward drive, 3. Transient deflections associated with the expulsion and recoil of discharge, or 4. Further reduction in exit velocity when 2. or 3. occur when 1. is already present, an extensible sleeve is incorporated to constrain the extracorporeal barrel-catheter to a straight condition.

As shown in FIG. XX, the forward drive stabilizing linkage consists of an articulated succession of discontinuous or interrupted tubular barrel-catheter sleeving or sheathing segments that surround the extracorporeal barrel-catheter in vertebral relation and are maintained in stiff longitudinal coaxial alignment by a linkage that allows the tubular segments be axially drawn (extended) or closed (compacted) over the extracorporeal portion of the barrel-catheter. Portions of the extracorporeal barrel-catheter over which the linkage has been drawn are removed from susceptibility to deflections from the longitudinal axis whether steady state (sagging) or transient.

Shown fully compacted (collapsed, contracted, closed), the linkage, which embodies mechanical features similar to those of draw drapes and an expansion watch band (but not spring-loaded to contract), allows the tube segments to be pulled apart and thus extended over a greater length of the barrel-catheter with minimal bending, or pushed closer together to cover a shorter length of the barrel-catheter and thus allow the forward (distal) portion of the barrel-catheter previously covered by the linkage to be introduced into the body.

In both barrel-assemblies designed solely for implantation and those to function independently for angioplasty, hand-grip XX is provided as a torquing (catheter rotation) device, or torquer. Hand-grip XX is permanently fastened at its proximal end to the distal face of stop-and-lock ring 75 and at its distal end to the proximal face of forward drive stabilizer linkage XX, with the front end left free to extend along the barrel-catheter to its extensible limit. In barrel-assemblies designed to function independently for performing angioplasties, the hand-grip mounts an independent onboard angioplasty control panel and unless battery powered as preferred, connectors for the cord supplying power to the internal components.

The tube segments are cut from tubing that is unfinished (rough surfaced, unpolished, unplated) on the side to face the exterior, such as steel coated for corrosion resistance after cutting, or a strong alloy of a nonferrous metal such as copper or aluminum. To avoid sticking (catching, seizing) whenever the linkage is extended or compacted, the internal diameter of the tube sections is not made flush to the outer surface of the barrel-catheter but allows sufficient clearance for extension and contraction. This clearance makes possible reducing the number of linkages with distinct internal diameters and thus the cost of production.

Arms XX of linkage XX are die cut from sheet metal of the same material and joined by solid or clevis pin type round head rivets with spring washers, preferably of the Belleville disc ring type, interposed between the heads of the rivets and the arms of the linkage. The ends of the linkage are clamped about the barrel-catheter by means of subminiature spring collars unitized to the ends of the linkage. Linkage arm posts XX are fastened to the outer surface of tube segments XX and miniature spring collars XX to the ends XX of linkage XX by micro resistance, or fine spot-welds. Small wires intermittently strung between successive links serve to prevent extension in such degree that compaction is restrained. To save machining, the wires can be wound about the rear ends of rivets rather than separately fastened to adjacent arms of the linkage.

### Limited-Purpose Single Barrel (Monobarrel) Radial Discharge Barrel-Assembly

Essentially enclosing a simple pipe within a torpedo shaped shell, a radial discharge muzzle-head presents no sharp distal tip as necessitates sufficient space to maneuver it in order to avoid injuring the lumen wall. The cylindrical conformation of a radial discharge muzzle-head allows its introduction into a lumen slightly smaller in diameter than the muzzle-head itself without the risk of stretching injury or the need for a angiotonic relaxant (angiotensin counteractant, hypotensive agent). The outer surface of the muzzle-head body is made lubricious to prevent clinging even during rotation. When made of nonferrous metal, to minimize adhesion to the lumen wall the muzzle-head of any radial discharge barrel-assembly is preferably coated with a fluoropolymer such as polytetrafluoroethylene.

Since it is limited to one radius or maximally eccentric and relatively small in diameter, and thus less likely to exhibit strong resistance to twisting when torqued, but still includes tractive electromagnets, a radial discharge monobarrel benefits most from a turret-motor. The ability to aim the tractive electromagnets with facility allows the resting or steady-state trap retraction field force to be reduced reducing the risk for extracting a miniball that has been correctly implanted, and the side-sweepers, installed normal to the longitudinal axis of the muzzle-head, can be used rotationally as well as during transluminal movement.

Any radial discharge barrel-assembly must be usable in the vasculature and must therefore incorporate means for preventing 1. The introduction of gas into the bloodstream during discharge, 2. Admitting an amount of blood into the muzzle-head sufficient to affect either the exit velocity or the internal equalization of pressure significantly, and 3. Preventing the loss of a miniball implant that could be carried downstream. Accordingly, if the size of the patient or preliminary fluoroscopic examination reveals that the airway or distal portions thereof are too small in lumen diameter to manipulate a simple pipe, then a single barrel radial discharge barrel-assembly is used. Such a barrel-assembly is the same as one used in the vascular tree. Owing to the small diameter of most vessels and the eccentricity of most vascular lesions, the single barrel radial discharge barrel-assembly, because it can be made to the smallest diameter of any barrel-assembly, has the widest applicability, multibarrel embodiments serving to reduce operative time.

Even though working in the airway does not impose the demands for gas containment and nonsusceptibility to thrombose or clog that necessitates the use of a radial discharge device as in the bloodstream, lumen diameters that are too constraining to use a simple pipe necessitate the use of a radial discharge muzzle-head. Since the degree of anatomical differentiation in the airway becomes less distad, this is not a problem; however, an occasional dog with collapsed trachea will be so small that a simple pipe can be used for no more than the proximal segments.

The single barrel radial discharge barrel-assembly is similar to the simple pipe barrel-assembly in that the barrel-catheter and barrel are one and the same. The minimum diameter of the muzzle-head necessarily limited by the number of barrels, the single barrel radial discharge barrel-assembly allows access to vasculature and ducti very small in gauge, allowing treatment more deeply or distal into the vascular or tracheobronchial tree. Access to vessels and ducts less than a millimeter in lumen diameter also extends applicability to neonates and small veterinary patients. For this purpose, a rotary is more versatile than a linear feed magazine clip in allowing successive implants of different mass.

### Radial Discharge Barrel-Assembly Working Arc

The working arc is the practical range of rotational excursion of the muzzle-head to either side of the turret-motor set point as limited by the deformation tolerance of the barrel-tubes in use. The working arc is thus the arc through which the turret-motor is limited in rotating its specific muzzle-head or the distance to which the muzzle-port group can be rotated to either side of its center as point of reference and thus the arc of the lumen in enfilade, or the arc of the lumen circumference accessible to discharge, within the beaten zone. Since unlike radial discharge barrel-assemblies, a simple pipe-type muzzle-head does not a. Conduct barrel-tubes internally in concentric relation that must not be deformed (bent) at the curve preceding the muzzle-port to the extent that jamming results so that its rotation must be limited, or b. Require remote rotation to either side of the center or zero-point of a positional control system, it can be freely rotated by hand, so that the concept of a working arc does not apply to it.

The limited rotatability of the muzzle-head cannot be overcome by any adjustment to the onboard components, such as by redefining or electronically moving the setpoint at the numerical position translator. The arcuate limit established by the twisting limit imposed by the barrel-tubes, adjustment by this means will only shift the setpoint so that a portion of the working arc that would have remained accessible had the setpoint not been shifted is removed from access. A detent arm that projects from the rear rim of the muzzle-head and stop-tab secured to by ringing (banding) about the barrel-catheter prevents over-rotation of the turret-motor during direct manual control, the incorporation of a circuit breaker or warning signal for the purpose thus rendered redundant.

When the muzzle-ports are placed about the muzzle-head in radial symmetry, any port can be taken as the reference index for rotation. However, since an eccentric muzzle-head groups the muzzle-ports more closely about the circumference, this center point or reference index for turret-motor rotation is taken as aligned to the most central muzzle-port in the group. Using four or more muzzle-ports places every point about the lumen circumference under the rotational arc of two muzzle-ports, which would eliminate the need to rotate the airgun were discharge limited to quadrant placement.

In fact, by blanking rotary magazine clip holes, performing one transluminal run, indexing the muzzle-head by the circumferential distance from the first run desired, then reversing direction in a second run, it is possible to lay down the implants in any pattern; since a lesser degree of rotation will bring one or another of the muzzle-ports to overlie or subtend any arc about the muzzle-head, increasing the number of evenly spaced muzzle-ports reduces the need to rotate the working arc. The use of rotary magazine clips is described below.

However, an eccentric muzzle-head can gain the advantages of less complexity and greater speed, can, for example, lay down several closely spaced rows in a single run. When it is difficult to ascertain whether a suitable starting angle (working arc center; control set point) as establishes the center of the working or treatment arc has been achieved, preliminary discharge for effect allows this angle to be determined, to reveal, for example, whether the correct two out of four rotary magazine clip-holes properly received the load.

Within the working arc, the turret-motor provides finely adjusted rotation essential to uniformly place implants at intervals to evenly distribute the magnetic traction. While the muzzle-head must be rotatable, the barrel-tubes are continuous from the airgun chamber to the muzzle-ports and can be bent only so much before deformation interferes with discharge. Furthermore, for safety and to achieve precision fitting, the components of the barrel-assembly are made unadjustable or fixed in rotational alignment, a means for gross rotational adjustment, that is, for rotating the assembly as a bodily whole is necessary.

This limits the extent to which the muzzle-head may be rotated. With the proximal end of the barrel-assembly locked in position within the airgun, no free proximal end as can be manually rotated when a angioplasty capable barrel-assembly is used independently before connection to the airgun is present. To make eccentric barrel-assemblies with muzzle-port groups in different quadrants, for example, with the unrotatable stop-and-lock connection made is unacceptable, since with such working arc limited muzzle-heads, to rotate the working would necessitate removing the barrel-assembly arranged around one angle and replacing it with another.

### Rotation of Working Arc

To avoid such limitation, the airgun is mounted to afford an additional degree of freedom, viz., the ability to rotate about the longitudinal axis passing through the center of the barrel-catheter, and therewith, rotate the working arc. As shown in FIG. XX, rather than to rotate the barrel-catheter or airgun barrel separately, the mounting used to allow the barrel-catheter about the long axis through the airgun barrel preferably consists of an inverted U-shaped bracket XX bent into heavy gauge strip steel stock by a brake. The vertical side to side connecting segment or bridge portion of the bracket is screwed down to the upper surface of the linear positioning table XX.

A small threaded coaxial shaft that has been resistance or spot-welded onto the back of the airgun cabinet along the axis of airgun rotation fits through the hole toward the upper end of rear arm of U-bracket XX. A Belleville disc ring spring washer is placed over the shaft, the shaft then passed through the hole in the front arm of the U-bracket. A dial marking off the degrees in the upper semicircle affixed to the rear surface of the airgun cabinet in concentric relation to rear shaft XX allows the rotation of the barrel-catheter to be measured. A knurled knob with pointer overlying the degrees scale is screwed onto the front of the shaft.

The knob is tightened so that the rotational angle of the airgun, which is stabilized in angle of rotation by friction, can be adjusted by hand. At the front of the airgun cabinet, the barrel passes through a hole that journals by friction fit a ball bearing that holds the airgun barrel in surrounding relation. To measure and render observable the extent of linear travel of the linear positioning table, horizontal joint between the base and moving platform of the linear positioning table XX is calibrated in millimeters. A failure to discharge will be evident and thus can be distinguished externally, as discussed in the section on modes of failure.

Less desirably, the airgun discharge components proper —CO₂ or compressed air cylinder, valve body, chamber, and barrel—can be separately mounted within the airgun cabinet for rotation on a U-bracket that is mounted on a linear positioning table, which then contained within the cabinet at the bottom, even when made of transparent polycarbonate plastic with a hinged or removable top that may be left open to allow access to allow adjustment to the valve body slide as described below, is then more likely to obscured from view by reflection. Such an arrangement thus reduces the observable action of the linear positioning table, of which the incremental moves, at both airgun barrel cabinet portal and entry into the body, are minute and not readily observable. Since this would make a malfunction less noticeable, it is not preferred.

### The Radial Discharge Barrel-Assembly as a Separate and Independent Angioplasty Device in General

An object of the invention is to provide a single means for angioplasty and stenting such that the barrel-assembly having been introduced for angioplasty even when stenting had not been planned, stenting can nevertheless proceed without the need for withdrawal and reentry. To proceed with stenting must require no more than to insert the free (extracorporeal, proximal) end of the barrel-assembly into the airgun. Accordingly, to the extent that such compatibility allows, the radial discharge barrel-assembly is devised to be usable as a separate device for angioplasty without the need to reconfigure it in order to allow its use with an airgun to initiate stenting. Angioplasty-capable barrel-assemblies, to include those incorporating side-sweeping brushes, means for thermal angioplasty, and combination-forms that incorporate a rotary burr or excimer laser are described below under the section entitled Angioplasty-capable Barrel-Assemblies.

For example, the body of the muzzle-head distal to the turret-motor is not made inflatable, various balloons having finger or bristle-like protrusions having long been available. In use for angioplasty, the barrel-tubes serve with the blood tunnels, centering devices, and the free insertion or removal down unused barrel-tubes or the central canal of the barrel-assembly of separate tubing of any pliancy, and such use of a cooling catheter described below as passive stiffeners, the number, material, wall thickness, and diameters of barrel-tubes and absent number, the same variables as pertain to the barrel-catheter representing various variables that contribute to barrel-catheter stiffness, which accordingly covers a wide spectrum.

Use as an independent device for angioplasty requires that the barrel-assembly be provided with airgun-independent sources of electrical power and control. As shown in FIG. XX, a separate onboard control panel provides the ability to manipulate the strictly angioplasty components that do not involve positional control. These include the body of the muzzle-head with or without the assistance of an external hand-held electromagnet, the turret-motor stator as a heating element, the side-sweeper-scraper brushes with debris trap-filter, and when present, a laser catheter or rotational atherectomy burr.

Use for angioplasty manual, power is necessary for the turret-motor as a thermal but not a positioning device, so that the onboard control panel omits positional drive control electronics, to include the servoamplifier. Rather than to augment the effect of brushing by using the turret-motor to rotate the side-sweeping brushes diametrically in their longer dimension, the lesion is repeatedly traversed by transluminal passes. Rather than as shown in FIG. 37 to place the electrical connections for the side-sweeping brushes and the turret-motor stator as a heating element at the proximal end of the barrel-catheter to connect to the power source through the proximal end of airgun barrel, the electrical connections and onboard angioplasty control, panel are situated along a segment of the barrel-catheter that lies in front of the airgun muzzle.

To minimize extracorporeal extension, the barrel-assembly is selected according to the actual intracorporeal length required, the forward drive stabilizer described below assisting to avert extracorporeal bends or kinks as would result from the mass posed by the electrical connections or a battery pack and onboard angioplasty control panel toward the free end. Whether connected to an external source of power by wires or an associated lithium-polymer battery pack, the barrel-catheter can continue as independently powered even when inserted into an airgun.

### Specific Advantages in the Elimination or Minimization of Connection to the Airgun (Tethering)

Since an ablation and angioplasty-incapable barrel-assembly is used only while engaged in an airgun, the elimination of tethering pertains only to angioplasty-capable barrel-assemblies. During an angioplasty, the barrel-assembly is powered from the on-board lithium-polymer or silver-zinc battery pack in the hand grip at the proximal end, and as preferred for freedom of movement, untethered, hence, physically independent from the airgun or any other apparatus. Battery power is always used for the side-sweepers and use of the turret-motor and/or electomagnet windings in their secondary nonpositional function as heat sources for thermal angioplasty. Angioplasty-capable barrel-assemblies must be tethered only when the source of high or low temperature is a vortex-tube based gun that must be supplied with compressed air from a canister (cylinder, air tank). Such connection is not through the airgun but rather directly to the air tank through a very pliant hose.

Even though placing all of the control electronics, the propulsive gas supply cartridge, and other components within the airgun cabinet necessitating that the barrel-assembly be left engaged within the airgun barrel would realize economies by avoiding the need for microminiaturization, the functional superiority of independent operation outweighs this economy. Completely independent angioplasty capability also avoids multiple the need for connection for several other purposes. Thus, were an angioplasty-capable barrel-assembly to lack inmate (on-board, self-constrained, internal) control electronics and an end-plug for connecting a nitrous or oxide or carbon dioxide cartridge, connection to the positional and temperature control components through the airgun during an angioplasty would be necessary frequently for:

1. Temporarily connecting the turret-motor to the drive-controller through the airgun to:
a. Rotate the muzzle-head in order to:
(1) Redirect a thermal window, especially when heating is eccentric through the use of a single electromagnet or motor winding.
(2) Redirect the side-sweepers, so that a different type brush can be applied, such as rotating from one with microshavers to one with microbristles.
b. Oscillate the muzzle-head either by detuning the turret-motor drive-controller velocity loop for random response, or as mentioned above in the section entitled Concept of the Extraluminal Stent and the Means for Its Placement, programming the servocontroller for controlled oscillation in order to:
(1) Free the muzzle-head if stuck, the use of a lubricant, if necessary, achieved by injection through a service channel catheter.
(2) Assist in passing a tortuous course of the ductus if and only if the risk of serious injury is judged not to be present. The intrinsic lubricity of the muzzle-head and the ability to lubricate and oscillate the muzzle-head if necessary make the need for surgical removal hardly possible.
(3) Apply a side-sweeper brush in a vibratory manner.

### However,

a. Connection (coupling) of a laser, rotational, or other mechanical-type atherectomy cutting head cable in a combination-form or barrel-assembly that incorporates such (below) is not through the airgun but rather to the respective control console.
b. Connection of a cooling catheter (below), which is usually prepositioned within the barrel-assembly for immediate use, or connection without a cooling catheter directly to the central canal or a spare barrel-tube (below) is directly to the vortex tube, which in turn is connected by a hose to a tank of compressed air or a nitrous oxide or carbon dioxide cartridge attached to the back (proximal) end of the barrel-assembly.

When a vortex tube (below) is used for thermal or cryogenic ablation or angioplasty, it is mounted to the outside of the interventional airgun cabinet (enclosure). The air tank or canister containing the filtered compressed air for the cold (or hot) air gun is mounted to the airgun cabinet supporting stand. Exceptionally, the use of a vortex tube does not allow an ablation or angioplasty-capable barrel-assembly to be completely disconnected; however, connection by a pliant hose to the air tank need not impede freedom of movement. Thus, except in this circumstance, the proximal end of the barrel-assembly is unconnected and freely movable. In an angioplasty-capable barrel-assembly with a 3-phase motor drive servocontrol microchip incorporated into the hand-grip shaped battery pack, the muzzle-head can be oscillated or rotated without the need to insert and thus electrically connect it through the airgun.

Of these three functions that necessitate the predischarge insertion of the barrel-assembly into the airgun or connection of the barrel-assembly to a cable, the first, to draw control current for rotating the turret-motor, can be eliminated by incorporating a nonvariable speed microcircuit drive-controller that draws power from the inmate battery into the hand-grip. Companies who produce or are able to produce micromotors and microcircuit controllers include Maxon Miniature Motors, Burlingame, Calif.; Solitron Devices, West Palm Beach, Fla.; Contec Microelectronics, Osaka, Japan (San Jose, Calif.); Micromot Controls, Maharashtra, India; Precision MicroControl Corporation, Carlsbad, Calif.; Precision MicroDynamics Incorporated, Victoria, British Columbia; Propex Incorporated, Santa Ana, Calif.; and the Xajong company, Taichung, Taiwan. Considering each in turn:

1. When for simplicity and economy, the incorporation into the hand-grip of an auxiliary drive-controller consisting of a highly miniaturized version of the Data Device Corporation PWR-82332 or SatCon 8314C type, for example, for delivering polyphase current to the turret-motor windings to rotate the turret-motor is not contemplated, and the muzzle-head cannot be manually rotated with the necessary or without risk of stretching injury or dissections, then rotating the muzzle-head requires temporary connection of the turret-motor to the drive controller housed within the cabinet of the interventional airgun. Temporary connection of the turret-motor is through a set of terminals located in the chamber as shown in FIG. 37 or just to the front of the airgun muzzle as shown in FIG. 40. Such an angioplasty-capable barrel-assembly is suitable for procedures not likely to require rotation of the muzzle-head.
2. A combination-form angioplasty-capable barrel-assembly that incorporates a rotary burr or excimer laser requires a power cable that terminates proximally at the bottom of the barrel-assembly or in a socket at the rear (proxmal) end of the central canal for temporary connection to the control console. Albeit inconsequentially, during intervals when the atherectomy component is connected, complete freedom of movement is curtailed.
3. If the turret-motor and/or tractive electromagnet windings had just been used for thermal angioplasty, the counter-thrombogenic requirement to immediately cool the muzzle-head down to body temperature following thermal use applies no less in this situation.

### Angioplasty-Capable Barrel-Assembly Slidable Hand-Grip

The hand-grip of an angioplasty-capable barrel-assembly contains the power source, usually a lithium-polymer battery, alternatively a power cord to a separate wall outlet plug-in power supply, and the control circuitry for rotation of the muzzle-head and thermal angioplasty, and has the angioplasty control panel mounted on its side. Upon withdrawal, a hand-grip that is fixed in position along the length of the barrel-catheter would increase the extracorporeal length of the barrel-catheter from the hand-grip to the introducer sheath making use awkward and inviting buckling over the intervening length.

An angioplasty-capable barrel-assembly is thus made so that the hand-grip can be slid over the portion of the barrel-catheter that is likely to become extracorporeal during a given procedure. Since the barrel-assembly must be immediately insertable into the airgun to commence stenting, conductors cannot exit at the end-plate and fold around to the hand-grip that surrounds the barrel-catheter. Furthermore, an arrangement of sliding contacts (electrical contact shoes, paddle shoes, brushes, wire slide-shoes) running along miniature hot rails (linear sliding contacts, linear electrical contact linear slip-‘rings’) affixed about the circumference of the barrel-catheter as to be exposed would disallow introduction into the ductus of any portion of the barrel-catheter that mounted these.

A sliding hand-grip is provided by bringing the conductors within the barrel-assembly to end-plate terminals that match the terminals on the inside of a transparent friction fitting removable cap keyed to align the electrical terminals. The separately insulated bundled conductors leading from these terminals continue through the top of the cap in a coiled extension or power cord that leads to the hand-grip in slidable encircling relation to the barrel-catheter. The cap contains holes to allow access through a spare barrel-tube or the central canal as a service channel. Thus, for example, when access through the central canal to the muzzle-head is desired, the power cord emerges out of the top of the cap off-center. The cylindrical passageway through the center of the hand-grip is frictionally fitted for positionally stable sliding movement along the barrel-catheter.

### The Muzzle-Head

In overall conformation, the barrel-assembly is devised to minimize obstruction to the circulation. This is accomplished by elongating rather than widening the windings of the turret-motor to obtain sufficient torque, in center-discharge embodiments, narrowing the spindle to a flared throat, and providing blood-grooves over the ejection-head. In models equipped with side-sweepers, when the monitored indicia indicate an unacceptably low level of oxygen in the blood beyond the muzzle-head (ischemia), the side-sweepers can be deployed on one side to nudge the lumen wall away and the muzzle-head toward the opposite side thus allowing more blood to pass. To prevent recoil deformation at the curves where the barrels approach the barrel-ports, which would dissipate much expulsive force (kinetic energy, momentum, impact force) according to the deformation and brake if not jam the spherules upon exiting, polymer barrel-tubes are discontinued, the barrels instead continued as barrel-channels drilled through an ejection-head machined from a solid block of nonferrous metal.

Muzzle-heads conform to either of two configurations, one with barrel-tubes longitudinally centered, the other with barrel-tubes more peripheral to allow a laser catheter or rotary burr to be incorporated at the center. Both include a convoluted elastomeric segment that serves as a damper and point of flexion. Elongation of the muzzle-head through use of a turret-motor winding that is enlarged in length rather than diameter allows a level of torque to be developed that would otherwise deny access to narrower vessels or other ducti and results in a longer contact area with the lumen wall over which directable angioplasty tools such as thermal windows and side-sweeping brushes can be made to apply.

### Muzzle-Head (Barrel-Assembly Distal) Access Barrel

A spare or extra barrel-tube and muzzle-port as already contained within the barrel-assembly allow access to the muzzle-head for the delivery of fluid substances, generally, with a simple pipe, medication and with a radial discharge barrel-assembly, medication or a lubricant. A ramrod or testing rod (below) with outer diameter just smaller than the internal diameter of this muzzle-head service channel, whether the central canal or a spare barrel-tube, allows the substance to be delivered to the muzzle-head by pushing the rod behind the substance down the barrel. To minimize the loss of material by spreading along the inner wall of the barrel, the ramrod is preferably made of a fluoropolymer such as polytetrafluoroethylene. Since any barrel-tube and muzzle-port can be used for discharge, when a barrel-tube is to be reserved for such use, a barrel-assembly including one more barrel-tubes than needed for discharge is used. That is, barrel-assemblies are not made to include a barrel-tube in excess of those that can be used for discharge.

Selecting a barrel-assembly with one more barrel-tubes than is needed for discharge is then preferable to the use of a barrel-tube used for discharge, because unless an additional cleaning step is undertaken, the deposition of a film along the walls and its accumulation along the bottom of the barrel-tube will affect exit velocity and carry some of the deposited material into the intratissue or wound trajectory. Thus, when the number of barrel-tubes needed for discharge and diameter of the implants necessitate a barrel-assembly that with an extra barrel-tube would bring the barrel-assembly to too large a diameter, one or more of the discharge barrel-tubes is used and any problematic film coating left along the inside of the discharge barrel-tube or tubes is wiped down with a second ramrod having an absorbent felt or cotton covering.

### Monobarrel Radial Discharge Muzzle-Head

FIG. 24 shows an external view, and FIG. 25 a view partially in longitudinal section, of a single barrel radial discharge barrel-assembly. While a simple pipe type monobarrel barrel-assembly is intended for use primarily in the tracheobronchial tree and never in a duct or blood vessel, a radial discharge type barrel-assembly can be designed for use in a narrow ductus with substantially undifferentiated anatomy of the lumen wall, and provided certain features are added, in the bloodsteam. Ischemia capable of inducing a midprocedural infarction a primary risk, a barrel-assembly for use in the bloodstream incorporates blood-tunnels, and in the muzzle-head, blood grooves to minimize obstruction to the flow of blood. The use of a multiple-barrel barrel-assembly, especially when automatically advanced, rotated, and withdrawn by means of a positional control system, is not just to achieve precise placement of the miniball implants in a formation, but to achieve operative speed in order to reduce the risk of infarction. Means for avoiding stretching injury and abrupt closure are discussed elsewhere.

To be usable in the bloodstream, the gas pressures generated during discharge must be prevented from entering the bloodstream as gas embolisms. This necessitates enclosing or jacketing about the barrel-tubes over the entire length of the barrel-assembly and providing gas recursion channels so that such pressures are dissipated within the enclosure. The barrel-catheter represents this jacket up to its distal extremity, and the ejection-head at the front of the muzzle-head contains gas return tubes to channel the pressures back into the barrel-catheter central canal. A one-way safety valve, usually an elastomic slit-valve in end-plate 99 (not shown) is present to outlet higher pressures. In small diameter ducti, the muzzle-head enclosure additionally serves to prevent injury by an exposed pointed muzzle.

The enclosure additionally incorporates a shock absorbing joint both to lend flexibility for tracking and dissipate any recoil upon discharge. The parts shown in FIG. 24 are marked to clarify the parts of the distal (forward) portion of the barrel-assembly, or muzzle-head as consisting of 1. A barrel-catheter journaling turret-motor collar; 2. A turret-motor, 3. A flex-joint; 4. A spindle, consisting of a. A neck portion journalled within the turret-motor), b. A spindle throat, c. An ejection-head, and 4. A muzzle-dome consisting of a. An electromagnet assembly and b. A nose-cap portion of which the facing aspect is the nosing.

Spindle 77 of muzzle-head 70 must be able to rotate through 180 degrees to either side, i.e., clockwise or counterclockwise. While a continuous length of very pliant tubing barrel made of a polymer such as vinyl and given enough slack can be rotated through a semicircular arc without distorting the ‘bore,’ most materials are not so flexible and therefore necessitate a rotary joint. When the momentum of the miniballs on exiting and the strength of the barrel-tubing material allows, both lumens of a double lumen extruded tube can proceed to the inner surface of muzzle-head 70 with only one of the two actually open to the exterior through muzzle-port 71 as barrel-tube 74. The second lumen is then sealed by the internal surface of the muzzle-head and is placed in communication with the first through a distal hole. The pressure built up in the lumen used as a barrel-tube then returns through the second, sealed off lumen.

The accompanying lumen adds strength; however, when the barrel-tubes are continuous from one end of the barrel-assembly to the other, a lengthier arc for bending required, deformation becomes more pronounced as the number of barrel-tubes is reduced and the angle of rotation increased, so that with one barrel-tube, reduction in exit muzzle-port 71 and a susceptibility to jamming increases in likelihood. This is ameliorated by providing a reciprocating and rotating flush joint wherein the internal diameter of the ‘bore’ remains constant in a less prominent or reduced ejection head.

Referring now to FIG. 25, rotary joint 58 is formed by transecting division of barrel-catheter 44 flush to the distal surface of clamping collar 59, clamping collar 59 in turn being immovably affixed to the rear of turret-motor housing 61, so that only the distal segment of barrel-catheter 44 journaled within through-bore rotor 60 rotates with rotor 60, and since muzzle-spindle 77 is attached to the distal end of the distal end of the distal segment, spindle 77 is rotated. Viewed from the outside, rotation is seen only distal to rotor 60, which portion constitutes spindle 77, denying visibility of the rotor journaled distal segment as the rotor insertion stem of spindle 77, giving the impression that the rotary joint is at the circumference between the front of the turret-motor and spindle 77. A simple pipe monobarrel-type barrel-assembly is manipulated by hand and does not incorporate a remote actuator to rotate muzzle-head 70 by wire remote control.

In a single-barrel radial-discharge barrel-assembly, barrel-catheter 44 is synonymous with the one and only barrel-tube. Whether such or conducting a plurality of barrel-tubes in a multiple barrel radial discharge barrel-assembly, the segment of barrel-catheter 44 distal to rotary joint 58 and journaled within through-bore rotor 60 of the turret-motor rotates in coaxial relation to the stationary segment of barrel-catheter 44, which is proximal to rotary joint 58 clamped by collar 59 to the rear of turret-motor housing 61. By contrast, the multiple barrel-tubes in a multiple barrel barrel-assembly (which is always of the radial discharge type), continue without break through the encircling rotary joint 58 in the barrel-catheter, passing therethrough off-center or arranged at a slight distance around the longitudinal center of barrel-catheter 44, so that when these insert into the ejection head at their distal ends, rotation of muzzle-ports 71 is driven by rotation of the ejection head

It is thus apparent that the proximal segment of the barrel-catheter, clamping collar, and motor housing 61 remain stationary while the distal segment of the barrel-catheter that follow the rotary joint rotates. Since in a multibarrel (multiple barrel) radial discharge barrel-assembly the barrel-tubes are continuous through the rotary joint and When the barrel-catheter is of a material and thickness that becomes too soft when heated to 90 degrees centrigrade by the turret-motor stator at stall while used for thermal angioplasty, the clamping collar 59 is made of a low heat conductivity and transmissive material such as XXXX or must be lined with a thermal insolent, such as XXXX. The direct-current silver wire-wound subminiature through-bore torquer turret-motor is described in greater detail under the section on motorized turret muzzle-heads below.

The neck or segment of the barrel-tube distal to this cut is journaled in the through-bore rotor 60 of the turret-motor and thus freely rotated by the turret-motor. The elements of the muzzle-head distal to this joint are unitized or monolithic, so that the portion of the barrel-tube within rotor 60 functions as a shaft that rotates the spindle and electromagnet assembly as a unit. As seen in FIGS. 24 and 25, the unitized spindle and electromagnet assembly portions of the muzzle-head distal to the swivel motor rotate about joint 63. As shown in FIGS. 24, 25, and 27, a segment of convoluted tubing XX intervenes between the spindle throat or level where the spindle flares radially outward and the neck, or segment of the barrel-catheter that is distal to the rotary joint and within the turret-motor rotor. The multiple functions and bonding of this convoluted segment to the spindle and nect are described below.

Since unlike a simple pipe, some single barrel radial discharge barrel-assemblies must be usable in the vascular system where the loss of a miniball must be prevented, the tractive electromagnet assembly 64 consists of two electromagnets of opposing orientation, of which only that toward the viewer is shown in FIG. 22. The magnet assembly 64 and independent control of each electromagnet is described below in the section devoted to electromagnet assemblies.

FIG. 26 is a full-face sectional view of the nose or distal end of the single barrel radial discharge barrel-assembly showing the tractive electromagnets 65 and blood grooves 66 to permit some transmission of the pulse past the muzzle-head, which continuous with the indentation formed by the front of the one of the two magnets of the magnet assembly 64 indicated in FIG. 23 are made as deep as possible to run longitudinally along the entire length of the muzzle-head without encroaching upon the barrel-tube, which in a single barrel barrel-assembly is the same as barrel-catheter 44, muzzle-port 71, or magnet assembly 64. The antemagnet chambers 67 behind the spring-loaded trap doors 68 are described under the section on magnet assemblies below. Arrows 69 indicate the path of a recovered miniball, which may have become loose or required to be extracted having been misplaced upon implantation.

The single barrel radial discharge barrel-assembly is similar to the multiple barrel radial discharge barrel-assemblies to be described in materials, in incorporating a motorized turret to rotate the tip, in having paired trap-extraction electromagnets 64, and in the overall form of the muzzle-head 70, which differs only in including but a single muzzle-port 71. As with a simple pipe barrel-assembly, the miniballs fed to such a single pipe barrel-assembly can be delivered from a linear queue type magazine clip, which unlike a rotary magazine clip is, however, limited to one miniball per discharge. It is the only barrel-assembly where rotation is by an axial rotary joint.

The barrel-catheter 44 of a single barrel radial discharge barrel-assembly for use in the airway such as shown in FIGS. 24, 25, and 26 has a motorized turret to rotate the muzzle-head inside the lumen and therefore does not depend upon a particular choice or combination of tubing materials to achieve torqueability. Thus, the same single barrel radial discharge barrel-assemblies made for use in the vascular tree can also be used in the tracheobronchial tree.

When made of a sufficiently nonelastic and slippery material such as polytetrafluoroethylene having a larger wall thickness or as the outer layer in a coextrusion, the distal ends of the barrel-tubes are inserted with no bonding into the flush sockets that represent the start of the barrel-channels in the ejection-head. This allows free rotation and reciprocation of the distal ends of the barrel-tubes. The inner edge of the sockets are beveled to minimize the effect upon the exit velocity of a miniball that might strike the edge.

When, however, the barrel-tubing is sufficiently pliant that the bore is not unduly distorted or its distal end dislodged from the socket during discharge, bonding is essential. When made of polytetrafluoroethylene-coated nylon, the outer surface of the barrel-catheter is primed by etching with a special purpose chemical such as Acton Technologies, Inc. FluoroEtch®, and coated with an adhesive suitable for bonding etched polytetrafluoroethylene to stainless steel, such as Master Bond, Inc. EP42LV, a two component, low viscosity room temperature curable, epoxy adhesive.

While the withdrawal of one barrel-assembly and insertion of another is acceptable in the airway when space affords unobstructed maneuverability and the possible complications of a luminal entry wound are not a factor, entry in the vascular system is best singular. Unless the benefit in operative speed gained with a multiple discharge radial barrel-assembly justifies replacing it with a barrel-assembly of smaller diameter having fewer barrels, minimizing the risk of entry point complications usually discourages such technique. Thus, even though a vessel or duct may admit a muzzle-head of larger diameter and more barrels upon entry, access to the smallest gauge of the vessel or duct should dictate the diameter of the barrel-assembly used; that is, treatment is best accomplished by using a muzzle-head of a diameter that will allow access throughout the length to be implanted.

The description of multiple barrel radial discharge barrel-assemblies taken up below, as well as with a single barrel radial discharge barrel-assembly, eccentric lesions that change in circumferential placement along successive segments or at different levels can also be negotiated either with a muzzle bit having muzzle-ports separated by less than 45 degrees or by inserting a rotary magazine clip that blanks out barrel-tubes in a multiple barrel muzzle-head the turret-motor used to angle the muzzle-head in either event.

Factors that Affect the Length, Hence, the Working Reach of the Muzzle-Head

Whereas elongation in the nose or elements of the muzzle-head forward (distal, anterior) of the muzzle-ports reduces the forward reach or depth of access for discharging implants in a lumen of given diameter, elongation proximal to the muzzle-port or ports does not. Increasing the diameter, however, instantly limits penetration down the vascular tree to deny access to smaller vessels. Thus, when the barrel-assembly incorporates side-sweeping ablation brushes, the increased torque required is achieved by enlarging the motor windings longitudinally rather than diametrically.

A loss in forward reach notwithstanding, embodiments that necessitate extension of the nose to house a trap-filter are extended when side-sweepers are installed. As discussed above under the section entitled Concept of the Extraluminal Stent and the Means for Its Placement, the benefit in distal embolic protection filters remaining controversial, embodiments without side-sweepers installed are provided without a distal embolic protection filter, hence, without the loss in distal reach such incorporation produces in most muzzle-heads.

While ducti requiring treatment over much of their length will seldom be consistent in lumen diameter, to the extent possible, the muzzle-head body should match in diameter the most constricted or stenosed stretch of lumen. In an angioplasty-capable barrel-assembly, matching these diameters brings the heat-windows and muzzle-ports to the endothelium so that in a blood vessel, heat is conducted through the smallest amount of intervening blood, and the risk of a miniball being deflected prior to penetration is minimized. Also, side-sweeping brushes then need protrude only slightly beyond the surface of the muzzle-head body, allowing the wells into which these are retracted to be shallower.

The primary limiting factor in reducing the diameter of the barrel-assembly is the diameter of the motor, which unlike the distal components of the barrel-assembly cannot be channeled or blood-grooved to allow at least some blood to flow past it. To reduce to the extent possible any opportunity for ischemic complications, the turret-motor is made somewhat smaller in diameter than the rest of the muzzle-head, and to compensate for the loss in torque that this reduction in diameter of the stator and rotor effects, the turret-motor is extended longitudinally rather than radially.

The turret-motor is located at the rear of the muzzle-head to allow the components that require immediate access to the lumen to reach as far forward (distally) as possible and not deduct from the working reach of the muzzle-head down the vascular tree, especially when the lumen is decreasing in caliber. Placed to the rear of the contacting components, the proportional increase in motor length essential to preserve torque does not precede the muzzle-ports to deny depth of access for implantation, for example. The administration of vasodilating medication allows some further access down the vascular tree, just as the administration of bronchodilating medication does down the bronchi. When not circulating (systemic), such medication is injected through a service channel.

Whether in center-discharge or combination-form angioplasty-capable barrel-assemblies, the incorporation of a side-sweeping brush module as discussed below, necessitates the installation of a distal thromboembolic protective trap-filter as well. Since the release of debris is probable with side-sweeping but possible at any time, the trap-filter, as well as deployable simultaneously with the side-sweepers, is deployable independently. Provided no laser or burr is installed, the distal portion of the central canal in a combination-form (edge-discharge) muzzle-head with longitudinally arranged recovery electromagnets is available as a sleeve or silo for storing the trap-filter, the overall length of the muzzle-head in that case reduced thus increasing the working reach compared to a muzzle-head with extended nose. However, installed thus, the central canal must not admit ductus contents whether blood into the ejection head even when the trap-filter is deployed. This kind of muzzle-head configuration can be used with either an edge- or center-discharge muzzle-head.

In a combination-form muzzle-head with a rotational atherectomy burr or laser cable installed, the central axial position is already occupied, so that a sleeve or silo recess into which the trap-filter can be retracted while not deployed must be placed adjacent to the burr or laser, which latter occupies the central canal, making the recess eccentric (off-center). Whether installed in the central canal or off-center to accommodate an excimer laser or atherectomy burr, the silo must have sufficient capacity to retract several miniballs. As seen in the cross-section of FIG. XX, the longitudinally arranged recovery electromagnets with antechambers and spring-loaded trap doors facing radially outward to either side of the laser or burr cable afford sufficient room between them and adjacent to the cable to place the trap-filter silo. Accordingly, neither must one of the recovery electromagnets be shortened nor the nose elongated as would further reduce the working reach in comparison with the sidewise arranged electromagnets in the center-discharge muzzle-head shown in FIG. XX.

Incorporation of a Bounce-Plate into Radial Discharge Barrel-Assemblies

The value in a proximad redirection or trajectory reversal capability pertains to advanced cases of collapse or stenosis of the trachea with its structured luminal wall that includes cartilage rings and ligaments. Other ducti are not this structurally differentiated, so that the insertion of implants to given trajectory end-points or target locations can proceed unidirectionally with uniformly distad or forward-inclined trajectories and without the need for reversal to proximad or backward-inclined trajectories. Furthermore, since for a given number of barrel-tubes, a radial discharge barrel-assembly should achieve minimization in the outer diameter of the muzzle-head, and the addition of bounce-plates, requiring the muzzle-ports to be recessed, would add diameter if within, and would be likely to cause scraping injury to the lumen wall if mounted to the outside of the muzzle-head. In a multiple discharge barrel-assembly, these consequences are unacceptable.

Even the introduction and withdrawal of a simple pipe barrel-assembly provided with a protective rubber surround extension at the distal end through the larynx must be performed with caution. Requiring to avoid the need for multiple withdrawals and reentries that increase the chances for entry wound complications, a radial-discharge barrel-assembly configured for use in the circulatory system would demand remotely deployable bounce-plates. Of little value, these would add to the cost. The essential structural uniformity or homogeneity of the average lumen wall obviates the need for such a capability.

The structured character of the tracheal lumen is larger than the structurally undifferentiated lumens of vessels and ducts, making observation of the tracheal lumen more important and less difficult. The usefulness of radial discharge barrel-assemblies capable of reversing the direction of the trajectory in the vascular tree or in ducts, whether provided in a single embodiment or by changing to either of two embodiments, is thus recognized as feasible but anatomically unjustified. In the airway of a small dog or human neonate, such a radial discharge barrel-assembly could achieve precise aiming only tediously and laboriously, while in the vascular tree, no such reverse aiming capability is necessary. For these reasons, the incorporation of means for reversing the direction of the trajectory in radial discharge barrel-assemblies is discounted.

### Use of the Barrel-Assembly as an Aspirator or Transluminal Extraction Catheter

Once every miniball has been implanted, rather than to withdraw the barrel-assembly and introduce an aspirator to remove any debris such as mucus, the barrel-assembly can be disconnected from the airgun and connected to a vacuum aspirator using the same mechanical connection. Except for use in the largest diameter ducti, to incorporate additional components into the barrel-assembly as would allow it to also function as an ultrasonic aspirator handpiece for the fragmentation and emulsification of tissue currently exceeds practical capabilities for the miniaturization required.

### Treatment of Tracheal Collapse in the Cervical Segments, i.e., Cephalad, or Anterior, to the Thoracic Inlet

The treatment of tracheal collapse in anterior segments where the trachea has not yet plunged into surrounding mediastinal tissue is preferably by means of a jointed stent-jacket following ballistic miniball implantation with a simple pipe type barrel-assembly. In basic contrast to the standard procedure that individually sutures rings cut from a syringe casing about the collapsed trachea to serve as prosthetic cartilage rings necessitating access over the entire length of the treatment area for suturing, the stent-jacket is held in position by its textured internal surface or gauze lining and by the magnetic attraction of ferrous implants just within the outer fibrous layer of the trachea.

The possible sequelae of such a procedure include infection, dysphagia, and stimulation of the cough reflex; however, these should prove medically manageable. Averting the risk of asphyxia is considered worth any discomfort due to magnetic attraction between tissues or, when magnets have been inserted subcutaneously or suprapleurally, in relation to metal objects in the environment, which the patient will never be too weak to leave and become conditioned to avoid. That any medical procedure must be tested extensively and over a long period is considered superfluous.

If thought necessary to avert migration, the placement of suture is through and in line with this incision. Requiring the extension of the incision to allow for suturing separate rings eliminated, insertion of the stent-jacket and its fixation in position are through an incision that is a small fraction of the length required for the conventional procedure, materially reducing trauma and extending treatment to patients too impaired to withstand the standard procedure. Extension of treatment to the distal bronchi is preferably by dorsolateral ballistic implantation into the bronchial ceiling with an eccentric two-way radial discharge barrel assembly with the ceiling to be suspended by subcutaneously or suprapleurally implanted magnets (patch-magnets, clasp-magnets).

Dependent upon a small absolute diameter of the trachea for symptoms to appear, the patient suffering from tracheal collapse will almost always be a small dog. Distad, the lumens of the bronchi are likely to become reduced to no more than a few French. Such a severe reduction in working space may necessitate dispensing with a simple pipe and continuing with a one-way radial discharge or monobarrel-assembly of the kind ordinarily used for vascular and ureteric applications. Adaptability in the use of barrel-assemblies, and airguns that support different barrel-assemblies are significant cost reduction factors.

Use of a Magnet-Wrap about the Esophagus to Treat Tracheal Collapse in Small Dogs

Where the esophagus and trachea course together in dorsoventral relation, tracheal collapse can be treated by tacking the collapsed membrane (dorsal membrane; musculus trachealis; tracheal muscle) to the underside of the esophagus. To suspend the dorsal membrane thus, a simple pipe barrel-assembly is used to implant miniballs at the junctions of the annular ligaments toward the dorsolateral edges of the cartilage rings. A compliant and nonconstricting magnet-wrap placed about the esophagus containing magnets at intervals along ventrolateral longitudinal lines suspends the miniballs. The esophageal magnets are not ballistically inserted magnetized miniballs, because the esophageal periphery tends not to be sufficiently hard and the otherwise unaffected esophagus should not be involved much less traumatized at the risk of inducing dysphagia.

If the testing means and method described below reveals that the ceiling is too weak or soft (malacic, malacotic) to prevent the implants from penetrating and perforating the tracheal ceiling, then the procedure is stopped and an endotracheal stent is inserted. While peristalsis moves the esophageal ventrum or floor vertically, which could pull against the dorsal membrane in a corresponding undulative wave, it moves the sides laterally, and this lateral movement affects the distance between the attractants slightly at most. If the tracheal implants are centered in relation to the lateral excursion of the peristaltic wave, then there will be no vertical displacement of the dorsal membrane, which is suspended as a side slung carriage.

Magnets within a magnet-wrap at intervals along ventrolateral longitudinal lines running beneath the esophagus are advantageous over subcutaneously or suprapleurally placed magnets in presenting a magnetic field much weaker and local to the treatment site and therefore effectively isolated from metal objects in the surroundings. The spherical contour of the implants essential for ballistic insertion presents a relatively poor gap for magnetic flux. When collapse has already extended to the bronchi, the decision to use subcutaneous or suprapleural magnets should be weighed against more conventional endobronchial stenting and the need for the patient to become conditioned to avoiding immediate contact with metal objects in the environment such as kitchen appliances.

This nuisance must be weighed against the obstruence of an endoluminal stent within the tiny secretory and macrophage-swept lumen. Any slack in the dorsal membrane resulting from the stretching caused by inspiration and expiration while the rings had continued to lose resilience and the ceiling increasingly collapsed is taken up and drawn out laterally between the dorsoventrally interfacing implants, draped over the side of the trachea, and thus clamped outside the lumen. As is true in other ducti, the use of a clasp-wrap to position miniballs along ventrolateral longitudinal lines along the esophagus is intended to avoid placing soft tissue under compression and restraining the passage of peristaltic contractive waves along the esophageal floor.

Whereas the trachea is active constantly, peristalsis normally occurs in the esophagus only during deglutition, and is substantially confined to its ventral or inferior (in man, anterior or rostral) two thirds. The very malacic condition of the rings renders the tracheal ceiling sufficiently flaccid to comply with the peristaltic movement of the esophagus to which it has been suspended without interference to the mucociliary function of the trachea. Because the longitudinal lines of tracheal miniball implants are placed just within the outer fibrous sheath or adventitia of the trachea and the miniballs in the clasp-wrap are positioned along ventrolateral lines, esophageal peristalsis should be unaffected following healing, and coughing no longer presages eventual suffocation.

During deglutition, the peristaltic waves are impressed upon the tracheal dorsum; however, breathing is never simultaneous with deglutition and the very flaccidity of the collapsed trachea affords motile compliance. Nevertheless, some peristaltic induced coughing while eating is to be expected. If coughing is due to ‘tickling’ that triggers the cough reflex rather than to occlusion, then it is disregarded as a nonthreatening annoyance. If associated with residual occlusion, then ventrolateral implants can be placed over the segment affected or an intraluminal stent that is much shorter than would have been required were it the sole treatment is inserted.

Barring immediate flush contact with a metal appliance or vehicle, the subcutaneous or suprapleural magnets are not so powerful as to prove problematic with metal objects in the surroundings. The patient eventually becomes conditioned through experience to avoid snuggling up against such objects. In conditioning to avoid certain postures, acclimatization to new sensation, and to allow healing, the procedures to be described for the palliation of tracheal collapse, while reversible, should be allowed to remain in place until failure is certain. Where, as in the extremities, a vessel is embedded in tissue, some special consideration or complication must discourage the use of a conventional intraluminal stent to justify the use of a stent-jacket peripherally.

The miniballs in the clasp-wrap placed about the esophagus are placed at the average anteroposterior interval by which the rings are separated and the trachea is then pulled slightly toward the anterior or posterior to align the tracheal and esophageal implants. Even though both esophageal and tracheal miniball implants have been inserted through the mouth, the trachea has been restored to patency without significantly reducing the cross-sectional area to less than normal, coughing has been reduced, the threat of suffocation and the morbidity of incision and sutures has been eliminated, and once healed, esophageal function is not significantly affected.

To accomplish the same repair by the conventional means of suturing prosthetic rings about the trachea requires approach through a cervical incision of considerable length, to place the sutures opposite to the incision is awkward extending the duration of the procedure, and when collapse has already progressed to extend into the bronchi, the lateral thoracotomy needed is untenably traumatic for the patient, whom the condition has long impaired. Gross motility of the trachea in terms of overall bodily movement is reduced in detail by suspension from the esophagus; however, these normally move together.

Most conditions of collapse should be remediated by a running dorsolateral magnetic tacking of miniballs implanted in the tracheal ceiling to a magnet-wrap about the esophagus as mentioned above. To avoid further stretching or ripping of the dorsal membrane, this is done through the annular ligament toward the ends of the rings. The existing grade of the condition, which is always progressive, should be projected to increase and extend posteriad over time. Therefore, regardless of the existing grade and distribution of collapse, treatment should be extended beyond the area affected.

Collapse of given grade at a level where the trachea is bent, especially when the convexity is directed to the posterior, can be more serious than when the course of the trachea is substantially vertical or the convexity anteriad. If more pronounced, a posterior convexity may necessitate the placement for a length along the bend maximum of implants along the edges of the ventrolateral quadrant of the tracheal floor as seen in cross-section, with subcutaneous magnets to draw these implants ventrolaterally, and esophageal tacking along the edges of the dorsolateral quadrant of the tracheal ceiling for the adjacent segments.

In advanced cases where collapse is such that the trachea becomes folded flat when the head is raised, combining the present method with the placement of prosthetic rings still makes it possible to considerably, perhaps critically, reduce the extent of surgery. Unless uniform tacking of the tracheal ceiling to the esophageal floor is insufficient, the use of subcutaneous magnets, especially in the cervical area, should be avoided as annoying the patient when the head is turned. Generally, following the tacking of the dorsal membrane as described herein, an interval should be allowed to see if the patient can simply learn to avoid aggravating postures before taking any further steps.

Single barrel discharge as used in the airway does not require the use of a rotary magazine clip which provides multibarrel discharge. Instead, semiautomatic operation is supported by a caliber-adapted spring-loaded or gravity fed magazine clip as described below. An otherwise ordinary gas operated pistol, or hand airgun, that has been adapted in caliber or gauge and lowered in exit velocity to the required range can be used. Next to a jointed stent-jacket immediately surrounding the implanted trachea, which is always preferred, the closest structure from which the collapsed dorsal membrane might be magnetically suspended is the floor of the esophagus.

### Treatment of Tracheal Collapse in the Thoracic Segments, i.e., Caudal, or Posterior, to the Thoracic Inlet

When suspension of the collapsed dorsal membrane is by esophageal tacking rather than through the use of a stent-jacket, once trachea and esophagus diverge, magnetic suspension is attained through the subcutaneous placement of magnets overlying the affected area. By contrast, a single stent-jacket may continue distally to the bronchial bifurcation, proximal portions of the bronchi as may be accessed without thoracotomy can be either stent-jacketed or suspended by subcutaneous magnets, or all portions of the bronchi can be supported by subcutaneous magnets. An object of the procedure is precisely to eliminate the need for a thoracotomy using a form of stent that is placed outside of the airway and od not susceptible to accumulating or clogging with mucus as to require reinspection, withdrawal, and replacement.

An evaluation of the procedure to be used must consider the course of the trachea in different body positions and not just when the patient stands or sits. When the trachea is recurved, subcutaneous magnets placed ventrolaterally to pull nonmagnetized or magnetized implants placed ventrolaterally in the anterior wall of the cervical trachea may occasionally be necessary to increase tracheal patency. Since this produces the annoyance of sudden clamping with movement, it is best avoided. Posterior to the neck, however, such use need not be discouraged. The combination of methods, here the use of intraluminal stents in the bronchi, should always be considered.

The following is limited to the repair of tracheal collapse without the need for incision of any kind or, if subcutaneous magnets are used, incisions that are very few, small, and shallow. The preferred treatment as delineated above requires a small incision through the integument along the bottom of the neck or cervical ventrum and insertion of a jointed stent-jacket. The best treatment for a given patient must rest with the clinical judgment of the veterinarian.

### Detailed Use of a Simple Pipe Barrel-Assembly to Palliate Tracheal Collapse in Small Dogs

Procedure for palliating tracheal collapse in small dogs:

1. The patient is fully-evaluated bronchoscopically and radiologically to confirm collapse as the cause of the symptoms and to observe the extent or grade of collapse as well as to determine the working diameter afforded by the anatomy. If the patient is too small, then the procedure is discontinued, and if justified by life expectancy and judged medically competent to withstand open surgery, the standard procedure to implant polypropylene prosthetic cartilage rings is performed. If not, then one or more deformation-resistant intraluminal stents are inserted. If not too small, the patient is evaluated for the variant of the procedure described herein, the medication to be used, and the dosages to apply.
2. A narrow gauge or small diameter pediatric bronchoscope is lashed to the barrel-catheter, care taken to avoid bending the catheter, and the patient preoxygenated. To clear the visual field for the operator, aspiration may have to be intermittent or “tubeless,” spontaneous respiration used as the smallness of the airway dictates. Tiny patients may require percutaneous transtracheal jet ventilation or a preliminary tracheotomy with or without jet ventilation.
3. The patient is anesthetized and positioned supine on a cushion that allows the airway to be straightened with head dorsiducted and mouth gagged open as not to interfere with breathing or with mobility of the neck, which may then be positioned as necessary during the procedure. An adjustable stage or intervening platform with adjustable pitch for placement on the operating table gives improved access.
4. The bronchoscope is used to locate the cartilage rings and the conducting tube to insert one shot each into the anterior junction of each successive cartilage ring with the annular ligament bilaterally along the imaginary lines that demarcate the lateral edges of the dorsal quadrant of the trachea were it circular. Such placement not only takes advantage of the histology, but introduces prosthetic support at the normal intervals. To well seat the shot in each junction, the lamina propria is undercut by lightly pressing the 45 degree tip of the barrel-catheter flush against the endotracheal lining at a distance of ¼ inch anterior to each cartilage before triggering the shot. If the one miniball at each ring is suspected to sufficiently palliate the collapse and only dorsolateral subcutaneous or esophageal miniball magnets are to be used, then this concludes the endotracheal portion of the procedure, which should proceed directly to either the placement of the subcutaneous or esophageal magnets.
5. If increased suspension is considered urgent enough and worth the additional time and swelling in one procedure, then using a reverse 45 degree rebound tip or bounce-plate, a second pass is performed to insert one shot into the corresponding positions of each posterior junction bilaterally. The presence of a magnetic body inside the anterior and posterior junctions of each cartilage ring with the annular ligament through which the magnetic lines of force course lifts each ring under the pull of the magnets. The object is to arrange that the magnetic lines of force course through both shot implants of each ring to create a virtual bit, sling, or cross-pin that passes beneath and lifts each ring. If only dorsolateral subcutaneous disk or esophageal miniball magnets are to be used, then this concludes the endotracheal portion of the procedure, which should proceed directly to placement of either the subcutaneous disk or esophageal miniball magnets. If the need for double implants is not considered urgent, then a second set of posterior junction miniballs can be added at a later date; nonmagnetic, polarity will pose no problem.
6. If the collapse folds flat when the patient raises its head so that dorsal mending alone is predicted not to sufficiently palliate the collapse, then gag but not the bronchoscope or barrel-assembly left in place, the patient is now turned prone, chin resting, head dorsiducted with nonbinding support that allows the neck to be circumflexed. The same process is then used to insert implants in the same pattern along the imaginary lines that demarcate the lateral edges of the ventral quadrant of the trachea were it circular. This concludes the endotracheal portion of the procedure, which should proceed directly to the placement of the magnets whether subcutaneous or esophageal.
7. If the esophageal tacking method is intended and step 5 has been skipped, then gag left in place, the patient is now turned prone, chin resting, head dorsiducted with nonbinding support that allows the neck to be circumflexed. A magnet-wrap is introduced through an incision of minimal length as described above and placed about the esophagus so that the magnets are situated along imaginary lines that demarcate the lateral edges of the ventral quadrant of the esophagus. Fluoroscopy is used to assist in aligning the tracheal miniballs and esophageal miniball magnets in vertically interfacial relation.
8. If subcutaneous disk magnets are to be placed dorsoventrally or both dorso- and ventrolaterally, then to optimize the positioning, strength and size of each disk magnet placed dorso- or ventrolaterally, each magnet is first pressed downward into the muscle to the depth of the muscle fascia by a trained assistant while the operator observes the effect on the miniballs that have been implanted in the trachea. Since the disk magnets will be fastened to the laterally stable fascia, the repulsion of neighboring like poles is not felt and serves to isolate or render noninteractive the parallel magnetic circuits formed with the respective miniball implants; the coursing of the field from the south pole of one magnet to the north pole of an adjacent magnet of like orientation to produce a diagonal pulling force as resultant is precluded.
9. If neither a stent-jacket about the trachea or the use of a magnet-wrap about the esophagus is wanted, magnets to suspend the collapsed tracheal ceiling can be placed subcutaneously at an angle to draw the ceiling upwards with minimal compression to the esophageal ventrum. Fur that interferes with such preliminary positioning is trimmed away. Trying different magnets, the minimum pull required at each level is determined. Testing for different degrees of neck flexion, the operator uses the bronchoscope to observe which combination of smallest magnets urges the cartilage rings sufficiently erect to clear the airway, and marks the magnet to be placed in each position on the pelage and if necessary, on pressure sensitive adhesive backed labels temporarily placed on each magnet. Within the effective distance, raising the magnets reduces the pull, reducing, not increasing, any upward pressure upon the esophagus that would produce discomfort in swallowing. The fur is shaved at the prospective locations of magnet insertion. In the cervical region, use of the smallest magnets most dorsally positioned will eliminate or minimize the force of clamping sufficient to retain any dorsal membrane slack between the miniball and magnet when the neck is flexed. The same procedure is used to position ventrolateral magnets if needed.
10. A longitudinal incision through the integument on either side of the line of implants allows fastening the subcutaneous magnets to the surface of the muscle fascia. The prongs at the top or bottom are engaged and the fascia pinched so that the prongs at the other end engage when released. The incision is closed with surgically approved long-chain methacrylate cement and swabbed with antiseptic ending the procedure.
11. Routine recovery measures to include the administration of a local anesthetic to the prong sites as alertness is regained, oxygenation and the administration of anti-inflammatory medication are administered. If, as is common, steroids are to be avoided, then postoperative swelling is managed with an NSAID such as Voltaren® (Novartis diclofenac sodium) or Cataflam® (Novartis diclofenac potassium) or a proteolitic enzyme NSAID such as Danzen® (Takeda Chemical Industries) in enterically coated tablet form or SerraZyme® (Health Australasia Limited serrapeptase; serratia peptidase). The administration of antibiotics is in accordance with routine. Once ferromagnetic metal has been implanted, magnetic resonance imaging must not be used.

A cough usually persists for an interval following conventional surgery, and here as well, time should be allowed following each procedure to be described to ascertain whether a cough will subside and the patient become acclimated to the strange sensation. If not, the miniball implants are simply left in place as bioinert, a magnet-wrap applied to the esophagus withdrawn, and an intraluminal stent or stents inserted (see Moritz, A., Schneider, M., Bauer, N. 2004. “Management of Advanced Tracheal Collapse in Dogs Using Intraluminal Self-expanding Biliary Wallstents,” Journal of Veterinary Internal Medicine 18(1):31-42; Gellasch, K. L., Da Costa Gomez, T., McAnulty, J. F, and Bjorling, D. E. 2002. “Use of Intraluminal Nitinol Stents in the Treatment of Tracheal Collapse in a Dog,” Journal of the American Veterinary Medical Association 221(12):1714, 1719-1723; Hwang, J. C., Song, H.-Y., Kang, S.-G., Suh, J.-H., Ko, G.-Y., Lee, D. H., Kim, T.-H., Jeong, Y.-K., and Lee, J. H. 2001. “Covered Retrievable Tracheobronchial Hinged Stent: An Experimental Study in Dogs,” Journal of Vascular and Interventional Radiology 12(12):1429-1436; Radlinsky, M. G., Fossum, T. W., Walker, M. A., Aufdemorte, T. B., and Thompson, J. A. 1997. “Evaluation of the Palmaz Stent in the Trachea and Mainstem Bronchi of Normal Dogs,” Veterinary Surgery 26(2):99-107; and Sawada, S., Tanabe, Y., Fujiwara, Y., Koyama, T., Tanigawa, N., Kobayashi, M., Katsube, Y., and Nakamura, H. 1991. “Endotracheal Expandable Metallic Stent Placement in Dogs,” Acta Radiologica 32(1):79-80). In so doing, the type of stent must be chosen carefully (see, for example, Madden, B. P., Loke, T. K., and Sheth, A. C. 2006. “Do Expandable Metallic Airway Stents Have a Role in the Management of Patients with Benign Tracheobronchial Disease?,” Annals of Thoracic Surgery 81(2):274-278.

Multiple Radial Discharge Barrel-Assemblies with One to Four or More Way Radial Discharge Muzzle-Head for Vascular and Ureteric Applications

Preferred are mono and multibarrel barrel-assemblies that are unitized components which include a proximal end-plate, the barrel-catheter containing the barrel-tubes, a motorized turret if present, and a muzzle-head which includes the proximal muzzle-ports through which the projectiles or miniballs are expelled and a distal tractive electromagnet set to recover any loose or misplaced miniballs, and a forward hemispherical nose to minimize the risk of perforations. For barrel-assemblies within a given range in diameter, simple pipe or single barrel and multiple barrel radial discharge embodiments are preferably engageable by the same airgun when the suitable airgun bore-reducing liner is inserted. The use of rotary magazine clips greatly facilitates the ability to change the caliber and thus allow one airgun to support numerous applications. Muzzle-ports that face in opposite directions not only accelerate the process of implanting ducti that unlike the airway, lack structural differentiation, but inherently cancel the reaction to miniball discharge or recoil associated with transit through a curve to discharge when not counterbalanced.

Turning now to FIG. 27, shown is a four-way or four barrel radial discharge barrel-assembly consisting of a barrel-catheter 72, muzzle-head 73, and four barrel-tubes 74 of which only two are shown in this middle longitudinal section, and stop-and-lock ring 75, which engages a ring with complementary interlocking projections on the muzzle of the airgun. Muzzle-head 73 includes turret-motor 76, and rotating muzzle spindle 77, which includes the tractive electromagnet assembly 80 at its front or distal end. Turret-motor housing 78 is bonded to the outside of barrel-catheter 72 by clamping collar 81. When the barrel-catheter is of a material and thickness that becomes too soft when heated to 90 degrees centrigrade by the turret-motor stator while used for thermal angioplasty at during stall, clamping collar 59 is lined with a thermal insolent, such as poyurethane. At 95 are centering devices and at 96 a blood-tunnel, both described in sections that immediately follow.

Muzzle-head body 73 is preferably micromachined in proximal (rear, turret-motor housing) and distal (front, ejection-head) portions under computer numerical control from a solid block of nonmagnetic stainless steel of material as specified above and hardened by heating and quenching. When made thus, each pair of barrel and pressure relief channels are machine or laser-drilled diagonally and radially toward the longitudinal axis from the same aperture. Effectively a segment of the barrel, the outer surface of the proximal portion of the electromagnet assembly housing must be longitudinally aligned to the central arc of the barrel-channel. To prevent the gas pressure of discharge from forcing gas into the bloodstream, paths of least resistance to the flow of the pressurized gas are placed in communication with the muzzle-ports to return the gas to the peribarrel space.

The cross-sectional area of the return path is equal to or larger than the sum of the cross-sectional areas of the barrel-tubes. When appearing narrower than this in cross-section, it is because the barrel-channels are elliptical normal to the view. ‘Nonessential material’ referring to metal that is not needed to provide sufficient strength to resist deformation or fracture in normal use and thick enough to withstand hardening, to obtain the maximum flow-through of blood, nonessential material is removed from the spindle portion of the muzzle-head distal to the engagement of spindle neck XX within motor rotor 82 and the entry after radially splaying, i.e., outwardly curving or flaring, of barrel-tubes 74 into flush joints XX that to counter deformation of the ‘bore’ as would impede if not jam ejection, allow rotation and reciprocation, the front or distal end of barrel-tubes able to move up and down in the manner of a piston. As shown in FIGS. 24, 25, and 27, this coaxial relation, which may be supported with centering devices, allows the barrel-tubes to continue through rotor 82 and spindle 77 in alignment for insertion in divergence into the holes in ejection head 84.

As shown in greater detail in FIG. 27, the proximal length of barrel-catheter 72 and its contents, motor housing 78, and turret-motor stator 83 are fixed together and stationary. That is, only spindle 77, consisting of flex-ring XX, and distal metal portions, which are unitized by bonding with the segment of the barrel-catheter journaled in rotor 82, rotate, the barrel-tubes continuous through the bore of motor rotor 82 and inserting at their distal ends into the ejection-head. Rotary joint 79 divides barrel-catheter 72 between the proximal portion clamped in clamp collar 81 fixing it in position and distal portion journaled within motor rotor 82. The distal portion of the barrel-catheter that is journaled in rotor 82 as the spindle stem or neck is bonded to the proximal end of convoluted tubing or elastic flex-ring XX. Clamp collar 81 fixed to the rear of through-bore configured turret-motor 76 locks pre-rotary joint proximal barrel-catheter 72 in coaxial relation with the distal segment of the barrel-catheter journaled in rotor 82 for rotation as the stem or neck of spindle 77.

For a four-way radial discharge muzzle-head, rotation is limited to 22.5 degrees in either direction for discharge and 90 degrees for electromagnet assembly extractions of misplaced miniballs. Muzzle-head detail FIG. XX can also represent a two-way radial discharge muzzle-head, except that for the rotation of the barrel-tubes by 90 degrees, in either direction, the length, i.e., the recess and distance of reciprocation within flush joints must be slightly longer and the barrel-tube material used more pliant without deforming upon discharge. To prevent air from leaking out of the gas recursion channel and thus allowing blood to enter the muzzle-head, the barrel-assembly and airgun chamber must airtight except through the barrel. The polymer of the barrel-tubes, which may consist of many different materials and compound tubing, must be sufficiently thick and strong that jerking and deformation do not significantly affect discharge. Preferably, there is little change in exit velocity as the rotational displacement is varied.

Upon entry into the muzzle spindle 77, barrel-tubes 74 enter the splay-chamber, which allows the barrel-tubes to bend while flared centrifugally and to maintain the consistent association of each with its respective muzzle-port 88, situated about the periphery of the muzzle-head. Depending upon eccentricity of the lesions to be treated, the muzzle-ports may be equidistant or eccentric. So that the barrel-tubes can counterdeformatively rotate and reciprocate, or move up and down within the barrel-channels in the muzzle spindle 77 sufficiently as not to become distorted or kink when the muzzle-spindle 77 rotates, the joint 84 between the terminus of the barrel-tubes and the metal spindle is flush fit but not fastened. The extent of this rotation and equivalent compensatory longitudinal excursion in the barrel-channel of the distal ends of the barrel-tubes, which terminate at muzzle-ports (that facing the viewer being 88), is slight, the maximum required being 180 degrees for the tractive electromagnets 80 to be directed at any angle.

Radially situated barrel-tubes 74 must be free to bend in response to the axial rotation of the spindle and therefore cannot be encased in metal. With nonessential metal removed, the proximal portion of the spindle between rotary joint 63 and the rotating and reciprocating flush joints XX into which the distal ends of the barrel-tubes are inserted define a space, the splay-chamber, having a generally flared shape, the outer surface thus allowing blood to pass all round into blood-grooves 66 along the broadest segment and so entirely past the barrel-assembly.

As shown in FIGS. 24, 25, and 27, a segment of convoluted tubing XX to which the spindle is bonded with a long chain methacrylate cement just distal to its neck in the turret-motor rotor and the throat or level where the spindle flares radially and forward serves to:

1. Improve steerability by allowing radial flexion of the muzzle-head distal to the turret-motor at the convoluted segment.
2. Allow more blood to flow past.
3. Absorb and dampen the shock of discharge recoil, especially in an eccentric, hence, force-imbalanced, monobarrel or when ejection is eccentric or not precisely simultaneous in a radially symmetrical multibarrel.
4. Insulate and so temperature isolate the rear portion of the muzzle-head when heated by increasing the current to the turret-motor stator to allow thermal angioplasty.
5. Reduce the contact area of the external surface of the muzzle-head with the lumen wall, thus reducing any resistance to rotation of the muzzle-head by the turret-motor.

To both flex and damp as necessary, the material of the convoluted segment must comply with lateral forces applied gradually, such as in tracking, but resist those applied suddenly, such as discharge recoil. The flexion provided by this joint, which is made of tubing of a thickness and material, to include coextrusions, that is optimized for the barrel-assembly, affords improved steerability in tighter anatomical bends when the barrel-assembly is advanced or withdrawn. Since the amount of rotation for a given muzzle-port configuration is limited, the airgun can be discharged during semiautomatic control using the linear positioning table while the barrel-assembly continues moving, with no distortion of the barrel-tubes as might affect the exit velocity occurring. More significant recoil shock absorption and damping is obtained by incorporating a second elastic disk or annulus between the ejection head and electomagnet assembly.

To ‘tune’ this second damper for the multiple reaction modes essential to defray the recoil characteristics associated with discharge from one or a plurality of barrel-tubes from one and the same barrel-assembly, the second damper can be simple, i.e., comprise a single elastomer, or interpose different elastomers over its area, can be compounded or laminated, and be ridged, serrated, triangular, square, or sawtooth-waved in contour on one or both faces. Upon emerging from the neck of the spindle journaled within rotor 82, the barrel-tubes 74 remain unattached until engaged in the barrel-channels XX. The barrel-tubes are accordingly rotated at their upper or distal points of attachment alone. Alternately, the barrel-tubes can be continuous up to and attached directly to the muzzle-ports without the interposition of an upper spindle portion; however, this tends to result in less than completely dependable bonding of the distal ends of the barrel-tubes to the muzzle-ports as required.

When the barrel-tubes can be rotated by the turret-motor without distortion or kinking when their distal termini are fixed in position, the junction with the barrel-channels in the spindle portion of the muzzle-head can be bonded as described below. The roof of the diametrically opposed electromagnet chamber is seen at 85, the spring-loaded door 86 leading to antemagnet chamber 87. The nose, i.e., the distal or front end, of the muzzle-head is like that of the radial discharge monobarrel seen in FIG. 26, which shows the nose (front end) at the center with the plane of the drawing receded above and below to show in section the recovery electromagnets of an ablation and angioplasty-incapable barrel-assembly, meaning a barrel-assembly capable only of discharge, and thus lacking a trap-filter in the nose, side-sweepers, laser, or burr radial discharge barrel-assembly.

With such a system, one to four or more miniball rotary magazine clips can be used in the same airgun and one to four or more miniball discharge barrel-catheters can be plugged into the airgun. By blanking out unneeded barrels at the rotary magazine clip, a barrel-assembly with more barrel-tubes than required can be used with fewer barrels. In barrel-assemblies of three or more barrels, the muzzle-ports are generally equally spaced about the muzzle periphery. The applicability of equidistant muzzle-ports with and without blanked rotary magazine clips is considered sufficient to omit a capability to circumferentially situate muzzle-ports; for eccentric lesions, barrel-assemblies with muzzle-ports fixed in eccentric positions are used.

Detachability of the muzzle-head from the barrel-catheter could pose a risk of detachment while deployed in a vessel, and with the muzzle-head unitized with the barrel-catheter, the durability of the two components in a unified embodiment is sufficient not to jeopardize losing the proportionately much greater value of the muzzle-head due to breakage. For tight control under fluoroscopic and angioscopic viewing, the use of polytetrafluoroethylene in radial discharge barrel-assemblies, which consist of barrel-tubes, the barrel-catheter containing these, and the muzzle-head, should impart a torque or turning ratio of 1:1. A 1:1 turning ratio is essential to position a two-way, partial or not fully encircling, magnet resilient tube mounting over the miniball implants. Positioning is assisted by adding angular displacement indicating tick marks to the radiopaque markers about the barrel-catheter used to indicate the length of catheter introduced.

Should recovery by means of an electromagnet be necessary, all miniballs are radiopaque. Length of entry markers are a standard feature of angioplastic guide catheters. Polytetrafluoroethylene being a stiffer material for catheters, greater pliancy may be imparted by making the barrel-tubes and barrel-catheter of polyurethane or polyethylene. Bending steerability or trackability within the radius of curvature unobstructive of miniball propulsion can be assisted through the use of an external electromagnet. The muzzle-head is wetted with heparin-saline solution and the rest of the barrel-assembly lubricated as specified above. Since the magnet antechambers at the front of the muzzle-head are closed off by spring-loaded doors, these are pushed open to allow wetting with heparin-saline solution. To impart a mild curvature to the barrel-channels in a solid block of metal would require that the solid block first be halved and then be quartered along its long axis twice to allow half barrel-channels to be milled into either face of the mating faces. The quadrants would then have to be fastened back together by means of an adhesive, which is not preferred.

Due primarily to the elasticity of the lumen wall in healthy tissue as a standard, the range of impact forces functional in implanting the miniballs subadventitially is wide; within this range, a difference in exit velocity affects the distance the miniball travels through the media and the length of the slit it cuts through the external elastic lamina, which is negligible when the force of impact is properly set. That only diseased tissue warrants treatment, and such tissue is capable of wide and unpredictable deviation from the normal is responded to by preliminary tissue puncture resistance testing means described below. With tantalum coated miniballs, the distance traveled along the inner surface of the tunica adventitia or outer fibrous jacket of the structure is observable fluoroscopically. However, the close observation and recording of wound production by such means necessitates the use of a high-speed camera such as a Redlake's MotionPro® HS Series, EG&G 549-11 Microflash® or Cordin Dynafax® 350 using preautolytic excised tissue under laboratory conditions.

To minimize the risk of stretching injuries from resistance to advancement, withdrawal, and traversing a tortuous stretch of vessel, barrel-assemblies made of materials other than polytetrafluoroethylene are coated with an external lubricious coating such as ACS Microslide®, Medtronic Enhance®, Bard Pro/Pel® or Hydro/Pel®, or Cordis SLX®. Just before introducing the barrel-assembly into the bloodstream, the muzzle-head is wetted with a heperine-saline solution, and if the barrel-catheter is not coated with or made of polytetrafluoroethylene, the rest of the barrel-assembly is wetted with a light coating of a well tolerated ophthalmic type lubricant such as 1% or 2% single-chain hyaluronic acid (sodium hyaluronate; oxycellulose; hydroxypropyl methylcellulose; hyaluronan) sold under such trade names as Healon®, Adatocel®, Amvisc®; IAL®, or Biolon® diluted with saline solution; or glycerin diluted with water.

For trackability or steerability to allow femoral or brachial entry and thus eliminate the need for open exposure, the tubing for a catheter to represent the barrel-tube in a single miniball discharging barrel-assembly with a radial discharge muzzle-head or a barrel-catheter containing multiple barrel-tubes, or the material used in both, to be described, ideally have high combined pliancy, or flexion without kinking or folding. The need for pliancy, usually expressed in terms of flexural strength or flexural modulus as defined by American Society for Testing and Materials standard Document D790-03 entitled “Standard Test Method for Flexural Properties of Unreinforced and Reinforced Plastics and Electrical Insulating Materials,” rises with the number, diameter, and wall thickness of barrel-tubes, four barrel-tubes contained within a barrel-catheter, for example, posing a stringent flexibility requirement.

A number of medical grade, flexible, high fatigue strength, bioinert, nondegradable, uncoated, and nonleaching nonchemical-absorbing polymers that are free of polymerization process chemicals and do not give off acidic plasticizer gas when sterilized are suitable for use as barrel-catheters and tubes in barrel-assemblies. Suitable barrel-tube materials include polytetrafluoroethylene, which is lowest in friction but relatively stiff, polyamide such as Dupont Nylon Zytel®, or a polyurethane elastomer, such as Dow Pellethane® 2363 and numerous similar products.

Due to the propulsive force of the airgun before the relief control retrofitted or built in is used to bleed off or moderate the pressure generated within the valve body, tubing material with a higher coefficient of friction than polytetrafluoroethylene but greater pliancy, such as low density polyethylene, vinyl, or nylon are readily usable through vascular bends, whereas stiffer tubing is not. For anatomical bends that due to the relative stiffness of polytetrafluoroethylene tubing make steerability resistive, a superior solution is to use barrel-tubes of highly pliant polymers such as Nylon 12 and Nylon 66, already approved for use within the body, but lubricated as stated above and lined with a thin coating of polytetrafluoroethylene for low friction, or slipperiness. By varying the relative thickness of polytetrafluoroethylene and polyamide in compound tubing, combinations of pliancy and stiffness suitable for application to the apparatus to be described may be obtained in different diameters over a wide range of tube internal wall friction.

Flexibility of the barrel-assembly over the length to remain outside the patient equal to that applicable to the length to be introduced is undesirable as gratuitously increasing the rolling resistance to the miniballs unpredictably. At the same time, flexibility sufficient to track anatomical bends is imperative for distal lengths typically introduced into the body. If made of the same material, then according to the softness of this material, the internal barrel-tubes and barrel-catheter containing these must not exhibit friction in the ‘bores’ proportional to the pliancy. A suitable combination of pliancy and stiffness in the barrel-tubes can be obtained by coextruding an inner layer of polytetrafluoroethylene within a soft outer polymer where the relative thickness of the polytetrafluoroethylene diminishes, incrementally changes, or discretely changes at one distance distad.

For increased stiffness, the barrel-catheter and barrel-tubes can be made of different polymers or coextrusions over the length of the barrel-assembly to remain outside the body. The pliancy of the barrel-tubes can also be varied along the length of the barrel-assembly by using centering devices (FIGS. 24 thru 29) that vary the distance from the longitudinal central axis of the barrel-catheter to the central axes of the barrel-tubes. Specifically, over the distal length of the barrel-assembly where flexibility to track anatomical bends in the vascular system is essential, the barrel-tubes are perforated, imparting greater flexibility to this length. Reduction in stiffness in the distal portions of the barrel-assembly to be entered into the body is also obtained by perforation of the barrel-tubes and by using centering devices to be described to position the barrel-tubes farther from the longitudinal axis of the barrel-catheter.

The internal pressure generated by discharge of the airgun is dissipated by using barrel-tubes with perforations over the distal portion of the barrel-assembly to be introduced into the body. These pressure relief perforations provide a path for the relief of the airgun discharge pressure within the barrel-assembly that is less resistant than the pressure that would be required for the gas to enter the bloodstream. These perforations must be too few, too spaced apart, and too small to cause folding or kinking of the barrel-tubes as the barrel-assembly is advanced transluminally. By situating these barrel-tube perforations toward the distal end of the barrel-assembly, the distal segment that must be more flexible to follow the curves of vessels are rendered more flexible. The number, shape, and location of these perforations is a factor in determining the pliancy of the barrel-tubes and the barrel-assembly as components therein.

To place blood flow side-holes in the barrel-catheter as well and thus achieve even greater flexibility is disallowed by the need for this space to equalize the pressure within the barrel-assembly during discharge while immersed in blood without introducing gas as bubbles into the blood stream. While to reduce friability due to rotary magazine clip-hole adhesion or barrel friction and any tackiness, a light coating of a well tolerated ophthalmic type lubricant such as 1% or 2% single-chain hyaluronic acid (sodium hyaluronate; oxycellulose; hydroxypropyl methylcellulose; hyaluronan) Healon®, Adatocel®, Amvisc®; IAL®, or Biolon® diluted with saline solution; or glycerin diluted with water may be applied to miniballs with a medicated outer coating of dried syrup, separate lubrication as might variably accumulate along the barrels introducing mechanical uncertainties and possibly forming a film over the muzzle-port by surface tension that might additionally congeal not permissible.

Pliancy, however, tends to vary inversely as the turning or torque ratio, which with a passive or nonmotorized muzzle-head, is ideally 1:1, and the coefficient of friction of the barrel tubing material, which is significant as the guideway for the miniballs. The coefficient of friction is determined with the aid of an Instron® or similar tester, such results provided by the tube maker. Turning ratio is more significant when the implants must be placed at particular circumferential angles. Since the airguns used generate sufficient propulsive force to project almost any interventially functional number and diameter of miniballs through respective barrel-tubes from the point of entry, usually inguinal, to the treatment area, such as at the heart, through rolling resistance comprised of bends in polymeric tubing of any coefficient of friction, and this force can be controlled, pliancy is the dominant consideration. As is conventional with guide catheters, barrel-assemblies are marked off in distance increments and indicate resistance to rolling per unit length at a standardized degree of bending or radius of curvature that must be coordinated with required force of impact data for different tissues.

Barrel-assemblies with plural barrel-tubes display a number or other distinctive marking for each produced by inclusion in the mold used to make the proximal end-cap and by engraved the same mark next to each muzzle-port. When the barrel-assembly must be coursed along a different route than the preparatory, angioplasty, access through a separate incision is not significantly traumatic and has been made relatively safe. Broadly, conventional stenting is preferable in geriatric and terminal patients, whereas application of the methods and apparatus described herein are preferable where life expectancy recommends avoiding sequelae. Barrel-assemblies with multiple barrels are for use in smaller diameter vessels and ducts, especially in the arterial tree, where operating time should be kept to a minimum. For this reason, it is desirable to discharge multiple miniballs at once and in quick succession. The structure of the barrel-assembly thus allows radial discharge and the airgun is semiautomatic. At the same time, dependable means for preventing miniballs from escaping into the blood stream must be provided.

Turning now to FIG. XX, the barrel-assembly must be freely introducible into the bloodstream and freely movable forward and backward within a lumen that matches the muzzle-head in diameter. For this purpose, the muzzle-head has longitudinal blood grooves midway between the barrels on its outer surface, and the barrel-catheter is about 10 percent smaller in outer diameter than the muzzle-head with the turret-motor slightly larger when the distal end of the barrel-catheter is not narrower to accommodate it. The blood displaced by the barrel-assembly when introduced into the bloodstream thus moves past the advancing barrel-assembly and out through the entry incision. Once inserted, the blood can move out of the way of and past the barrel-assembly whether advancing or withdrawing, whether antegrade or retrograde.

The blood grooves continuous over the muzzle-head to include both the proximal or muzzle-port and distal or electromagnet sections allow some pulsation to pass the barrel-assembly. Some limited transmission of the pulse through the barrel-assembly is obtained by means of side holes through the wall of the catheter barrel (see De Bruyne, B., Stockbroeckx, J., Demoor, D., Heyndrickx, G. R., Kern, M. J. 1994 “Role of Side Holes in Guide Catheters: Observations on Coronary Pressure and Flow,” Catheterization and Cardiovascular Diagnosis 33(2):145-152). The side grooves or side hole tunnels are connected by means of peripheral tangential tunnels that course diagonally relative to the longitudinal axis. The placement and angles of these flow through tunnel tubes can also be used to buttress and stiffen the barrel-assembly over designated segments along its length.

Several different materials and manufacturing techniques can be used to produce the one to four or more way radial discharge muzzle-head. The preferred embodiment consists of micromachining and micropolishing a solid block of stainless steel into front or ejection head and rear motor housing portions. The structure of the muzzle-head is the same regardless of the number of barrels or circumferential angle of the barrel exit ports, and a variety of double barreled or two-way barrel-assemblies in various sizes may be necessary to allow variation in implantation angles. Passivation to remove surface contaminants or to improve the appearance of the muzzle-head, which is highly polished, is unnecessary, but is desirable for enhancing corrosion resistance. Made thus, the barrel-tube channels that contain the barrel-tubes in the muzzle-head are machine or micromachine drilled, hence, straight throughout their length.

To allow the mild curvature of the barrel-tubes necessary to diverge, that is, to splay or veer outwards to be redirected from the parallel orientation in the barrel-catheter to the circumferential placement of the muzzle-ports about the periphery of the muzzle-head, a splay space or splay chamber is interposed between the barrel-catheter receiving recess or socket at the proximal end of the muzzle-head and the entry of the distal ends of the barrel-tubes into their respective similar but smaller sockets in the proximal surface of the upper or distal portion of the muzzle-head which continues the barrels to the muzzle exit ports. The length of this space or splay chamber depends upon the wall thickness and pliancy of the barrel-tube material. The underside of the upper, barrel-channel portion of the muzzle-head, or ceiling of the splay chamber has four openings to receive the four small gauge barrel-tubes one each into a barrel-channel. The proximal or bottom end of the muzzle-head includes a collar or neck to receive the barrel-catheter containing the barrel-tubes.

Most often the pliancy of the barrel tubing material will necessitate that the distal ends of the barrel-tubes be able to rotate and reciprocate within flush joints XX of barrel-channels XX of the metal portion of the muzzle-head that terminates with the muzzle-ports. However, provided the outer surfaces of the polytetrafluoroethylene barrel-tubes in contact with the walls of the barrel-channels have sufficient slack to avoided distortion of their bores when rotated by the turret-motor, these are primed or etched with a special purpose chemical such as Acton Technologies, Inc. FluoroEtch® and coated with an adhesive suitable for bonding etched polytetrafluoroethylene to stainless steel, such as Master Bond, Inc. EP42LV, a two component, low viscosity room temperature curable, epoxy adhesive, then threaded up through the bottom holes of the splay chamber ceiling until exiting the vertically oblong muzzle barrel openings.

The ends are then cut and polished flush to the surface of the muzzle. The barrel-catheter is then slipped over the set of four barrel-tubes until it is brought just short of the socket or receiver for it formed by the collar at the proximal or bottom end of the muzzle-head. The outer surface of the barrel-catheter to engage the socket at the bottom or proximal end of the barrel tip is etched and the same adhesive applied before its upper end is inserted into the socket. An alternative method for producing two and four-way muzzle-heads over a range of diameters from about 7 to 10 French is polytetrafluoroethylene thermoforming by resin transfer-molding, which is well known to those skilled in the art of plastic molding. Made thus, the polymeric muzzle-head should nevertheless contain ferromagnetic inclusions to preserve steerability and abutment with the assistance of external electromagnet.

Since the barrel-catheter and barrel-tubes as well as the barrel tip are all likely to be made of polytetrafluoroethylene, conventional polytetrafluoroethylene resists self-bonding, and the barrel-catheter and barrel-tubes would not be molded in one piece with the barrel tip, a special polytetrafluoroethylene molding material such as E.I. Dupont de Nemours Teflon NXT® is used. If the muzzle-head is cast, then positive inserts in the mold can be used to yield mildly curved barrel-tube channels eliminating the need for a splay space. Preferred is a barrel-assembly which comprises the barrel-catheter and muzzle-head in a single unit that plugs into the barrel of the airgun. The barrel-assembly comes with rotary magazine clips containing test miniballs and should never be used with test miniballs of different specification. To incorporate the rotary magazine clip chamber at the proximal end of the barrel-catheter as unitized is not preferred, because this necessitates a communicating arm or intermitting pawl from the airgun.

As a matter of terminology, the switch or trigger actuator and pressurized gas cylinder or canister represent the minimum distinctly airgun portions of the apparatus described herein. The barrel-catheter and barrel-tube sockets consist of internal diameter flush joints, meaning the wall thickness of the inserted tubes is accommodated by the surrounding material of the receptacle so that there is no change in the internal diameter of the lumen at the joint. The end of the barrel and proximal end of the barrel-assembly are keyed to assure proper alignment, which the 1:1 turning ratio of the barrel-assembly supports. In an off the shelf airgun modified for use in accordance with the objects set forth herein, the barrel insert used to reduce the caliber stops half way down the barrel to allow the proximal end of the barrel-assembly to be inserted. Barrel-channels that avoid angles throughout their course yield a smoother or more linear relation of propulsive force to exit velocity.

An angle in the barrel-tubes too slight to stop the miniball at lower velocities nevertheless dissipates its momentum or propulsive force. While a steep gap in exit velocities separates implantation force of impact values from puncture of the outer fibrous layer of the ductus, variation in exit velocity directly effects the distance of miniball travel along the inner surface of the fibrous outer layer before it is brought to a stop. Overshooting or overtravel is readily compensated for by withdrawing the muzzle-head by the proportionally corresponding distance, but is to be avoided as imposing needless additional cell damage and edema. Because the exit velocities smaller than that necessary to reach a nonspecific subadventitial position while not puncturing the outer fibrous jacket or tunic of the ductus cover a range of impact force values, adjustment in the exit velocity need not achieve inordinate exactitude merely to achieve subadventitial placement while not posing a risk of puncture.

At the same time, changes in distance or depth into the vascular system are not accompanied by any change in the chamber to muzzle-head length of the barrel-assembly. Thus, the small decrements in depth required to withdraw the muzzle-head to place successive discharges exert no effect on the ability to place the miniballs at the prescribed relative distances along the ductus. However, the subpuncture range of exit velocities equate to equivalent impact forces that while spread out in value by the tunica adventitia, or outer elastic lamina, and consistent from discharge to discharge as to allow successive termini to be accurately placed as to related position, nevertheless propel the miniballs to correspondingly different distances within the softer tunica media along its inner surface. To achieve a certain miniball trajectory terminus in relation to distance from the muzzle-head and therefore avoid needless smooth muscle trauma requires tight or precise control.

This combination of factors means that from a risk of puncture standpoint, the initial discharge, while preferably purposeful in terms of treatment, is subcritical and allows the distance traveled by the miniballs to be noted with the aid of the several imaging techniques available. Any over- or under-shooting is then corrected by adjusting the exit velocity in proportion to its extent. Since the pathology is likely to have changed the mechanical properties of the tissue, a preliminary test discharge at a distance from the lesion to be treated is not recommended. As any site aside from that diseased will present different properties, an initial test discharge anywhere but into the diseased tissue to be treated will not yield dependable data. For this reason, implantation is begun with the airgun set for the exit velocity or force of impact value predicted with the aid of tables supplied with the apparatus to seat the miniball subadventitially without overshooting along the inner surface of the fibrous outer layer. Due to the tiny diameter and bioinertness of the miniballs, puncture with resultant entry into a body cavity of a miniball will rarely prove other than innocuous, and the hole in the vessel wall should, through coagulation and swelling, quickly seal itself minimizing hemorrhage or the leakage of lumen contents.

Miniballs are all highly radiopaque and retrievable by means of an endoscope introduced through the incision made to place the stent-jacket. The results of the initial airgun discharge are carefully evaluated and used to adjust the discharges to follow. The means for preventing the accidental release of a miniball within the tracheobronchial tree or vascular system and removing any spillage of intestinal contents or bile following inadvertent puncture is discussed below. Restraint of the ductus wall from radial excursion at and about the point of impact would reduce the impact force required to puncture the wall; however, both wall and material of the stent jacket are sufficiently elastic to comply with movement of the wall at and about the point of impact. The stent-jacket is compliant both internally as to absorb the displacement of the wall in response to the impact of implantation, and as a result of its overall conformation, which includes a slit cut entirely along one side, elastic in response to the gross movement of the smooth muscle in the ductus wall. Spillage of contents into the surrounding body cavity as the result of accidental puncture of the urinary tract or bile ducts is responded to by aspirating the spillage through the incision made to insert the stent-jacket.

Since it is compliant, the stent-jacket, or extravascular surround component of the stent, can be placed either before or after the intravascular component consisting of the full complement of miniballs has been implanted. Inserted before the miniballs, the stent-jacket may exert a slightly nonuniform effect upon terminal velocity and eccentric compression of the lumen wall about the lumen circumference against the muzzle-head but assists in retaining the miniballs. This attraction assists in preventing rebounds or failures of impact force as would release a miniball into the lumen. The accidental release into the lumen of a miniball is minimized when the muzzle-head fits snuggly within the lumen, damping eccentric reactive accelerations or recoils without compressing the wall. Excessive compression of the wall interferes with the ability of the wall to comply with the impact and thus the ability to properly implant the miniballs. The mechanical and electrical connections of barrel-assemblies to airguns is discussed under sections to follow.

### Tube Polymer Non Intrinsic Barrel-Catheter Flexibility Altering Elements

### Centering Devices (Centering Disks)

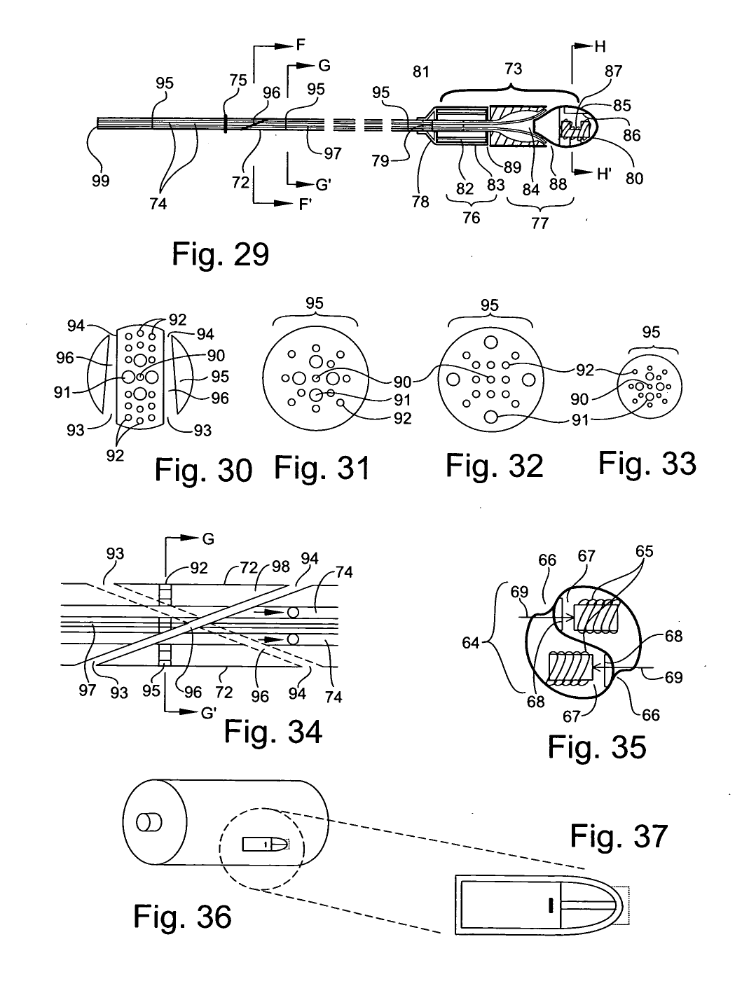

For fixing barrel-tubes in radial distance from the longitudinal central axis of the barrel-catheter, a centering device as shown for use in a four-way radial discharge barrel-assembly in FIGS. 28, 29, and 30 is used. The centering devices have a central hole 90 to pass the electrical conductor for supplying power to the turret-motor and tractive electromagnets, barrel-tube holes 91 to pass the barrel-tubes, and gas pressure equalizing perforations 92, which allow the discharge pressure that escapes through the perforations in the length of the barrel-tubes within the body to access the entire peribarrel space seen in FIGS. 31 and 32 as 98 and so become equalized within the barrel-catheter. Incorporation into the barrel-assembly of a laser necessitates an additional hole as seen in FIGS. XX and XX. FIG. 27 provides a side view of centering devices 95 as well as a blood-tunnel 96 in a four-barrel radial discharge barrel-assembly shown in mid-longitudinal section, FIG. 35 providing greater detail.

At intervals along the barrel-assembly sufficient to prevent sagging of the barrel-tubes, the outer edges of the centering devices are bonded to the inside of the barrel-catheter, and the edges of the holes for the barrel-tubes 91 are bonded to the barrel-tubes by means of an adhesive. The center hole 90 for the wire insulation is not bonded. Depending upon the materials of which the centering device, barrel-tubes, and barrel-catheter are made, the adhesive used is, for example, cyanoacrylate cement, Master Bond® EP42LV, or Loctite Hysol Cool Melt®. By allowing the radial spacing among the barrel-tubes to be increased as in FIG. 33, the centering devices allow the flexibility of the barrel-assembly to be reduced. This factor is used to reduce the tendency of portions of the barrel-assembly outside the body to flex and increase the rolling resistance to discharge of the miniballs. Toward the leading or distal end of the barrel-assembly, the radial distance of the barrel-tubes is reduced for increased trackability around anatomical bends as shown in FIG. 32, to which the pressure equalization perforations in and toward the distal ends of the barrel-tubes contributes.

The torque ratio of the barrel-assembly can be adjusted over a wide range and varied for different segments of the barrel-catheter according to the interval used to separate the centering devices and whether these are bonded to the internal surface of the barrel-catheter. Along with the materials and dimensions of the parts of the barrel-assembly, the flexibility of the barrel-assembly may be adjusted over different segments by changing the radial spacing of the barrel-tubes by using differently configured centering devices. To create paths for blood to pass, metal in the spindle distal to the spindle neck, which is journaled in the through-bore rotor of the turret-motor, is removed. The periperhal openings to these passages or blood-ports are machined for continuity with the blood-grooves described below that course longitudinally along the outside of the muzzle-head.

For additional flexibility for a given combination of barrel-catheter and barrel-tube materials, the diameter of the barrel-catheter normally introduced transluminally, can be reduced as represented by the centering device shown in FIG. 34. The joint between the larger and smaller diameter barrel-catheters, which can also differ in material, or if coextruded, then both the inner and outer material, is made by telescoping the end of the wider proximal length of barrel-catheter tubing into the narrower length with the difference between the two filled with either a segment of tubing of intervening diameter and wall thickness or with tape, such as vinyl pressure-sensitive tape. The ledge created at the joint between the different diameter tubes is bevelled and rounded off to prevent abrasion or pulling of the lumen wall upon withdrawal.

If necessary, an external hand-held electromagnet is used to assist in steering the muzzle-head through sharper turns by attracting the turret-motor and magnet cores in the otherwise nonmagnetic muzzle-head. The barrel-assembly is made stiffer by 1. Using a barrel-catheter of larger diameter, 2. Making the barrel-tubes and barrel-catheter of stiffer materials, 3. Distancing the barrel-tubes farther radially from the longitudinal axis, and 4. Incorporating blood-tunnels, as described below. Incorporation into the barrel-assembly of a photo-ablation laser also adds stiffness. The caliber of the implants is decided purely on the basis of the medical requirement and never manipulated merely to change the stiffness of the barrel-assembly. Change in these factors also changes the torque ratio of the barrel-assembly, which is further variable by leaving the outer edges of the centering devices unbonded or bonded to the internal surface of the barrel-catheter.

Ischemia Impeding Elements that Also Serve as Ube Polymer Nonintrinsic Barrel-Catheter Flexibility Altering elements

### 1. Blood Grooves

In a radial discharge monobarrel, the diameter of the barrel-assembly is small, posing less obstruction to the circulation in a vessel of given caliber than with multibarrel embodiments. By the same token, multibarrel embodiments minimize the period that the barrel-assembly must remain in the lumen, and when the muzzle-ports face in the opposite direction, cancel out most recoil. When the motor housing is already as narrow as possible, blood-grooves cannot be routed or impressed therein. When, however, muzzle-head 70 is deliberately wider than necessary to fit flush within the lumen round and about, then as shown in FIG. 24, blood-grooves 66, to serve in a manner similar to the side-holes in conventional catheters, are incorporated to expedite the flow of blood past the muzzle-head. Blood grooves 66 are continuous with the ports and passages cut through the muzzle-head and when present, the spaces above the shelves represented by the floor of each enclosed tractive electromagnet chamber seen from the opposite side.

### 2. Blood-Tunnels

Blood-tunnels are tubes within the barrel-catheter that inlet at a point at the periphery of the barrel-catheter, and closing off the space these contain from the surrounding space within the barrel-catheter, or peribarrel space, course longitudinally at an angle or diagonally, to outlet at an arcuate or circumferentially removed end-point distal to the inlet. The blood able to pass through a blood-tunnel will largely depend upon its internal diameter, which thus becomes a factor in spacing the barrel-tubes by means of centering devices within and sizing the barrel-catheter. The barrel catheter is usually smaller in diameter than the muzzle-head, vitiating the need to create a passage to achieve a function equivalent to that of side or perfusion holes in conventional catheters, this being to allow some oxygenated blood, albeit negligible, to pass the transluminal component occupying the lumen.

Referring now to both FIGS. 31, 32, and as best seen in FIG. 35, the flow of blood is antegrade from left to right, and the direction of miniball discharge, likewise left to right, is indicated with arrows, making the proximal inlets 93 of blood-tunnels 96 distinguishable from the distal outlets 94. The barrels 74 in FIG. 27 pass through the barrel-tube holes 91 shown in FIG. 36 in the centering device 95, in which 92 are gas pressure equalization holes and 90 is the central aperture through which wires 97 for the electromagnets (64 in FIGS. 25 and 80 in FIG. 27), turret-motor if present, and a side-sweeping option if present, passes. The incorporation of blood-tunnels necessitates the use of a larger diameter barrel-catheter, reducing the flexibility of the barrel-assembly on both scores. While a barrel-catheter that is narrow may be increased in diameter to allow the incorporation of blood-tunnels with more robust walls as stiffening buttresses, no portion of the muzzle-head is increased in diameter merely to incorporate blood-grooves. Larger in diameter, multibarrel radial discharge barrel-assemblies can incorporate not only blood-grooves but blood-tunnels, which are shown in FIGS. 27, 31, and 32 as 96 within the barrel-catheter 72 and described below.

While a monobarrel radial discharge barrel-catheter can be small enough in outer diameter to serve as a kind of ‘guidewire’ for follow-on devices, devices that use guidewires normally serve functions that precede rather than succeed stenting; however, where the muzzle-head passes readily through the lumen as to dispel concerns about stretching, dissection, or perforation, the muzzle-head can be used to implant miniballs upon withdrawal. Depending upon the angle at which the blood-tunnel tubes course through the barrel-catheter and the material or materials of which the blood-tunnel tubes are made, the blood-tunnels allow some transmission of the pulse through the barrel-catheter. Made of more rigid materials, the blood-tunnels can, according to number and spacing, also act as structural buttresses to stiffen the barrel-catheter. Placing blood-tunnels in longitudinal sequence along one radius of the barrel-catheter, for example, will bias the bendability of the barrel-assembly away from that direction toward the perpendicular or normal direction. Angular uniformity in the stiffness of the barrel-assembly thus requires a circumferentially complementary and balanced distribution of blood-tunnels.

When made of a pliant material or materials, such as vinyl, and bonded by means of an adhesive to the wall of the barrel-catheter with acutely angled mitered ends, the blood-tunnels can course in substantially adjacent relation to the concave surface of the barrel-catheter. Made of rigid material, such as polystyrene or high density polyethylene or polypropylene, the blood-tunnels are straight as to appear geometrical cords in cross-section, and buttress the wall of the barrel-catheter, making it stiffer. With the barrel-catheter and barrel-tubes bonded to the centering devices, no significant increase in stiffness is realized by coursing the blood-tunnel tubes through and bonding these to the centering devices.

In the portions of the barrel-assembly to remain outside of the patient, the elimination of nonfunctional bends that detract from control over the rolling resistance to the miniballs is necessary to achieve accurate exit velocity and impact force. Thus, when the materials of the barrel-catheter and barrel-tubes are highly pliant, the incorporation of blood-tunnels in the barrel-catheter serves to minimize flexion in the proximal portions of the barrel-assembly. By comparison, in the distal portions of the barrel-assembly introduced into the patient, flexibility sufficient to track anatomical bends with little resistance is preferable.

Accordingly, blood-tunnel tubes are not incorporated toward the fore; however, the flexibility sought for this portion recommends the use of centering devices that allow the diameter of the barrel-catheter to be smaller, and this in itself will allow some passage of the pulse up to and through the blood grooves in the sides of the muzzle-head. The elimination of centering devices in the distal portions of the barrel-assembly does not negate the need for a peribarrel space sufficient in volume to releave the pressure of discharge so that no gas will be ejected into the bloodstream. Since the barrel-catheter must accommodate this need for sufficient peribarrel space, a barrel-catheter of minimum internal diameter must be provided. The omission of centering devices thus has only the result of the dropping of the barrel-tubes to the floor of the barrel-catheter.

### The Barrel-Assembly End-Plate

The centering device at the proximal end of the barrel-assembly is the end-plate, shown in FIG. 37 as part number 99. Unlike the internal centering devices distal to it, to prevent the inflow of blood or other bodily fluid when the muzzel-ports at the sides of the muzzle-head are introduced into the lumen, the end-plate must render the proximal end of the barrel-assembly airtight. It therefore does not have gas pressure equalization holes 92 as shown in FIGS. 28 thru 32. Since the airgun chamber must be airtight to prevent the loss of propulsive gas pressure, the proximal end (end-plate) of the barrel-assembly may be seen as redundant in this airtightness, which is precautionary. More specifically, as clarified in the section above entitled Airgun and electrical connections and controls of barrel-assemblies by functional type, an ablation and angioplasty-incapable barrel-assembly is never used without its proximal end engaged in the airgun chamber.

Reciprocally, so long as it remains in separate use during an angioplasty, an angioplasty-capable barrel-assembly never performs a discharge function as to generate internal pressures that must be dissipated within it to conserve propulsive gas pressure and to avoid gas embolism if discharged in the vascular tree. A barrel-assembly of latter type is provided with a slit-valve of a stiffer elastomeric sheeting material at its end-plate to allow entry into the central canal for use as a service channel and affords an opening for a cooling catheter. When engaged in the airgun chamber, the latter makes the proximal end of the barrel-assembly airtight.

The end-plate fixes the barrel-tubes in position for exact alignment with their respective holes in the rotary magazine clip. Electrical conductors 97 that course through the longitudinal axis of the barrel-catheter exit proximally through central aperture 100, with the conductors passing radially in bonded relation to the face of end-plate 99 to end on terminal 101. End-plate 99, molded in any suitable plastic, such as polyethylene terephthalate or polystyrene, contains a simple slit valve made of elastomeric sheet material, such as polyurethane, chlorosulfonated polyethylene, silicone, or fluorosilicone. The slit valve serves both to relieve excess pressure in the peribarrel space and as an entryway through which to admit a test rod, lubrication injection catheter, or turret-motor and/or electromagnet assembly rapid cooling catheter (cooling capillary catheter) when necessary, as described elsewhere herein.

For nonthermal angioplasty using side-brushes (above and below), the turret-motor is connected to the drive control electronics to rotate the muzzle-head only when the anatomy makes manual rotation difficult or risky. Except that when the turret-motor stator has just been used as a heating element for thermal angioplasty, a brief interval must be allowed for cooling, end-plate connection of the turret-motor positional drive control electronics has the advantage of allowing a barrel-assembly that battery powered is nontethered and otherwise independent of the airgun to be immediately and intermittently connected to the drive controls only when the turret-motor is needed for rotation. During discharge, the turret-motor must be connected to the drive control electronics. Alternatively, the electrical connection of the barrel-assembly can be by means of terminals on the side of the barrel-assembly just distal to the proximal length of it inserted into the airgun barrel.

### The Muzzle-Head Turret-Motor (Turret-Servomotor)

A motorized muzzle-head monobarrel turret (rotary joint, swivel) is shown in FIG. 25, wherein motor housing 61 contains motor rotor 60 within motor stator 62, and a motorized muzzle-head multibarrel turret rotary joint is shown in FIG. 27 as 76, wherein motor housing 78 encloses motor stator 83 and rotor 82. Switching the recovery electromagnets in the nose of the muzzle-head between thermal ablative or angioplasty and miniball recovery functions and/or the turret-motor between thermal ablative or angioplasty and rotary positioning functions is accomplished at the switch used to toggle between and thus select the function desired, the circuit of the function not selected being disconnected and thus disabled.

One advantage in mounting the muzzle-head in a motorized turret is that remotely controllable, it is no longer necessary to rotate the entire barrel-assembly merely to rotate the muzzle-head, and this allows the use of more pliant materials in the barrel-assembly, so that rotary maneuverability ceases to pose a significant risk of stretching injury. If necessary, the muzzle-head, to include both port and magnet assembly portions, is wetted with a lubricious material as specified above to minimize resistance to rotation by the motor. Another advantage is that rotation of the muzzle-head allows combining the use of barrel-blanked clips with rotation making it possible to treat each successive segment of a vessel discriminately as to the circumferential placement of the implants with no need to withdraw an inserted barrel-assembly and replace it with another.

Yet another benefit of the ability to rotate the muzzle-head by means of a motorized turret is elimination of the need for a variety of muzzle-heads having different numbers of muzzle-ports at different angles. A monobarrel barrel-assembly that is unassisted, that is, lacks a motorized pivot, or a multibarrel turret that lacks a motorized turret, requires a turning or torque ratio sufficient to rotate the port or ports to the potential target angle most distant angularly from its starting position without jamming during discharge. As stated under the section on multibarrel radial discharge barrel-assemblies above, a four-way muzzle-head, for example, demands to be rotated up to 22.5 degrees in either direction to target any circumferential angle about the lumen and its diametrically opposed two trap-extraction electromagnets up to 90 degrees in either direction to target a misplaced miniball implant for extraction, and this angle can be significantly larger with a barrel-assembly having eccentric barrels. Since a means for recovering any loose or mispositioned miniballs is imperative, the lesser turning angle cited of 45 degrees to rotate a four-way muzzle-head for discharge is superfluous, an angle of rotation for the magnet assembly taking priority.

This demands corresponding pliancy through the combined effect of the barrel tubing material and the length of the splay chamber. When the barrel-assembly is rotatable by means of a motorized rotary turret and any rotary magazine clips that reduce the number of miniballs discharged at a time as necessary are available, then any circumferential placement of miniballs up to the caliber that the lumen diameter will permit is possible without a significantly greater risk of intraluminal stretching injury or the need to withdraw and replace a barrel-assembly of one configuration in order to replace it with another mid-procedure. With muzzle-heads with eccentric ports, the angular displacement of the muzzle-head is dependent upon the pliancy of the barrel tubing and the slack available in the splay chamber. The rolling resistance presented by increasing curvature as the ports are approached must be compensated for by adjustment of the airgun settings. Rotating the muzzle-head purely to reduce the exit velocity is not considered practical, the motorized turret provided to assist in accurately placing the miniballs into eccentric lesions.

This notwithstanding, the incorporation of a motorized muzzle-head that can be rotated to any angle eliminates the need for rotating the barrel-assembly, and this has the advantage of allowing the barrel-assembly to be made of more pliant tubing material. FIGS. show barrel-assemblies with built in motors that allow the muzzle-head to be rotated. Such an apparatus is intended for use only when the pathology is of variable circumferential eccentricity over the course of vessel to be treated. The applicability of a motorized muzzle-head is limited by the diameter of the motor that can be achieved to generate the torque essential to rotate the muzzle bit through an arc in either direction of up to 44 degrees for the caliber of miniballs appropriate for the vessel to be treated. When a two-way or three-way muzzle-head with muzzle-ports at angles other than 90 degrees meets the procedural requirement so that withdrawal and replacement of the barrel-assembly will be unnecessary, then these are used. Otherwise, rather than to withdraw one barrel-assembly in order to replace it with another, a four-way barrel-assembly is used and torqued slightly to position the ports used by the rotary magazine clip as necessary.

### Muzzle-Head Servomotor Modes of Operation

The turret-motor must provide three distinct modes of operation that are selectable and adjustable with selector switches to the motor controller. These functions are heating, and oscillation, which pertain to angioplasty with an angioplasty-capable barrel-assembly independent of the airgun, when to allow free manual movement, any form of tether would be objectionable, so that power is by an onboard battery, and positional, which pertains to implant discharge with the barrel-assembly inserted into the airgun, when the need for free movement in manual use, hence, the fact of tethering and connection to a servomotor drive amplifier controller is not an issue. That power is from a battery disallows conventional solutions, such as producing oscillatory performance by detuning the velocity loop in, or programming a set of oscillatory (vibratory) frequency modes to be executed by, the servocontroller without, for example, infrared transmission of the control signals.

More specifically, these modes of operation include I. Rotation in either direction as arc-limited according to the number and twisting limits of the barrel-tubes, 2. Heat generation to serve as a heating element for thermal angioplasty (whether in coordination with like use of the recovery electromagnets distal to the ejection head), and 3. Oscillation useful for a. Assisting to free the muzzle-head should it cling to or seize against the endothelium, or b. Obtaining vibratory action of the side-sweeping brushes. Angioplasty seldom resumed once the barrel-assembly, even when angioplasty-capable, has been inserted into the airgun and discharge initiated, should the desirability for further angioplasty become apparent following airgun insertion, the control of angioplasty functions—individual or combined use of the turret-motor and recovery electromagnets as heating elements for thermal angioplasty and deployment and retraction of side-sweeping brushes—can still be controlled from the control panel onboard the barrel-assembly as an independent apparatus.

However, combination-forms that additionally incorporate a rotary burr or laser are more costly and less likely to be adapted for conventional console-remote control, and more likely to remain tethered prior to insertion into the airgun. Unlike the linear positioning table or stage used to insert and retract the barrel-assembly in submanipulable millimetric increments as described below, which are available from many manufacturers incorporating several different techniques, turret-motors must be custom made to afford adequacy of through-bore internal diameter within the severely constrained and isolation of the magnetic fields within the motor from implants.

Older technology brushed through-bore torque motors of outrunner configuration are not preferred as incorporating a wound armature or rotor at the center and permanent magnet stator in surrounding relation thereto, which allows a through-bore of large diameter but encircles the windings amid the surrounding permanent magnets; the wound armature in a brushed motor of conventional or inrunner configuration is the rotor and in a brushless (electrically commutated) three-phase synchronous motor, the wound stator, the rotor being the field assembly. Direct drive brushless ring synchronous torque motors of inrunner configuration with a proportionally large through-bore are made in conventional sizes by Etel Incorporated, Mötiers, Switzerland, for example. While some angioplasty-capable barrel-assemblies can have an inmate polyphase drive controller microcircuit, most do not, necessitating that a drive-controller amplifier be provided in the airgun enclosure.

The brushless type torque motor with windings peripheral rather than encircled at the center is inherently better suited to the present application than is the brushed or mechanically commutated type in several key respects. Significantly increased power density, or power-to-size ratio, supports the extreme miniaturization essential, brushless operation provides much more precise and uniform control at low speed, and the peripheral location of the windings, if inadvertently, confers additional utility in allowing circumscribed areas at the surface of the muzzle-head (heat-windows) to be used for thermal angioplasty. While in industrial applications the generation of heat peripherally is beneficial for dissipating the heat, here the reverse is true, the peripheral generation of heat used to advantage. As described below, a prepositioned or inserted rapid cooling catheter is introduced through the barrel-assembly to the muzzle-head to return the ablating surface to body temperature.

Since positional use of the turret-motor is too intermittent to generate any significant heat and the motor is never used positionally and as a heating element at the same time, heat build up does not limit torque output. Sine wave driven brushless direct current (permanent magnet alternating current) through-bore torque motors have windings that encircle the permanent magnet rotor providing more direct transfer of heat through a heat-window (below) and further distancing the rotor magnets from the implants, adding a measure of protection against disruption due to magnetic leakage despite the closed magnetic circuit of the housing. Such a motor is able to provide a bore that is large enough to provide a gas pressure relief path (above) and the passage of barrel-tubes. The small external diameter of the motor necessitates maximum torque for the size, dictating the use of a direct current (permanent magnet alternating current), motor, which for reasons already stated, is made long relative to width.

The turret-servomotor is preferably a three-phase brushless direct current, direct-drive (transmissionless, gearless) limited angle through-bore torque motor or torquer with high axial, radial, and torsional stiffness, and high stall torque (stand-still torque; hold-fast torque) that develops its highest torque at low speed. The closed-loop feedback signal is generated by three digital Hall-effect commutation sensors that indicate the instantaneous position of the rotor. A once-per-revolution index sensor indicates the reference angle (home angle, home location, rotational reference datum). Alternatively, some control drive differentials or comparators require position and velocity feedback from a coaxially mounted resolver (analog) or optical encoder (digital), or a potentiometer.

To generate sufficient torque in a motor that the portions of the arterial system most often demanding treatment can limit to 2.5 millimeters in outer diameter, the motor stator is wound with silver wire and the rotor and stator are made proportionally longer, (i.e., greater in axial length) relative to diameter, generally in the ratio of 5:1, such as 2.5 mm in diameter and 12.5 mm in length. Such is unconventional in torquer motors, which are usually ‘pancake’-configured. Thermal angioplasty-capable muzzle-heads necessitate thermal insulation about the heat-window or windows which, consisting of outer coatings of silicon aerogel and polytetrafluoroethylene, for example, present minimal thickness to limit even more severely the diameter of the muzzle-head and therewith gauge of the vessel that may be treated.

To achieve contact all around the muzzle-head without stretching the lumen wall serves to assist in maintaining direct thermal window-lumen wall contact for thermal angioplasty that uses the turret-motor as the heating element while reducing the risk of thrombogenesis due to interposed blood and avoid 1. Discharge through intervening lumen contents allowing more equal impact force among miniballs discharged at different radii as applicable, 2. Compressing the media or the equivalent making subadventitial placement difficult if not impossible, and 3. Stretching injury.

Closed-loop control of the turret-motor is not intrinsically necessary but arises by default in that alternative through-bore remote positioners or drivers other than direct-current motors, such as torque synchros and stepper motors, have drilled shafts, which are unable to provide a through-bore of sufficient diameter in motors of the millimetrically incremented outer diameters required (generally 2.5 to 5 millimeters). Additionally, through-bore direct-current torque motors are familiar, whereas through-bore torque synchros and stepper motors are novel, and would increase the design problems of miniaturization even if alternative drivers could be made with bores of sufficient diameter.

Control of the turret-motor is accordingly closed-loop, digital incremental, and point to point. It being preferable for a given application to maintain contact with the lumen wall circumferentially, the turret-motor, hence, the barrel-assembly, is generally chosen on the basis of diameter as well as the functions required. Since a condition of sliding contact against the lumen wall entirely around the circumference must vary, the load placed on the motor will vary. Except when used as a heating element in barrel-assemblies designed for thermal angioplasty as described in the section to follow, the turret-motor is connected only intermittently in positional use and therefore does not generate thrombogenic heat. The direct-drive motor provides the backlash-free operation to allow the servostiffness and bandwidth essential to achieve instant accelerations, stops, and settling times.

This suddenness of operation affords the frictionless endothelial breakaway and quick stops necessary to preclude tissue adhesion and stretching injury, which the lubricity of the fluoropolymer coated muzzle-head enhances. For such point-to-point control, a feedback loop for velocity is omitted, only displacement controlled. Additional operation of the turret-motor (and tractive electromagnets) for thermal angioplasty recommends quick heatability and dropping from the less thrombogenic temperature of 90 degrees centigrade (reference provided below). Elongation of the motor housing-lumen wall interface thus allows sufficient torque in a motor of small diameter making the device passable farther down the vascular tree at the same time that it affords more surface contact area in support of the angioplasty function.

In barrel-assemblies designed for thermal angioplasty, elongation of the muzzle-head not only compensates for the limitation imposed on motor power output by severe limitation in diameter, but allows heat to be generated from three more independently controllable sources of heat, as described below. In side-sweeping barrel-assemblies, a longer muzzle-head not only compensates for the limitation imposed on motor power output by severe limitation in diameter but makes possible the use of longer longitudinally deployed side-brushes. Direct drive through-bore torque motors with limited angle control have been manufactured in conventional sizes and shapes by the Kollmorgen company (through-bore pancake torquer models S200 and S300 (not designations for driver controller amplifiers), now a brand of Danaher Motion, Inc.

With a four-way motorized muzzle-head, the maximum angle of rotation required to direct the muzzle-ports is 44 degrees and the tractive (trap) electromagnets 90 degrees in either direction. The motor is made to fit a barrel-assembly of a certain diameter and required range in rotational angle, at most, one full circle, which allows aiming a radial discharge monobarrel in any direction and provided the barrel-tube is made of sufficiently pliant material, precludes rotation beyond the bore deformation tolerance before discharge becomes impeded or is prevented. For use in an eccentric muzzle-head, the motor is restricted in rotary angle to allow the muzzle-ports to be directed to any angle.

Control of Muzzle-Head Turret-Motor Angle within Working Arc

Due to the small size of the distances to separate the implants, control over the positioning of the turret-motor to adjust the muzzle-head rotational angle and airgun linear stage mounting to adjust the transluminal displacement, cannot be left to direct manual control. Instead, a numerical positioning system intervenes between the operator and the movements of the turret-motor and airgun table mounting. Such control is indirect and semiautomatic, in that the controller sets knobs for the action to be accomplished, and the positioning drive electronics then execute the motion commanded. Closed-loop control of the subminiature dc through-bore torque muzzle-head turret-motor is conventional, differing from programmed numerical control only in real-time setting of the angle by the operator. The same joystick is rotated clockwise or counterclockwise to the angular displacement of the turret-motor sought.

When such point-to-point repositioning is to place the muzzle-head for successive discharges of the airgun, the duration at each intervening point need not be coordinated with miniball transit time, because the pause between the increments is prefixed to allow for the longest barrel length, typically 140 centimeters, this length being a critical factor in setting the exit velocity and provided with the apparatus. The output angle of the turret motor can be controlled with any digital motion controller-amplifier capable of driving a three phase brushless dc motor. The airgun closed-loop semiautomatic system is assembled from commercially available components, to include a motor controller, such as a Danaher Motion, Inc. S200 drive, and controlled from the control panel mounted to the airgun. To position the two diametrically directed electromagnets to face along any given diameter, rotatability of 90 degrees in either direction is required; however, to aim a monobarrel at any angle about the lumen circumference necessitates rotatability of 359 degrees. The material and thickness of the barrel-tube, and the curve it describes as it approaches the flush joint socket in the ejection-head must allow this degree of rotation without distortion to the bore as would retard or jam discharge.

The turret-motor control circuit includes a circuit-breaker to prevent overload or burn-out. Excessive resistance to rotation arising within the mechanism or in the relation of the mechanism to the lumen wall is thus truncated. For example, resistance to the action of side-sweepers when present would typically be presented by plaque that was calcified outside the area cleared by atherectomy. Exceeding the torque limit value set for any reason would shut down the turret-motor averting dissection. The distal end of the barrel-catheter is clamped within the collar at the proximal end of the through-bore turret-motor housing. In a radial discharge monobarrel, the barrel-catheter and singular barrel-tube are one and the same, and the distal curved segment of the barrel-tube is journaled within the rotor, which accordingly serves as a rotary joint. In a multiple barrel barrel-assembly, such axial rotation is not possible, so the neck of the spindle is journaled in the rotor.

A motorized muzzle-head eliminating the need for a tighter turning ratio or turning torque in the barrel-assembly, more pliant materials can be used for the barrel-catheter, enhancing steerability and eliminating the possible if infrequent need for the aid of a hand-held external electromagnet. The resistance to twisting more pliant barrel-tubes for the length of these in the splay chamber is less than with a stiffer tube material such as polytetrafluoroethylene. Dots of more brightly radiopaque contrast marker, such as Danfoss Tantalum Technologies Danfoss Coating® just beneath each muzzle-port, assists in positioning the muzzle-head. The greater pliancy of catheter tubing afforded by a motorized muzzle-head increases the potential for using conventional fixed shape guide catheters as barrel-catheters.

Vascular bends and angles of intersection or branching proximal to the lesion that are too acute to allow the smallest diameter barrel-assembly acceptable for stenting a given ductus to pass necessitate open exposure. When the barrel-assembly is connected to a modified off-the-shelf hand airgun, power is provided to the motor from a remote power supply with connector mounted beneath the pistol grip. The placement of the power supply is different in modified and dedicated airguns as will be described under the section on airguns. Two small single pole single throw push button type switches are mounted to the pistol grip just above and beneath the ball of the thumb, so that slightly raising the thumb and depressing the upper allows the muzzle-head to be gradually and controllably rotated up to the rotational displacement necessary clockwise and depressing the lower switch allows rotation counterclockwise without the need to reposition or look at the airgun.

### Trap and Extraction Recovery Tractive Electromagnets for the Recovery of Loose and the Extraction of Mispositioned Miniballs

A barrel-assembly of any kind must include means for both trapping any miniballs that are loose or that have been mispositioned upon implantation. To recover miniballs, the forward or distal end of a barrel-assembly, whether simple pipe or radial discharge, is equipped with an electromagnet assembly that consists of two direct-current tractive electromagnets as large in size as the dimensions of the muzzle-head will allow. The U- or generally horseshoe-configured core with elongated bridge is made of vanadium permador (vanadium permendur) or silicon iron steel, and the winding of braided alumina-silica or alumina-boria-silica fiber ceramic-insulated silver wire. For generating a field to trap loose miniballs, the electromagnets are controlled as a pair rather than independently. Reversing the polarity of either exerts little practical effect, a miniball never remaining positioned exactly at a point where the fields theoretically null or cancel; instead, one or the other field will always dominate at the points described by the miniball, which will always be seized by that electromagnet.

Continuously varying the amperage to the elecromagnets allows varying the magnetic field strength and magnetomotive force from zero to the maximum. An optional laser catheter incorporated into the barrel-assembly by positioning it along the longitudinal axis to end at the center of the nose is without ferromagnetic content and unaffected by the magnetic fields it traverses. Metal-capping the front ends (tips) of the optical fibers has been reported to yield better results (destruction or atheromatous lesions with least injury to the lumen wall) than bare-tipped fibers (Litvack, F., Grundfest, W. S., Papaioannou, T., Mohr, F. W., Jakubowski, A. T., and Forrester, J. S. 1988. “Role of Laser and Thermal Ablation Devices in the Treatment of Vascular Diseases,” American Journal of Cardiology 61(14):81G-86G; Yang X M, Manninen H, Soimakallio S. 1991. “Laser Ablation Ability of Different Fiber Tips on Human Arteries. The Role of Photothermal Effect,” Chinese Medical Journal (English Edition) 104(9):721-727). A metal tip or probe for the present purpose must be nonferrous.

A means for trapping any loose miniballs must balance the forward extendability toward a blind end or narrowing lumen diameter to which the muzzle-head can proceed with the need to retrieve any errant miniballs to this depth. The diameter of the muzzle-head varies according to the diameters of the lumens in which each is to be used. For use in the coronary arteries, these are on the order of 7-10 French. The tractive electromagnet in a simple pipe barrel-assembly, seen as 46 in FIG. 19 is enclosed within nonmagnetic housing 56 in FIGS. 17 and 18, is singular, whereas the tractive electromagnets in radial discharge barrel-assemblies (FIGS. 22, 23, and 24) consist of a pair in diameterical opposition. Because the simple pipe is for use in the airway, wherein the recovery of a radiopaque loose miniball does not pose the risk of such loss in the bloodstream, providing the electromagnet with a sping-loaded door and antemagnet chamber is not considered essential.

The same basic magnet structure is used to trap any loose or to extract any misplaced miniballs, whether the barrel-assembly is of the simple pipe type with one electromagnet or a radial discharge type with two. The dimensions and maximum tractive force of the electromagnets is proportional to the respective barrel-assembly. The use of electromagnets allows adjusting the field strength to a steady or resting level to recover loose miniballs, raise the current and thus the field strength to extract implanted miniballs, then lower the field strength to zero and so prevent the dislodging of well placed miniballs as the barrel-assembly moves past these upon withdrawal. In the airway, wherein the miniball implants will usually be somewhat larger in diameter than in the vascular system or in ducta, will adhere to the lumen wall rather than passing antegrade if loose, and are individually discharged as not to escape notice if failing to implant or implant as misplaced, the electromagnets can be left off or set to a steady protective trap or subextraction field strength, then raised to extraction field strength if needed.

Discharge into the vascular system, however, is always performed with the tractive electromagnets set to a protective trap field strength to prevent a loose miniball from passing downstream. The resting field strength of the tractive electromagnets normally sweeps up any loose or lost miniball. However, where such an exigency would pose inordinate risk, the ability to locate a loose or lost miniball is increased by using miniballs coated with tantalum for increased radiopacity. In radial discharge barrel-assemblies, the electromagnets 64 in FIGS. 22 and 80 in FIG. 24, are mounted within chambers in a housing at the distal or forward end of the muzzle-head. To allow tantalum coated miniballs trapped in the antemagnet chambers to be observed flouoroscopically, magnet housing is preferably made of a transparent material, such as polycarbonate. Seen head-on as in FIG. 23, the magnet assembly, shown in FIG. 22 as 64 and in FIG. 24 as 80, constitutes the anterior, i.e., distal or front portion of the muzzle-head, and is divided two compartments, seen as upper and lower chambers as positioned in FIG. 23.

The horseshoe-configured single working face tractive electromagnets are contralaterally offset to allow a space in front of each which is enclosed behind corner plastic-hinged center-opening double doors that are recessed from the lumen wall, are stopped by opening further by contact with the magnets to the sides of the poles, and urged into closed position by means of plastic torsion springs at the hinges. Small tabs prevent the torsion springs from opening the doors outwards or away from the magnet past the position to close the antemagnet chambers. The force with which a loose or mispositioned miniball is drawn toward the magnet exceeds the restorative force of the torsion springs that otherwise urge the double doors into a closed position against the stop tabs. The strength of the magnetic field and restorative force of the torsion spring are sufficient to pull the miniball through and close the doors regardless of the entry into the antemagnet chamber of mucus, saliva, or blood.

Recessing the double doors reduces the chance that these will be opened by brushing up against or scraping the lumen wall. Since the attraction of a miniball already under recovery forces the double doors open, to provide a sensor to sound an alarm at the door hinge or spring is considered moot. As the front portion of the muzzle-head and barrel-assembly, all external angles of the magnet assembly are ground and polished smooth so that the front end, i.e., the distal nose or face, is completely convex or rounded, smooth, and continuous. Two of the blood grooves that run longitudinally midway between the muzzle-ports are aligned to the spaces in front of each double door, and ground and polished so that the groove continues into the space smoothly. The other two blood grooves are continued over the outside of the magnet assembly. The muzzle-head, to include the motorized turret collar, port portion, and magnet assembly, is preferably encapsulated within a lubricious coating, such as ACS Microslide®, Medtronic Enhance®, Bard Pro/Pel® or Hydro/Pel®, or Cordis SLX®.

The pistol grip or dedicated airgun controller thus has knobs to separately adjust the potentiometers that control the exit velocity and two others to adjust the field strength of the recovery electromagnets. Each magnet in the pair or magnet set is separately controllable over their range of magnetic field strength. For this reason, the same electromagnet set can be used with a low or resting field strength to catch any loose miniballs or to extract miniballs that have already been implanted but misplaced. To extract a miniball that has already been implanted, one of the electromagnets is aligned alongside the misplaced miniball and the amperage raised until the miniball is pulled to the magnet. Increasing the amperage gradually to only the electromagnet positioned and directed toward the specific miniball to be extracted, the effect on other miniballs is minimized.

The range of propulsive force available with any airgun and the immediate interchageability of different kinds of rotary magazine clips makes it possible to place a different number of barrel-tubes, each of variable diameter, within a barrel-catheter of given diameter for use in the same airgun. Up to a certain limit in the diameters of the miniballs required, the barrel-catheter may be of a given size, which if exceeded, will require the use of an airgun of larger bore or the removal of a smaller and replacement with a larger diameter airgun barrel liner. With an airgun of maximum bore, airgun barrel liner adaptors and the ability to change the kind of rotary magazine clip in a moment allow connection to any barrel-assembly. In such a universal airgun, because the diameter of the airgun muzzle does not change but only different bore-reducing or increasing liners are inserted, the flange connector is of standard size such that any barrel-assembly can be connected to the same airgun. Accordingly, mechanical connection of the simple pipe barrel-assembly to the airgun is preferably the same as that to be described for single and multiple barrel radial discharge barrel-assemblies.

By removing one kind of rotary magazine clip and inserting another, the airgun can be quickly converted for use with barrel-assemblies having from one to four or more barrel-tubes that are alike. Different bores require changing the airgun barrel liner. Regardless of the number or diameter of the barrel-tubes, the barrel-assembly must always be precisely aligned so that each barrel-tube is positioned before its respective hole in the rotary magazine clip. The need to replace an airgun due to a malfunction should not necessitate the withdrawal of a barrel-assembly already placed within the lumen.

To this end, interchangeability of a given barrel-assembly into different airguns is of distinct benefit. Reciprocally, when the diameter of the airway becomes too small for a simple pipe, to switch to a single barrel radial discharge barrel-assembly should not necessitate changing the airgun that is already adjusted to the proper setting. Apart from these advantages in uniformity of connection, the expense to the practitioner is reduced. Barrel-assemblies should be interchageably connectible to an airgun regardless of whether the airgun has been modified from one sold on the market or was originally made for interventional use.

The interchangeability of airguns and barrel-assemblies allows one and the same airgun to support any number of different barrel-assemblies, which is advantageous whether only one kind and size of barrel-assembly is used, other airguns being usable in the event of a malfunction, or barrel-assemblies of several different kinds and sizes are used, where each airgun can be equipped with a different bore reducing barrel liner. Accordingly, a single flange-connector size according to the largest bore is preferred, a second airgun of still larger bore reserved for large zoo mammal veterinary use. The rotary flange or twist-to-lock connector limits the distance to which the barrel-assembly can be inserted into the barrel of the airgun. This places the end-plate before the rotary magazine clip with the least interval separating the proximal ends of the barrel-tubes and the hole in the rotary magazine clip respective of each barrel-tube.

### Engagement of the Barrel-Assembly in the Airgun

The barrel-assembly, such as the two-way barrel-assembly depicted in FIG. 27, is capped off at its proximal end with an end-plate 99 that receives and holds the proximal ends of the barrel-tubes, which open through the end-plate, and must be positioned in precise alignment with their respective holes in the rotary magazine clip or with a monobarrel, before the miniball to be discharged. In order to maintain this precise alignment, the length of the barrel-catheter that is introduced into the airgun barrel must fit flush to the airgun bore. In a noncombination-form barrel-assembly with center discharge muzzle-head, the slit valve in end-plate 99 is used for insertion of a cooling catheter as described above. The barrel-catheter is inserted into the airgun barrel to a distance just short of, as not to come into contact with, the rotary magazine clip as it advances by indexed rotation from one discharge load to the next.

Referring now to FIG. 39, barrel-catheter 72 is inserted into the barrel of the airgun 107 up to the limit allowed by stop-and-lock ring 75, which engaged within the female component of the twist-and-lock connector that is mounted to the muzzle of the airgun places the end-plate just short of contact with the rotary magazine clip 15 with barrel-tubes 74 perfectly aligned to the hole in the rotary magazine clip respective of each barrel-tube. Increased interchangeability of barrel-assemblies and airguns raises the risk for a barrel-assembly not designed for use with a given airgun or an airgun that has been configured for different operation, such as the use of a different rotary magazine clip or different bore insert. Barrel-assemblies must therefore bear clear compatibility indicia. Since a tag will be removed, this is best accomplished by molding specification on every barrel-assembly and color coding adaptors for insertion in the airgun. Making the chamber of transparent plastic allows the color of the rotary magazine clip to be seen from the outside.

### Electrical Connection of the Barrel-Assembly to the Airgun

Generally, only the barrel-tubes are conveyed entirely through the barrel-catheter to the end-plate, electrical connection of the receiver to the barrel-assembly most often circumventing the rotary magazine clip by placement on the outside of the barrel-assembly distal to or in front of its junction with the barrel of the airgun as shown in FIG. 40. This allows the barrel-assembly to be removed and reinserted for manual use without a loss of power. Mounting a rechargeable battery pack local to the electrical terminals at the outside of the barrel-assembly allows both the removal and reinsertion of the barrel-assembly as needed without the need for a cable dangling from side.

a. Barrel-assembly with electrical terminals on the outside of the barrel-assembly in front of the airgun muzzle Provided the conductors are adequate in length, when the electrical connectors are outside the airgun barrel as shown in FIG. 37, the operator is free to remove and reinsert the barrel-assembly to manipulate it manually without electrically disconnecting the turret-motor, side-sweepers, or electromagnets from the power supply within the airgun cabinet.
b. Barrel-assembly with electrical terminals inside the airgun chamber When electrical connection is inside the airgun chamber, to use the barrel-assembly independently of the airgun requires an alternative source of power. End-plate connection as shown in FIG. 40 requires an electrical connection separate from the mechanical connection and clipping the conductor to the airgun, which would otherwise hang from the side of the barrel-catheter, in some embodiments, for greater ease and speed of use, it is preferable to have the conductors continue to the end-plate 99 in FIG. 37 and terminate on the outside of the barrel-catheter.

While FIG. 38 shows terminal 101 as having some thickness for clarity of illustration, in order not to produce eccentricity, the terminal must be countersunk into the outside polymer wall of the barrel-catheter 72 to present a smooth surface. With such an electrical connection, the twist-and-lock connector consisting of stop-and-lock ring 75 and muzzle fitting 103 establishes the correct rotational angle not only for aligning the barrels but to bring the electrical connectors into sliding contact. The simple pipe barrel-catheter (FIGS. 17 and 18) requires no turret-motor, but is equipped with a trap-extraction electromagnet assembly that is connected to the airgun by means of a two-wire or double conductor cable. Any of the exemplary connector types specified below for six conductor connectors, to include former military specification C-5015 subminiature D type connectors containing two conductors for each trap-extraction electromagnet, can be used. Power is provided by a remote power supply, which in a modified marketed airgun is a separate component but integrated into a special-purpose interventional airgun.

The two wire conductor plugs into a socket toward the proximal end of the barrel-assembly where the latter exits past the outside of the airgun barrel. The wire is fixed to the underside of the barrel-catheter with cyanoacrylate cement. As with the electrical connection to a radial discharge or multiple barrel barrel-assembly, rather than allowed to drop from the side of the barrel-catheter, the wire or cable is held by clamps to the actuation handpiece or pistol grip. The multiple barrel barrel-assembly has a turret-motor and trap-extraction electromagnet assembly, each of which must be independently controllable. The conventional six-conductor with circular connectors or discrete wire ribbon cable connected to the remote power supply contains two wires each for the turret-motor and each of the tractive electromagnets and terminates in a plug, which can be of the strip header or long latch and eject header kind.

In the four-way radial discharge barrel-assembly shown in FIGS. 24, 28 and 33, an eight-conductor cable 97, of which two wires are for the turret-motor, two wires each for either separately adjustable electromagnet, and two wires are to actuate the side-sweeper, passes down the central canal of the barrel-assembly. Distad, the wires for the electromagnets continue past the port portion of the muzzle-head through the nose or anterport projection. These barrel and electrical connections end on the proximal outer surface of the barrel-assembly with a six-contact terminal which receives these lines from the remote power supply. Connection of eight-conductor cable 97 which approaches from the distal electrified components within the barrel-assembly through the central canal shown in FIG. 37 and represented in FIG. 26 is to an eight-post terminal 106 mounted to the outside of the barrel-assembly at a small distance from the airgun muzzle. Once fully inserted, the dangling cable 109 is fixed to the side of the airgun barrel by means of clips permanently affixed to the underside of the airgun barrel with screws 110 or by rubber bands.

In a modified hand airgun, the electrical cable is continued down the front of the pistol grip to the separate power supply. In an interventional airgun, the power supply is contained within the same cabinet as the other components, insulation, heat sinking barriers, and distance used to separate sources that generate conflicting temperatures. To allow a barrel-assembly having contacts at the end-plate as shown in FIG. 70, which would otherwise lose electrical connection when disengaged from the airgun barrel, to be freely removed for manual use with free movement, the far or opposite face of connector 106 provides a round socket configured to connect to the barrel-assembly whether connection is at the end-plate or at the side. The cable plugs into a socket toward the proximal end of the barrel-assembly where the latter exits past the outside of the airgun barrel.

While the trap-extraction electromagnets use ceramic woven insulated silver wire to generate magnetic field intensities sufficient to extract mispositioned miniballs despite their small size, the conductors connected to these are of greater gauge and not susceptible to melting by heat conduction. The 6 conductor-3 pair plug and socket may be any of many kinds, to include those stated above for ribbon cable; minirectangular; modular; or registered RJ12 or RJ25. These electrical lines connect on the outside of the receiver-barrel-assembly junction by means of connectors as shown in FIG. XX. The power supply of a modified airgun is remote, whereas that in a dedicated airgun is an integral component. Inside the barrel-assembly, the cable is not ribbon but round and courses distad through the central canal defined by the barrel-tubes, the four conductors for the trap-extraction electromagnets continuing through the center of the muzzle-head splay chamber.

### Rechargeable Battery Pack Local to the Electrical Terminals

An ablation and angioplasty-capable barrel-assembly must be usable independently of the power supply in the airgun. To provide the longest life with the least weight, the battery pack onboard the barrel-assembly is of the rechargeable silver-zinc or lithium-polymer type.

### Ablation and Angioplasty-Capable Barrel-Assemblies

As introduced in the section entitled The Radial Discharge Barrel-assembly as a Separate and Independent Angioplasty Device above, an angioplasty barrel-assembly can be used to perform an angioplasty regardless of whether this is followed by insertion into an airgun for stenting implant discharge. Because this necessitates the incorporation of several additional components, the dimensional constraints become severe. When angioplasty in a coronary artery is to be followed by stenting discharge to place the implants, the barrel-assembly is not withdrawn following angioplasty but inserted into the airgun to initiate stenting. Then, even though the apparatus has been devised to minimize procedural time and to the extent possible allow blood to flow past, the minimum diameter attainable and indwelling time are such that if circulation is unduly obstructed, the inducement of a myocardial infarction is virtually certain.

The muzzle-head body itself can exert balloon-like compression on protrusive plaque, but as is true with a balloon, this can dislodge vulnerable plaque, and the deployment of a trap-filter ahead of the muzzle-head has itself been implicated in dislodging plaque. Angioplasty-capable barrel-assemblies may be used independently of an airgun to significantly reduce if not eliminate plaque prior to the insertion of a conventional or endoluminal stent, or following use for angioplasty independently of an airgun, can be inserted into an airgun to initiate stenting without withdrawal from the ductus. For both angioplasty and stenting functions, extreme limitation in diameter and a severe requirement for steerability as a functional combination of flexibility and stiffness represent the primary constraints imposed upon such components as may be devised.

Enhanced versatility and freedom of movement of the angioplasty-capable barrel-assembly as independent of the airgun imposes greater expense. That is, an angioplasty-capable barrel-assembly when optimized in free-standing ability requires insertion into the airgun only for discharge. During an angioplasty as a distinct procedure that may or may not be followed by stenting, the need to insert the barrel-assembly into the airgun to draw power from the power supply rather than an inmate battery or to connect the turret-motor to a drive-controller within the cabinet of the airgun rather than to an onboard polyphase current-generating microcircuit, involves connection that reduces independence and freedom of movement. Except where connection must be continuous to draw power, such connection is temporary but still comes as an interruption.

Whether occlusion is associated with atherosclerosis, fibromuscular dysplasia, stenosis attributable to other vasculopathy, or a combination of causes, the susceptibility of a muscular artery to obstruction varies inversely as the cross-sectional area of the lumen. The same may be said of many instances of stenosis in other type ducti, some treatable with the same apparatus, which is rarely if ever without concurrent medical (drug) treatment. That the principal factor predisposing to occlusion is smallness in lumen diameter makes severe limitation in diameter the chief design constraint upon any catheter-based device. Any effective mechanical device for intervening in this occlusive process must be able to reach such sites.

Because plaque tends to accumulate at points in the vasculature that are intrinsically subject to turbulent flow—at angular turns at the entries or ostia of branches, bifurcations, at convolutions and over tortuous stretches, in the extremities where increased distance from the heart results in the reduction of propulsive force and an increase in the effects of gravity—the protrusion of plaque into the lumen only makes flow past such points even more turbulent, hence, thrombogenic. The propensity to favor twists and turns adds steerability to restriction in diameter as a basic requirement for barrel-assemblies; essentially, the more difficult it is to reach a certain point, the more probable is it that that will be a point that has to be reached.

For post-acute event patients resistant to medical treatment, thrombectomy will usually be essential, and for any patient predisposed to an acute event by advanced occlusive disease, an atherectomy may be recommended. However, severe limitation in diameter precludes incorporating two different rotating tools, one for thrombectomy and the other for atherectomy, in the same barrel-assembly. Unlike power burrs, which are suited to removing hard plaque but not soft material, a laser can remove both thrombi and all but exceptionally calcified plaque. This leaves only the occasional need to cut through very hard plaque as necessitating the antecedent use of a separate conventional rotational atherectomy or other mechanical rotational ablation, or rotablation, device. Simple balloons can compress softer plaque and place an expandable stent, but not in a single operation as would allow entry and withdrawal only once.

Furthermore, the need for more than one stent is common, and to place these with a balloon necessitates reentry to place each stent. Using existing means for clearing the lumen and stenting, entry and withdrawal is required at least twice, because the lumen must be cleared of plaque before stenting can commence. A device that having been introduced transluminally but a single time can remove and not just crush plaque up against the lumen wall and can proceed to effect stenting at multiple locations along the same vessel clearly reduces operating time and the risk of complications. Whereas balloon angioplasty is often performed to expand the vessel following thrombectomy, here the muzzle-head, while not forcibly inflated to risk dissections, still imparts some straightening and expansion likely to suffice in less refractory cases.

When the balloon cannot pass the lesion without the need for rotablation, the residual plaque may still be too hard to safely mash against the lumen wall. The balloon may necessitate the preliminary opening of a channel large enough for the balloon to be entered, but then does not provide means for the intermediate removal of hard plaque following rotablation and preceding angioplasty. Here, in addition to initiating stenting, the barrel-assembly can provide ancillary means for expanding a post-rotablated lumen with less risk of producing dissections in the form of side-sweepers and a laser, and here too, the muzzle-head, while not inflated, in and of itself still imparts some straightening and expansion.

Existing combined function devices, such as those used in directional atherectomy, which include a balloon, can remove and compress plaque and can also expand the lumen, but cannot additionally stent in a single operation. A stated object here is to minimize operating time and therewith the time of interrupted oxygenation. While extraluminal stenting necessitates separate access through an incision to place the stent-jacket, this is usually possible under a regional if not local anesthetic at a later date. Over and above the desirability of reducing entries and withdrawals, when anesthesia is general, reducing procedure time is conducive to a more favorable outcome. Thus, currently no device combines an atherectomy or atherotomy and an angioplasty capability with the ability to initiate stenting in a single device as does the barrel-assembly with side-sweepers described below.

Slow-acting and not to be used when the barrel-assembly is moved, side-sweepers are only suited to assist a primary mechanism, whether a burr or laser, for the removal of occlusive matter. Instances inevitably arising whereby to scrape all the peripheral plaque as may remain would simply take too much time, such use is always discretionary on the part of the operator. In integrating a means for the ablation of lesions atheromatous or otherwise into the muzzle-head, the cardinal desiderata remain safe trackability and minimized operative duration. While combining atherectomizing and the intravascular component of extraluminal stenting means in the same transluminal device can significantly reduce if not eliminate the need for withdrawal and reentry and thus reduce the duration of the procedure and the risk of entry wound complications, to accomplish this at the expense of increased risk of ischemia because the barrel-assembly has been increased in diameter is counterproductive.

The most widely accepted means for opening occluded vessels are the rotational atherectomy burr, such as made by Boston Scientific and the laser catheter, such as made by Spectranetics. Neither device can eliminate plaque up to the lumen wall, because to do so risks injury that can result in abrupt closure or perforation; the prior art makes it clear that the incorporation into the barrel-assembly of a laser is limited for practical reasons to the longitudinal or central axis of the barrel-assembly. This is no less true when either of these devices are incorporated into the barrel-assembly. However, the incorporation of side-sweeping brushes into the muzzle-head provides a followup mechanism for removing residual plaque once the burr or laser has passed. Broadly, to afford clearance for the passage of the blood demands minimizing the diameter of the barrel-assembly, which in turn demands reduction in the number and/or diameter of barrels, hence, of the diameter of the miniballs that may be used.

The need to use miniballs of smaller diameter in the wall of a vessel must always be compensated for with a higher density distribution in order to more uniformly distribute the magnetic traction and thus reduce the risk of an eventual vessel wall perforation by an isolated miniball of a tissue insinuative diameter under excessive magnetic traction. Essentially adapted from the high-speed drills used by dentists, rotational and directional atherectomy devices use a cutter rotated by an air turbine. Whereas a laser or the rotational ablation (‘rotablation’) type can only cut directly ahead, directional atherectomy uses a contralateral balloon to press a side-cutter into cutting abutment against the lumen wall to the side opposite the balloon, and so can accomplish clearing both by plaque removal and the application to the lumen wall of outward radial force controlled by changing the pressure of balloon inflation. Due to the difficulty in detecting the depth of side cutting, directional atherectomy has lost favor.

Other examples of combined function devices are powered cutting balloons that atherectomize and passive rotational cutting balloons that atherotomize. Powered rotational burrs, passive cryogenic, thermal, ultrasonic, and laser devices are single-function atherectomy devices that remove hard prominences, reducing the risk of dissections and restenosis as would otherwise be more likely to result from the angioplasty performed next (see Safian, R. D., Freed, M., Reddy, V., Kuntz, R. E., Bairn, D. S., Grines, C. L., and O'Neill, W. W. 1996. “Do Excimer Laser Angioplasty and Rotational Atherectomy Facilitate Balloon Angioplasty? Implications for Lesion-specific Coronary Intervention,” Journal of the American College of Cardiology 27(3):552-559). When the muzzle-head is of the type having the gas-return path rather than a laser catheter at the center, one to four separate laser catheters depending upon the unbranched diameter at the distal working tip pass through the barrel-catheter and midway between the barrel-channels to the nose. Short of terminating at the working end, the fibers of the separate cables can merge to form a unitary tip or divide and merge to form a unitary tip of larger diameter.

### Barrel-Assembly Hand Grip

The barrel-assembly is equipped with a hand-grip to serve as a torquing device or torquer. The hand-grip is coated for traction as with vinyl or a nonallergenic synthetic rubber. In an angioplasty barrel-assembly, the hand-grip mounts an independent angioplasty control panel, and in untethered embodiments, the hand-grip contains a cylindrically configured lithium polymer battery pack.

### Barrel-Assembly Onboard Ablation and Angioplasty Control Panel

For unrestricted movement, an ablation and angioplasty-capable barrel-assembly is devised for use independently of an airgun. While not preferred, it could be used thus and then set aside for conventional (endoluminal) stenting. Used as intended, once the ablation or angioplasty has been completed, the barrel-assembly is inserted into the airgun to commence implantation discharge. Accordingly, the control panel on an ablation and angioplasty-capable barrel-assembly is onboard the barrel-assembly and includes the controls needed for an ablation or angioplasty. In addition to on-off and action abend (stop) switches, these include controls for 1. Turret-motor temperature (current); 2. Electromagnet winding 1 temperature (current); 3. Electromagnet winding 2 temperature (current); 4. Turret-motor rotation (typically by means of a digital encoder manually rotated with a knob (not a joystick as on the airgun control panel) having a pointer that moves over an upper semicircular calibration with apical or centered O-point and marked off in 5 degree increments to either side); 5. Side-sweeper 1 deployment (release, extension, unstow)-retraction (recovery, stow); 6. Side-sweeper 2 (or if more than 2, then the appliable number) deployment (release, extension, unstow)-retraction (recovery, stow); and trap-filter (release, extension, unstow)-retraction (recovery, stow). The need for rotation of the muzzle-head arises because the turret-motor heat-window may be in the form of a slot or slit, hence directional, and because the side-sweepers may be different and the lesion eccentric.

Since more angioplasty-capable barrel-assemblies are used manually before insertion into and while separate from the airgun, the on-board control panel does not have a control for the linear positioning table, which is used for the precise intraluminal positioning required for higher density implantation. A vortex tube cold or hot air gun or cryogenic gas (CO₂ or NO₂) cartridge connected to the back end of the barrel-assembly will usually have controls for these mounted on those devices, as will the laser, atherectomy, or thrombectormy devices incorporated into combination-forms as described above under the section entitled Combination-forms: barrel-assemblies that incorporate means for thrombectomy, atherectomy, or atherotomy.

A minimally ablation and angioplasty-capable barrel-assembly requires only controls for the nose-cap heat-window, which is to say, one control to adjust the temperature of both recovery electromagnet windings as one. When not excessive in extent, intraluminal movement is accomplished with the minimal capability barrel-assembly in the airgun using the linear table, which is electrically connected as shown in FIG. 37, or alternatively FIG. 40, in which case the conductors must present sufficient slack to allow the movement required. An intermediate capability barrel-assembly is generally used for ablation or angioplasty prior to insertion into the airgun and therefore has an on-board control panel and power source (battery pack) but fewer controls than a fully capable barrel-assembly as defined above.

The onboard ablation control panel is mounted on the hand grip just in front of stop and lock ring (75 in FIGS. 27, 39, and 40). The muzzle-head positioning and airgun discharge control panel is separately mounted in the top either of the airgun cabinet or of the stanchion with weighted base (see following) so as to present the joystick and control knobs at a height adjustable for the individual operator. As labeled in FIG. XX, these control the delivery of current to the turret-motor stator as a thermal angioplasty heating element, and the delivery of current to the thermal expansion wire used to deploy each of the side sweeper-scrapers. The run-ahead or downstrream trap-filter is simultaneously deployed with any side sweeper-scraper.

Muzzle-Heads with Side-Sweeping Brushes for Removing Soft (Noncalcified) Plaque

Incorporation of a side-sweeping capability is intended to assist in the removal of softer peripheral atheromatous tissue after removal of prominences by a primary atherectomy device, whether inmate laser or separate rotating burr used before the barrel-assembly (see Safian, R. D., Freed, M., Lichtenberg, A., May, M. A., Juran, N., Grines, C. L., and O'Neill, W. W. 1993. “Are Residual Stenoses after Excimer Laser Angioplasty and Coronary Atherectomy Due to Inefficient or Small Devices? Comparison with Balloon Angioplasty,” Journal of the American College of Cardiology 22(6):1628-1634). Since atheromatous plaque accumulates beneath the endothelium, to access and eliminate the plaque involves injury to the endothelium; however, such injury in the elimination of plaque gains more than injury by a balloon that only forces the plaque into the media. Side-sweeping brushes do not appropriate sufficient circumferential coverage to completely check the flow of blood. The burr is superior for the removal of hardened plaque but can furrow or even perforate if it comes into contact with the lumen wall. A laser can as well but is effective with both soft and relatively hard but not very hard plaque.

While alternative to balloon angioplasty have been cited as “exhibiting a “lack of compelling trial data suggesting that the atherectomy devices offer better outcomes in a stand alone or even an adjunctive role,” (Carrozza, J. P. 2006. “Coronary Complications of Coronary Atherectomy and Excimer Laser Angioplasty,” UpToDate http://patients.uptodate.com/topic.asp?file=chd/17957, others dispute this position, and the barrel-assembly does not lend itself to incorporation of a balloon in order to effect plaque removal. Hard plaque can be impossible to pass with a balloon, and even when possible, compressing hard calcified plaque against the lumen wall is likely to produce dissections. Antecedent atherectomy is thus either necessary or advisable. Whereas atherectomy would ordinarily be followed by a balloon angioplasty, using the apparatus described herein, when the lumen diameter is less than that of the muzzle-head, the muzzle-head itself compresses plaque against the lumen wall, albeit not with the radial force of a balloon.

However, with the aid of the side-sweepers to be described, the need for a balloon angioplasty in preparation for stenting can be avoided. Along with reducing operating time, this factor makes incorporating a means preceding the muzzle-ports for removing plaque desirable. Using inmate side-sweepers, the hardness of the material that may be scraped away depends upon the output torque of the turret-motor. While primarily angioplastic, side-sweepers and heat-windows can be used to remove other obstructive tissue or matter in blood vessels or ducti of other types, such as XXXX. When, as in the intrapulmonary bronchi, intraluminal matter such as fibrotic obstructs the lumen, the ablative function of thermal angioplasty-capable barrel-assemblies can be applied in lieu of alternative intraluminal ablative apparatus, such as lasers or burrs, to the reduction or removal of the obstruction prior to intraluminal stenting.

While combination-form angioplasty barrel-assemblies incorporate a laser or burr, conventional devices such as a laser can, however, access the distal portions of a lumen where the diameter has become too small for a barrel-assembly. Low-level radio-frequency and microwave energy devices already available for the purpose, a purely thermal catheter head in the absence of any discharge capability is not contemplated. When the character of the occlusive tissue as indicated by intravascular ultrasonography if necessary, is unlikely to result in a release of occlusive debris, a supplementary ability to scrape the lumen wall can make it possible to effect clearing without the need for an antecedent angioplasty.

### Side-Sweeping Brushes, Bristle and Shavers Types

Here the term ‘brush’ is used to denote a collection of projections mounted in close proximity to the same block or backing where the configurations of the ends or tips of the individual elements or filaments can be other than bristle-like. These brushes, with various tip configurations over a wide range of stiffnesses, longitudinal and arcuate lengths, and projection extensions are swept along the lumen wall to remove diseased tissue or debris. Portions of the recess not stowing a brush are roofed or blanked over. The side-sweeping brushes are housed within recesses in a special module on the muzzle-head for joint or separate projection and retraction as needed. Incorporating brushes of different stiffnesses and tip configurations over different arcs about the barrel-assembly allows the discretionary differential treatment of eccentric lesions. Individual brushes can be flat or wallpaper-brush configured or square and the filaments of the bristle or metal or plastic shaving tool type, which can have inclined tips. Side-sweeper modules can be configured to accommodate brushes of different shape and/or type, and to accommodate different shapes and types about the circumference, generally two.

Independently elevating one of two brushes, for example, allows use of the turret-motor to direct the preferred brush toward the eccentric pathology, the heat-windows being independently heatable to assist in viewing the rotational angle if necessary. Regardless whether control of the turret-motor is by a drive servocontroller microcircuit inmate in the hand-grip or by electrical connection requiring insertion of the free end of the barrel-assembly into the airgun, either a single control on an angioplasty barrel-assembly allows causing the motor to oscillate as unstable by detuning the velocity loop, or, in a more advanced independent angioplasty barrel-assembly, a choice of programmed oscillatory movements can be selected. In either case, the action can be used with side-sweeping brushes. While in an angioplasty-capable barrel-assembly in use for an angioplasty, the turret-motor is usually limited to use as a heating element, when the side-sweeping brush module is divided between different brushes for use in sequence in the treatment of eccentric lesions and the path to the treatment site precludes rotation of the barrel-assembly, the turret-motor must be used to rotate and thus select one brush for use at a time.

In a battery-powered angioplasty-capable barrel-assembly intended for use independently of an airgun to perform an angioplasty, this requires incorporating the positional control into the onboard control panel mounted to the hand grip-shaped battery pack at the proximal end of the barrel-assembly. Manual or remotely switched motor-driven longitudinal or rotatory sweeping of the lumen wall is provided. Gross transluminal or rotatory movements are normally manual, finer longitudinal movements accomplished by means of the linear positioning table stepper motor, and rotational movements by means of the turret-motor. Unlike a solid trowel-like blade which inflexible sees the maximum resistance along its contact edge, a brush sweeps away plaque that is soft and sweeps over plaque that is hard compared to bristle stiffness. The turret-motor circuit breaker prevents any extra-bristle source of resistance from producing tears of the endothelium and intima.

Calcification resistive to side-sweeping, computed tomography allows plaque calcification and other kinds of adhesions or protrusions into the lumen not only to be qualitatively confirmed but characterized as to shoulder to base distribution and percent content of calcium and hydroxyapatite (see, for example, Rumberger, J. A., Sheedy, P. F. 2nd, Breen, J. F., Fitzpatrick, L. A., and Schwartz, R. S. 1996. “Electron Beam Computed Tomography and Coronary Artery Disease: Scanning for Coronary Artery Calcification,” Mayo Clinic Proceedings 71(4):369-377; Thompson, B. H. and Stanford, W. 2004. “Imaging of Coronary Calcification by Computed Tomography,” Journal of Magnetic Resonance Imaging 19(6):720-733; Raggi, P. and Berman, D. S. 2005. “Computed Tomography Coronary Calcium Screening and Myocardial Perfusion Imaging,” Journal of Nuclear Cardiology 12(1):96-103; Huang, P. H., Chen, L. C., Leu, H. B., Ding, P. Y., Chen, J. W., Wu, T. C., and Lin, S. J. 2005. “Enhanced Coronary Calcification Determined by Electron Beam CT is Strongly Related to Endothelial Dysfunction in Patients with Suspected Coronary Artery Disease,” Chest 128(2):810-815) or multi-detector CT-angiography (Miralles, M., Merino, J., Busto, M., Perich, X., Barranco, C., and Vidal-Barraquer, F. 2006) “Quantification and Characterization of Carotid Calcium with Multi-detector CT-angiography,” European Journal of Vascular and Endovascular Surgery 32(5):561-567.

Along with the compressive effect of the spindle body itself, side-sweeping with a debris filter trap to prevent downstream embolism to be described can be used as a primary means of angioplasty or as an adjunct to a primary means of angioplasty or atherectomy. Angioplasty, stenting, and using a combination-form that includes a laser catheter to be described, even atherectomy and stenting can be accomplished with a single entry. Brush-reaming is useful for localized lesions susceptible of removal by bristle-scraping, which excludes hardened plaque. Dependent upon its prominence and extent, stoney plaque will necessitate a bypass graft or rotary ablation. The force with which the brushes are projected and held beyond the surrounding outer surface of the muzzle-head is limited by the force exerted and pliancy of the thermal expansion wire or bimetallic tang used to raise or project the brushes. By varying the materials and dimensions of these elements, the resistance of the brushes to being depressed with their recesses is made kept small enough to prevent gouging injury.

### Types and Number of Side-Sweeping Brushes

Turning now to FIGS. XX and XX, side-sweeping brushes XX are housed within a special module XX incorporated into the muzzle-head. The module is concentric to and distally coterminal with the barrel-catheter at the level where the barrel-catheter is joined to convoluted segment XX. Side-sweeping brushes cover a wide range of bristle tip face areas in length and width, making the number of brushes in a given angioplasty-capable barrel-assembly variable such that those shown are intended to be taken as exemplary. The side-sweeping brush module can contain one or more brushes of like or different kinds. The interchangeable brush inserts can be variously configured as subminiature curved or semicircularly tipped wallpaper smoothing (smoothers) or square-head type brushes. Using any of a number of different materials in different thicknesses and lengths, the rigidity of the bristles is widely variable. Brushes for treating plaque of a given distribution and hardness of calcification as predetermined by computed tomography (preceding section) are provided with interchangeable lift-gate handles to snap into the brush recesses. Polyamide (nylon) being but one of many suitable polymers, the material of the bristles must not be friable or susceptible to fatigue fracture.

Bristles of coextruded tubing expand the mechanical performance obtainable. The detailed view of one bristle of such a brush shows that to prevent stabbing vectors when reversing direction causes the bristles to stand erect, the end is blunted. The sharpness around the periphery of each bristle may be varied to affect its action as a drag-scraper. When the bristle tips are round and sharp but the lesion is soft so that dragging such a tip along the surface of the lesion only undercuts the surface to either side without actually removing the plaque, the faces of the bristle tip normal to the long axis of the lumen can be formed into a sharp-edge cup the cutting edge as shown in FIG. XX. If tomography reveals that the plaque or other obstructive matter is hard, then such bristles, which would then grab hold of and pull at the material, is not used. Diametrically opposed brushes can differ in bristle materials, conformation to include thickness, and stiffness. As shown in FIG. XX, the lift-gate includes a check or stop to limit the distance that the brush can extend beyond the outer surface of the muzzle-head. To allow blood to pass when deployed, the bristles of the side-sweeper brushes should be grouped into separately ferruled bundles that flare laterally from the point of insertion within the lift-gate as brush-handle (rib, backing) to provide gaps.

### Control of Side-Sweeping Brushes

Whether deployed during transluminal or rotatory movement, control of the side-sweeping brushes is manual. With a lumen wall that is malacic and brushes that are harder, the side-sweeper bristles could incise the lumen wall, the operator must specifically override the default retracted condition of the side-sweepers. Thus, another limitation is that the radial blades should not be deployed while the barrel-assembly is advanced or withdawn. Described below is a switching arrangement that instantly cuts off current to the scraper blades causing these to recede should the barrel-assembly be urged forward or backward as well as a circuit-breaker to stop current to the turret-motor when the set threshold value for maximum torque is exceeded. To allow somewhat greater depth of access into the vascular tree, when the muzzle-head reaches a segment wherein the lumen is the same in diameter, deployment of the side-sweeper blades allows blood to pass. To orient the blades face-rather than edge-on to the bloodstream would further obstruct the flow of blood past the muzzle-head.

Plaque removal capability is added to the barrel-assembly by purchasing burr and laser devices as finished products and incorporating these into the barrel-assembly along the longitudinal axis to end at the center of the nose. To provide side-sweepers in barrel-assemblies not for thermal angioplasty, miniature lift-gates of the dam or inverted porticullis kind die-cut from thin nonferrous sheet metal, such as copper or aluminum, are positioned in the lateral or lumen-facing and rear walls of each electromagnet chamber. In barrel-assemblies for thermal angioplasty, the lift-gates must be made of less heat conductive materials, such as nonferromagnetic stainless steel as specified above in the sections on The Concept of the Extraluminal Stem and the Means for Its Placement, Subcutaneous and Suprapleural Patch Magnets, and Miniballs (Miniature Balls, Spherules, Minispheres). Lift-gate XX is positioned in and free to lift and drop along slot-ways to either side. Receding into flush relation to the surrounding outer surface of the muzzle-head when not in use, the incorporation of side-sweeping blades does not increase the diameter of the muzzle-head when not deployed. During use, the blades are directed edge-on to the circulation.

Positioned about the magnet assembly, the blades are distal to the muzzle-ports and so assist in the removal of plaque prior to stenting. Since until a brush or the brushes have been retracted or returned to the seated condition as undeployed or stowed, the barrel-catheter cannot be moved without side-sweeping the lumen wall, and the side-sweepers are raised (deployed, unstowed) by delivering current to the non-high temperature nickel-manganese-lead high thermal expansion alloy wires (see, for example, Bauer, H. J. 1977. “Mechanical Motions in Small Inaccessible Volumes,” Journal of Physics E: Scientific Instruments 10(4):332-334; Radvel, M. P. and Evdokimova, O. I. 2004. “Alloys with a High Coefficient of Thermal Expansion Based on the Mn—Pd System,” Metal Science and Heat Treatment 16(5):403-405 [original in Russian 1974, Metallovedenie i Termicheskaya Obrabotka Metallov 5: 36-38]) to which the brushes are mounted, retraction of the side-brushes is accelerated through the use of a cooling catheter, as described above.

Negligible ferromagnetism in the thermal expansion wires is tolerable. The 90 degrees centigrade equivalent limit current for the turret-motor windings and tractive electromagnets established by the direct current circuit from the inmate battery in the hand grip of a thermal angioplasty capable barrel-assembly, which can be used apart from an airgun for this purpose, is not used to actuate the thermal expansion wires used to elevate the side-sweepers, and since temperatures above 90 degrees centigrade are thrombogenic and the expansion of the wire requires no radiation of heat, the thermal expansion wires XX are insulated and lift-gate enclosure XX serve as an effective heat barrier. Portions of the internal wall of the muzzle-head aside from the heat-window or windows are coated with thermal insulation, such as squares of polytetrafluoroethylene impregnated thin glass fabric glued to the internal surface with high temperature silicone adhesive. The application of current to the horizontally positioned coil of thermal expansion wire on which each lift-gate sits overcomes the downward urging force exerted by fine wire torsion springs fastened by means of eyelets to their faces close to their sides, causing the upper edge of the lift-gates to rise and so extend beyond the periphery of the muzzle-head. The muzzle-head swivel or turret-motor is then used to reciprocally rotate the muzzle-head, so that the scraping blades allow the muzzle-head to be used as a lumen reamer.

Upon the removal of current (to the thermal expansion wires that underlie the lift-gates), the thermal expansion wires revert to their dimensions at room temperature. This causes the lift-gates, under the restorative force of the torsion spring to either side, to recede with upper reamer edges returned into flush relation with the outer surface of the muzzle-head. To reduce the risk of incising the lumen wall, the lift-gate carries side brushes that are as thick as the size of the muzzle-head will allow. When separately controllable, the lift-gates can be used to nudge the muzzle-head eccentrically within a lumen of suitable diameter or, depending upon the hardness of the lumen wall, which can be significantly increased by even partial positive modeling, to allow blood to pass; unlike a balloon, the radial force exerted by the lift-gate is slight but accomplished without obstruction of the lumen while deployed. When deflated, the balloon occupies less of the lumen and thus allows circulation superior to a barrel-assembly, which provided with a turret-motor, cannot be channeled through or simply condense in diameter. If plaque against which the lift-gate presses is too soft, the lift-gate will press into the plaque with no effect upon the muzzle-head.

Even at a brush thickness greater than 1.0 millimeter, to prevent longitudinal incision by brushes, especially those configured longitudinally, deployment or raising of the lift-gate side-sweeper brushes is used only at a standstill. The automatic retraction of any deployed side-sweeper upon resumption of transluminal movement may be accomplished with the incorporation into the deployment circuit of a movement sensor input switch. Limited to a small range in lumen diameters for nudging and the need to remain stationary, the side-sweeping feature does not negate the potential utility of an external hand-held electromagnet. When the muzzle-head is pulled against with the aid of an external hand-held electromagnet, the side-sweeping brushes must not be deployed or raised toward the pulling direction. Sticking of the lift-gates during lifting and lowering is prevented by lining the slideway slot to either side and coating the side edges of the lift-gates with a low friction polymer, such as nylon or polytetrafluoroethylene. Except for the upper corners, which are die-cut as rounded off at the upper ends to prevent cutting into the lumen lining, the upper edges of the faces and side edges are left squared.

The upper edges of the lift-gates are not undercut-routed to create sharp cutting edges along the faces to front and back. The object is to obtain a scraping action with little risk of catching or incision into the lumen wall. If the muzzle-head is prevented from rotating with the lift-gates deployed, these are allowed to recede before proceeding. Except at the rounded upper corners to each side, when the lift-gates are not deployed, the upper edges are flush to the outer surface of the muzzle-head. Where a rotational atherectomy burr or an excimer laser has eliminated hardened prominences, the side-sweeping feature can substitute for an angioplasty balloon in eliminating the balance of plaque, which is often less calcified at the periphery [REFERENCES?]. A supplementary plaque removal capability is of value, because both an excimer laser or a burr incorporated into the barrel-assembly must be centered in the muzzle-head and no larger in diameter than such devices are when independent.

As both burrs and lasers must leave the lumen periphery untreated to avoid injury, both of these devices may be followed by a balloon angioplasty to reduce the residual plaque. Here the side-sweepers are available for this purpose. To minimize the risk of embolization, excursion of the upper edges of the side-sweeper brushes beyond the outer surface of the muzzle-head is slight, generally no more than 1.5 millimeters. Provided the action does not result in obstruction of the lumen longer than 2-3 minutes, where the muzzle-head exceeds the lumen in diameter, the muzzle-head can itself be used to compress soft plaque or soft plaque scraped off the lumen wall. This is done by advancing and withdrawing the muzzle-head over the scraped area. The incorporation of conventional means of atherectomy makes achieving a minimally occlusive diameter more difficult, but reduces operating time.

### Automatic Disabling of Implant-Discharge, Side-Sweepers, and Filter-Trap During Transluminal Movement

If deployed during transluminal movement, the side scrapers can incise the lumen wall. The inevitable human error of simply forgetting to allow the side-sweeper brushes to recede before moving the barrel-assembly is precluded by incorporating a switch that instantly cuts off the current to the thermal expansion lifter wires when the barrel-assembly moves and continue to signal the operator so long as recession has not completed. Collaterally, a circuit-breaker stops the turret-motor when overloaded by a lesion posing resistance above a set threshold.

### Trap-Filter in Muzzle-Heads for Use in the Vascular Tree

The reservations that pertain to the use of a trap-filter in the vascular tree are addressed midway through the section entitled Concept of the Extraluminal Stent and the Means for Its Placement above. The current concensus favors the use of a distal embolic protective filter when preliminary intravascular ultrasonography, catheter-based thermography (near-infrared spectroscopy), or optical coherence tomography indicates the presence of vulnerable plaque. When not used following an angioplasty and on the first entry pass with an angioplasty-capable barrel-assembly, the muzzle-head, with a nose-cap that is a heat-window, releases heat to the surrounding lumen wall to suppress ruptures. When concern for the presence of vulnerable plaque distad to its reach is not an issue, the trap-filter can also be deployed to protect against the release of embolizing debris.

Filter deployment is also independently controllable and the filter membrane selected for resistance to modification in material properties by exposure to the release of heat from the nose. A number of recent advancements have been made toward the noninvasive detection of vulnerable plaque, to include multidetector row or multislice computed tomography scanning with iodinated nanoparticles dispersed with surfactant in a product called N1177 produced by Nanoscan Imaging of Lansdale, Pa. as contrast agent (see Hyafil, F., Cornily, J. C., Feig, J. E., Gordon, R., Vucic, E., Amirbekian, V., Fisher, E. A., Fuster, V., Feldman, L. J., and Fayad, Z. A. 2007. “Noninvasive Detection of Macrophages Using a Nanoparticulate Contrast Agent for Computed Tomography,” Nature Medicine 13(5):636-641). Vulnerable plaque contains more macrophages and is higher in temperature and acidity than healthy arterial wall tissue.

Noninvasive imaging methods do not yet characterize plaque but do make arterial inflammation and stenosis clear enough to signal the need for deploying a distal embolic protective filter. These technologies include molecular resonance imaging with gadopentic acid contrast agent (see Briley-Saebo, K. C., Mulder, W. J., Mani, V., Hyafil, F., Amirbekian, V., Aguinaldo, J. G., Fisher, E. A., and Fayad, Z. A. 2007. “Magnetic Resonance Imaging of Vulnerable Atherosclerotic Plaques: Current Imaging Strategies and Molecular Imaging Probes,” Journal of Magnetic Resonance Imaging 26(3):460-479; Amirbekian, V., Lipinski, M. J., Briley-Saebo, K. C., Amirbekian, S., Aguinaldo, Weinreb, D. B., Vucic, E., Frias, J. C., Hyafil, F., Mani, V., Fisher, E. A., Fayad, Z. A. 2007. “Detecting and Assessing Macrophages in Vivo to Evaluate Atherosclerosis Noninvasively Using Molecular MRI,” Proceedings of the National Academy of Sciences of the United States of America 104(3):961-966), multichannel, high-resolution laser scanning fluorescence microscopy (see Pande, A. N., Kohler, R. H., Aikawa, E., Weissleder, R., and Jailer, F. A. 2006. “Detection of Macrophage Activity in Atherosclerosis in Vivo Using Multichannel, High-Resolution Laser Scanning Fluorescence Microscopy,” Journal of Biomedical Optics 11(2):021009), intravascular fluorescence spectroscopy (see Tawakol, A., Castano, A. P., Anatelli, F., Basilian, G., Stern, J., Zahra, T., Gad, F., Chirico, S., Ahmadi, A., Fischman, A. J., Muller, J. E., and Hamblin, M. R. 2006. “Photosensitizer Delivery to Vulnerable Atherosclerotic Plaque: Comparison of Macrophage-targeted Conjugate Versus Free Chlorin(e6),” Journal of Biomedical Optics 11(2):021008.), and positron emission tomography (Tawakol, A., Migrino, R. Q., Bashian, G. G., Bedri, S., Vermylen, D., Cury, R. C., Yates, D., LaMuraglia, G. M., Furie, K., Houser, S., Gewirtz, H., Muller, J. E., Brady, T. J., and Fischman, A. J. 2006. “In Vivo 18F-fluorodeoxyglucose Positron Emission Tomography Imaging Provides a Noninvasive Measure of Carotid Plaque Inflammation in Patients,” Journal of the American College of Cardiology 48(9):1818-1824; Elmaleh, D. R., Fischman, A. J., Tawakol, A., Zhu, A., Shoup, T. M., Hoffmann, U., Brownell, A. L., and Zamecnik, P. C. 2006. “Detection of Inflamed Atherosclerotic Lesions with Diadenosine-5′,5′″-P1,P4-tetraphosphate (Ap4A) and Positron-emission Tomography,” Proceedings of the National Academy of Sciences of the United States of America 103(43):15992-15996).

To prevent the passage downstream of dislodged atherothrombotic debris as would result in distal embolization, deployment of the side-sweeping brushes is accompanied by the automatic deployment of a distal embolic protective trap-filter. The filter deployment mechanism is part of the barrel-assembly, no portion thereof discarded. Replacement filters are meant to be discarded after one-time that precedes the expiration date stamped on the package. Replacement filters are packaged after sterilization with ethylene oxide gas and attach toward the distal end of the vanadium permador (vanadium permendur) or silicon iron pin armature (slug, plunger, core) of the subminiature (micro) dc tubular push-type solenoid described in the section that follows. To achieve the necessary force of plunger expulsion, the coil is wound with silver wire. The prepositioning distally of a debris trap is especially important when performing an angioplasty on an artery with chronic total occlusion or a graft, often a saphenous vein, that has become occluded.

In these situations, a principal concern is the release of thromboemboli into the collateral circulation that had sustained perfusion despite the lack of canalization or luminal obstruction (see, for example, Meier, B. 1989 (reprinted 2005). “Angioplasty of Total Occlusions: Chronic Total Coronary Occlusion Angioplasty,” Catheterization and Cardiovascular Diagnosis 17(4):212-217; Kahn, J. K. 1995 (reprinted 2005). “Collateral Injury by Total Occlusion Angioplasty: Biting the Hand that Feeds Us,” Catheterization and Cardiovascular Diagnosis 34 (3): 65-66; Stone, G. W., Kandzari, D. E., Mehran, R. Colombo, A, and 23 other authors 2005. “Percutaneous Recanalization of Chronically Occluded Coronary Arteries: A Consensus Document: Part I,” Circulation 112(15):2364-2372; Stone G W, Reifart N J, Moussa I, Hoye A, Cox D A, Colombo, A., Bairn, D. S., Teirstein, P. S., and 19 other authors 2005. “Percutaneous Recanalization of Chronically Occluded Coronary Arteries: A Consensus Document: Part II,” Circulation 112(16):2530-2537).

Furthermore, chronic total occlusion notably affects the coronary arteries, which grudging of working depth, discourage the use of a transluminal device that requires significant longitudinal extension down the lumen. Accordingly, a long filter of the kind proposed in 2003, which sought to combine the strongest features of the best filters then on the market, at the least demands modification for use in the coronary vessels. In a center-discharge barrel-assembly, the concave recess for storing the trap-filter in the nose. A combination-form muzzle-head without center burr or laser cable installed affords a central canal for the trap-filter. When a cable is installed, the nose must be extended forward (distally) and the trap-filter recess located to a side of the distal terminus of the cable. The trap-filter deployment mechanism is disabled while an installed laser is in use. Side-sweepers can be deployed during an angioplasty that is performed with an angioplasty-capable barrel-assembly where the apparatus remains independent of an airgun unless and until the barrel-assembly is inserted into an airgun to initiate stenting.

However, trap-filter deployment may also be desired during discharge of the airgun as a measure in addition to energization of the recovery electromagnets for preventing the passage of a loose miniball downstream when the magnetic field intensity would best kept to the minimum. Since a discharge barrel-assembly may not be angioplasty-capable, a trap-filter is desirable in angioplasty-capable barrel-assemblies and may be desirable in some ablation and angioplasty-incapable barrel-assemblies. For this reason, an additional circuit independent of that used to energize the thermal expansion wires is provided to deploy (elevate) the side-sweepers for deployment of the trap-filter alone when desired during discharge as added if redundant protection against the escape of a miniball down the bloodsteam. Since a trap-filter reduces working depth when deployed, it is best stowed when not required but readily deployable and retrievable without the loss of debris. While a combination-form barrel-assembly that incorporates a laser has the ability to disintegrate debris that passes before it, compulsory activation of the laser whenever the side-sweepers are used is unacceptable as posing a risk of perforation.

Trap-filter-deployment solenoid end-of-travel impact shock as would tear the filter membrane and jolt the trap-filter so that the outer nitinol ring kicked the lumen wall, is checked by an elastomeric bumper-washer that surrounds the plunger exit orifice to lightly clutch about the plunger and thus slow down and dampen plunger ejection and spring return. Further to reduce the risk of tears and kicking, one continuous strand of polyurethane suture is diametrically wrapped entirely about the outside of the filter membrane to begin and end at the head of the solenoid plunger. A second such wrapping around or continuation with the same strand at right angles to the first produces what appear to be four struts. Even though the trap-filter-deployment solenoid is enclosed within the ejection head as to release heat through the muzzle-head nose-cap, the periods of energization (duty cycle) involved would produce thrombogenic temperatures unless the bumper-washer cooperated with the rest of the solenoid recess lining as a thermal insulator, the solenoid is heat-sunk, and the cooling catheter is used to prevent over-, or for that matter, under-heating.

### Trap Filter Deployment and Retrieval Mechanism

As shown in FIG. XX, polyurethane filter membrane XX having 120 micrometer pores is of a length that is recoverable or restowable into silo recess XX with additional space for retracting several miniballs, albeit improbable of necessity. The trap-filter is deployed by a subminiature push-type solenoid until the removal of current causes the extension spring within the solenoid to reseat the plunger causing the trap-filter to restow. The trap-filter is deployable on demand but automatically deployed with the side-sweepers; more specifically, the trap filter solenoid is energized at the same time as are the thermal expansion wires that deploy the brush-rib lift gates. Self-expanding nitinol wire loop XX allows 360° apposition. In an angioplasty-capable barrel-assembly used independently of an airgun, the trap-filter is controlled from the on-board control panel with power drawn from the inmate battery pack with circuitry likewise contained within the hand-grip. By contrast, an ablation and angioplasty-incapable barrel-assembly does not function independently of an airgun but draws power from the power supply upon being connected to the airgun either by the connection depicted in FIG. 37 or FIG. 40. The miniature parachute or umbrella-configured trap-filter shown in FIG. XX can also be configured as a windsock, or for dealing with eccentric lesions in type ducti other than vascular, trawler type fishing net-configured.

### Thermal Conduction Windows (Heat-Windows) and Insulation of the Muzzle-Head Body in Thermal Ablation or Thermal Angioplasty-Capable Barrel-Assemblies

For the purpose of allowing the muzzle-head to be used for performing a thermal angioplasty, heat transfer from the windings within the muzzle-head is by conduction, the small internal diameter of the different systemic vasa to require treatment imposing stringent limitations upon winding diameters and the thickness of the insulation that can be used. Albeit consisting of fine silver wire, ninety degrees centrigrade is not so hot as to necessitate extraordinary insulation of the recovery electromagnets, turret-motor stator (armature), trap-filter deployment solenoid coils, connections, or cabling. Even though these components are positionally disabled during thermal use, heat-conducting windows of silver, which has a heat transfer coefficient at 25 degrees centrigrade of 429, or of copper, 401, in the otherwise thermally insulated muzzle-head body allows heat to be directed from the nose cap (nose dome) heat-window to the surrounding lumen wall and from the turret-motor toward lesions that may be circumferentially asymmetrical or eccentric. The higher temperature of a heat-window allows its position to be viewed through thermal imaging, and, if redundantly, allows the deliberate heating of a certain winding to assist in pinpointing not only the location of the muzzle-head but the orientation of the window.

To allow blood to flow past a muzzle-head flush fit to the lumen, the turret motor must be smaller in diameter than the portions of the muzzle-head that lie to the fore. Contact with the wall of the lumen requires that the turret-motor heat-window slightly protrude with smooth edges beyond the rest of the circumference of the turret-motor. Some blood can then flow around the heat-window past the motor body into the blood grooves to the fore. The nose-cap heat-window of the recovery electromagnets is flush-mounted to the muzzle-body. The turret-motor and tractive electromagnets represent three independently controllable heating elements, each in its own circuit, in relation to which the muzzle-head body can incorporate heat-radiating windows of any shape, extent, separation, or connection. However, except for ablation and angioplasty-incapable barrel-assemblies, to avert the disruption of vulnerable plaque by contact with the muzzle-head, all muzzle-heads for use in the vascular tree have a heat-window in the form of a forward end encompassing nose-cap and turret-motor heat window that is directed (circumscribed).

Using the recovery electromagnets separately, band, strip, or slit-shaped windows along either segment of the muzzle-head body along one side behind (proximal to) the nosecap heat window, for example, can be heated by the motor proximally and/or the magnets distally. Since in a combination-form barrel-assembly, the central canal is occupied by an atherectomy burr or laser cable, this space is unavailable, necessitating the use of an edge-discharge muzzle-head, which requires that a spare barrel-tube be used as a cooling catheter entry and service channel. The need to appropriate a barrel-tube affects the maximum diameter of the barrel-tubes, hence, the caliber of the miniballs that may be used when all the barrel-tubes are to be used for the discharge of miniballs; however, the diameter of the miniballs to be implanted will seldom be forced smaller by this factor, and then only when a multiple discharge barrel-assembly is preferred.

When the central canal in a multiple-discharge barrel-assembly, such as a four-way radial edge-discharge barrel-assembly, is already occupied by a laser or burr cable or by a trap-filter, access to the central canal for use as a service channel to insert a cooling catheter of larger diameter is preempted. This necessitates the use of a spare barrel-tube as service channel, limiting the cooling catheter to capillary tube gauge, typically 0.38 millimeters in outer diameter. Even though made of polytetrafluoroethylene for rigidity, to feed this fine catheter down to the ejection head and confirm its correct position represents a distraction and interruption that is avoided by prepostioning the catheter. Whether used for cooling or the delivery of medication or a lubricant, catheters requiring access through a service channel are prepositioned. Solid rods of graduated stiffness preferred for the purpose, catheters would seldom be used merely to stiffen or straighten the barrel-assembly, as when having passed a tortuous stretch.

Limitation to a barrel-tube for the insertion of a cooling catheter means that the conduction path for chilled air to the turret-motor, the side-sweeping brushes deployed by thermal expansion wires, and the recovery electromagnets is asymmetrical, gives less effective conduction to the recovery electromagnets, and since the material of the barrel-tube, even thin-walled, is thermally insulative, necessitates the extension of the gas return path perforations along the sides of the barrel-tube to include the entire segment of the barrel-tube parallel to the area to be cooled. For these reasons, a center-discharge barrel-assembly with its larger diameter central canal and ejection head cooling catheter insertion channel is superior to an edge-discharge barrel-assembly for thermal angioplasty.

The effect of a fever on clotting with clot-suppressing (blood thinner, anticoagulant), and dissolving (thrombolytic) drugs conventionally used in angioplasty have been administered calls for further research. Disregarding the administration of such medication, which infrequently produces significant bleeding complications Cote, A. V, et al. 2001 and Jong, P. et al. 2001, cited under Objects of the Invention), to reduce the thrombogenicity of the arterial wall when heated, a temperature of 90 degrees centigrade (Celsius; 194 degrees Fahrenheit) or more serves to denature collagen and von Willebrand factor (Post, M. J., de Graaf-Bos, A. N., Posthuma, G., de Groot, P. G., Sixma, J. J., and Borst, C. 1996. “Interventional Thermal Injury of the Arterial Wall Unfolding of von Willebrand Factor and Its Increased Binding to Collagen After 55 Degrees C. Heating,” Thrombosis and Haemostasis 75(3):515-519; Humphrey, J. D. 2003. “Continuum Thermomechanics and the Clinical Treatment of Disease and Injury,” Applied Mechanics Reviews 56(2):231-260; Bos, A. N., Post, M. J., de Groot, P. G., Sixma, J. J., and Borst, C. 1993. “Both Increased and Decreased Platelet Adhesion to Thermally Injured Subendothelium is Caused by Denaturation of von Willebrand Factor,” Circulation 88(3):1196-1204; Borst, C., Bos, A. N., Zwaging a, J. J., Rienks, R., de Groot, P. G., and Sixma, J. J. 1990, “Loss of Blood Platelet Adhesion After Heating Native and Cultured Human Subendothelium to 100 Degrees Celcius,” Cardiovasc Research 24(8):665-668).

Thermal angioplasty windows, window-slits, and window slots are microrouted or electrical discharge machined into the sides of the proximal (rear) and distal (front) muzzle-head shells in a center-discharge muzzle-head and the proximal shell in edge-discharge or combination-form barrel-assemblies. The window openings or apertures are then covered over with silver or copper sheet which is inset or lapped into the outer surface of the opening or openings to create an edge that is flush to the surface of the muzzle-head body. The turret-motor slit or slot heat-window slightly protrudes or stands in relief of the surrounding surface, while the nose-cap heat window is flush or filet fit. The window overlays are bonded along the overlap with a long-chained cyanoacrylate cement not subject to liberate toxic substituents upon degrading.

To allow good slippage, or the noninjurious movement of the muzzle-head body against the endothelium, the heat-windows are masked for immersion (dip) or sputter coating with a thermal insulating polymer. A fluoropolymer is preferred for a low coefficient of friction and little tendency for clinging to the endothelium. Since to prevent a temperature gradient about the heat window is impossible, the use of thermal insulation is intended to better focus the heat. Where the heat foci do not pass, anticoagulant medication reduces the risk of thrombus formation due to temperatures that intervene between body temperature and 90 degrees centigrade (Post et al. 1996, Thrombosis and Haemostasis 75(3):515-519 cited above). While at 3 millimeters (9 French) in outer diameter, most muzzle-heads will not allow any further coating, for additional insulation, the polytetrafluoroethylene coating on the outer surface of portions of the muzzle-head shell other than those cut away for the heat-windows, slits, or slots may be further coated with a thin layer of silica aerogel.

As shown in FIG. XX, the thermal window-slits in the front and rear shells of the center-discharge muzzle-head follows the blood grooves along the deep portion or valley. In FIG. XX, the thermal windows represent special heat radiating sectors in the muzzle-head body or shell. While the thinness of the thermal insulation required in the body or shell of the muzzle-head makes thermal isolation difficult, the differential thermal conductivity of the window or windows in relation to the speed with which the control electronics and materials employed allow the target temperature to be attained and receded from affords a level of thermal isolation sufficient for thermal angiolasty with minimal risk of thrombogenicity due to heat retentive temperature gradients surrounding the radiative window or windows. When current is sent to the turret-motor and/or either electromagnet winding for use as a heating element, there is a brief interval or lag until the corresponding temperature is attained and stabilized at the heat-window. Also, the longer the current is applied, the more heat accumulates within and is conducted through the muzzle-head. As a practical matter, the application of heat during the course of a thermal angioplasty is ordinarily intermittent rather than continuous to the degree that it would allow a significant thrombogenic buildup of heat.

When the area for treatment is considerable but the overall procedural time is not unduly extended, the operator deliberately shuts off heat delivery and uses the prepositioned (previously inserted within the barrel-assembly) cooling catheter intermittently to avoid the excessive buildup of heat. However, in an angioplasty barrel-assembly for use with plaque so extensive that to treat it within a limited procedural time would require inordinate heat on-time, a negative feedback control microcircuit contained within the hand-grip shaped battery pack is used to surge the current when heating is begun and then gradually drop off the current with time, thus maintaining the temperature substantially constant until the cooling catheter is used to cool down the muzzle-head. To avoid the additional complications of using a single such circuit to control the temperature whether one or both recovery electromagnets and/or the turret-motor is used as the heating element for thermal angioplasty and endoluminal ablation, each winding is provided with an independent control circuit (see, for example, Prosser, T. F. 1966. “An Integrated Temperature Sensor-Controller,” Institute of Electrical and Electronics Engineers Journal of Solid-State Circuits 1(1):8-13); however, space within the barrel-assembly being at a premium, the circuit employed preferably senses temperature indirectly on the basis of electrical indicia.

Generally, the turret-motor winding is treated as singular despite the resistance intervening between these, the heat-windows depended upon for directing the heat. Only embodiments that are thermally anisotropic as to be thrombogenic aside from the heat-window or windows warrant the limitation of heating current to one motor winding. With independently heatable recovery electromagnets, three heat servocontrol microcircuits are required in the hand-grip in addition to the 3-phase turret-motor drive-control microcircuit. In most situations, for the interval over which the heating current is left on at any one time, adaptive control is considered to exceed the medically meaningful requirement. Accordingly, for simplicity and economy, rather than to measure and adaptively control the temperature intraoperatively, the current to quickly bring the temperature to 90 degrees centrigrade, which due to the use of different materials in different dimensions and thicknesses, is different for each type of muzzle-head, the control of temperature is provided on the basis of empirically pretesting the specific muzzle-head for current-to-temperature equivalency with fine distinctions of temperature rise time and accumulation discounted.

A precision thermocouple consisting of a fine bimetallic thermal expansion strip, such as one made of invar steel and brass or aluminum welded together, that thermomechanically completes the circuit for current flow-through by making contact-connection only when its temperature corresponds to 90 degrees centrigrade for angioplasty and the other temperatures specified herein for ablation at the heat-window-endothelial interface, can be used. More specifically, the control of temperature is based upon empirical trials, with the exact combination of components to be used, and detailed instructions, such as for the setting and time for the delivery of cold air through the cooling catheter, provided on a specification sheet that is supplied with the apparatus.

Further to reduce the number of components that would consume precious space and an overall complexity that would significantly increase costs:

a. Rather than to provide a local current-actuable insulating layer or movable insulating cover in surrounding relation to the heating elements (turret-motor and recovery electromagnet housings), the momentary temperature rise-time is discounted, anticoagulant medication, which can be delivered in higher than the circulating or systemic concentration through a catheter passed through a neighboring barrel-tube or service channel (below) depended upon to minimize unwanted coagulation; and
b. The same rapid cooling catheter or cooling capillary catheter (following section) is used in center and edge discharge muzzle-heads within the range of common diameters (2.5-4.0 millimeters), the placement and interval of time using a specific vortex tube or other means for supplying cold air required in each instance to drop the temperature from 90 degrees centrigrade, or if used for thermal ablation in a ductus other than vascular, then the temperature that pertains, back down to body temperature (98.2 degrees Fahrenheit or 36.8 degrees centigrade) provided on a specification sheet that is supplied with the apparatus for setting the vortex tube timer, which interval of time has been predetermined empirically based upon multiple trials.

The low thermal conductivity of the materials used; rate of cold air delivery from the cold air gun, CO₂ or NO₂ cartridge; tight fit of the rapid cooling capillary catheter within the passageway employed whether peribarrel space, gas-return path, or spare barrel-tube used as a service-channel; and interval to pressure equalization among the holes toward the distal end of the cooling catheter is such that in a combination-form edge-discharge muzzle-head as discussed in the section to follow, extension proximally of the perforated distal segment of the fully inserted rapid cooling catheter to the turret-motor does not result in a significant lessening of the cooling effect at the turret-motor.

### Heated Turret-Motor, Brush Lifting Thermal Expansion Wire, and Recovery Electromagnet Rapid Cooling Catheter and Cooling Capillary Catheter

To quickly return the thermal angioplasty turret-motor, one or both brush-lifting thermal expansion wires, and one or both recovery electromagnets to body temperature, a cooling catheter is passed down the barrel-assembly so that its cold air is delivered in adjacent relation to these components. Because in a combination-form barrel-assembly, the longitudinal center is taken up by an atherectomy burr or excimer laser cable, an edge-discharge muzzle-head must be used, so that only a spare barrel-tube is available for passing the rapid cooling capillary catheter up to the turret-motor. A noncombination-form barrel-assembly with center-discharge muzzle-head, however, provides a central canal to allow a cooling catheter of larger diameter and an ejection-head channel for insertion of the distal end of the cooling catheter for more direct access to the recovery electromagnets. The gas return paths prevent cooling gas from entering the bloodstream. The mechanism by which the side-sweepers are controlled is described below under the section entitled Control of Side-sweeping Brushes. A cooling catheter is needed when the internal configuration of the muzzle-head fails to obstruct the release of pressurized gas into the bloodstream.

Using a center-discharge muzzle-head, when the turret-motor is sent heating current to quickly raise to and hold the temperature at 90 degrees centigrade for thermal angioplasty, preferably the slit valve but possibly a spare barrel-tube is used to admit the rapid cooling extruded polytetrafluoroethylene capillary catheter. With such a muzzle-head, when one or both miniball recovery tractive electromagnets are used for thermal angioplasty, the rapid cooling capillary catheter is advanced up through the central canal and into the ejection head rapid cooling capillary catheter insertion channel. In a combination-form angioplasty barrel-assembly, the slit valve is eccentric. Because the electromagnets are contained within chambers that directly communicate with the bloodstream beyond the gas return paths, blowning cold air directly on the electromagnets would risk introducing gas into the bloodstream. For this reason, the end of the capillary catheter is closed and side-holes are used. Cooling of the electromagnets is through the metal of the ejection head, the larger diameter of the insertion channel allowing the cooling gas to circulate against the interior walls of the insertion channel and exit through the central canal.

Using an edge-discharge muzzle-head, the cooling catheter is moved forward until its distal tip closes off the muzzle-port of the barrel-tube through which the cooling catheter was passed. If only one electromagnet was used for thermal angioplasty, the cooling catheter is passed through a barrel-tube proximal to that electromagnet, which can be identified, because each barrel-tube is marked on the end-plate (99 in FIG. 37). The asymmetry of this position and separation by metal surrounding the electromagnets mean that the rate volume of chilled air delivery must be greater than in a center-discharge muzzle-head. The use of more than one cooling catheter in an edge-discharge muzzle-head to ameliorate the less effective cooling associated with the asymmetry of cooling catheter placement is practicable when the caliber of the barrel-tube or barrel-tubes used for discharge are not affected. Combination-form barrel-assemblies with edge-discharge muzzle-heads can be deliberately designed to be cooled in this way. The functionality of providing a service channel for access to the muzzle-head for various purposes is discussed above.

Referring now to the detailed view of a center-discharge muzzle-head shown in FIG. XX, for ease of insertion of the rapid cooling catheter into the ejection head rapid cooling catheter insertion channel XX, the distal end of the rapid cooling catheter or cooling capillary catheter is closed and angled as chamfered or conical. Round side-holes surround the distal segment of the cooling catheter over the length to be cooled, i.e., that of the muzzle-head, this being the distance separating the proximal end of the turret-motor from the proximal end of the recovery electromagnet assembly. Chilled air is delivered through the cooling catheter by connecting its free proximal end with a diameter-reducing adapter to the nozzle of a cold air gun that uses a vortex tube supplied with compressed air, such as manufactured by Vortec Division, Illinois Tool Works Air Management; Airtx International; Exair; and Pelmar Engineering. The base of the cold air gun is fastened to the side of the interventional airgun cabinet.

Alternatively, high purity 1,1,1,2-tetrafluoroethane (R134a) cryogen spray is blown through the cooling catheter side-holes under low pressure by inserting the free proximal end of the cooling catheter into the spray nozzle hole of the aerosol can containing the tetrafluoroethane, the need for a diameter-changing adapter depending upon the diameter of the cooling catheter. Testing has revealed tetrafluoroethane to be safe (Emmen, H. H., Hoogendijk, E. M., Klopping-Ketelaars, W. A., Muijser, H., Duistermaat, E., Ravensberg, J. C., Alexander, D. J., Borkhataria, D., Rusch, G. M., and Schmit, B. 2000. “Human Safety and Pharmacokinetics of the CFC Alternative Propellants HFC 134a (1,1,1,2-tetrafluoroethane) and HFC 227 (1,1,1,2,3,3,3-heptafluoropropane) Following Whole-body Exposure,” Regulatory Toxicology and Pharmacology 32(1):22-35; Gunnare, S., Ernstgård, L., Sjögren, B., and Johanson, G. 2006. “Toxicokinetics of 1,1,1,2-tetrafluoroethane (HFC-134a) in Male Volunteers After Experimental Exposure,” Toxicolology Letters 167(1):54-65).

To allow the rigidity necessary for very thin, for example, 0.39 millimeter, cooling catheters to be passed to the muzzle-head through an available barrel-tube, the cooling catheter is made of polytetratluoroethylene. In a combination-form barrel-assembly that must have an edge-discharge muzzle-head the use of an available barrel-tube for this purpose is unavoidable. Although the effect is slight because of the small diameter of the cooling catheter, this affects the flexibility, hence, trackability of the barrel-catheter. Since inserting the cooling catheter can assist in advancing the barrel-assembly, so long as care is taken to avoid stretching injury, this can be used to advantage. Tubes and solid rods made of many different materials and covering a wide range of flexibility can likewise be inserted to stiffen or straighten the distal end of the barrel-assembly. A hand-held electromagnet can also be used to aid in steering the muzzle-head.

A dividing and diameter changing adaptor allows the use of multiple cooling catheters with a single cold air gun; the use of a separate cold air gun for each cooling catheter is generally not necessary. Provided trackability is not impaired, to eliminate insertion time and allow immediate retreat from the 90 degree centigrade target temperature for thermal angioplasty back down to body temperature, multiple cooling catheters are prepositioned. Otherwise as many cooling catheters are prepositioned as do not affect trackability. Using a center-discharge barrel-assembly, the main and larger diameter cooling catheter is passed down the central canal and into the ejection head channel with another, usually capillary gauge, cooling catheter passed down the barrel-tube closest to the heated element.

Turret-Motor and Tractive Electromagnet Insulation, Leads, and Control when Used as a Heating Element in Muzzle-Heads Designed for Thermal Angioplasty

Turret-motors and tractive electromagnets to serve as a heating elements for thermal angioplasty must incorporate special features of thermal and electrical insulation, to include winding insulation that is effective as an electrical but not as a thermal insulator, such as Masterbond EP34AN epoxy adhesive/sealant (thermal conductivity 22-24 BTU/in/ft²/hr/° F.), pyrolytic boron nitride, or boron nitride, for example. Since atheromatous lesions are usually asymmetrical, restricting the radiation of heat from the muzzle-head allows less heat to be directed toward less affected radii, hence, more discretionary treatment. This is approximated by providing that only a delimited arcuate sector of the proximal portion of the muzzle-head shell or body serving as motor housing will radiate heat, the balance of the housing being thermally insulating. The turret-motor as thermal angioplasty heating element can be supplemented through use of the tractive electromagnet(s) for the same purpose.

Ideally, once the heat within the muzzle-head body met or exceeded 90 degrees centigrade, the heat transmission window would radiate only 90 degrees centigrade. The sparsely intermittent duty of the turret-motor in positional control and the fact that the motor is fed current only when needed mean that little heat is generated. This allows a sector of the muzzle-head shell to exceptionally be made of a material without value as a thermal insulator. Since atheromatous lesions are usually asymmetrical, the capability to differentially heat the turret motor over a restricted arc of its circumference gives more discretionary control, but also requires that lower thrombogenic temperatures are not transmitted to the adjacent arcs, as discussed below. Silver and copper having been specified as the materials preferred for the heat-windows on the basis of maximum thermal conductivity, the selection and thickness of the materials used in heat-windows does not take into account such alteration in thermal conductivity as results from the fact that the exterior surface of the heat-window will be wetted with blood or some other bodily fluid.

The ideal turret-motor as heating element would quickly rise from room temperature to 90 degrees centigrade, quickly drop from 90 degrees to room temperature, and be enclosed within a motor housing having a copper window slot through which the heat would be conducted, other portions of the entire turret-motor housing surface made of a material such as polytetrafluoroethylene or stainless steel having a markedly greater heat transfer coefficient or thermal condctivity the motor tolerating the heat otherwise retained without significant radiation to the surrounding lumen. Angioplasty performed manually before initiating stenting by inserting the proximal end of the barrel-assembly into the airgun, the motor would not, however, be used to rotate the muzzle-head during use as a heater.

For use as a heating element for the thermal angioplasty of a delimited arc of the lumen wall, a substantially temperature isolated arc of the turret-motor must be quickly heatable from a cool condition to 90 degrees Centigrade, necessitating wire and winding insulation that resists melting failure in small gauges depending upon size, especially when anticoagulant medication is contraindicated making the avoidance of thrombogenic temperatures intermediate between zero and 90 to be avoided. To keep the nonangioplastic operating temperature of the turret-motor well when used for positional control below thrombogenic levels, the control circuit delivers current to the motor only when the motor is used.

To avoid the intevening thromogenic temperatures, the subminiature silver wire wound motor must be capable of being quickly elevated to 90 degrees centrigrade, the initiating of heating commencing with a current surge that gradually levels off to maintain a constant temperature as mentioned in the preceding sections entitled Concept of the Extraluminal Stem and the Means for Its Placement and Thermal conduction windows (heat-windows) and insulation of the muzzle-head body in thermal ablation or thermal angioplasty-capable barrel-assemblies. Overheating normally results from excessive starting torque or torque at elevated speeds, whereas here heat is deliberately applied when the motor is not in use as a driver. That heat must, however, be dealt with when the motor is used as a driver. Since fluoropolymers are effective thermal insulators, when the muzzle-head is or coated with a thin layer of a fluoropolymer to obtain a no-stick surface, the turret-motor is given a thinner or no such coating.

However, if the coating has microscopic gaps, a thinner coating over the motor may still allow sufficient heat to pass through to the lumen wall. Even though rotation is intermittent or discontinuous and angle-to-angle within a circle or semicircle, use as a rotary driver immediately following use as a thermal angioplasty heater is better accomplished with a turret-motor that dissipates the excessive heat previously required quickly and thus averts rotatory instability (see, for example, Basu, A., Moosavian, S. A., and Morandini, R. 2005. “Mechanical Optimization of Servo Motor,” Journal of Mechanical Design 127(1):58-61). Heating the stator to perform thermal angioplasty represents a separate mode of turret-motor operation and is not employed when it is necessary to rotate the muzzle-head.

For example, to combine thermal and side-sweeping angioplasty, the action is carried out transluminally under manual control with one or both of the side-brushes—which may be the same or different in bristle stiffness and tip conformation—deployed at a fixed rotary angle with the turret-motor heated at stall. Then to rotate the side-brush or brushes, the manual action is suspended, the turret-motor switched for rotation, the brush or brushes rotated, and the turret-motor switched back to heat while stalled in order to resume thermal angioplasty at the new brush rotary angle. The insulation of the turret-motor must tolerate sufficient current for thermal angioplasty without melting, and the turret-motor must be well temperature insulated from the more forward elements of the muzzle-head or extremes of temperature will produce a temperature gradient that will result in thrombogenic temperatures in these more forward elements at and around 50 degrees centrigrade (122 degrees Fahrenheit) (Post et al. 1996). For thermal angioplasty, the turret-motor stator must quickly pass the thrombogenic range and reach a temperature of 90 degrees centigrade when sent higher current while stalled, and must just as quickly cool to room temperature when the current is removed.

Combination-Form Barrel-Assemblies: Barrel-Assemblies that Incorporate Means for Thrombectomy, Atherectomy, or Atherotomy
Forward-Directed Clearing (Ablation) Means for Integration into the Muzzle-Head

The rearward extension of the muzzle-head imparts a longitudinal aligning effect, and the barrel-catheter with internal components is made sufficiently stiff to track with no buckling without the need for a guide or ‘buddy’ wire, the muzzle assembly itself effectively a fixed wire, as opposed to an over the-wire device. Any catheter-based means for the removal of plaque can be integrated into the muzzle-head at the front end so that the implantation of the miniballs can follow immediately upon the removal of plaque. However, a preferable means would not necessitate either thermal insulation or extension in length of the muzzle-head as to deny depth of implantation access. While the longitudinal elongation exerts a canal-aligning effect that reduces the risk for an exposed sharp rotational atherectomy cutter or burr at the front end to go off-course producing furrows if not perforations, so long as the burr remains exposed, this would always loom as a possibility. Thus, to use a rotational burr, the muzzle-head would have to be extended forward in length so that the burr could be held within a recess at the front end until used.

While such extension would consist of only about six millimeters, this length could prove significant if moving down the vascular tree, the lumen diameter had become sufficiently narrow to require stretching and the likelihood of dissection if advancement were to continue. For this reason, the incorporation of a rotational atherectomy burr, which is also less capable than alternative devices, is discounted. Conventional or balloon-based thermal and cryogenic devices introduce temperature and thus dimensional instability that to protect against erratic performance would necessitate the incorporation of insulation for which space is lacking. In the coronary arteries, for example, placing balloon based devices in tandem with the muzzle-head would untenably deny transluminal depth of access.

The least obtrusive and disruptive means of ablation suitable for integration into the muzzle-head are ultraviolet xenon-chlorine excited dimer or ‘excimer’ lasers and continuous wave neodymium yttrium aluminum garnet or Nd:YAG, and carbon dioxide or CO₂ lasers. Nevertheless, demanding an increase in the outer diameter of the muzzle-head, incorporating a laser does reduce the lumen diameter, hence, the extent of the vascular tree that may be accessed with any one muzzle-head. For this reason, the incorporation of a laser into a barrel-assembly relates more to those with one or a few barrels. In contrast to thermal and cryogenic balloons, the optical fibers connected to the console leading to the small diameter probe tip are mechanically and thermally passive.

Barrel-Assembly with Excimer Laser

While various mid-course divisions or divergences and confluences of laser fiber optics would allow the fibers to be coursed about other components, incorporation of an excimer laser into a barrel-assembly is advantageously and preferably obtained without the need to significantly modify an off-the-shelf laser catheter. Laser atherectomy actually cavitates and vaporizes all but highly calcified plaque rather than merely compressing plaque as does balloon angioplasty, which is, however, accomplished more quickly. In the present context, however, the side-sweeping capability, the laser in variable sequences, and—when the lumen diameter is the same or smaller than that of the muzzle-head—coordinated use of the muzzle-head much as a balloon, allow greater speed of tissue reduction and removal than any of these components alone. All catheter-based procedures can induce abrupt closure by spasm. Other problems encountered with lasers alone, notably the inducement of spasm and promotion of fibrin deposition, are similarly moderated by immediate follow-up with implants (spherules, miniballs) that have been medicated to counteract these complications. Since both laser atherectomy and ballistic implantation can induce such responses, making antispasmodic and anticoagulant-coated miniballs routinely available is advisable.

Referring now to FIG. 36, shown is the front or distal end of a muzzle-head that incorporates a pulsed ultraviolet photo-ablation excimer laser having optical fibers, each consisting of a core, cladding, buffer, and containing sheath or catheter, with distal end centered in the nose. To prevent plaque that extends to the center of the lumen from being undercut closer to the lumen wall and freed as pieces toward the lumen axis to pass down the bloodstream intact, the distal ends of the optical fibers close to the surface of the muzzle-head can follow the outer contour of the muzzle-head slightly bent toward the central axis, while the outer fibers are directed straight ahead. The muzzle-head of a barrel-assembly that incorporates a laser must have a nosing that places the distal ends of the optical fibers at a distance from the front end and the plaque to be fully effective. The optical fibers are omitted from positions about the circumference that would cause the fibers to course over blood-grooves, blood-tunnels, the entry ledges before the doors on the traction electromagnet chambers, the muzzle-ports, and side-scaper ablators.

Remote rotation of the muzzle-spindle by means of a swivel or turret-motor requires that the optical fibers be cut at the level of the rotary joint between the distal end of the motor and proximal end of the muzzle-spindle. However, since rotation is no part of the plaque removal process but rather part of the stenting function which does not commence until plaque removal has been completed, the proximal and distal portions of each fiber do not move in relation to one another until their use has ended. To minimize losses to refraction and scatterting at the interface where the portions meet, the segments of the fibers are made to interface as flush as will not interfere with the use of the swivel or turret-motor. For atherectomy by means of photo-ablation, the xenon-chlorine laser is set to a wavelength of 308 nanometers with a rate of pulsation ranging between 25 and 80 repetitions per minute. The fiber optics in the barrel-assembly are connected to an excimer laser control console, such as the Spectranetics CVX-300® excimer laser system. Since the laser vaporizes plaque laying directly to the front of the distal ends of the fibers, better coverage is obtained when the fibers are more radially distributed about the nose of the muzzle-head.

### Direction of Muzzle-Head on Discharge as Prograde (Advancing, Forward, Distad) or Retrograde (Withdrawing, Backward, Proximad)

The trap-extractor magnet assembly necessarily positioned distal to the muzzle-ports, prior to the first discharge when placement of the stent-jacket is to follow, the magnetic field generated by the trap-extractor magnet assembly interdicts the passage of any loose miniball down the lumen. To avoid the risk of disrupting any miniballs that have already been implanted, the barrel-assembly is advanced forward from discharge to discharge thus causing the magnetic field to move away from rather than to pass these. A parachute or trawler type fishing net configured filter deployed ahead of (distal to) the leading end (nose) of the muzzle-head as described above is also available to trap loose miniballs. When the procedure has been completed and the barrel-assembly is to be withdrawn, the amperage through the electromagnets is reduced to zero and the barrel-assembly withdrawn.

If any miniballs failed to implant, then the rotary magazine clip hole for the number a position or barrel-tube to implant the missed positions is used to place those missed when withdrawing. If a miniball or miniballs had been retrieved, then the amperage is reduced to the lowest value that will allow the trapped miniballs to be held through any radial accelerations or recoils of the muzzle-head to follow and the barrel-assembly to be withdrawn without disrupting the miniballs that have been implanted, and once these are past, the amperage may be returned to a higher value. If not matching the lumen in diameter, the barrel-assembly can be nudged into contract with the lumen wall with the aid of an external hand-held electromagnet as described below and blanked out rotary magazine clips used to implant only the side in contact.

### Sequence of Stent-Jacket Placement and Implantation

Various circumstances that recommend placement of the stent-jacket prior to initiating stent miniball discharge, such as to prevent perforations and limit rebounds are addressed above in the section entitled Stent-jacket Linings for Containing or Preventing Perforations and for Reducing the Momentum and Misdirection of Rebound While perforation (through-and-through penetration with the extravascular exit of a miniball) is always to be avoided, the diameter of the miniballs is such that this would rarely lead to significant injury, such perforations quickly sealed spontaneously. Neither would the permanent lodging within tissue of a miniball with its bioinert jacket likely prove harmful, the exception being that escape into the bloodstream must be prevented.

However, when proximity to a vulnerable neighboring structure dictates, perforation is prevented from leading to the puncture of the neighboring structure that would ensue were the perforation not interdicted at the adventitia. This is accomplished by previous placement of the stent-jacket, which then serves to block a perforating miniball from continued travel. In such use, the internal lining of the stent-jacket must exhibit the resilience to minimize rebound, which could result in the entry of a miniball into the lumen. Another circumstance in which the stent-jacket is placed prior to discharge is in order to allow the magnets on the stent-jacket to assist in minimizing if not eliminating rebound and in retaining the implanted miniballs in position.

Provided the muzzle-head contacts the lumen wall round and about, even eccentric discharge, or discharge not force counterbalanced, produces no appreciable abrupt accelerations, jerking, or recoil as might shake loose a miniball that had become trapped between the muzzle-port and the lumen wall. Yet another reason for placing the stent-jacket prior to initiating discharge is to take advantage of the site-highlighting radiopacity of a tantalum-coated stent jacket. The loss of a miniball from the recovery magnet antechambers at the front of the muzzle-head is precluded both by the pull of the electromagnets and the fact that the antechambers are closed off by spring-loaded doors. When a trapped miniball could be lost due to brushing against the lumen wall or due to jerking of the muzzle-head, the resting field strength must be increased. In this circumstance, to reduce the risk of dislodging implanted miniballs, the stent-jacket is placed prior to withdrawal.

### Sequence of Stent-Jacket Placement and Implantation in Relation to Trap-Extractor Electomagnet Field Intensity

To avoid abrupt shifts of the muzzle-head as it moves among the magnetic fields of the bar magnets mounted to the surrounding stent-jacket, the stent-jacket is usually placed after the miniballs. Otherwise, sudden displacement and differential compression occur regardless how axially centered and flush to the internal surface of the lumen wall the muzzle-head appears fluoroscopically. Nevertheless, there are instances in which it is preferable to place the stent-jacket before beginning implantation. The attractive force on the muzzle-head can be used to assist flush placement of the muzzle-head against the lumen wall. The effect of the bar magnets on the muzzle-head can, however, largely be moderated or neutralized by varying the polarity and intensity of the fields generated by the trap-extractor magnets.

So long as compression is not such as to preclude implantation altogether and does not produce a nonuniformity or eccentricity in the ability of the wall to respond to a given impact force that exceeds the range when uniform, the nonuniformity is treated no differently than is differences in the effect of impact with a given force due to variation as the result of pathology. When a nerve or vessel adjacent to the vessel or duct to be implanted would be especially vulnerable to injury in the event of a puncture, the stent-jacket should be placed prior to implantation. Placing the stent-jacket before effecting implantation is an effective method for reducing this possibility, but is not recommended when this obstructs a clear view of the work area. In some instances, it will be desirable to place the stent jacket first because a segment of the vessel or duct wall is in a weakened state and the stent-jacket prevents puncture of the vessel or a strike against an adjacent structure. Empirical procedure is mandated by the variables, which include the mechanical properties of the tissue to be implanted as altered by the pathology, making careful examination of the result of the intial discharge important. A transfer-molded muzzle-head of polytetrafluoroehylene would avert both the unfavorable as well as the beneficial effects of magnetic attraction to include the use of an external electromagnet to assist in steering the muzzle-head through tighter curves or to bring the muzzle-head into contact or closer contact with the tissue to be treated.

Such use of an external electromagnet need not and must not continue once miniballs have been implanted. If the pathology presages complications due to excessive circumferential nonuniformity in the lumen wall, then the stent-jacket, rather than contributing yet another basis of nonuniformity, should be placed after implantation. Placing the stent-jacket only after the ballistic placement of the intravascular component has been completed allows adjustment to respond to a complication that may result during that process. For example, while a puncture that resulted in bleeding would seal itself in short order with little risk of clotting obstruction, wetting the interior of the stent-jacket with a hemostatic solution or coagulant before placement would serve to obturate the puncture.

This application of a topical coagulant to the exterior of the vessel would exert no effect upon the concurrent use of heparin or other anticoagulants in the circulation to reduce the risk of thrombogenesis. Since the procedure is not continued until the results of the first discharge have been evaluated, and the displacement along the length of the ductus would usually be the same or almost the same for the set of miniballs in the discharge, the same applies regardless of whether a two or four-way discharge were involved. Dealing with exigencies thus does not impose the need for any kind of invasive technique that was not a part of the minimally invasive procedure as originally conceived.

While disease process-induced circumferential or longitudinal eccentricities in the mechanical properties of the vessel wall following angioplasty may be a consideration to the extent of the kind of intraluminal stent deployed, such stents are circumferentially uniform and radially symmetrical, so that all portions of the lumen lining surrounding the stent are treated nondifferentially. By contrast, using the various implementations of the means described herein, the condition of the lumen interior can be differentially treated, recommending detailed examination. The availability of intravascular ultrasound (NUS) to allow evaluation of the vessel wall may be a valuable adjunct to angioscopy, which allows evaluation of the lumen, except this generally adds five to fifteen minutes to the procedure.

When the risk of puncture is considered too slight to warrant this time, and response by sealing any puncture with the stent-jacket is considered a sufficient countermeasure, then an initial test discharge into the affected area is performed without such viewing. Local inflammation along the penetration path or trajectory cooperating with clotting, a pinhole-sized puncture through the wall of an artery is quickly sealed, even with anticoagulant and blood pressure-reducing medication administered. Unless immediately retrievable or posing a risk of problematic enstonement, errant miniballs as may have dropped into or landed in the pericardial space or a body cavity remain innocuous as boinert are best disregarded.

Whether due to pathology, the apparatus, or both, if the initial discharge of the airgun reveals nonuniformities in the impact force required to achieve implantation at various points or arcuate segments about the lumen wall that exceeds the range for proper implantation, the eccentricity is dealt with out withdrawing the barrel-assembly but rather by changing the rotary magazine clip in the airgun chamber to allow separate implantation into the differing arcuate segments about the lumen circumference. The exit velocity can be adjusted for each rotary magazine clip. With the barrel-assembly in place, instead of the full complement of four miniballs per discharge, rotary magazine clips that allow only one to three of the barrels to be used at a time can be exchanged.

Thus, with the four-way radially discharging barrel-assembly having already been introduced into the lumen, this allows a one-way to three-way discharge with one rotary magazine clip, and a second one-way to three-way discharge with a different rotary magazine clip. Using this method of separate discharges using the different barrels of the barrel-assembly allows implantation of different circumferential segments at the same longitudinal displacement along the ductus where one impact force and size and mass of miniballs can be delivered to certain circumferential arcuate segments with one force of impact and another segment can be implanted with another impact force or with differently sized miniballs, which may be alike or different in mass. Vascular lesions almost always eccentric, each procedure is best prearranged to avoid the need to withdraw one barrel-assembly and replace it with another.

An ordinary barrel-assembly of a given number of barrels, such as a four-way barrel-assembly, discharges miniballs one each into a quadrant of the surrounding lumen. To target only certain circumferential arcs or smaller areas while avoiding others for eccentricities in the pathology, barrels of a given type barrel-assembly, such as a four-way, can be blanked out. Depending upon the anatomical bends traversed, the barrel-assembly, with or without barrels blanked out at the rotary magazine clip, may be rotatable without twisting, or torqueable. If passage through a tortuous vessel is involved, then a barrel-assembly with turret-motor to rotate the muzzle-head by hard-wired remote control is used. Without a motorized muzzle-head, barrel-assemblies of eccentric configuration can be employed. Muzzle assemblies can be produced with ports in any angular arrangement.

### External Hand-Held Electromagnet

Assistance in steering the muzzle-head can in infrequent instances benefit from the assistance of an external electromagnet. The external hand-held electromagnet is generally of the form manufactured by the Oakley S. Walker Company-Bux Schrader as the MiniMag I hand-held lifting magnet, but smaller. The ability of a magnet to draw an object is bistable, in that a certain threshold field strength is attained at which the object is drawn; to gradually vary the field strength will not gradually draw the object. However, since the objects to be drawn, the muzzle-head and miniballs, are within the body, excessive field strength is to be avoided as posing the risk of intravascular injury. For operative purposes, it is preferable to find the threshold value for attracting the object not by gradually moving closer to the treatment site but rather by equipping the hand-held electromagnet with a rheostat to continuously vary the field strength.

### Use of Barrel-Assemblies

Even though ideally the muzzle-head lightly contacts the lumen wall during discharge, the pressurized air forced before the discharging miniballs must be given a path of least resistance to prevent air from being forced into the bloodstream upon discharge. At the same time, to prevent the back-flow into the barrel ports of blood when the muzzle-head is immersed in the bloodstream, resistance must be posed to the displacement of the air in the muzzle. In other words, the flow of gas must be biased in favor of nondisplacement from without while at rest and in favor of recursion back into the barrel-assembly under conditions of the sudden pressurization of expulsion.

The volume of air in the barrel-assembly and chamber, together airtight except through the barrel, is constant. The higher pressure of the blood and angle of entry make complete prevention of blood backflow through the muzzle-ports at the moment of immersion difficult without barricading the muzzle-ports. Because the gas pressure recursion channels and relief space must become completely filled with blood before resistance to the passage of gas equals that presented by the column of blood at the muzzle-ports, blockage for this reason midprocedure is unlikely. Should the mechanism become fouled, the barrel-assembly is withdrawn and purged with pressurized distilled water.

In most instances, because the barrel-assembly is airtight, this is accomplished by placing a finger over the muzzle-ports facing upwards and thus not allowing air to be displaced by blood. When the placement of the muzzle-ports does not allow these to be blocked to the air with a fingertip, the muzzle-head is dipped into distilled water so that the muzzle-ports are filmed over by surface tension. The muzzle-head is then stored in a freezer. To be certain that the film does not break by cold contracture and is sufficiently thick, dipping and freezing may be repeated several times. Upon insertion in the bloodstream, an interval is allowed for the temperature of the blood to melt the film of ice and the muzzle-head to assume body temperature.

In an embodiment that includes side-sweepers, the thermal expansion wires used to raise the lift-gate brush handles can be sent current to accelerate melting. Whether the brushes remain deployed during transluminal movement to the diseased portion of the ductus is at the discretion of the operator. Initial transluminal movement is usually to a point beyond the segment of the ductus to be treated with the procedure carried out in withdrawal with movement over larger distances directly manual, over small distances by manual control of the linear positioning table stepper motor, and discharge over lesions by manual direction of automatic sequences. Once immersed in the bloodstream, the airtightness of the barrel-assembly prevents the inflow of blood.

The single barrel or simple pipe barrel-assembly is not for use in the circulatory system. The barrel-tubes in a barrel-assembly with two or more barrels are perforated, removing barriers within the spaces defined by the different tubes within the barrel-assembly. The perforations present no burrs or irregularities on the inner surface of the barrel-tubes, which must be smooth. The air throughout the barrel-assembly now in communication or effectively continuous, the displacement of air anywhere within this space is minimally resistant to internal movement or redistribution, minimizing recoil externally in relation to the vessel and internally in relation to the likelihood of dislodging other miniballs on the same rotary magazine clip. As seen in the cross section view of FIGS. XX, pressure equalization (pressure relief, pressure diversion) channels are drilled through the muzzle exit ports so as to diverge or bifurcate in the proximal or backward direction from the muzzle barrels.

The outflow of these pressure diversion channels is directed proximally or backwards to the canal formed by their convergence which is continuous with the central canal amid the barrel-tubes. Following puncture and expansion of a vessel, the heparine-saline solution wetted muzzle-head is introduced and advanced to the sites slightly short of preceding angioplastic treatment. that will result in the shots, which are discharged at an acute angle, coming to rest beneath the atheroma and plaque removed. The muzzle-head is chosen in a size equal or slightly larger in diameter than the internal diameter of the segment of the vessel short of the area to receive the miniball implants by the length of the trajectory. Since the placement of a conventional intraluminal stent may squeeze away remaining plaque, the preliminary angioplasty should be thorough.

### Procedure for the Extraluminal Stenting of a Smaller Vas Using the Two to Four Way Apparatus Described Herein

In intravascular applications, the decision tree and techniques routinely attendant upon angioplasty and stenting, such as whether to enter percutaneously through the brachial or femoral artery, or through open exposure of the femoral, popliteal, or brachial artery; whether to use a particular combination of fluoroscopy or biplane fluoroscopy, angioscopy, intravascular ultrasound, magnetic resonance angiography, carbon dioxide angiography, endovascular ultrasonography, or spiral computed tomography; the intravenous administration of antibiotics and anticoagulants such as 20,000 units of heparin and 81 milligrams of aspirin, 1% lidocaine (lignocaine) injection in the puncture area; incision through a 1 to 2 mm stab wound with a Number 11 scalpel; the use of a mosquito clamp to expand the puncture site; of open arterial access to avert embolization and control outflow arteries; and so on remain unaffected. Here, however, the entry site is widened to admit the muzzle-head and barrel-catheter. The combination of angioscopy and intravascular ultrasound computerized processing is recommended as allowing greater accuracy than does any external imaging technique.

In intravascular use, as the site of the lesion is approached, the introduction and advancement of the apparatus provides peripheral blood grooves to serve much as the blood flow or perfusion side-holes in catheters or; as with a baloon, deflation to reduce the diameter, for preventing the complete cessation of blood flow along its length. Nevertheless, by keeping operating time to a minimum, the risks of thrombogenesis and ischemia are reduced. To allow operative speed, the interventional airguns to be described are designed for semiautomatic repeat action radial discharge of from one to four or more miniball implants and adjustability to the exact exit velocity sought without criticality in the use of one control. To achieve this, the apparatus intercepts the means of airgun propulsive force development at numerous points and introduces a control at each. Also to avert thrombogenesis, heparin alone or in combination with the drugs specified above is administered until the activated clotting or coagulation time (ACT) rises above 300, as has been routine with two guidewires and a baloon in use. At the same time, the dosage of anticoagulant must seek to avoid complications at the entry site. Also routine is the application of the anticoagulant to the tip of the catheter, here the muzzle-head of the barrel-assembly. Preoxygenation through an oxygen mask is recommended.

### The Airgun

From one distance along the ductus to the next, the tissue of a lumen wall can be either substantially uniform in mechanical properties, as is usually true of the subacute or pre-Grade IV collapsed trachea, or variable, as when vascular disease has differentially affected different portions of the arterial or venous wall. For the trachea, which is relatively large in internal diameter, a single miniball discharging airgun is appropriate. In the trachea, the miniballs are implanted along a dorsolateral longitudinal line in relation to the cartilage rings, relatively seldom at other points about the lumen circumference, and the working space affords maneuverability. In advanced collapse where secondary inflammation and infection may have altered the mechanical properties of the tissue, miniballs of one kind are implanted in one pass, and those different in a second pass.

Alternatively, if distinctions in mechanical properties of the tracheal tissue are present due to a different level of expression of primary pathology or due to unrelated comorbidity with areas that exhibit various conditions, then the single-shot airgun load queue can be strictly sequenced according to a prescribed load list to include miniballs of different mass coated with different medications. Of these approaches, the first is to be preferred as minimizing the need for frequent adjustment of the airgun propulsive force resulting in a longer operation with increased possibility of errors. Because the barrel-assembly will obstruct blood flow, the time of any procedure in arteries, the coronary arteries in particular, is acutely time sensitive. While in the bloodstream, the barrel-assembly must not be discharged unloaded.

Unlike the rotary magazine clip which makes it possible to apply a consistent propulsive force to miniballs that differ in mass by means of securing each miniball in its clip hole with a dried solution or syrup of sugars, corn starch, or molasses (treacle) of a formulation and thickness to offset the lesser resistance to expulsion of any miniball or miniballs in a set to be discharged together, a single-shot airgun must be adjusted in propulsive force every time there is a change in the mass of the miniball to be ejected. With a rotary clip, the propulsive force may have to be varied with the sum of miniball masses. The solution or syrup is run about the groove formed by the perimeter of the miniballs and rotary clip holes by surface tension, and to prevent debris from moving down the barrel, the composition of this adhesive should have good self-adhesion.

Except in hole pattern, which is generally for a group of miniballs rather than one, the rotary magazine clips are conventional. Except in the circulatory system, where once introduced the one barrel-assembly, even though demanding adjustment for different miniballs, should not be removed and replaced, changes in miniball mass are accomplished by withdrawing the barrel-assembly and introducing one of another airgun. Alternatively, the barrel-catheter can be removed and one of different caliber connected to a second airgun of like caliber. While prior to fixation of the miniball within the rotary clip ring hole and as a separate operation, medication can be applied in a sugar or syrup coating that has been heat- or freeze-dried, the miniballs are produced to meet such special requirements. The construction of the airgun includes a conventional rocker-arm stop that prevents the premature entry of a miniball into the barrel at the same time that it prevents a second miniball from partially entering the chamber before the chambered miniball has been expelled.

A single ‘shot’ semiautomatic airgun can be queued with miniballs of like caliber but different mass, but were differences in mass to exceed the range over which the impact force would implant the miniball subadventitially without unacceptable under- or overshooting, the procedure must be interrupted to adjust the propulsive force. To adjust the single ‘shot’ airgun after one or a few discharges, is, however, not recommended as contrary to minimizing operative time; to minimize the number of adjustments, hence time necessary, all implant discharges of like mass are completed before proceeding to miniballs of different mass. Using a simple airgun, the propulsive force can be adjusted after the complement of miniballs of like mass have been implanted or the barrel-assembly, which in the trachea consists of a simple catheter pipe barrel, is left in the patient, and the proximal end of the barrel-assembly is detached from the airgun and inserted into another airgun preadjusted to discharge miniballs of different mass.

To change the caliber, however, requires withdrawing the barrel-catheter and inserting another of the new caliber. The same airgun can be adjusted or another already connected to the barrel-catheter can be used. Using a single shot airgun, the more complicated matters of mixing miniballs of different mass in a single shot as differentially distributing the propulsive force does not arise, because the airgun is not capable of multiball discharge. At the extreme of demand is the diseased coronary vessel where to avert the risks posed by interruption in the circulation much less the use of a heart and lung machine, the time of the procedure must be kept to a minimum under considerably more difficult working conditions. This justifies the expense of certain refinements that make it possible to discharge multiple miniballs simultaneously in a radial pattern, to do this with quick repeatability, and to change the exit velocity to a value desired quickly by alternative means of control used individually or in combination. This is accomplished by a radial discharge muzzle-head that delivers a plurality of miniballs, usually four, one each into each quadrant of the circumference.

The requirement to vent the air inside the barrels with the muzzle-head immersed in the bloodstream without introducing any gas into the bloodstream precludes the use of side holes as allow the blood to continue to flow if obstructed in guide catheters. Such an airgun is loaded by inserting the miniballs in a rotary or wheel magazine clip, that indexes the miniballs into ‘firing’ position in front of the propulsive gas outlet. The barrel-assembly built as an integral unit, and the muzzle-head usually radially symmetrical in internal structure, the caliber of the barrel-tubes and their respective muzzle-head exit ports in any one muzzle assembly are the same, even though specialized rotary magazine clips and barrel-assemblies could be made to combine different calibers in each discharge. Any change in the caliber or mass of one or more miniballs in the set to be discharged together must be offset by adjusting the resistance to propulsion of each miniball by changing the consistency of ingredients of a quick-dried syrup used to hold or clinch the miniballs in the rotary clip holes.

Every rotary magazine clip loaded into the airgun should be visually inspected and lightly shaked to be certain no miniball is loose. Vigilance exercised, premature entry of a miniball into a barrel is unlikely to result from looseness in the rotary clip hole, but rather because of imperceptible inequalities in the clip hold retentiveness of the miniballs of a set to be discharged at the same time, which can allow the propulsive gas to leave chambered or dislodge miniballs other than one offering critically less resistance. The improper apportionment of resistance to expulsion is minimized though rigorous testing and tight quality control. The miniball is retrieved by disconnecting the barrel-assembly from the airgun and passing a mildly magnetized guidewire down the barrel-tube. The calibers of the miniballs in a given rotary magazine clip and the barrel-assembly must match, individual discharges that include miniballs different in caliber requiring the use of a special purpose clip and barrel-assembly to match these in caliber.

Differences in mass for any reason, to include the addition of an outer layer to deliver medication or radiation, can be accommodated in single discharges. Since the propulsive gas will find the path of least resistance, miniballs of less mass must be equalized in clip hole retention by syrup bonding. Simultaneous discharge assumes that the distinction in mass is negligible; if the difference in mass is significant, the more massive miniball may not be propelled at all. The parallel use of different airguns to propel the miniballs in the different holes of the discharge set is not contemplated. Reloading by inserting a new rotary clip or by detatching the barrel-assembly from one airgun and inserting it into another can be done quickly. To change the caliber, however, requires withdrawing the barrel-assembly and introducing another, and this negates the practicality in an airgun with multiple output ports or barrel-assembly fittings of different caliber. With the barrel-assembly exchanged, a rotary clip containing miniballs of different caliber can be inserted in the same airgun. Barrel-assemblies and rotary clips can be produced to discharge from one to four or more miniballs in a radial pattern.

The ability to produce barrel-assemblies and matching rotary clips other than radially symmetrical increases the lower is the number of barrels or barrel-tubes. In situations where the number of miniballs to be discharged at a time changes, proceeding with the barrel-assembly already in position is preferable to withdrawal and insertion of another. Changing the number of miniballs to be discharged at once is physically similar to differences in mass among a complete set, which likewise differentially distributes the sum propulsive force. In this case, however, the difference in mass will require an adjustment in propulsive force, which may take the form of adjusting the airgun in use or switching to an airgun preset to the required value. When the number of miniballs to be discharged at one time is less than the number of barrel-tubes in the barrel-assembly, a miniball mounting hole on the rotary clip is not left vacant but rather reduced in diameter to bring the force applied to the miniballs that are present to a sum value within the range that this would have been were a miniball present.

### Modification of Marketed Airguns

Certain air pistols modified as specified below are loaded or fed miniballs from a line-feed type magazine clip. These are limited in use to single miniball or monobarrel discharge, specifically, simple pipe and single miniball radial discharge barrel-assemblies. An airgun that uses a rotary magazine clip can accept any barrel-assembly whether a simple pipe or a multibarrel radial discharge barrel-assembly. The first of the two modified commercially available air pistols now to be described uses a queue or linear sequential spring-loaded clip and is therefore suitable for use with monobarrel barrel-assemblies whether of simple pipe or radial discharge type. The more capable air pistol to follow achieves greater versatility by virtue of incorporating a rotary magazine clip, which allows either a single or a number of miniballs to be discharged at one time and thus the ability to support either a monobarrel or multibarrel tube barrel-assembly.

Additionally, a rotary magazine clip type airgun allows the portion of the airgun overlying the chamber to be removed and replaced with transparent plastic allowing the failure of one or more of a set of miniballs to be discharged at once to be seen. However, the discharge mechanism of an airgun that loads queued miniballs one at a time is incapable of multiple miniball discharge, and the failure of a single miniball to discharge would be immediately evident without such viewability. A rotary clip makes possible the projection of multiple miniballs per discharge and therewith. Whereas the successful projection of a single miniball is instantly evident, the failure of one of four to be propelled is not. Fluoroscopy and angioscopy used to confirm the placement of miniballs, it is additionally helpful to have the chamber retrofitted with a roof made of a suitable transparent polymer such as polycarbonate. This allows inevidence of a miniball to implant properly to be immediately traced to a failure within the airgun rather than loss in the patient.

Simple Airgun with Limited Application

Monobarrel capability is adequate for any application for which single discharge is suited, notably in larger parts of the airway with a simple pipe, or in any ductus that is too small in lumen diameter to admit a simple pipe, with the radial discharge barrel-assembly. Rather than an electrical plunger switch as trigger, a modified commercially available air pistol (hand airgun, air handgun) uses the original triggering mechanism and has mounted along the slide, or in a model that has no slide, the corresponding location, a potentiometer to adjust the current, hence, the field strength of the tractive recovery electromagnet or alternatively, a three-way toggle switch with settings for recovery magnet off, retrieve (a dropped miniball), and retract (a mispositioned miniball).

With the barrel-catheter in a simple pipe representing the only barrel, no course through the interior of the barrel-catheter is available for the conductors, which would obstruct the miniballs. At the same time, the external surface of the simple pipe will come into contact with the endothelial lining of the airway. For this reason, the simple pipe-type barrel-assembly is connected to the airgun battery by wires that are fine in gauge and attached to the outside of the barrel-catheter in virtually flush condition by means of thin nonallergenic adhesive tape with connection as shown in FIG. 40. The battery used to power the electromagnet is mounted to the inside of the pistol grip and for comfort, is of rectangular conformation as seen in a common nine volt battery.

Energization of the electromagnet XX is similar to that described above in the section entitled Stent-stay insertion (injection) tools. The primary object in such an embodiment is to provide veterinary specialists with a simple and relatively inexpensive hand-held airgun that with suitable imaging equipment and a barrel-assembly with clearly visible (bright, radiolucent) markers can be used to ameliorate the intermittent aphyxia (suffocation) or airway throttling that is symptomatic of collapsed trachea in small dogs without the need for a thoracotomy. The simplest airgun usable with the methods described herein delivers one miniball per discharge and connected to a simple pipe barrel-assembly, is suitable only for procedures in structures such as the trachea, while connected to single-barrel radial discharge barrel-assembly or monobarrel, is suitable for use in closed ducts and vessels.

Ducti that are relatively large in diameter and open to the exterior afford greater accessibility and maneuverability but exhibit differentiated histological and anatomical structure. Speed in the airway must always remain consistent with a distinct aiming capability. The lumen of a closed vessel that is additionally diseased is accessed with relative difficulty, affords significantly less maneuverability, and is substantially undifferentitated with respect to its normal condition. The closed vessel therefore poses the opposite set of factors for attaining operative speed. For the latter, speed that comes with multiple simultaneous radial discharge of miniballs in rapid succession. A modified airgun of the queue loaded or line-fed type is not suitable for applications in the vascular system where the ductus is closed to the exterior, the lumen diameter is usually two or three millimeters, and the disruption to the delivery to the cells of oxygen by the circulation calls for completion of the procedure in the least amount of time.

A single shot semiautomatic repeat action airgun can be provided by modifying an off-the-shelf or commercially available hand airgun in bore to project small caliber miniballs and allow the propulsive force to be variably controlled. The modification of commercially available airguns must be carried out by trained personnel under stringent quality control. Of airguns currently available, the Daisy 93/693 is suitable as clip-loading 15 shots, minimizing interruption for reloading midprocedure and as not presenting a reciprocating slide to interfere with the addition of a permanently positioned control lever. The break breech design also makes the insertion of a testing rod shortly to be described simpler, the rod inserted at the back of the barrel, making insertion of the key easier.

Such an airgun can be provided by modifying an off-the-shelf or commercially available semiautomatic repeat action single miniball discharge hand airgun to project small caliber miniballs. The modification of commercially available airguns must be carried out by trained personnel in a special facility subject to stringent quality control. Such modification is discussed with greatest applicably in generic terms, actual models available being many, differing in inconsequential details, and constantly subject to discontinuation, while new models are frequently introduced. There are two major types of off-the-shelf or commercially available single miniball discharging hand airguns, the first made to discharge miniballs, the second pellets. Both kinds accomplish repeat action semiautomatically, the first by admitting or feeding one miniball at a time into the chamber from a queue or line contained within a spring-loaded loading clip into the chamber by the action of the preceding discharge.

A rocker check arm prevents the entry into the chamber of more than one miniball at a time. The adaptation of higher power airguns allows higher exit velocities for resistive diseased tissue, such as that considerably calcified or ossified in patients for whom resection is not advisable. The higher propulsive force of such airguns is readily reduced to any lesser force by bleeding off the propulsive gas as will be described or by increasing the rolling resistance. No suggestion is intended that less forceful airguns, referred to as ‘airsoft’ or ‘softair,’ or that use the force of a spring to propel the spherule or ‘BB,’ or that are used to play paintball cannot be adapted for applications that demand impact forces less than the maximum for the airguns specified.

Representative of the many spring-clip line-fed miniball hand airguns are those manufactured by Maruzen Kabushiki Kaisha according to the specifications of and sold by the Daisy Outdoor Products Company. That bearing model number 15XT requires loading the miniballs one at a time; model number 93 since discontinued; and model number 693, now redesignated model number 93/693, which is clip loaded, and the Crosman-Walther PPK/S, all semiautomatic 4.5 millimeter (0.177 caliber) and powered by a 12 gram CO₂ cylinder referred to as a ‘powerlet’ or ‘pistolet.’ An advantage of using the spring-loaded line feeding type clip to chamber successive miniballs semiautomatically, airguns designed to discharge miniballs rather than pellets are capable of a larger number of successive discharges or ‘shots’ without reloading, typically fifteen.

Some makes or models of line-fed or spring loaded linear queue type magazine clip loaded miniball hand airguns, such as the Industrias el Gamo V3, also semiautomatic, 4.5 millimeter (0.177 caliber), and 12 gram CO₂ cylinder-powered, incorporate a triggering mechanism that to simulate the appearance and action of an actual automatic pistol firearm, includes a reciprocating slide that travels over the valve body and chamber, which are integral to the clip at the top thereof, the valve body above the gas cylinder and the chamber above the miniball queue. This slide does not preclude the retrofitting of a simple low-cost control mechanism presenting a lever to the outside of the airgun to allow the propulsive force or exit velocity to be adjusted without the need to remove the clip; however, because two layers of plastic slide past one another, the view into the chamber even with transparent material is obscured. The modification of any commercial airgun includes the mounting to the front end of the muzzle of a barrel-assembly connecting socket as described above.

To modify the Gamo-type mechanism is more difficult than the models that do not have a reciprocating slide. Daisy model 15X Thas no reciprocating slide making it easier to modify, but unlike Daisy model 93/693, is not clip loaded, instead requiring that the miniballs be loaded one at a time, effectively necessitating that several be preloaded for any one procedure. Clip loaded and lacking a slide, Daisy model 93/693 is superior for use with a simple pipe barrel-catheter for tracheobronchial procedures. While it is easily within the capability of a rotary magazine clip airgun to be described, to use different caliber miniballs with one and the same spring-loaded queue fed airgun would require changing the inserts throughout the miniball delivery path inviting inaccuracy and malfunction. Such a low cost embodiment is preferably sold as permanently modified for use with a fixed caliber not to be changed by the purchaser.

### Modified Simple Airgun of Wider Application

The second type of airgun, some originally made to shoot pellets, uses a rotary magazine or wheel clip that typically provides fewer discharges than the line-loaded type clip, typically six successive ‘shots’ or discharges, before a spent clip must be replaced with a loaded one; however, replacing a spent rotary clip takes but a moment. Modified as described below, the rotary magazine clip can be used to discharge from one to four or more miniballs per discharge, making it considerably more versatile for realizing the objects stated above. As previously described, using either type of clip, different miniballs can be variously coated to deliver medication or radiation. A means for adapting either a spring-loaded queue or a rotary magazine clip type airgun with a testing mechanism is described under the heading ‘universal means of testing’ that follows. Rotary clip airguns made in the form of rifles rather than handguns generally use rotary magazine clips that typically hold twelve pellets, and therefore use rotary magazine clips that are larger in diameter, affording a larger number of multiple miniball discharges and reducing the frequency of reloading regardless of the fact that custom clips are used.

An example is the Crosman Model 1077, likewise semiautomatic, 4.5 millimeter (0.177 caliber), and powered by a 12 gram CO₂ cylinder, with an AirSource® adaptor available for 88 gram (3.1 ounce) AirSource® cylinders. Yet other airguns, such as the model 617X made by Maruzen Kabushiki Kaisha according to the specification of and sold by the Daisy Outdoor Products Company, are available that are capable of shooting either miniballs or pellets, the added capability due to the mere incorporation into the rotary clips of a slight circumferential ridge to prevent miniballs from rolling out into the barrel before discharge. All airguns that are able to discharge either pellets or miniballs use rotary clips that hold either miniballs or pellets of like caliber; none changes the repeat action to switch from clip rotation to discharge pellets to fixing the rotary clip in position for the opening aligned to the valve body outlet to serve as a miniball chamber.

Daisy model number 622X, which shoots 0.22 caliber pellets, is identical to Daisy model number 617X, which shoots 0.177 caliber pellets or miniballs using the same rotary clips, and except for the fact that the rotary magazine clips supplied for the 622X lack a slight circumferential ridge at half the distance through the bore, would be equally able to shoot 0.22 caliber miniballs using the same clips. Provided with a slightly larger caliber, the 622X also uses clips that larger in diameter, afford greater latitude in the multiple miniball sets per discharge and caliber of the miniballs that can be accommodated in the custom rotary magazine clips to replace the original. The following description of the modifications essential to make a commercially available airgun suitable for the repair of tracheal collapse by veterinarians as previously described presupposes a semiautomatic repeat action hand airgun wherein the miniballs are successively forced up into the chamber by a spring-loaded line-feed clip.

To this end, the models specified above are mentioned in a purely exemplary sense; similar airguns produced by several manufacturers exhibiting much the same construction and capable of being modified to serve the present object equally well. Modification of the existing hand airgun is accomplished by placing caliber-reducing polytetrafluoroethylene tube inserts in the spring-fed clip and barrel, and a caliber-reducing liner plugged into the chamber loading tube, which is situated toward the forward end of and integral with the clip. The clip insert requires that the spring and plunger that drives the line of miniballs upwards into the chamber when the rocker arm lifts be replaced by proportionately smaller versions. Reducing the caliber from the original 4.5 millimeters to 1.0 millimeter, for example, increases the number of miniballs that can be loaded from 15 to 67. The number of cartilage rings in the dog trachea being 40 plus or minus 5, an extensive procedure necessitates reloading twice.

The quickest way to accomplish this mid-procedure is to disconnect the barrel cathether from the airgun without removing it from the patient and reconnecting it to another fully loaded airgun, whereupon the first airgun is reloaded. A smaller hole through which to reload miniballs and a side slot to return the spring and allow the number of miniballs loaded to be seen must be cut into this tube, which is positioned concentric to the original loading hole or entry into the chamber at the top of the chamber loading tube. If the wall thickness of the inserts placed in the barrel and miniball feeding channel in the spring-loaded line-feed clip in the grip does not center these as concentric within the original diameters, then tape is used at intervals along the length about the circumference to achieve a snug fit. Alternatively thin sheet of a polymer with a low coefficient of friction such as polytetrafluoroethylene can be wrapped about the insert tubing to avoid the bunching up at the entry experienced with other materials.

The barrel insert tubes used to reduce the caliber of the barrel stops half way down the barrel to allow the proximal end of the barrel-assembly to be inserted. The insertion of the caliber-reducing polytetrafluoroethylene tube insert in the barrel covers over the rifling. The chamber insert liner properly centers the smaller miniball in relation to the propulsive gas entry hole directed to its apex at the rear, the miniball entry hole in the chamber floor, and the barrel to the fore. The small finger at the top of the loading spring that pushes the last miniball up into the chamber through the floor must be replaced with one that is longer to pass the smaller miniball up through the hole in the chamber floor which has been made thicker by the liner. The original rocker check arm that admits only a single ball into the chamber at a time at the front bottom of the chamber must be removed and replaced with another proportionately smaller in size in the equivalent position to lap over the exit or muzzleward hole floor drop off at the center front of the chamber insert liner.

The chamber insert liner has a hole at the center of the rear through which the propulsive gas is released from the valve body directly against the rear of and causing the miniball to travel down the barrel. This hole is proportionately smaller than the original hole behind it, the two holes positioned in flush concentric relation. The small CO₂ cylinder or canister that fits into the clip adjacent to the spring-loaded miniball feed line is engaged by forcing it up against a hypodermic-type inlet pipe of the valve body at the bottom thereof by means of a screw beneath the cylinder. This connection by intermission affords no junction as would allow the insertion of a valve or regulator. This leaves the components that affect the propulsive force after the propulsive CO₂ has entered the valve body and chamber to introduce means for adjusting the propulsive force and therewith the exit velocity.

Of the various means for effecting a reduction in the exit velocity in such a retrofit of a manufactured airgun, examined from the standpoint of greatest simplicity have been reducing the delivery of propulsive gas from the valve body as affected by the time and force of depression of the valve pin by the hammer. Others have been to effectively increase the volumetric dimensions of the valve body and so reduce the pressure inside of it by means of a bleed slot continuously variable in area, a similar slot cut into the chamber, and lengthening and curving the barrel-assembly to increase rolling resistance disproportionately to the increased propulsive force of the extention in barrel length represented by the barrel-assembly.

Of these possible points of interception to obtain the control necessary, replacing the hammer with a push-type solenoid of which the striking force and time is variable and introducing a bleed opening with adjustable cover in the chamber have been discounted as needlessly complex and costly in a modified airgun intended to be merchantable at relatively low cost. The former is employed in more precise multipurpose embodiments originally produced to obtain the present objects, and the latter discounted as resulting in a mechanism so tiny as to be too difficult to readily adjust and test manually during an interventional procedure. Introducing a slot with sliding cover in the side of the valve body, however, allows a mechanism sufficiently large for practical use and can be added at reasonable expense.

Control of Propulsive Force or Exit Velocity by Means of a Calibrated Slide Cover Over a Slot Cut into the Valve Body

In modifying an airgun for the present purposes, the internal structure of the valve body remains unaffected, the addition of an external control being less complicated and more readily accomplished. That the modification is least variable from one model airgun to the next is a significant factor in reducing errors in preparation. As seen in Fig. XX, a slot covering an area to deplete the pressure delivered from the valve body to a value below that required to produce the miniball impact force desired, such as 2 millimeters in width and 1.5 centimeters in length, is cut longitudinally into the valve body toward its front end. The dimensions of the slot depend upon the pressure in the valve body, so that, for example, the slot in a Crosman 1077 based modification would be larger than that in a Daisy 622X. The 12 gram cylinders in standardized use with commercially available miniball airguns deliver CO₂ at a pressure of 837 psi at 70 degrees Fahrenheit; however, the exact dimensions of the slot depend upon the volume of the valve body or its adjustment by the maker, which varies from one model airgun to the next.

In an airgun with a reciprocating slide, the valve body is integral with the CO₂ cylinder beneath it in the clip, the lever must be disengagable or foldable to allow the clip to be removed from the grip for reloading, or a pin must be placed through a slot and into a depression in the slide. A slide frame or slideway in which the slide will be contained and longitudinally slid to continuously open and close the slot in a manner similar to some flat sliding door bolts is, in a retrofit as opposed to an original design, applied to the outer surface of the valve body. The slideway is produced by die cutting and mold pressing thin stainless steel sheet to the curvature of the valve body. The sides or wings of the slideway extend outward enough to include a calibration in the pressing. A die-cut rectangular slide or tang is pressed to flush conform to the curvature of the valve body side wall and the slide frame. The slide includes a calibration mark and a hole in which to insert and fasten a small control handle, the extension of the handle on the underside acting as a stop.

To minimize sticking resistance, the slideway and slide or tang may be given a thin coating of polytetrafluoroethylene; however, the closeness of fit of the slideway and slide must be such that resistance to being slid must never allow the sudden jolt of discharge to displace the slide. The slide is positioned over the slot and covered over by the slideway, which is then fastened to the valve body by blind or pop rivets of 1.0-20 millimeters in flange outer diameter, in any of several types manufactured by Textron Fastening Systems, Emhart Division of Black and Decker, and other manufacturers, or threaded inserts made by Emhart. The slide is now retained within the slide frame against the outer surface of the valve body so as to freely slide forward and backward over the slot. Retracting the slide uncovers the slot from a fully closed hermetically sealed position to a continuously variable open position that allows CO₂ to bleed out of the valve body reducing the propulsive force driving the miniball through the barrel.

The use of engaging depressions or dimples in the slideway and protuberances in the slide to act as detents at the calibration marks is discounted as suggesting that these settings have a favorable status. The height of the slideway above the outside of the valve body is such as not to come into contact with the gun body. In an airgun such as the Gamo V3 with a reciprocating slide on the receiver, a slot can be cut in the side of the airgun body slide to clear a handle of the pressure adjusting slide. However, as a handle would have to be removed or folded to withdraw the clip from the grip, a depression close to its leading edge or slot closing end can be made to allow a pin to be inserted as a removable handle to adjust the slide by aligning the calibration on it to that alongside the slideway and to act as a stop. To introduce a miniature electric motor inside the valve body to move the slide that is powered by a battery and control at the butt of the grip is discounted as inconsistent with the object of providing a simple limited-purpose retrofit at relatively low cost.

The modified airgun is sold with a table relating the calibration to the exit velocity and indicating the range of settings suitable for the tissue to be treated in a patient of given species and size, and providing instructions for measuring and finding the best impact force to use. A printed table that sets forth the settings for a given tissue affected by specific disease is consulted and the airgun adjusted to this setting. The end position of the first implant is carefully observed fluoroscopically and if in a vessel, angioscopically, to confirm the setting and to apply any adjustment needed before proceeding to the next discharge. In use, the table is consulted for the recommended exit velocity and impact force data for the barrel-assembly and miniballs to be used for the tissue to be treated, the airgun is test discharged against impact force registration paper such as Pressurex® produced by Sensor Products, Incorporated, which is a film of Mylar®, E.I. du Pont de Nemours and Company, for biaxially oriented polyethylene terephthalate (boPET) polyester film treated to provide a certain color indicative of the impact force.

For higher resolution, a ballistic pendulum is used to measure the impact force. Then the exit velocity is adjusted by means of the slot slide introduced into the valve body, and the airgun output measured again. This process is repeated until the impact force has been optimized for the tissue within the predictable limits. To expedite adjustment in use, preliminary testing must also plot the relation between the slide adjustment and the impact force. The effect of the initial discharge is carefully examined before proceeding and the exit velocity adjusted accordingly. Such viewability also allows confirming the precise alignment of the barrel-assembly with the configuration of the miniballs in the rotary magazine clip. Airguns in current production that use rotary magazine clips do not have a slide that reciprocates forward and backward along the top of the receiver making it possible to retrofit the chamber with a roof made of a suitable transparent polymer such as polycarbonate.

The simplest airgun for use with a simple pipe barrel-assembly must be equipped with a potentiometer having a control knob where the thumb contacts the grip or alternatively, a three-way toggle switch with settings for recovery magnet off, retrieve (a dropped miniball), and retract (a mispositioned miniball). to adjust the magnetic field strength of either trap-extraction tractive electromagnet in the most distal portion of the muzzle-head from zero to the maximum. For use with a barrel-assembly with a motorized turret to rotate the muzzle-head, a bidirectional rotation control is also required in this location. A critical increase in versatility is provided in commercially available hand airguns suitable for modification that provide semiautomatic operation by means of a rotary magazine clip or wheel clip rather than a single ball advancing spring-loaded feed line. With a rotary magazine clip, multiple miniballs can be positioned in front of the barrel-tubes at a single time. Rotary magazine clips are intended for projecting pellets rather than balls but can of For projecting plural miniballs simultaneously, or multishot semiautomatic, operation, the use of a rotary magazine clip is preferred.

A representative example of such a hand airgun is made by Maruzen Kabushiki Kaisha to the specifications of and sold in the United States by the Daisy Outdoor Products Company under the trade name Powerline 622X. This model is designed to shoot 0.220 inch (5.5 millimeter) pellets. The rotary magazine clips provided with this airgun are designed to hold pellets rather than miniballs, but as is demonstrated by Daisy model 617X requires only the addition of a slight midcircumferential ridge along the internal surface of the hole to achieve retention of a miniball rather than a pellet. Interchangeable the rotary magazine clip mechanism is the more versatile, allowing one to four miniballs to be poised for discharge at a time. If a rotary magazine clip that is conventional in diameter but adapted to hold millimeter miniballs will not allow the number of discharges required for a procedure, then the rotary magazine clip must be replaced during the procedure, an automatic mechanism to remove and replace the rotary magazine clip exceeding the present scope.

### Docking Stations for Modified Commercial Airguns

Since airguns can be specially made for interventional use at no more expense than is necessary to modify a commercial airgun and provide it with a docking station that includes a linear positioning table for automated incremental transluminal movement, the application of modified airguns is limited for practical reasons to applications in the airway and larger ducti, and almost always with a simple pipe.

### Dedicated Interventional Airguns

The object in providing special-purpose or dedicated airguns for interventional use is to reduce operative times by providing multiple points for adjusting the exit velocity or impact force so that this can be accomplished more quickly between successive discharges. To this end, dedicated interventional airguns include means in addition to a slidably adjustable relief slot in the valve body. Adjustment in exit velocity is never without testing the target tissue as described below, and for reasons of safety, this function is never given over to automatic control. Shown in FIGS. XX and XX are interventional airguns with gravity fed queue or sequential miniball loading or feed that are capable of discharging but a single miniball at a time. These airguns are therefore suitable only for use with monobarrel simple pipe-type or radial barrel-assemblies. Monobarrel radial discharge barrel-assemblies are generally unsuited to use in the bloodstream and therefore lack an internal pressure equalizing jacket or enclosure.

These airguns, unlike the fully capable embodiment to be described below, are not provided with positional control. For such use, required are a plunger or dead-man trigger switch, a toggle switch to energize the electromagnet in a simple pipe or either electromagnet in a radial discharge monobarrel from off at the center setting to either ‘Lo’ for recovery of a dropped miniball or ‘Hi’ to extract one that has already been implanted, a potentiometer control knob for adjusting the solenoid timing, and a manually adjustable slide-cover in the valve body. Not required for such use are such features as rotary magazine clips suitable for feeding miniballs to multiple barrels (barrel-tubes) and controls for positioning a linear stage (table) and turret-motor as are required in barrel-assemblies for use in the circulatory system and various ducts.

Such apparatus is for use in the airway or in any ductus for which the distancing between successive implant discharges does not demand a level of precision beyond that attainable by hand. As shown, these monobarrel supporting guns are gravity fed, a conventional rocker arm (not shown) used to admit one miniball into the chamber at a time. More points of control are provided than in a modified air pistol as described above. Manual positioning necessitates some freedom of movement, and as with a modified air pistol, this is attained by providing adjustability in the exit velocity to compensate for some bending in the barrel-assembly. Such presumes that the operator has fully tested the exit velocity or impact force at various levels of sag prior to operating. Dedicated interventional airguns incorporate the same valve-body slideway as modified airguns as the basic means of control, but to expedite and refine adjustments, offer additional control points.

Interventional Airgun with Liquid Vaporization-on-Release Cartridge or Compressed Gas Cylinder Connected Directly to the Valve Body Inlet Suitable for Use Over a Range of Exit Velocities (Forces of Penetration) in Quick Succession with Moderate Redundancy as to Points of Control

Gradual curves in the barrel-assembly reduce the exit velocity and sharper curves can do so to a significant extent by percent in relation to the range of exit velocities effective in placing the miniballs subadventitially before puncturing the outer fibrous layer of the ductus. The use of thicker tubing in the barrel-catheter and barrel-tubes, a larger diameter barrel-catheter, use of centering devices to place the barrel-tubes farther from the central axis, and blood-tunnels to increase the stiffness of the barrel-catheter over the length usually not required inside the body have been mentioned above. A description of an interventional airgun that incorporates automatic positional control will be found following a description of components such as a linear positioning table used to execute the positioning.

The incorporation of a photo-ablation excimer laser also adds stiffness to the barrel-assembly. A radius of curvature that is severe without kinking or distorting the barrel-tube in internal diameter demands greater propulsive force to achieve the same exit velocity as were the barrel straight. Operative speed being a crucial factor in achieving a good result, to expedite adjustment of the apparatus to achieve the exact exit velocity desired, provided are interventional airguns with several control points, some redundant. Interventional airguns are always to be provided with guidelines for selecting and testing exit velocity for a specific application. Due to the greater variability in mechanical properties of tissue once diseased, the automated coordination of the separate controls of dedicated interventional airguns to instantly bring the gun to preset exit velocities must afford fine adjustment if not continuous variability. Such control would not be particularly advantageous, and is not considered to be worth the additional expense.

In a simple embodiment intended for use with a refillable cylinder of compressed air that has been pressurized for a given tissue, an electropneumatic valve consisting of a push-type solenoid actuator and valve body is used to admit and within a small range compared to a regulator, control the pressure of the gas used to propel the shots, hence the exit velocity and force of impact (FIG. 34). The control knob adjusts the voltages that regulate the extent and duration that the electropneumatic valve opens to the pressurized gas. Providing dedicated interventional airguns with an additional foot control switch to trigger discharge is not preferred. A conventional electrical foot-switch must be adapted to incorporate a safety pin that must be released by depressing a lever with the toe of the opposite foot, and limited to triggering only, the foot switch is too limited. The incorporation into a foot operated control panel of all the controls necessary to use the apparatus is rejected as inviting unintended actuation.

FIG. 51 is a block diagram, not to proportion, of a gas-operated surgical miniball implant insertion airgun with compressed gas cylinder connected directly to the valve body inlet. While represented this and the dedicated interventional airgun next to be described are represented as gravity fed as suited to use with a simple pipe barrel-assembly, it is to be understood that either can also use rotary magazine clips and so accommodate any kind of barrel-assembly. In addition to the valve controls provided, different delivery tubes friction fit to the end of the barrel can be used to variously reduce the barrel exit velocity, hence, the force of impact. Such an embodiment, using a single cylinder of compressed gas without the additional expense of a regulator, is suitable for use where the a range of exit velocities or forces of penetration is required, as when treating a single tissue to a single depth. Under normal circumstances, a disposable delivery catheter designed for the particular application is provided. A device as shown above and in the following figure allows continuous variability in the force impact, which expedites testing tissues for the purpose of disposable catheter design.

The compressed gas can be supplied, for example, from either an internal prefilled disposable CO₂ or by means of piping from an external CA compressed air cylinder. Whereas CO₂ delivers 837 psi at 70 degrees Fahrenheit, a compressed air cylinder can be filled to a preferred pressure. With the interposition of a small adaptor, either a CO₂ or CA cylinder can be connected to the valve body inlet. Using a single source of compressed gas without regulator keeps the design simple and economical. A small CO₂ cylinder inserted within the enclosure makes the single-purpose airgun self-contained and compact. Containing nonliquified gas, a compressed air cylinder is larger and therefore connected from outside through a hose but can be filled to any pressure within its design specification. With or without a regulator, control with a single source of compressed gas is limited to reduction in the outlet pressure (also referred to as a canister or tank). With this basic design, variability in shot impact force is limited to adjustment in the field strength and duration of push-type solenoid actuation. Preserving this simplicity and economy limits the pressure-reducing features that can be built into the airgun.

Nevertheless, by connecting compressed air cylinders filled to different pressures, even the simple airgun can be used to treat different tissues to different depths of penetration. In such use, multiple cylinders of compressed air are connected and switched among manually by means of a pneumatic or electronically by means of an electopneumatic station valve. This can be done at no great expense when switching is manual; however, the parts necessary to switch among different cylinders with electronic valves loses the economic edge over a design that affords continuous variability in pressure through the use of a regulator. A warming jacket containing a heating element or coil about the gas delivery tube with thermostat or pyrometer control can be used to change the temperature and so adjust the pressure. Since conventional CO₂ cylinders are rated for up to 1800 psi, the range of pressure control gained in this manner is much less than it is with compressed gas cylinders, which can withstand thousands of psi. For improved visibility, the pressure gauge P, temperature gauge or pyrometer T, and voltmeter V are housed separately from the table-top or stanchion-mounted main unit.

PSOS full-wave rectified regulated power supply output switch. The take-offs for the different components are voltage divided by a bleeder resistor, each circuit controlled by a variable resistor. EPOT electronic potentiometer remotely operated from the remote hand control. In a simpler version, the potentiometer is mechanical, in the same position in the circuit, but mounted on the chassis rather than the hand control. VCTDR voltage-controlled time-delay relay. Essentially, there are two circuits, one pneumatic, the other valving the passage of gas through the pneumatic circuit. The combination of the push-type solenoid and the gas valve constitute a special purpose impulse-actuated electropneumatic valve. Whereas a enclosure-mounted manually operated potentiometer is less costly and assumes operation by an assistant, an electronic potentiometer in the remote hand control affords the operator direct control; both can be connected in series.

Depressing the remote control ‘dead-man’ or plunger type trigger switch at the top of the joystick control connects the power supply through the EPOT and VCTDR to the direct current push-type solenoid, energizing the solenoid coil. This causes the solenoid plunger (slug, armature) to punch the spring-loaded valve inlet pin forcing open the valve within the valve body for the interval set by the VCTDR. Use of the plunger switch trigger always requires release of the safety by retracting a pin intromitted into the side of the control button or key which is placed at one end of a spring-loaded lever retracted by pressing the opposite end with the ball of the index finger. The gas thus admitted to the rear of the mini ball implant in the receiver propels the implant as a projectile through the barrel and delivery tube at the target tissue. Adjustment in the output of the power supply through the potentiometer varies the actuation field strength of the solenoid, varying the punching force of the solenoid plunger against the valve pin. Increasing the force of plunger impact upon the valve pin also slightly increases pin excursion, hence, valve open-time.

Valve open-time is thus determined both by the interval that the switch connects the solenoid to the power supply and by the voltage. This timing may be controlled as a structural or mechanical feature of the switch contacts or through a separate electronic time-delay relay. Absent such a solenoid actuation time mechanism, the solenoid plunger would not retract until the switch was released, which interval is too long. The discontinuous character of the function, which involves the intermittent discharge of sudden shots, does not lend itself to servomechanical control; instead, a V voltmeter indicating EPOT output on the enclosure serves to implement human feedback. The acrylonitrile butadiene styrene (ABS) enclosure with a thermal conductivity between 0.14 and 0.21 W/mK and 97 cubic feet per minute (cfm) fan with plastic vanes and frame prevent the undesired buildup of heat that could materially alter the gas pressure and therefore terminal ballistics. Adjusting the fan speed and thus the volume of air moved through the enclosure by means of a thermostat is another way that the temperature of the gas can be controlled to obtain variability in pressure.

Conventional means exist for preventing the temperature to exceed a set limit, and even were such to malfunction, all compressed gas cylinders incorporate a pressure relief mechanism. Thus, even using a single cylinder, and even when the cylinder contains CO₂, which is not normally viewed as affording variabiity in pressure, numerous variables are available to control the pressure and therefore the force of impact and depth to which the shot will penetrate given tissue. Of these, the least costly embodiment shown here employs those variables that govern valve open time. In an embodiment that must afford a wide range of penetration forces for a single procedure, a regulator capable of continuously adjusting the gas pressure is used. Whereas a regulator and the control means shown allow pressures less than that to which the cylinder is pressurized, increasing the temperature allows the cylinder pressure to be exceeded. The power controlled from the remote control hand piece is represented as controlling both the output from the power supply through the electronic potentiometer and the input power proportional time delay.

That is, the same potentiometer is used to vary the input to the solenoid and the time-delay relay to continuously vary the force and interval that the valve is held open, both of which factors increase valve open-time. Separate control of the time delay does not significantly extend control variability. Whether manually adjusted in a simpler model or electronically in one more costly, a regulator is usually controlled separately. In such an embodiment, the regulator is in effect the gross adjustment, whereas the controls shown here serve for fine adjustment. To avert disruption due to malfunction, more than one such relatively simple apparatus, each adjusted to the same settings, should be present. If more than two are available, differently adjusting these in pairs allows treating different tissues.

Interventional Airgun Suitable for Procedures Involving the Treatment of Different Tissues to Different Depths in Quick Succession with Redundant Points of Control to Adjust the Exit Velocity

FIG. 52 is a block diagram, not to proportion, of a gas-operated interventional airgun suitable for procedures involving tissues that differ widely in resistance to penetration where there is the need to penetrate to different depths, necessitating adjustability over a wide range of shot impact forces. The airgun is shown with a gravity queue-fed magazine implying use with a simple pipe, but is capable of loading rotary magazine clips. The use of an interventional airgun that lacks automatic positional control where precise distancing between successive discharges is essential, as is often true in the vascular tree, for example, is not preferred. Such an airgun is suitable for use, for example, with a simple pipe-type barrel-assembly in the airway or in any ductus operable manually as not to demand precise distancing between successive implant discharges.

Interchangeable single, or individual ‘BB,’ and multiple-shot or shotgun shell-adapted pellet casing loading mechanisms allow the same propulsion apparatus to be used analogously to a rifle or shotgun, where the object is likewise similar—either to deliver individual shots with discretionary placement in suture mode or distribute shots over an area in stent mode. The latter is far more frequent, uniform distribution spreading the magnetic attraction and therewith any risk of pull-through, and with the need for precision reduced. For improved visibility, the large digital pressure gauge P, temperature gauge or pyrometer T, and voltmeter V are housed separately from the table-top or stanchion-mounted main unit. PSOS power supply output switch. EPOT electronic potentiometer remotely operated from the remote hand control. In a simpler version, the potentiometer is mechanical, in the same position in the circuit, but mounted on the chassis rather than the hand control. VCTDR voltage-controlled time-delay relay. EPR remotely controlled electropneumatic regulator; as with the potentiometer, using a manual regulator with control knob mounted on the enclosure considerably reduces the cost of the apparatus. Whereas manual control makes maximum use of an assistant, remote control allows immediate adjustment by the operator. The compressed gas is stored in a disposable CO₂ cylinder prepressurized and unrefillable at 837 psi at 70 degrees Fahrenheit.

CO₂ cylinders are available in a variety of sizes, to include 9, 12, and 88 grams or 9, 12, or 20 ounces. The cylinder is connected to the valve body through a continuously variable regulator that allows a range of gas pressures for use with any suitable tissue. A manual regulator can be used for economy but an electrically controlled regulator allows direct control by the operator. The use of a regulator eliminates the need to connect differently pressurized cylinders or change the temperature whenever treatment moves from one tissue to another. A time-delay relay is still required to limit the interval that the solenoid is actuated, but a potentiometer, while still desirable, is not essential for control. While less costly, a manual system that interswitches among plural cylinders does not achieve equivalent continuity or smoothness of operation. To employ an electrical means of switching among cylinders would lose much of the economic advantage of switching compared to a manual if not an electrical regulator. A manually operated regulator can be controlled by means of a knob on the enclosure. To avert disruption due to malfunction, more than one apparatus should be present and adjusted to the same settings as the procedure unfolds. With a regulator, other means for altering the force of impact, as by changing the delivery tube to one of different length or a material of different coefficient of friction are unnecessary.

### Single Axis Linear Positioning Table Airgun Mount

The airgun linear positioning table (linear platform, linear stage) mounting is purchased as a complete subassembly, to include both the linear positioning table and actuator. Numerous types of linear positioning tables are available, some open-loop controlled with stepper motors, others closed-loop controlled with dc servomotors. The actuator can be linear or rotary, and in either category, any of several different kinds. The table itself requires no modification; rather, adaptation for the present purpose consists of its positional control programming. One suitable linear table is the Parker Hannifin MX80S miniature linear motor stage with stepper motor under open-loop point-to-point control, which may be used with a Compumotor® 6K controller. The operator brings the muzzle-port or ports to the starting position for the programmed discharge pattern and sets the point-to-point distance (interval) to separate the successive equidistant discharges and the overall length (segment, stretch) of the duct or vessel to be implanted. Such is translated as increments to separate the starting and ending positions of the linear table.

The operator then uses a joystick or cyclic-like control arm as described below to initiate the execution of this number and distance of moves forward or backward. Once the pattern is confirmed to have been set correctly, the joystick is used to indicate the direction, the safety is removed from the plunger switch at the top of the joystick, and the plunger switch depressed to execute the pattern that was set. Control is addressed in the section below entitled Airgun stenting (position and discharge) control panel. The patterns may consist of discharges along a straight line or lines (advancing or withdrawing), or straight lines followed by rotation and return (reversal of direction), or the repetition of the latter after the muzzle-head has been rotated to longitudinally (transluminally) pass over unimplanted arcs. While the use of a muzzle-head having a larger number of radially directed muzzle-ports so that all arcs are implanted simultaneously is preferred, such rotation and reversal allows the use of a barrel-head containing fewer barrel-tubes, which may be larger in diameter and thus allow the delivery of larger miniballs.

Positioning of the Muzzle-Assembly with the Linear Positioning Table and Turret-Motor

### Type and Efficiency of Control

Both the closed-loop control of the turret-motor and open-loop control of the linear table are initiated by the operator with the joystick, forward to move the table forward, backward to move it backward, and clockwise or counterclockwise to move the turret motor to the corresponding angle. Move and discharge operation is limited to the linear table or transluminal positioner. Transluminal runs consisting of translation by the linear table, holding fast while the timing relay signals the airgun hammer push-type solenoid to operate, then executes the following increment, are performed one at a time, direct observation and, action cancellation (override, interdiction) by the operator taking precedence over any automatic function. There is, therefore, no stack or register to store successive transluminal discharge runs, and no provision for the programming of successive runs is allowed.

Unless made to progress at a very slow rate, continuous positional control, whether by direct analogy whereby the muzzle-head would be made to move say, one millimeter for each move at the control of a centimeter, or by continuous directional incrementing, so that the muzzle-head would continue to increment in the direction of the control until the control was retracted, are both subject to constant overshooting. The form of control must not permit a condition such that every change in position requires to be corrected, much less several times. Wasted motion would soon fatigue, prompting sloppiness where this must not be tolerated. While the first of these forms of control is the most intuitive or consistent with spontaneous eye-hand coordination, and the second is more intuitive than control that is based upon strict adherence to a previous setting of controls to specify the number, size, and direction of the increments to be executed automatically, for interventional application, where losses in efficiency based upon essential design factors are unacceptable and impatience with constant overshooting might induce carelessness, the first of these forms of control is rejected and the second reserved for quickly positioning the muzzle-head at the starting position for automatic discharge.

Once initiated, however, the system requires that the number and size of the increments to comprise each movement be entered first and the joystick or cyclic used to indicate the direction of movement, the latter being singular in any one such discharge-run or compound action. The apparatus then automatically switches between the movers (turret-motor and linear table) and the airgun push-type solenoid used to strike the valve body pin, stopping long enough between increments to allow the implants to travel to the trajectory termini. Shifting the joystick forward moves the linear table stepper motor distad, backward proximad, tilting to the right or rotating clockwise moves the turret-motor clockwise, and tilting to the left or rotating counterclockwise moves the turret-motor counterclockwise. The joystick has a central null position through which changes from forward (distad) to backward (proximad) direction of the airgun mounting linear positioning table must pass, so that reversal cannot be immediate. Similarly, rotation of the turret-motor cannot be reversed immediately, because a null region separates clockwise from counterclockwise contact, and since forward-backward excursion passes through the rotatory null region, simultaneous actuation of the turret-motor and linear table is impossible.

Actuating the automatic discharge (autodischarge) rocker switch causes time delay relay XX to alternately switch between either the linear table stepper or turret-motor as mover to the airgun solenoid that when energized strikes the valve-body pin releasing CO, into the airgun chamber causing the implants to be ejected. The airgun is mounted on a linear positioning table that by moving the airgun bodily, transluminally advances or retracts (withdraws) the muzzle-head. The linear positioning table can be used to a. Accurately reposition the muzzle-head once the barrel-assembly has been inserted into the airgun barrel, which involves only control over the linear table and turret-motor as movers, b. Reposition the muzzle-head and then effect discharge semiautomatically, the operator manually triggering each discharge, which alternates between positional control and discharge, or c. Direct automatic repositioning and discharge, in which compound action the muzzle-head is manually directed to reposition by uniform distances (increments, stretches) stop at each conjunction by a fixed time that is long enough for the airgun to discharge with the longest barrel-assembly, and then discharge automatically at each stop, which requires the automatic and coordinated control of the movers and the airgun.

Turning now to the airgun control panel shown in FIG. XX, once angioplasty has been completed, the barrel-assembly is inserted into the airgun. The power supply is activated by pressing ‘ON’ button XX. To bring the muzzle-head to the starting position for discharge, the joystick is held in the direction required until the linear table and the turret-motor have incremented toward and positioned it thus. Semiautomatic discharge is appropriate for isolated discharge, but implantation for stenting demands a proximity and accuracy of adjacent placement that only machine controlled automatic discharge allows to be attained. Once the starting position has been reached, the airgun can be a. Discharged manually or semiautomatically by releasing the safety on the dead-man trigger switch and depressing the trigger, or b. Semiautomatic discharge initiated by using the upper dial to set the number of increments and the lower dial to set the length in millimeters of each increment. The automatic discharge rocker switch is then shifted to the on position, and the direction of automatically executed discharge is commanded by shifting the joystick for the equivalent or analogous intraluminal movement, meaning forward for transluminal advancement, backward for retraction, rotated clockwise for clockwise rotation of the turret-motor, or rotated counterclockwise for counterclockwise rotation of the turret-motor.

Since the preparatory angioplasty is generally carried out manually with the barrel-assembly independent of the airgun, automated positioning with the table ordinarily commences with insertion into the airgun barrel of the barrel-assembly for the purpose of placing the intravascular stent implants. So long as the barrel-assembly is used independently of the airgun for angioplasty, the turret-motor is seldom if ever used as a mover but rather as a means for generating heat for thermal angioplasty. When the barrel-assembly resists rotation manually, then depending upon whether connection of the control electronics to the barrel-assembly is at the end-plate or in front of the airgun muzzle, the free proximal end of the barrel-assembly can be temporarily inserted into the airgun to connect the turret-motor. The uniform increments, each a sum of component point-to-point steps of the table stepper motor, can be used to produce motion that is continuous while the airgun intermittently discharges, or the movement can be keyed, meaning coordinated in timing to, the successive discharges of the airgun, so that the muzzle-head is made to intermittently travel a certain distance, wait in place until the one discharge is completed, then resume travel to the next implantation site.

In fact, the length of the pause in such intermittent movement is variable from complete cessation that is initiated before each individual discharge is triggered until after the discharge has been completed to only a portion of the discharge cycle, such as during recoil when, for example, it is more likely that a miniball might escape. When positioning is not keyed to the individual discharges, the overall distance and number of discharges within this distance are specified. Provided a threshold for the minimum interval to separate implants is not violated, the control mechanism then spaces this number of discharges at equal intervals within this distance. To signal that implants have been placed too close together in linear sequence requires an ability to relate the action of the positioning table to the discharge of the airgun and consequent points of successive implantation and to use an out of tolerance condition to actuate an alerting device. As indicated, when positioning is keyed to the individual discharges rather than continuous, the positional cycle consists of movement in discrete translational sub-incremental steps of the stepper motor as table actuator from implantation site to implantation site, the muzzle-head held stationary while the airgun discharges before proceeding to the next implantation site.

Intermittent action comprehends two subcycles, which the positional control system is used to coordinate. One subcycle consists of the timing relations in the operation of the airgun and barrel-assembly, which are only slightly variable. The airgun is fully variable in the timing of the initiation of discharges but not in the time of each discharge. The other subcycle consists of the pattern of transluminal movement, which is fully variable. The airgun cycle consists of the release of CO₂ into the chamber, the transmitting of the barrel-tubes by the miniballs, the ejection of the miniballs through the muzzle-head, and the time for the miniballs to penetrate to the trajectory terminus. The transit time varies as the length of the barrel-tubes, but the absolute duration of this interval compared to that of the fixed rate of transluminal repositioning is small. Since control over the airgun mounting is fully variable while control over the airgun mechanism is only variable in its intragun discharge cycle characteristics and thus only slightly in the absolute overall duration of the cycle, the operation of the airgun mounting is made to subserve the timing dictated by individual discharge from chamber to implantation end-point.

In intermittent operation, the movement of the muzzle-head or the fixed duration of each stop can be keyed either to the actual or to the highest rate of discharge, or more specifically, to the full, some portional, or the maximum time for an individual discharge, the last providing an interval of time slightly greater than needed with the longest barrel-assembly, thus eliminating the need for adjustment in the feedrate and achieving relative simplicity. That is, fixing the muzzle-head pause time for the maximum discharge time, which is determined mostly by the barrel-tube transit time, eliminates the need to adjust the pause time even though with shorter barrel-tubes or a higher discharge velocity the duration of this pause could be reduced. Accordingly, to preclude human error, the pause time is fixed at the maximum needed for the longest barrel-assembly

Because the perforated barrel-tubes disallow any buildup of gas pressure, whether the result of premature discharge or in discharge with continuous movement of the muzzle-head, the shot-groups of successive discharges exert no effect upon the exit velocity of one another even though the miniballs of the previous discharge or discharges have not yet exited. Furthermore, with continuous movement of the linear table and muzzle-head during discharge, a malfunction resulting in premature follow-on discharge so that more than one shot-group traversed the barrel-tubes simultaneously would have no jamming effect. Accordingly premature discharge is to be avoided exclusively due to the misplacement of implants that results.

Interventional Airgun with Multiple Exit Velocity Control Points for Quick Midprocedural Adjustment, Using Rotary Magazine Clips, and with an Automatic Positional Control System Suitable for Implanting the Wall of a Blood Vessel

An interventional airgun suitable for use in the vascular tree incorporates automatic positional control that allows successive discharges to be placed at smaller distances than can be attained manually with accuracy. To achieve uniformly equidistant separation of the implants in a formation and thus reduce the risk of pull-through within a ductus having a caliber the size of the average muscular artery, for example, necessitates automatic operation, the muzzle-head as ‘tool’ advancing or rotating, discharging, and continuing according to the control input of the operator. Such an airgun is the same as that last described but supplemented with positional drives and controls and a rotary magazine clip that unlike the queue or linear succession spring-loaded clips in many commercial hand air pistols if not that type described above as more capable, and gravity-fed loading in the preceding interventional embodiments, which are meant for use with a simple pipe-type barrel-assembly, can load multiple miniballs in any combination for simultaneous discharge.

Since the second of the two kinds of modified commercial air pistols described above uses a rotary magazine clip, it is capable of discharging more than a single miniball at a time; however, lacking a positional control system, the targeting of each discharge must be achieved manually, which taking time, is unsuited to use in the circulatory system and in the coronary arteries in particular, where quickness is imperative for averting the ischemia that can induce a myocardial infarction. The same may be said of use in the carotid arteries where the risk of a cerebral infarction must be minimized. Of the two types of interventional airgun described above, neither uses a rotary magazine clip as allows the use of more than a single barrel-tube. In the second and more capable air pistol, the rotary magazine clip is rotated by a pawl that is mechanically linked to the trigger and engages notches about the circumference of the magazine.

In the mechanical system seen in a conventional airgun that uses a rotary magazine clip, the pawl that rotates the clip is moved when the trigger is pulled back, so that the rotary magazine clip has already rotated or indexed to place the next load before the CO₂ inlet when the trigger reaches the end of its travel (excursion, throw), at which point the hammer is released to strike the valve body pin effecting discharge. Here, the same sequence is reproduced through the use of a plunger switch that when partially depressed completes the circuit through a small electromagnet, and when fully depressed, completes the circuit through the push or punching solenoid used as a hammer. When triggering is electrical, the rotary magazine clip and pawl are no different than those used in a mechanical linkage to the trigger but differ in that the pawl is actuated by the small electromagnet that is energized by depressing the spring returned trigger consisting of a raised plunger (pushbutton, dead-man) double pole double throw normally open momentary close contact switch mounted within the top of the joystick. One point of control in this airgun governs valve body pin depression time. While this interval could be affected at the trigger switch, more precise and replicable control is achieved by the means to be specified.

### Airgun Stenting (Positioning and Discharge) Control Panel

Regardless of the type of barrel-assembly used, discharge and the transluminal and rotational movement associated with successive discharge occurs only while the barrel-assembly is inserted in the airgun. Accordingly, the control panel for these functions is mounted not on the barrel-assembly but as separate (remote) from the airgun, at the top of a stanchion with weighted base and stand that may be raised to the height most comfortable for the operator, or on the airgun. So that the operator can call for immediate assistance, duplicate airgun control panels are preferably in both locations, those mounted to the airgun requiring enablement from the stanchion control panel. The airgun itself may be mounted at the top of a stanchion or set on a table, but must be adjustable in height to level the barrel-assembly with the patient.

In contradistinction to positional control for discharge, the functions assigned to the barrel-assembly and more especially a fully ablation and angioplasty-capable barrel-assembly—such as the deployment of a side-sweeper in order to nudge the muzzle-head to one side of the lumen—are assigned to the control panel onboard the barrel-assembly. Discharge is either a. 1. Concurrent or 2. Delayed, and b. 1. Directly manual or 2. By executing preestablished patterns, described as semiautomatic, in that once the equidistant discharges have been manually set as to increment (distance of separation), indicating the direction for the action and depressing the plunger switch at the top of the joystick causes the pattern to execute.

Concurrent manual control over movement is obtained by holding down the plunger or dead-man switch at the top of the joystick at the same time that the joystick is used to move the barrel-assembly or rotate the muzzle-head, while delayed movement is obtained by first positioning the joystick forward to intraluminally advance, backward to withdraw, or to a preset angle to rotate the muzzle-head, and thereafter depressing the plunger switch. As a mode of semiautomatic operation, delayed execution is used to reduce the risk of human error in the form of overshots that would necessitate frequent if not irritating transluminal reversals in direction. As mentioned in the section above entitled Single Axis Linear Positioning Table Airgun Mount, the operator first sets the number and distance of point-to-point increments to separate the starting and ending positions for the linear table and then uses the joystick to initiate the automatic execution of this pattern. The apparatus then automatically moves the muzzle-ports forward or backward to the successive target locations for discharge, with or without rotation of the muzzle-head.

More specifically, the control panel includes control settings for initiating programs that automatically discharge miniballs in preestablished formations, that is, in preset discharge patterns that execute as unit routines. The patterns are obtained by coordinating the transluminal movement of the linear table and rotation of the muzzle-head with discharge. Positional control thus involves the coordination of the two drive axes, the one an open-loop controlled stepper motor that moves the linear positioning table for transluminal movement, and the other, a closed-loop controlled dc servomotor that rotates the muzzle-head with actuation of the airgun push solenoid used as a hammer to strike the valve body pin. Such action is obtained through a programmed sequencer or stack register that stores the sequence of instructions for executing the pattern as a unit or routine. The sequencer controls the two servodrive controllers (“amplifiers”). In practice, the servodrive is usually a two-axis unit that is able to control a closed and an open loop simultaneously. As indicated, a pattern can include transluminal and rotational reversal, with discharge effected at each stop.

An automatic pattern is selected with a control knob having a pointer that is moved to the pattern chosen, and then the direction as forward (intraluminally advancing) or withdrawing (intraluminally retreating) is indicated by moving the joystick forward of backward respectively, and the action initiated only after the plunger switch atop the joystick is taken off safety and depressed, at which time the sequence consisting of moves to a succession of discharge points proceeds automatically. Pushing a cancel or check button on the control panel instantly stops (abends) this action. Rotational repositioning of the muzzle-head is directed by the program and executes, and coordinated timing exceeding the capability of the operator, without the need to rotate the joystick. Rather than serving as a move execution switch to reposition the muzzle-head, in the control of discharge, whether directly manual to discharge one or a plurality of miniball implants or to initiate an automatic discharge pattern, the function of the plunger switch is as a trigger, and each terminus of travel a point for discharge. Whether to move or discharge, the on-off switch must be set to ‘on,’ and depressing the plunger switch always necessitates unlocking the safety.

As additional safeguards, discharge during movement is electronically disabled, and a check-action or cancellation button positioned beneath the thumb of the operator on the joystick handle instantly truncates the ongoing action. More specifically, depressing the ‘cancel’ button XX stops the flow of current to the turret-motor (rotatory axis), airgun linear table (transluminal axis), the pattern instruction program, and airgun discharge actuating push solenoid to instantly arrest action commanded before or once initiated. While discharge could proceed so that miniballs were released while the muzzle-head continued to move, needless complexity and risk is avoided by not allowing movement and discharge simultaneously; rather, movement electronically disables discharge, which is automatically enabled on reaching the end of travel. The positional controls mounted to the airgun are for use only with the barrel-assembly inserted and are intended solely to move the muzzle-head from one location for implant discharge to the next. As explained, when the implants must be placed too closely together for manual control, upon depressing the trigger, transluminal movements of several millimeters or degrees of muzzle-head rotation are accomplished semiautomatically.

In addition to the on-off switch, plunger switch safety lock, and action cancellation button, the airgun or stenting control panel typically includes controls for positioning 1. Turret-motor rotation (typically by means of a digital encoder manually rotated by the rotary or tilt right or left component of the joystick (cyclic) with a pointer moved above an upper semicircular calibration with apical or centered O-point (set point) and marked off in 5 degree error signal increments to either side); 2. An advancement and withdrawal control for direct manual transluminal movement with the linear positioning table by preset increments of a number of millimeters, the direction and execution controlled by moving the joystick forward to advance or backward to withdraw; 3. Advancing the intraluminal barrel-assembly in increments of two millimeters with the linear positioning table by pushing the joystick forward or backwards to withdraw; 5. Action cancellation or checking switch to instantly truncate the action whether mid-repositioning or mid-discharge, regardless of whether the action is direct manual or semiautomatic as a patterned sequence or collective unit formation; and controls for discharge 6. Discharge pushbutton (plunger, dead-man) switch at the top of the joystick for executing positional, discharge, and semiautomatic control; 7. Automatic discharge pattern selection knobs, transluminal (linear table) positioning generally in increments of two millimeters and rotation (turret-motor) generally in increments of 5 degrees; and 8. Recovery (tractive) electromagnets 1 and 2 low-off-hi toggles. The cancel and actuation keys on both the control console and an angioplasty barrel-assembly pose sufficient resistance to depression to minimize the risk of unintentional depression.

### Relation of Control Panels to the Turret-Motor and Airgun Linear Positional Table Axes, to Discharge, and to One Another

Control of the airgun linear positioning table consists of using the switches and joystick on the airgun control panel, which is mounted on a bottom-weighted stanchion. Although the barrel-assembly can be inserted into the airgun barrel for motor controlled positioning at any time, angioplasty can be entirely manual, the operator manipulating the barrel-assembly at its free proximal end. Motor controlled advancement and withdrawal of the intracorporeal barrel-assembly is thus essentially limited to implant discharge or stenting use of the barrel-assembly. That is, while some conditions will recommend stent-jacketing prior to implantation, in most instances, stenting will follow use of the barrel-assembly manually with the proximal end freely movable. Implantation then requires insertion of the barrel-assembly into the airgun, the rest of the transluminal positioning of the barrel-assembly performed by means of the linear positioning table, and rotation of the muzzle-head by means of the turret-motor. As seen on the control panel shown in FIG. XX, the operator or an assistant can use the table to advance or withdraw the muzzle-head continuously or by a certain number of steps where the size of each step is set with a neighboring control knob.

The airgun linear positioning table is preferably of the stepper motor-driven lead screw kind under open-loop control. A four-way radially symmetrical muzzle-head advanced by the linear positioning table will lay down lines of implants at uniform longitudinal distances to define the quadrants of the ductus. As an examination of FIG. XX, which shows the control panel will make plain, to produce a close formation of implants in order to evenly distribute the tractive force, the turret-motor and linear stage can be semiautomatically controlled to advance or reverse transluminal movement, the operator directing each run by entering the linear distance and number of discharges. At the end of each linear run, the turret-motor is used to adjust the rotational angle and the linear table is then used to linearly distance the discharges in the reverse direction. Alternatively, the turret-motor can be used to rotate the muzzle-ports at each level without reversing direction so that the muzzle-head advances intermittently but consistently distad or withdraws thus proximad.

### Automatic Close-Formation Pattern Implantation

While stereotypical or iterative pattern generation is a mainstay of numerical control in piecepart manufacturing, the functionality of automatic pattern generation for the present purposes would be inappropriate. In a clinical setting, complete flexibility subject to the medical judgment and immediate control of the operator is paramount. Detailed medical aspects of the actual lesions demanding treatment represent the primary factor in the decision process, mere niceties of technology impertinent. Discharge patterns would have to exist in so many variations of overall size and shot density that an absolute number of such patterns would be needed as would promote human error in selection. Any such capability would most likely promote a dependence upon a generalized patterns where these were not properly applied. In order to be adapted for any real set of lesions, prepackaged patterns would have to be variable in omitting or adding implants to an extent that would render the nominal patterns useless. Accordingly, even though it would be a relatively simple matter to make discharge of entire formations execute automatically as a complete pattern, the concept is discounted in principle.

### Positioning Modes of Operation

Positioning and operation at any given time may be fully manual, manual with direct control over the linear positioning table airgun mounting and/or turret-motor, or manual in initiating automatic sequences or discharge groups wherein once selected by the operator, the rotary angles and/or distance separating the individual discharges is accomplished automatically. During transluminal movement, the turret-motor and discharge but not the side-sweepers with trap-filter and any auxiliary device such as a laser catheter or rotary burr, remain disabled.

Positioning with a Simple Pipe

Because the structured anatomy demands the discretionary placement of each implant, use of a simple pipe in the airway is always under direct manual and never automatic control. The airway in all but the tiniest (veterinary or premature birth) patients affords sufficient space to maneuver the handpiece, the structures involved are observable endoscopically, ultrasonographically, or fluoroscopically. The tractive electromagnet is hand-operated. Airgun (not positional) operation in this or similar environment is semiautomatic as not to require reloading mid-magazine clip. Other than not having to reload the airgun between clips and energization of the tractive electromagnet, the simple pipe lacks the auxiliary functions required in a radial discharge embodiment suitable for use in the bloodstream which demands very fine and fail-safe retraction or deployment during transluminal movement near or over a lesion, which accordingly, is automated. Control of the tractive electromagnet, and the transluminal advancement, withdrawal, and rotation of the simple pipe are thus completely manual.

Automated Positioning with a Radial Discharge Barrel-Assembly

Most extraluminal stenting will, however, pertain to ducti no larger in lumen diameter than 3 millimeters, and except for the segments that necessitate treatment, have a relatively uniform structure. Discharge in smaller ducti seldom involves individual longitudinally disparate shots but rather the uniform incremental implant-carpeting of a lesion or the entire lumen. In this environment, safety measures to avert downstream embolization by escaped miniballs or plaque debris are actuated automatically even when longitudinal or rotary movement of the barrel-assembly is manual. When longitudinal or rotatory increments no greater than 2 millimeters are required, control over transluminal displacement, the rotary angle of the muzzle-head and thus the direction of the muzzle-ports, recovery tractive electromagnets, and the side-sweeping brushes demand measured control of machine accuracy.

For this reason, manual control as it pertains to the vascular system, for example, is substantially limited to discretionary direction over gross transluminal distances such as from the introducer sheath to the lesioned segment and back, angular displacement when noncritical, and the larger transluminal distances separating lesions. Manual control is otherwise pertinent to operator commanded automated actions, to include the distance separating successive discharges to apply across a given lesion and optionally, the number of individual discharges to constitute the discharge sequence or group. The airgun is provided with controls for the operator to direct discharge in groups or discrete sequences where each such group comprises a selected number and uniform spacing of individual discharges.

The duration of the manually controlled automatically unfolded operations is usually brief, the length of the ductus to be treated limited to the segments that are diseased or to be stented. Control of the airgun in executing these automatic discharge sequences is accomplished by switching relay and time delay components extrinsic to the airgun mechanism proper and is unrelated to the fully automatic operation of firearms. While the operator selects the number and distances to separate the discharges (individual shot or shot-groups) in a set, the coordinated timing of discharge and action of the linear positioning table stepper motor in incrementally moving the barrel-assembly and, if applicable, the turret-motor in angling the muzzle-head in each discharge group, is electronically coordinated.

In addition to positional adjustments of the muzzle-head that exceed the manual capability to directly manipulate or the imaging capability to clearly see, safety factors relating to the prevention of inappropriate discharge and the actuation of auxiliary functions, such as the deployment and retraction of side-sweepers and trap filter, disablement of the trigger-switch, inactivation of the linear positioning table stepper motor and side-sweepers, if present, during discharge, are not left to operator memory. However, not all functions are preferably automated, those configurational with respect to the apparatus applied preprocedurally for reasons of simplicity and economy.

Thus, as described above, the number of muzzle-ports and any eccentricity in the trajectories to characterize each discharge are achieved by preselecting a barrel-assembly of suitable configuration, while the number, caliber, and type of implants is prearranged by the choice of clips, the miniballs and blanks in each clip position, and the barrel-assembly used. While changes in the exit velocity could be automated to execute during a discharge set pattern, such adjustment is seldom required from one lesion to the next or even during a procedure, and to impart this function increases the cost of the airgun. Accordingly, if necessary, the exit velocity is adjusted once a short term automatic routine has terminated. A malfunction mid-routine abends the routine and disables the airgun. Immediately upon completion of the routine, the trigger switch is reenabled.

Thus, once the apparatus has been preconfigured for the specific procedure, manual control of a radial discharge barrel-assembly consists of gross transluminal movements to bring the muzzle-head within or away from close reach of the site to be treated, selecting the exit velocity, the number, and the distance of uniform increments to separate the automatically positioned and timed successive discharges applied to a given lesion, the deployment of side-sweepers if present, direct remote manual control over the transluminal position of the muzzle-head and rotational angle of the muzzle-head when not radially symmetrical, the electromagnet settings, and the deployment of side-sweepers, if present. For reasons of safety, the energization of the electromagnets at the resting trap-recovery field strength during discharge and the deployment of the trap filter at the same time as the side-sweepers are, however, not left to memory but made automatic.

### Modes of Failure

a. Failure to Properly Discharge

Use of the in situ test described below, which intrinsically tests the exit velocity of the apparatus to be used in relation to the tissue it is to be used upon, should avert incorrect settings of the exit velocity.

1. If the exit velocity is set too low, the miniball may fail to eject. Retrieval of the miniball is accomplished by running a barrel-tube ramrod with mildly magnetized tip down the barrel-tube.
2. If the miniball ejects without sufficient momentum to penetrate the lumen wall, whether it becomes stuck between the muzzle-head and the internal surface of the lumen wall or drops into the lumen, if not embedded within the soft inner layer by the outward force of the smooth muscle action of the passing pulse or peristaltic wave, the miniball is retrieved by the recovery electromagnets or trap filter, deployment of the latter being imperative in the bloodsteam. To time discharge for impact to occur at just the right moment when the wave passes is unrealistically difficult until several discharges allow this interval to be clocked.
3. If the miniball penetrates the lumen wall to too shallow a depth, it is extracted and recovered by increasing the current to the closer recovery electromagnet.
4. The reasons for placing a stent-jacket before initiating discharge are stated above under the section entitled Stent-jacket Linings for Containing or Preventing Perforations and for Reducing the Momentum and Misdirection of Rebound. If the miniball just punctures the adventitia at a tangent point, then its functionality for retracting the wall of the ductus cannot be depended upon and it must be replaced nearby.
5. If the miniball punctures the adventitia without sufficient momentum to rebound, it becomes embedded within the lining, and since the stent-jacket is applied to encircle the substrate ductus at its quiescent diameter, entrapment within the lining is accelerated by the outward forces of the smooth muscle action within the ductus. If the miniball interim finds a gap between the adventitia and lining, it innocuously either becomes trapped between the two and either forced into the inner softer layer of the lining or dropped into the body cavity.
6. If the miniball perforates the substrate ductus with sufficient residual momentum to strike the harder outer layer of the lining that is inclined (canted) outwards (centrifugally) moving ahead (forward, downstream, distad), the miniball will rebound to a functional location distal to that intended. The exit velocity is corrected and the miniball replaced. If the miniball is one of a plurality of miniballs radially discharged together, then depending upon the density of implants, the failure is disregarded or a rotary magazine clip with all but the one miniball position blanked is used with adjusted exit velocity to replace the miniball. Miniballs discharged in automated mode are by definition sufficiently dense to discount isolated discharge failures.
b. Shallow Termination into the Lumen Wall or Other Tissue of the Trajectory.

Even when coated with highly radiopaque tantalum, the miniballs are very small, generally 1.14 to 1.52 millimeters in diameter, making confirmation of successful implantation with imaging equipment difficult if not impossible. This is not, however, cause for concern. Since there is a range of forces that will substantially assure penetration through the luminal wall as not to terminate short of the more penetration-resistant outer tunic, which is harder and more elastic than the tissue subjacent to it, and since a value toward the upper end of this range is chosen to minimize the chance of shallow placement, only airgun malfunction or human error in setting its controls can result in shallow placement.

Shallow termination is not threatening, because the tractive force on the misplaced miniball is not sufficient to induce compression necrosis. If this is not the case, then the necrotic tissue before miniball will erode through the wall. Provided antibiotics are administered this is a self-correcting problem, the perforation spontaneously healing. Unless contaminated through negligence or mishap, all of the components involved are sterile, and antibiotics are routinely administered as a precautionary measure in any event. Fistulization occurs when infection or tissue necrotic due to chronic irritation erodes a pathway to the exterior and drains. Shallow termination does not, therefore, pose any significant risk.

When the operator sees that a miniball has landed short and negligible risk notwithstanding prefers to extract it, a tractive electromagnet at the front end of the muzzle-head is directed at the defective implant with the muzzle-head turret-motor, and the current to the electromagnet gradually increased until the miniball dislodges and becomes trapped in the tractive magnet antechamber. When the treated ductus abuts upon an interposed structure that limits its gross movement toward the direction of traction, and the implants are eccentric or toward one side, or in the longitudinal half of the lumen but not the other side, it is also feasible to pass an external electromagnet such as that described below over the defective implant.

Since the miniball or miniballs short of the termination intended will be lodged in softer, usually smooth muscle tissue, these can be pulled into the correct position, whereas those correctly placed will be prevented from perforating by abutment against the harder and more elastic adventitia. This process therefore has the effect of selectively forcing a shallow miniball or miniballs out to, but not through, the outer tunic. Trajectory overshot with perforation. Provided antibiotics are administered to control bacteria that are always within the body, this is a self-correcting problem, the perforation spontaneously sealing then healing. The miniballs are bioinert and sterile. If an overshot could penetrate another vessel to enter the bloodstream, then the stent-jacket or clasp-wrap to be used with a stent-jacket and the stent-jacket are applied before commencing to place the miniballs. Loss of a miniball or miniballs in the lumen. So long as the trap-extraction electromagnets in the front end of the muzzle-head are set to trap field strength throughout the procedure, any miniball or miniballs that become loose in the lumen are swept into an electromagnet antechamber.

The proper functioning and setting of the trap-extraction electromagnets are confirmed preoperatively, and once the barrel-assembly has been introduced, the ammeter on the airgun, as will be described, will immediately reveal a loss of current. Since in exceptional instances when collateral circulation is lacking the loss of a miniball in the circulatory system could result in ischemia and necrosis, a transfer switch to a temporary power source such as an automotive battery may be used to sustain current to the electromagnets in the muzzle-head during the interval until the generator comes on. This merely states that an emergency uninterruptible power source should always be on hand. The use of an external electromagnet of the kind described below can be positioned downstream to seize hold of any loose miniball or miniballs, which are then recovered by increasing the current to the electromagnets in the muzzle-head while reducing the current to the external electromagnet.

c. Perforations

When anticipated, perforations are precluded by prepositioning of the stent-jacket, which is usually of the double-wedge rebound type as described above in the section entitled Double-wedge Stent jacket Bumper-rebound Directing Linings. Without prepositioning the stent jacket, perforations by miniballs of 0.4 to 1.0 millimeters in diameter still have limited potential to cause significant injury. For the ductus treated, the site of puncture will be no more thrombogenic or less medically manageable than when implantation is successful. Barring human error in having set the exit velocity far too high, the residual momentum of the miniball after it has penetrated through the wall of the ductus treated is not likely to pose a significant threat for neighboring structures. For such an error to be so extreme that the miniball could penetrate to the interior of a neighboring vessel to become an embolism, for example, is fanciful. Such injury as could result is more realistically associated with nervous structures.

d. Jamming

Jamming is associated with firearms where the round (cartridge, projectile) is cylindrical and engaged in a rifled barrel, so that deviation from concentricity can cause the casing to seize against the inside of the barrel. Here, in marked contrast, the barrel-tubes are completely smooth, usually lined with a coating of polytetrafluoroethylene, and the miniballs spherical. This makes jamming extremely unlikely. Moreover, because the rotary or linear feed magazine clip is readily examined, the failure of a miniball to eject is immediately discernible. Except for the interior of the barrel-assembly not engaged within the barrel of a modified commercially sold airgun, a jam inside a barrel-tube is readily viewable fluoroscopically.

e. Premature Follow-on Discharge

Premature follow-on discharge with the muzzle-head moving from implantation point to point so that more than one shot-group traversed the barrel-tubes at the same time would not result in detention of the follow-on discharge. The perforated barrel-tubes disallow a buildup of gas pressure before the follow-on miniballs that could affect exit velocity even though the previous discharge had not yet exited. Premature discharge would, however, result in implant misplacement.

f. Endothelial Seizure

Because the diameter of the apparatus is preselected for the ductus to be treated, and the surface material of the muzzle-head is lubricious and may additionally be wetted with a lubricant, clinging or seizure of the muzzle-head against the surrounding lumen is improbable. Should such occur nevertheless, a lubricant such as ACS Microslide®, Medtronic Enhance®, Bard Pro/Pel® or Hydro/Pel®, or Cordis SLX® is injected through a catheter passed down a service channel, and an interval allowed for the lubricant to spread. The turret-motor is then used in oscillatory or chatter mode to work the lubricant between the lumen wall and the muzzle-head. Such eliminates the need for gross movements of the barrel-assembly that would be more likely to cause injury. Once confirmed free, the barrel-assembly is withdrawn.

### Need of a Means for Testing the Resistance to Puncture and Penetration of Diseased Tissue Requiring Treatment

The mechanical properties of the tissue to be treated and the structures surrounding this tissue are primary concerns. Even with anticoagulants administered, punctures produce swelling that serves to provide spontaneous self-sealing, and the inside of the stent-jacket may be wetted with a topical coagulant such as Gelfoam®, Gelfoam® with thrombin, Oxycel®, Surgicel®, Flo-Seal®, Avitene®, bipolar cautery, or argon beam coagulation to assist in sealing a puncture. Nevertheless, a puncture will be associated with an errant discharge that could injure surrounding structures, possibly damage nerves, and is to be prevented.

In testing, a continuous rod sees the expulsive force at its proximal end and directly transmits that force to the tissue at the distal end. There is little transmission loss in velocity, hence force of impact. By contrast, the miniballs are more subject to losses in momentum due to any differences in the bends and rolling resistance of the barrel-assembly that distinguish the conditions of the test from those of actual use. Provided the test is conducted under the same conditions of barrel-assembly bends and length as in the actual use for which the test is conducted, this can be compensated for by taking the test result as proportional For this reason, it is essential that the test be conducted under the conditions of bending of the barrel-assembly to apply in actual use.

In situations where the consequences of a puncture are less important than procedural speed, a preliminary discharge of a single miniball at the affected tissue with the force presumed necessary is performed, the result evaluated, and the appropriate adjustment made. Otherwise, no discharge of miniballs into diseased tissue should precede puncture strength testing. While intraoperative time constraints preclude pretesting for each individual miniball to be implanted, the puncture strength of the specific lesioned tissue in the specific patient should be determined. This value is ascertained by reproducing as closely as possible the effect of a miniball on the tissue to be implanted, and in this way, obtaining information reliable as may be had concerning its penetrability and puncture resistance.

### In Situ Tissue Puncture and Penetration Test

Due to the variability in mechanical properties of diseased tissue, a noninvasive test can provide approach a test that goes directly to the actual combination of histological and equipment factors involved. A preliminary noninvasive approach that involves first consulting a table for the probable range of exit velocity and impact force data for the barrel-assembly and miniballs to be used and tissue to be treated gives only a reasonable approximation for initially setting the airgun, which is then test discharged against impact force registration paper is described above in the section entitled Control of Propulsive Force or Exit Velocity by Means of a Calibrated Slide Cover over a Slot Cut into the Valve Body. The pretest provided is based on the principle that the momentum out is equal to the momentum in less friction, where friction is reduced to the point that for a practical spot check, it may be disregarded.

This allows the hardness of the lumen wall, which disease changes, to be evaluated quickly without a need for calibration, computation, or conversion. While quantitative findings would appear to be more dependable, not every point along the lumen wall to be implanted can be tested, and the hardness of diseased tissue is subject to wide variability that actually makes confidence in findings obtained from a different point ill-advised. Furthermore, by actually employing the discharge mechanism, the reading obtained from a force measuring gauge, mechanical force tester, or mechanical puncture tester need not be translated into the corresponding exit velocity opening the way for human error. The test is devised for simple, direct, and immediate results on an empirical and qualitative basis for practical use and makes no pretence to a level of precision required in the laboratory.

Shown in Figs. XX1 and XX2 is an empirical or purely observational means for quickly testing the penetrability and puncture strength of tissue in situ with any kind of airgun, regardless of the kind of clip used to load the airgun. Provided the operator initiates testing with exit velocities too slight to cause injury, increases the initial velocity slightly for each successive discharge, and avoids repeated testing at precisely the same spot, test discharge or discharge for effect, because it is empirical, that is, uses the actual tissue to be implanted and the actual apparatus to effect implantation, can afford dependable results quickly. Testing is never conducted other than immediately preceding actual discharge under precisely the set of physical conditions and depth of general anesthetization and any other medication to apply, and the projectile used for testing is different only to the extent essential to prevent its unrestrained projection beyond the muzzle-head. Where differences in tonus, pulse, or peristalsis sufficient to affect the test are possible, the operator should wait for the same moment in the action cycle to discharge the airgun.

While significant nonuniformities in the thickness, degree of calcification, and so on, of the diseased tissue warrant retesting before resumption, rather than to unduly detain completion of the procedure, lesions of a kind are assumed to have the same penetration resistance. To preclude flexion, a surrogate projectile limited in forward displaceability having a length equal to that of the chamber plus that of the barrel-tube is made of a solid rod or closed ended thick walled tube of self-bondable E.I. Dupont de Nemours Teflon NXT® polytetrafluoroethylene that matches in caliber or diameter the miniballs used with the barrel-tube. Using a tube, a miniball of the same diameter is bonded onto the front end of the tube by means of an adhesive such as surgical cyanoacrylate cement. Using a solid rod, the front end of the rod is shaped into a hemisphere to simulate a miniball, with radiopacity achieved by plating or capping the tip, first etching the interior of the cap with, for example, Acton Technologies, Inc. FluoroEtch®, then bonding the cap onto the front of the rod with an adhesive such as Loctite Hysol Cool Melt®.

The rod or tube can be inserted into the proximal end of the barrel-assembly when disconnected from the airgun, removal from and replacement in the airgun barrel of the barrel-assembly as needed accomplished quickly. The test rod or tube must be sufficiently pliant to pass entirely through the barrel-assembly without exerting any straightening effect as would distort the result, and should not interfere with rotation of the turret-motor. The testing rod or tube has a dorsal extension, a tab or key, toward its rear or proximal end within the chamber and is provided with a depth gauge type calibration over its distal segment. The key is made by inserting and bonding a tab of polytetrafluoroethylene that is pliant at the base of its faces into a slot cut into the rod or tube. The adhesive used to bond a cap at the front end and key toward the rear is Loctite Hysol Cool Melt®. The key has rounded and polished edges and fits into or engages a slot or groove milled or routed into the ceiling which begins at the distal end of the chamber so that the key can be inserted into and slid along the groove with no more than the intermittent and slight friction of aligning contact.

In a specially constructed interventional airgun intended to achieve deeper penetration, the ceiling groove must be longer and may extend past the front of the chamber into the barrel. The groove and key are made narrow as not to affect discharge. With a break-breech airgun, the test rod or tube is inserted into the barrel from the rear leaving enough length to insert the pliant key or tab toward into the slot or slideway in the valve body. The testing rod is pushed back into the chamber and the breech closed so that the front of the testing rod or tube is flush with the muzzle-port. Accordingly, the testing rod is inserted in the barrel-assembly prior to initial insertion into the vessel or duct to be treated. Thereafter, the testing rod can be freely removed or reintroduced whenever the target diseased tissue appears different in penetrability. When the design of the gun is such that the rear of the test barrel is inaccessible, the testing rod is inserted through the muzzle, then twisted until the key engages the slideway.

The ends or lands of the groove, typically on the order of two millimeters apart, thus represent stops that establish the limits of forward and backward movement or throw of the testing tube or rod and therewith the distance that the tube or rod can protrude out of the front end opening of the barrel-tube, or muzzle-port. When a motorized muzzle-head is to be used to rotate or torque the muzzle-head, the barrel-tubes will have a rotary curve superimposed upon or compounded with the curve that directs these from the axis to the outer edge. The rotary curve compounded with the splay curve poses additional rolling resistance for the miniballs, so that testing should also be conducted with the muzzle-head rotated to angles to be used in the procedure. In use, a table provided with the factory-calibrated airgun is consulted for the optimal impact force and range of impact force settings that proved optimal for such diseased tissue at immediate autopsy, to include those at various rotary positions of the muzzle-head.

Since the settings are determined by the maker for the specific model airgun by comparison of its discharge at each setting to the impact force values obtained with the aid of a ballistic pendulum at autopsy, no compensation for barrel-tubes of higher friction is needed. Normally, the force imparted to the testing rod or tube is transferred with insignificant loss to the diseased tissue to be tested. However, when, for example, entry is inguinal and the target coronary, the barrel-assembly and testing rod follow a long and tortuous path that can dissipate a proportion of the momentum sufficient to invalidate the impact upon the target tissue as a basis for setting the controls on the airgun. Such nonuniform resistance to projection of the testing rod or tube compared to a miniball are reflected in proportionally higher control settings specified in the table provided by the manufacturer. If the most common value in the range specified by the table results in puncture of the tunica adventitia, then the operator goes to the lowest value in the range.

If the lowest value in the range still punctures through the adventitia, then the operator goes to the lowest value of the airgun. The results of the test discharge are carefully noted, and as few as possible adjustments made until the depth sought is attained. The proper value for the instant diseased tissue is arrived at in this strictly observational manner, preliminary value gathering, quantification, and computation accomplished by the maker, so that testing conducted mid-procedure is always strictly observational or empirical and takes the least time. The use of tethered miniballs as testing devices is discounted as usable only over short distances along straight paths. Contacting the internal wall of the barrel-tube, which is unavoidable in a curve, tethered miniballs suddenly roll, begin to wrap their tether around them, which clogs and rubs against the barrel wall, abruptly and unpredictably yanking and braking the miniball. Such action unpredictably and unreproducibly consumes miniball momentum, completely invalidating the results of testing by such means. The testing method and apparatus described constitute a means of durometer testing living tissues whether normal or diseased in situ.

### Endoluminal Pretest for Adventitia or Media Delamination

In health, the layers or tunics in the wall of a ductus cohere despite tonic or peristaltic contraction and relaxation of the smooth muscle at the center of the wall and the orthogonal shear generated by the travel of this action along the wall. In disease, this cohesion may become weakened or undone. Atheromatous plaque, for example, separates the intima from the media. Therapeutic measures such as balloons and lasers can also affect this cohesion. In stenosed or constricted conditions where outward retraction of the outer layer or layers relieves the inward stenosing force that originates in these so that the force of the fluid within is then able to restore substantial patency, delamination, whether preexistent or caused by the vascular endomural implants may not matter. The repair of delamination warrants study.

When the luminal constriction is attributable to an inner layer, however, unless extraluminal implants can be placed to undercut and lift this inner layer, delamination is likely to result in a useless retraction of the outer layers that leaves the diameter of the lumen unaffected. Depending upon the specifics of the condition then, it may be best to obtain an indication as to the cohesion of the layers in the luminal wall. To empirically check the tunic and laminae for susceptibility to delamination from within the lumen, an adhesive delivery-capable testing catheter of the same diameter as the miniballs for insertion, typically 0.4 millimeters, with hemispherical tip at the front and exceeding the barrel-tube length by two to five millimeters is passed through the barrel-tube that is closest to the lumen wall on the side to be implanted.

The hollow test rod can be used to inject a commercially available radiopaque solution that will then fill the void in any separation between layers. While kept under view tomographically, the test rod or adhesive delivery-capable catheter is slowly forced through the intima and media to the adventitia-media interface. Continued force then reveals whether the adventitia will separate from the subjacent media under the force that would be exerted by a stent-jacket that would exert the minimum tractive force essential to make the ductus patent. In situations where an accidental perforation would not spontaneously seal itself promptly and it is not desired to access the exterior of the ductus through a keyhole incision, preferably the original, but alternatively another test catheter having a lumen through which a long-chain methacrylate tissue cement can be injected is used.

### Extraluminal Spot Test for Adventitia-Media Delamination

As stated in the preceding section entitled Endoluminal pretest for adventitia or media delamination, delamination is of concern when the stenotic condition is attributable to an inner layer that cannot be undercut for outward retraction so that to draw the outer layers outward would have no effect on the diameter of the lumen. When the exterior of the ductus can be accessed through a keyhole incision, a commercially available radiopaque solution is injected into the lumen wall with a very fine hypodermic needle. The ensuing pattern should allow a lateral spreading through a separation between the layers in the wall of the ductus to be distinguished from entry and flow with other contents through the lumen. Even if an accidental injury or open surgery has fully exposed the ductus, to reliably evaluate any delamination between its layers using only a forceps or probe and without transecting it is impossible, making the need for contrast clear.

### Muzzle-Head Adhesion Test

Since the implants are generally to be positioned uniformly at close intervals, clinging or adhesion of the endothelium to the sides of the muzzle-head, despite its nonthrombogenic fluororopolymer coating, with the risk of rotational stretching injury, must be avoided. The avoidance of adhesion is especially important during automatic discharge, which can proceed so quickly that the operator does not realize the problem to push the cancel button. For this reason, smooth movement over the run segment is confirmed before automatic discharge is initiated. A muzzle-head with fluoropolymeric coating is intrinsically lubricious and, if necessary, can additionally be coated with a lubricant as specified above prior to introduction. When the diameter of the lumen wall at the level to be implanted becomes smaller relative to that of the muzzle-head, especially when the condition of the wall promotes adhesion, additional lubricant may be necessary. Once at an appropriate depth into the vascular tree, rather than to test for adhesion by manually rotating the barrel-assembly risking rotational injury to the lumen wall, the turret-motor is used for controlled rotation too slight for such injury to become significant.

When the muzzle-head reaches the level of the ductus for implantation, the effect of attempting to rotate the muzzle-head to either side with the turret-motor is observed for free movement, the tantalum markings or indices on the muzzle-head assisting in this determination. While with either an angioplasty or nonangioplasty barrel-assembly, the turret-motor is not normally used before discharge, hence, before the barrel-assembly is connected to the airgun, the test for adhesion of the lumen wall to the muzzle-head and procedure for spreading lubricant about the muzzle-head once introduced through a muzzle-port makes use of the turret-motor prior to insertion in the airgun. Use of the turret-motor to check adhesion or to spread lubricant or medication prior to initiating discharge represents a distinct function of the turret-motor. If previously connected to the airgun, the barrel-assembly may be disconnected for such purpose.

### Midprocedural Delivery of Lubricant to the Muzzle-Head

If despite the measures incorporated to minimize such an eventuality, adhesion of the muzzle-head to the endothelium occurs, a hypodermic syringe is used to inject lubricant into the proximal end of a barrel-tube or tubes used as a distal access barrel-tube as described above in the section entitled Muzzle-head (Barrel-assembly Distal) Access Barrel, and a ramrod or test rod used to push the lubricant out through the muzzle-port. Rotation with the turret-motor as preferred for controllably minimizing the rotational displacement or by hand is then used to work the lubricant around the muzzle-head. A felt or cotton-coated ramrod is then used to remove any residual film from the walls of the barrel-tube or tubes.

### Followup Examination

Once implanted, the status of stent-jackets, stent-stays, clasp-magnets, clasp-wraps, and magnet-wraps must periodically be reinspected. Stent-jackets may lose resilience, vascular intramural implants (miniature balls, stays) and clasp prongs can be pulled through the intervening substance of the vascular wall, and magnetically retracted tunic or tunics may delaminate. Every stent known is subject to structural failure, migration, or both. Endoluminal (conventional) ureteral stents, for example, are known at the time of placement to require replacement, but are nevertheless often disregarded if not forgotten. That compared to an endoluminal stent, the failure and/or migration of a stent outside the ductus poses little threat of occlusion is inarguable; however, the loss in patency is no less serious. For examining an extraluminal implant, intravascular ultrasonography is of little value. However, advances such as dual-energy contrast-enhanced computed tomography allow visualizing the current status of the different implants described herein, with or without a die or tantalum indices on the surfaces of the implants, noninvasively.

### Sterilization

Sterility in the packaging and handling of extraluminal stenting components is basic as essential to prevent infection and fistulization. The various components of the apparatus set forth include sequential or line-feed preloaded clips; rotary magazine clips; stent-jackets; subcutaneous encapsulated magnets; barrel-assemblies, which may include a motorized turret; test rotary magazine clips, airguns, stays, and stay insertion tools. To preclude misapplication, miniballs are not sold loose or in bulk and are never meant to be loaded individually; rather, these are purchased in preloaded clips as units with package markings to indicate proper use. All of the disposable components are sold individually packaged to assure the preservation of sterility. Means for the sterile packaging of medical supplies are universally implemented throughout the medical packaging industry.

Of the foregoing, sequential or line-feed preloaded clips, rotary magazine clips, stent-jackets, and subcutaneous encapsulated magnets are sold in sealed fully labelled paper envelopes with laminated foil interior, are meant to be implanted, and not sterilized after opening. A more specialized and costly stent-jacket or subcutaneous magnet that is opened in error can nevertheless be sterilized chemically, as with ethylene oxide gas (reference, International Standards Organization standard 11135, Medical Devices—Validation and Routine Control of Ethylene Oxide Sterilization, peroxide plasma, electron beam, or gamma radiation. Alternative methods of sterilization should not be used with magnets. The high temperatures of boiling or steam autoclaving can degrade magnets, as can beta or gamma particle irradiation.

Barrel-assemblies, test clips, and airguns are permanent and require sterilization by nondestructive means. Barrel-assemblies are made of plastics, primarily fluoropolymers, with muzzle-heads usually made of steel and possibly motors that contain magnets; test clips consist of plastics and metals; and airguns include canisters containing compressed air or CO₂, the latter a liquid while contained and a gas when released, metals, plastics, and solenoids containing magnets. Chemical sterilization is preferred as applicable to all components. Suitable antiseptics include ethylene oxide and chlorine dioxide. Chemical sterilization cabinets or chemiclaves generally generate heat that falls safely below the Curie temperature of neodymium iron boron magnets.

### GLOSSARY OF TERMS

Adjusting stent-jacket—A stent-jacket (cf.) with an expansion insert (cf.) along one or both free edges facing across the side-slit (cf.). Using absorbable materials in order of absorption, the stent-jacket is made to close over time, ideally, in step with subsidence in the initially enlarged ductus; contracting stent-jacket.
Ablation[-capable] barrel-assembly—A radial discharge barrel-assembly (cf.) that can apply heat (electrocautery; thermal cautery) or cold (cryogenic cautery; cryocautery) to ablate lumen obstructive matter in any type ductus while physically independent from the airgun and without regard to subsequent stenting. Except for the use of different thermal or cryogenic temperatures, an alternative characterization for an angioplasty-capable barrel-assembly.
Angioplasty [-capable] barrel-assembly—A radial discharge barrel-assembly (cf.) that unlike a simple pipe or a an (solely discharge, limited-purpose) barrel-assembly, can serve to perform an angioplasty without regard to subsequent stenting. An angioplasty-capable barrel-assembly may incorporate only side-sweepers (cf.) with trap-filter (cf.) or it can be of a combination-form type that also incorporates a laser or rotary burr. Used manually without insertion in the airgun and prior to insertion in the airgun when ballistic implantation stenting is to follow, an angioplasty-capable barrel-assembly has a free extracorporeal end, and battery powered, it is untethered for free and independent manual use. Should the operator wish to advance or retract the angioplasty barrel-assembly in increments too small for manual control, the free end of the barrel-assembly can inserted into the airgun barrel to use its stepper motor positioned linear table; angioplasty barrel-assembly.
Ablation and angioplasty-incapable] barrel-assembly—A barrel-assembly (cf.), such as a simple pipe or solely discharge radial discharge barrel-assembly for insertion into an airgun as an endoluminally insertable extension of the airgun barrel; plain discharge barrel-assembly, limited purpose barrel-assembly.
Antemagnet chamber—The enclosed space in front of a miniball recovery and extraction recovery tractive electromagnet and behind the spring-loaded door, which is flush with the outer surface of the muzzle-head. The door springs urge the door to re-close outwardly after a loose or extracted miniball, drawn into and trapped within the space by attraction to the magnet, has pushed the door open.
Articulated stent-jacket—A stent-jacket that consists of separate segments of tubing connected by joints to allow flexion and thus compliant with gross movement or flexion as well as passing or longitudinal changes in gauge without buckling in the side-slits (cf.); jointed stent-jacket (cf.).
Anti-migration lining—A layer applied to the internal surface of a stent-jacket (cf.) in order to reduce if not eliminate lateral displacement. When the muscular forces intrinsic in the substrate ductus or its exposure to external forces recommend, additional protection against migration is obtained through the use of stent-jacket end tethers (cf.).
Anti-perforation lining—A layer applied to the internal surface of a stent-jacket (cf.) in order to eliminate the risk of escape outside the ductus and to reduce if not eliminate the intensity or residual momentum of rebound as could place the miniball (cf.) in the lumen.
Barrel-assembly—A barrel-catheter (cf.) with muzzle-head (cf.) made as a unit. An angioplasty-capable barrel-assembly includes components so that it can be used for angioplasty without regard to subsequent stenting, whereas a limited-purpose barrel-assembly can only be used to deliver miniballs.
Barrel-catheter—The tube that in a simple pipe or singular barrel-tube comprises the barrel-assembly, and in a two to four-way embodiment conveys separate barrel-tubes.
Barrel-channel—The portion of each barrel following or distal to the terminus of the barrel-tubes where continuation is through the rotating nonmagnetic metal or spindle portion of the muzzle-head. The junction of the barrel-tubes with the barrel-channels is always by a joint of constant internal diameter across the junction which is slidable or reciprocating when the pliancy of the barrel-tubes or a lack of sufficient slack results in distortion or kinking of the barrel-tubes but is otherwise bonded.
Barrel insertion segment—The proximal length of an angioplasty barrel-assembly that is inserted into the barrel of the airgun to initiate ballistic implantation. Arranging that the hand grip shaped onboard battery pack, which is used to power the barrel-assembly while used independently of the airgun and the airgun or a separate power supply for angioplasty, can be slid proximally along the barrel-catheter allows the proximal segment of the barrel-assembly to be used intracorporeally, reducing the overall length of the barrel-assembly required for a given procedure; overhang.
Barrel-tube—An airgun miniball propulsion channel. In a simple pipe, the barrel-tube comprises the barrel-assembly, which consists of a simple barrel-catheter, whereas in a two or four-way radial discharge embodiment, the two to four barrel-tubes are contained within the barrel-catheter. Its distal end is the muzzle-port, regardless of the materials of which some of its segments are comprised.
Base-tube—In a stent-jacket (cf.), the segment of tubing that serves as the pliant platform upon which the perpendicularly magnetized bar magnets are mounted. Its inner surface serves to set the limit to the excursion or distance from the central axis of the lumen to which the ductus wall can be drawn, and no more distant from the external surface of the ductus than is necessary to effect sufficient patency or normal blood (TIMI III) flow, prevents stretching injury.
Base-tube (slit) expander—A stent-jacket base-tube slit expanding hand-tool used to expedite placement into a surrounding or circumvascular relation of a stent-jacket and a vessel or duct.
Blanked [rotary magazine] clip—a rotary clip inserted to eliminate a number of barrel-tubes of the barrel-assembly from use, usually to treat eccentric lesions. The holes in the clip that would nomrally retain the miniball or miniballs for barrel-tubes to be eliminated are reduced in diameter or eliminated.
Blood-groove—Longitudinal furrows or running depressions that run along the outer surface of the barrel-catheter and muzzle-head portions of the barrel-assembly to allow some circulation of blood. The blood grooves or depressions are made as deep and as wide as the requirement to not encroach upon the internal barrel-channels allows.
Blood-port—An open space in the muzzle-head through which blood can flow created by removing nonessential metal.
Blood-tunnel—A channel through the barrel-catheter to provide a passage for the flow of blood that also stiffens the catheter.
Bounce-plate—A trajectory reflector mounted to the end of the muzzle-head of a simple pipe barrel-assembly to reverse the direction of implantation; rebound-plate; rebound-tip; ricochet-plate; ricochet-tip; strike-plate; strike-tip.
Brush holder—A receptacle in the muzzle-head for receiving an interchangeable side-sweeping brush.
Capillary catheter—A fine catheter passed down the barrel-assembly to quickly return the turret-motor and/or miniball recovery electromagnets to normal body temperature. The capillary catheter has a closed distal end and side-holes through which chilled air or high purity 1,1,1,2-tetrafluoroethane (R134a) cryogen spray is pumped at low pressure; rapid cooling capillary catheter.
Center discharge barrel-assembly—A barrel-assembly lacking a burr or laser atherectomy cable running down its center, which is thus free for use as peribarrel space, and the closer positioning together longitudinally of the barrel-tubes.
Center discharge muzzle-head—The muzzle-head (cf.) in a central discharge barrel-assembly.
Centering device—A disk with holes placed at intervals along the barrel-assembly, which is used to space the longitudinally disposed barrel-tubes that course through it at the concentric distances desired. The intervals along the barrel-assembly and slidability or bonding of the barrel-tubes to these allows considerable variability in the flexibility and torqueability of a barrel-assembly made of tubes of a given material; centering disks.
Central canal [of the barrel-assembly]—A longitudinal axial space amid the barrel-tubes containing the wires to the turret-motor and miniball recovery and extraction recovery tractive electromagnet assembly. In order to reduce the diameter of a barrel-assembly that includes a laser catheter, the barrel-tubes, rather than radially symmetrical about the longitudinal axis, are collected together within an arc to a side of the laser catheter.
Clamp-collar—A round clamp at the rear or proximal end of the turret-motor for securing the end of the barrel-catheter.
Clasp-magnet—A permanent magnet encapsulated within a chemically isolating envelope of plastic or metal for attachment to the muscle fascia or pleura; patch-magnet.
Clasp-wrap. —A wrap-surround (cf.) mounting ferromagnetic miniballs that is placed about a ductus that whether normally or as the result of disease, is incapable of being implanted with or retaining miniballs. It is literally extravascular (circumvascular, perivascular), but as a prosthetic outer layer of the ductus, ‘intravascular’ but not endoluminal (intraluminal) in concept; clasp wrap-surround, clasp-bandage, clasp-jacket.
Clip shot-group—The set of miniballs to be discharged together from the separate hole clusters (cf.) for these in the rotary magazine clip. The meaning is unrelated to use of the same term to denote a set of holes in a target produced with a firearm set to certain aiming adjustments.
Combination-form barrel-assembly—A barrel-assembly (cf.) conveying an atherectomy burr of laser along its central axis to its intracorporeal end. Such a barrel-assembly uses an edge discharge muzzle-head (cf.).
Combination-form muzzle-head—The muzzle-head (cf.) in a combination-form barrel-assembly.
Compound tubing—highly pliant polymeric tubing lined with a thin layer of polytetrafluoroethylene approved for medical use; co-extruded tubing; a coextrusion.
Control panel—Either 1. The positioning and discharge set of controls mounted to the airgun or 2. The ablation and atherectomy set of controls mounted on-board an ablation and angioplasty-capable barrel-assembly (cf.).
Cooling [capillary] catheter—A tube with a chamfered or conical front (distal) closed end and side-holes over the distal segment, which is positioned alongside or just short of the recovery electromagnets, side-sweeping brushes, and turret-motor when fully inserted into the central canal of a center-discharge or the barrel-tube of a center or edge-discharge barrel-assembly. The appellation ‘capillary’ pertains to cooling catheters for passing down a barrel-tube, which must be very small in diameter. To quickly return the heated components to body temperature, vortex tube-generated cold air is passed through the side-holes of the cooling catheter; rapid cooling catheter; chilling catheter.
Cooling [capillary] catheter insertion channel—A passage cut along the central axis proximal to the recovery electromagnets in a center-discharge muzzle-head ejection head for acceptance of the distal end of a cooling catheter.
Discharge set—A plurality of successive discharges belonging to a discrete sequence or group unit directed at a specific segment or lesion along the lumen. With a multiple barrel barrel-assembly, each discharge may implant multiple miniballs; discharge group; discharge sequence.
Discharge stack—An instruction stack that coordinates a. The two drivers consisting of open-loop controlled stepper motor operating the linear positioning table for transluminal movement and the closed-loop controlled dc servomotor that rotates the muzzle-head with b. Actuation of the airgun push solenoid (used as a hammer to strike the valve body pin) through the program sequencer and servodrive controller in order to execute a preset discharge pattern.
Distal—Farther from the operator than the point of reference.
Double wedge [stent-jacket] lining—A lining for a stent-jacket (cf.) consisting of complementarily tapered layers running in opposing directions that together constitute a rectangular block for directing any rebound by a miniball away from the lumen and into a functional subadvential or medial location.
Edge discharge barrel-assembly—A barrel-assembly having a burr or laser atherectomy cable running down its center, so that a central canal is unavailable for use as a portion of the peribarrel space that in a center-discharge barrel-assembly (cf.) can be used to insert a cooling capillary catheter (cf.) past the turret-motor (cf.) and into the ejection-head to cool these components when used as heating elements for thermal angioplasty; combination-form barrel-assembly.
Edge discharge muzzle-head—The muzzle-head (cf.) in an edge discharge or combination-form barrel-assembly (cf.) wherein the axial center is occupied by an atherectomy, burr or laser cable; combination-form muzzle-head.
[Muzzle-head] ejection head—The solid nonmagnetic metal distal or front portion of the muzzle-head (cf.), which accepts and fixes in position the distal ends of the barrel-tubes.
End-cap—A plate at the distal end of the barrel-assembly or proximal end of the spindle neck that is excluded in most embodiments.
End-plate—The centering device at the proximal end of the barrel-assembly.
[Stent-jacket end-tethers]—Outrigger anchors beyond the end of a stent-jacket for increased resistance to migration; side-tethers, side-straps; end-straps, end-tethers.
End-ties—Soft strings or straps of braided or multifilament suture at the ends of a stent-jacket, clasp-wrap, or magnet-wrap, which may be used to tie around the substrate ductus in order to prevent migration, or if not required, are snipped off.
Endothelial breakaway—The ability of a muzzle-head (cf.) to move without clinging to or seizing against the interior of the lumen. Sufficient lubricity and avoidance of a muzzle-head that is too large in diameter for the lumen minimize entrapment by adherence. A lubricant can be delivered to the stuck muzzle-head through a service channel (cf.). In more elaborate embodiments, the muzzle-head can also be oscillated to work it loose.
Esophageal tacking—The support of a collapsed tracheal ceiling by attraction to magnets retained along ventrolateral lines within a magnet-wrap (cf.) placed parallel to the segment of trachea affected about the esophagus.
Extended adjustment stent-jacket—A stent-jacket (cf.) (adjustment stent-jacket, cf.) with a stent expansion insert that includes constituents that take a longer time to be absorbed or require deliberate action, such as the use of a lithotriptor, to break down; extended contraction-time stent-jacket.
Exit velocity—The instantaneous velocity of the miniball upon discharge at the muzzle-port; the muzzle velocity. The term is necessitated by the fact that the muzzle-head and original muzzle are different parts of a conventional airgun that has been modified.
[Stent-jacket] expansion-insert—An arcuate segment of absorbable or percutaneous ultrasonic lithotriptor-destructible material applied to an edge of a stent-jacket side-slit or side-slot to allow the stent-jacket to gradually decrease in diameter as an originally enlarged condition of the substrate ductus subsides; stent-insert; stent-insert.
Expansion slit—The cut-line along the side of a stent-jacket (cf.) that providing free edges, allows compliance of the elastic base-tube (cf.) with movement in the wall of the vessel or duct.
Extraction [field] intensity; extraction field strength—the magnetic field strength used to remove a miniball that had been misplaced upon implantation.
Extraluminal stent—A stent that consists of subadventitially implanted intravascular sperules referred to as miniature balls or miniballs and a perivascular magnet surround, or stent-jacket.
Filter-deployment solenoid—A subminiature dc push-type solenoid in the nose of the muzzle-head (cf.) located behind the stowage silo of the trap-filter used to eject or deploy and retrieve the trap-filter after use. It is dampened to prevent abrupt jerks or jolts that could result in injury to the wall of the lumen.
Flex-joint—A ring of elastic material interposed between metal portions of the spindle (cf.) in the turret motor rotor and the splay chamber (cf.) to allow a predetermined amount of flexion; flex-ring.
Forward drive stabilizer—An extendable longitudinal scaffold or framework that holds the barrel-catheter straight, as is essential to eliminate sag and off-axis deflection when the airgun linear positioning table advances the airgun to set the distance between successive discharges.
Full-round stent jacket—A stent-jacket that is completely circular to allow encirclement of a ductus (a vessel or a duct), is made of resilient tubing to maintain clutching contact, and has a side-slit that allows full compliance with the smooth muscle action in the ductus wall.
Gas pressure relief channel—The portion of the gas pressure relief path (cf.) that consists of a passageway machined into the metal portion of the muzzle-head (cf.) spindle (cf.). In multibarrel embodiments, each barrel channel is provided with a feeder branch to the central channel in the muzzle-head (cf.) and peribarrel space (cf.). The channel serves to divert the gas pressure that builds up in the barrel-channel or channels (cf.) during discharge, preventing the gas from entering the bloodstream; gas return channel.
Gas pressure relief path—The passageway that diverts gas pressurized during discharge from being expelled into the bloodstream producing an air embolism. It takes the pressurized gas through the gas pressure relief or gas return channel (cf.) and then through the gas return tube continuous therewith and end-cap (cf.) for return to the peribarrel space; gas return path; gas recursion path.
Heat-window—A silver or copper sheet covered opening in the body of the muzzle-head for conducting heat to the lumen wall for performing a thermal angioplasty or ablation. The heat-window at the front or distal end of the muzzle-head is the fully blunt conical nose-cap heat-window. The heat-window or windows over the turret-motor are typically slit, slot, or rectangular rather than circumferential for treating eccentric lesions.
Jointed stent-jacket—A stent-jacket that consists of separate segments of tubing articulated to allow flexion as to preclude any buckling in the side-slits (cf.); articulated stent-jacket. Gas return tube end-cap —XXXX
In situ test—A test to predetermine the exit velocity (force of discharge) suitable for implanting miniballs into a given segment of a ductus with minimal risk of perforation. Due to the unpredictability of diseased tissue, in situ testing is necessitated regardless of whether a stent-jacket is to be placed prior to discharge. Implanting drug releasing and irradiating seed miniballs does not call for a stent-jacket, which could be justified only where a perforation could do serious harm.
Interventional airgun—A special-purpose gas-operated implement for implanting ferromagnetic, medicated, or gamma radiation emitting seed spherules into tissue by projection. Proper adjustment in the force of impact critical and capable of changing from point to point along the tissue, in situ tissue testing and the ability to reset the airgun quickly and precisely midprocedurally can be critical, prompting the development of special-purpose airguns having multiple control points for quick resetting.
Lift-gates—The rib or bristle-retaining backing of the side-sweeping brushes. The side-sweepers are automatically retracted into recesses about and with bristle tips flush to the outer surface of the muzzle-head during transluminal advancement or withdrawal. When stationary, these are reenabled to be projectable beyond the periphery to allow the removal by sidewise sweeping away of soft atheromas.
Limited-purpose barrel-assembly—A radial discharge barrel-assembly (cf.) which lacks components essential to perform an angioplasty.
Magnet assembly—An electromagnet or formation of miniball recovery and extraction tractive electromagnets at the front end of the barrel-assembly, one in a simple pipe and two in radial discharge barrel-assemblies. For trapping operation, or to prevent a loose miniball from passing down the vascular or tracheobronchial tree, these are set to a resting field strength. When paired, the oppositely oriented electromagnets are referred to as a magnet assembly. To allow the extraction of a miniball that settles in an unintended position, the magnets are adjustable together or individually; trap extraction magnet assembly (cf.), tractive electromagnet assembly, recovery electromagnet assembly.
Magnetic stenting—The use of tiny implanted bar magnets, which may be of any nonspheroidal three-dimensional shape, such as discoidal, cylindrical, or hexahedral, to attract ferromagnetic implants inserted within the wall of a stenosed or collapsed ductus whereby the resultant retraction outwards of the wall of the ductus serves to unblock the lumen.
Magnet-wrap—A bandage or wrap-surround (cf.) in the form of a stretchable cuff for encircling a ductus neighboring another that has become collapsed or stenosed. The magnet-wrap mounts permanent magnets for attracting ferromagnetic implants in or attachments to the failed ductus to which it runs parallel over the treated area for the purpose of exerting tractive force to maintain the failed ductus in a patent condition. The use of the esophagus to the support the collapsed trachea in a small dog is an example; magnet wrap-surround, magnet-cuff, magnet-bandage.
Medicated miniball—A spherule for implantation within the wall of a vessel that consists of medication or medication surrounding a spherical seed type source of gamma radiation; cf. seed miniball, tablet miniball.
Miniball [Miniature ball]—A spherule projectile such as that used in ‘BB’ guns but much smaller, for use in man, generally ranging in diameter from 0.25 to 2.0 millimeters, and most often 1.14 to 1.52 millimeters. Miniballs can be ferromagnetic, magnetic, consist entirely of medication, represent small spherical irradiating seeds, or within the caliber usable, combine these in concentric layers; spherule, miniature sphere, minisphere.
Miniball-hole—The opening in a rotary magazine clip in which a miniball is fixed in position for discharge. The distal and proximal holes describe the openings to a tunnel that runs through the clip, and an internal circumferential ridge midway along the tunnel prevents the miniball from dropping into the barrel. Various dried solutions of sugars and starches that differ in retentive strength are used for added adhesion until discharge or to differentially adjust the relative propulsive force essential to initiate the ejection, hence, the muzzle velocity, of a given miniball in a set of miniballs for simultaneous discharge as a set.
Miniball-magnet—A magnetized miniball, which can additionally be coated for the delivery of medication or radiation.
Monobarrel [barrel-assembly]—A barrel-assembly having one barrel-tube. A monobarrel can be of the simple pipe kind and thus end-discharging through a singular muzzle-port at the distal end or housed within a more or less shuttlecock or torpedo-shaped muzzle-head with the muzzle-port or ports about the circumference referred to as a radial discharge monobarrel type barrel-assembly. Whereas a simple pipe is for use in the airway, a radial discharge barrel-assembly is for use in ducti and embodiments for use in blood vessels must incorporate features to prevent the backflow of blood into the muzzle-head or the injection of gas into the bloodstream during discharge. Such features include pressure relief means in the form of a barrel-catheter (cf.) to provide a peribarrel space (cf.) within, and an extracorporeal one-way safety valve at the proximal end of the barrel-catheter, and gas recursion channels (cf.) in the spindle (cf.) to bleed off excessive gas pressures before these can arrive at the muzzle-port.
Motorized swivel joint—A remotely rotated junction in a radial discharge monobarrel (cf.). With only the one barrel-tube, rotation is concentric with no rotatory deflection.
Motorized turret joint—A remotely rotated junction in a radial discharge multibarrel (cf.). Since plural barrels must be off-center, rotation imparts a rotatory deformation and longitudinal foreshortening to the barrel-tubes, which must be compensated by pliancy, slack in the splay chamber (cf.), and reciprocal movement in the barrel-tube-(cf.) barrel-channel (cf.) joints.
Multibarrel [barrel-assembly]—A barrel-assembly with multiple (plural) barrel-tubes, which is always of the radial-discharge type; multiple barrel barrel-assembly.
Muzzle barrel—The portion of a barrel distal to the insertion and end of the plastic barrel-tube into its muzzle-head flush joint socket and start of the metal portion of the barrel.
Muzzle-head—The part at the front or distal end of the barrel-assembly containing the barrel exit port or ports and the trap-extraction magnet assembly; muzzle-probe. A distinct muzzle-head is characteristic of radial discharge barrel-assemblies; however, the term applies to simple pipe barrel-assemblies as well.
Muzzle-head access barrel—A barrel-tube used, for example, to allow a lubricant or medication to be delivered to the endothelium through a muzzle-port or a cooling catheter to be aligned in closer adjacency to a heated element; barrel-assembly distal access barrel-tube.
Muzzle-port—The miniball exit hole at the distal terminus of a simple pipe muzzle-head or radial discharge barrel-assembly barrel-tube.
[Muzzle-]spindle—The rotating part of the muzzle-head distal to the turret-motor housing; muzzle-head spindle.
Muzzle velocity—The velocity upon exiting the muzzle, which since it can be mistaken to refer the muzzle of the airgun rather than the muzzle-port at the distal end of the barrel-assembly, is best avoided; exit velocity.
[Muzzle-head] Nose—The front end of a barrel-assembly; more restrictedly, the face-on aspect of the front end of a barrel-assembly. Except in a thermal angioplasty- or ablation-incapable barrel-assembly, it is the nose-cap heat-window for minimizing the risk of disrupting vulnerable plaque by initial contact with the muzzle-head; nose-cap, nose-dome.
[Muzzle-head] Nosing—The conformation, always substantially domed (blunt, hemispherical) of the nose, or distal segment of the muzzle-head. When the barrel-assembly incorporates a laser, the distal end of the optical cable slightly protrudes at the nose center as a feature of the nosing.
One-over barrel-assembly—A barrel-assembly (cf.) with at least one barrel-tube more than is needed for implantation. The additional barrel-tube allows adjunct function catheters, such as a test rod (cf.) or cooling capillary catheter (cf.) to be passed down to the muzzle-head. For cooling, this is significant with edge-discharge or combination-form barrel-assemblies, which lack a central canal.
Partial [-ly round] stent-jacket—A stent-jacket with a longitudinal band or slot of the base-tube removed along the side-slit to afford clearance for a line of connective tissue that attaches the vessel or duct to be stented to another structure along one side; slotted stent-jacket.
Peribarrel[-tube] space—The space surrounding the barrel-tubes in the barrel-catheter. Made accessible to the gas that is pressurized during discharge of the airgun through perforations in portions of the barrel-tubes and centering devices inside the patient, this space allows the pressure to be transmitted from the front to the sides and back of the advancing miniball and thus equalized within the barrel-catheter without the release of gas into the bloodstream.
Perforation—The through-and-through penetration or puncture of the lumen wall as the result of excessive exit velocity (momentum) or impact force.
Plunger-piston—The multiflanged elastomeric cap on the adhesive cartridge that is inserted into the chamber above the stay load queue. It is forced deeper into the barrel of the cartridge to expel adhesive by air pump action.
Probe-rod—A fine rod for passing down a barrel-tube so that the lumen wall can be prodded to test its penetrability or deliberately punctured to a limited depth in order to ascertain the susceptibility for the layers of the ductus or tunics to delaminate. A measuring instrument may be used at the driven end to quantify these results.
Proximal—Closer to the operator than the point of reference.
Pull-through—Gradual (progressive) perforation of the adventitia or the layers of the ductus wall that separate miniball implants or stays from the internal surface of the stent-jacket under the tractive force of the base-tube circumsurfacial magnets. Pull-through can result from sustained nonuniform or disproportionate distribution of magnetic traction on one or a few miniball or stay implants of a formation; tear-through.
[Air] pump-piston—A disk closely fitted into the longitudinal chamber behind the stay load queue that is moved by an extension or trip connected to or continuous with the trigger or plunger insertion mechanism, which action causes it to reciprocate. This allows it to act as the air compressing surface of an air pump that is used to expel the adhesive used to seal the stay entry incisions.
Radial discharge muzzle-head—A barrel-assembly (cf.) wherein the barrel-tube or tubes (cf.) bend radially within the muzzle-head as to be unseen, and are therefore seen to discharge at the circumference and not, as in a simple pipe, which is exposed to present a sharp tip as needed to obtain accurate aim in an anatomically nonuniform or structured lumen, out the distal end. To uncut the tunicae intima and media, the barrel or barrel-tubes are angled forward. A radial discharge barrel-assembly is not compatible with the use of a bounce-plate; however, the latter would be benefit only in the airway of the smallest dogs.
Radial discharge barrel-assembly—A barrel-assembly with a radial discharge muzzle-head (cf.). It may be of the monobarrel (cf.) or multibarrel (cf.) kind.
Radial discharge monobarrel—A single barrel radial discharge barrel-assembly (cf.) (radial discharge monobarrel, radial m-barrel).
Ramrod—A tube or solid rod with a mildly magnetized tip for retrieving a miniball accidentally discharged from the airgun chamber with too little force to eject from the muzzle-head. A test shaft is never magnetized, and a ramrod is never used for in situ tissue testing; ram-rod.
Rebound lining—A shock absorbent layer applied to the internal surface of a stent-jacket, especially one intended for placement prior to discharge in order to avert the risk of perforation or rebound into the lumen. The resilience of the layer is based upon the thickness of the wall of the vessel, the angle of entry, to which the angle of rebound will be equal in the opposite direction, and the force of impact.
Rotary magazine clip hole—The hole for each miniball in the rotary magazine clip; clip hole.
Seed miniball—A gamma radiation emitting spherule for implantion within the walls of vessels to highly localize the region irradiated. Radiation exposure to passing blood is negligible and short-lived. A seed miniball can be jacketed or multiply coated with medication; miniball seed.
Service channel—A passageway through the barrel-assembly (cf.) and muzzle-head (cf.) allowing access to distal portions of the barrel-assembly and lumen through a muzzle-port (cf.) or when available, the terminus of the central canal (cf.) for maintenance purposes or for the local delivery of a lubricant or medication, such as an anticoagulant in more concentrated than the systemic level at thermal angioplasty sites, for example; muzzle-head access channel.
Side-slit—The longitudinal cut along one side of a stent-jacket (cf.) to allow its free expansion and contraction in response to changes in gauge due to tonic, pulsatile, or peristaltic forces. In a partial stent-jacket (cf.), the side-slit is expanded to clear the attachment of the vessel or duct to adjacent tissue following the clearing away of loose superficial fascia; stent-jacket side-slit.
Side-slot—In a stent-jacket, a side-slit that has been enlarged to clear a connective attachment or a branch of the ductus; a circumferential extension into a longitudinal arcuate gap of the side-slit (cf.) sufficient to clear a ductus attachment or a branch of the ductus; stent-jacket side-slot.
Side-sweepers—Brushes normally recessed about the miniball recovery and extraction tractive electromagnet assembly at the forward end of the muzzle-head that can be radially projected to scrape the lumen wall when the muzzle-head is rotated with the swivel or turret-motor (cf); lift-gates (cf), side brushes, side-sweeping brushes (cf.); side-sweeper/scrapers, scraper-brushes; reaming-brushes.
Simple barrel-assembly—A barrel-assembly not incorporating a laser or rotational atherectomy or atheroblation burr.
Simple pipe [barrel-assembly]—A single barrel or monobarrel barrel-assembly (cf.) with curved distal portion and a single trap-miniball recovery and extraction tractive electromagnet. A simple pipe may include a bounce plate for reversing the direction of the trajectory. The simple pipe barrel-assembly corresponds to a barrel-tube within a radial discharge muzzle-head (cf.) but is independent and larger. It can be made as a single length of tubing or as one length or segment for the main part of the barrel-catheter, a bent segment of stainless steel, for example, and soft elastomeric distal tip.
Simple stent-jacket—A stent-jacket (cf.) consisting of a single segment of tubing, i.e., a stent jacket that is not multisegmental and jointed, or articulated.
Single [double-, triple-, quadruple-, multiple-]-discharge—Said of a barrel-assembly with respect to the number of barrel-tubes, hence, the number of miniballs that may be discharged at the same time, not repeating ability.
Slotted stent-jacket—A stent-jacket (cf.) having a longitudinal arc of the base-tube (cf.) removed to accommodate a running connective tissue attachment along one side of the vas or ductus stented.
Spindle—The middle portion of the muzzle-head that receives the barrel-tubes and continues these toward the muzzle-ports as the barrel channels. It is usually machined from a single piece of nonmagnetic stainless steel, then through-hardened.
[Muzzle-head] spindle neck—The portion of the spindle that is journaled within the rotor of the turret-motor.
[Muzzle-head] spindle throat—the portion of the muzzle-head spindle between the neck journaled within the rotor of the turret-motor and the ejection-head.
Splay chamber—In the proximal portion of a muzzle-head, a cavity distal to the collar securing the muzzle-head to the distal end of the barrel-catheter that allows the barrel-tubes to bend or veer radially outward toward the muzzle-ports gradually as not to kink; barrel-tube splay-chamber.
Stay—An arcuate rib band for insertion into a collapsing or stenotic ductus to correct the condition. A stay that contains ferromagnetic material such as a core of soft iron for implantation into a ductus wall so that the wall will be retracted by an encircling stent-jacket carrying permanent magnets on its outer surface is a stent-stay.
Stay insertion tool—A syringe (pusher-type) or pistol (puller-type) configured hand tool for introducing (implanting) stent and other kinds of stays (cf.) perimedially (subadventitially) or medially into the wall of a ductus; stay injection tool; stay inserter.
Stent-implant—A miniball (cf.) or stay (cf.) with ferromagnetic core that has been infixed within the wall of a ductus for retraction by a stent-jacket (cf.). When the implants are miniballs, which are implanted ballistically, and vulnerable structures surround the ductus that could be injured were a miniball to penetrate entirely through the wall of the ductus, a double-wedge stent-jacket (cf.) that deflects such an otherwise penetrating discharge to an acceptable location in the wall of the ductus is placed first.
Stent implantation—The placement of ferromagnetic miniballs (cf.) or stays (cf.) in the wall of a ductus for retraction by a stent-jacket (cf.).
Stent-jacket—The extravascular (circumvascular, perivascular) component of an extraluminal stent that supports the magnets. A full (−y) round stent-jacket entirely surrounds the vessel or duct, whereas a partial stent-jacket encloses only that circumferential extent of the vessel or duct that is exposed without dissection of a line of connective tissue that attaches the structure to another or a branch that plunges to greater depth. To minimize the effect of the extraluminal stent upon relatively undiseased portions of an eccentric angiosclerotic lesion, the complete or partial extraluminal stent is blanked out for the unaffected arcuate segment by using a blanked out rotary clip implanting miniballs or positioning a bar magnet in this segment. A stent-jacket is a kind of magnet-wrap (cf.) in the literal sense, but incorporates an elastic base-tube as substrate rather than gauze and spandex and thus is not a kind of bandage or wrap-surround.
Stent-jacket applicator—A tool, usually a hand-tool, for opening the stent-jacket for encircling the substrate ductus. It may be designed for ease of use endoscopically or robotically rather than manually; base-tube retractor, side-slit retractor, side-slot retractor, stent-jacket expander.
Stein shot-group—An aggregation of miniballs implanted in close formation to prevent pull-through (cf.).
Stent-stay—A band of ferromagnetic metal cambered for concentricity with the ductus into which it is to be inserted subadventitially (perimedially) by means of a special insertion tool for retraction of the ductus wall by an encircling stent-jacket. Stays that do not include ferromagnetic matter are not stent-stays.
Stop-and-lock ring—A nonmagnetic metal annulus about the barrel-catheter that fixes the distance that the barrel-assembly can be pushed into the barrel of the airgun. Tabs that project from the ring periphery fit into slots within a complementary fitting affixed to the airgun muzzle. These tabs slide through ways to engage the barrel-assembly to the airgun. With tabs twisted into the rotary slideway in the female component of the twist-to-lock connector (cf.), the barrel-assembly is locked in position with its proximal end immediately before the face of the rotary magazine clip or miniball to be discharged and in the correct alignment.
Subadventitial—just inside the external elastic lamina or tunica adventitia as the outer layers of a ductus.
Subcutaneous strip patch magnets—A flat, usually disk shaped, magnet encapsulated within a bioinert outer covering for attachment to the deep or muscle fascia overlying implanted miniballs (cf.). The magnets are adjoined so that one or more can be removed for placement on the deep or muscle fascia overlying the implanted miniballs to be drawn.
Substrate [ductus]—The vessel or duct mantled about by a stent-jacket (cf.), clasp-wrap (cf.), or magnet-wrap (cf.).
Superport projection [of the muzzle-head]—The material of the muzzle-head distal to the ports.
Swivel-motor—The motor used to rotate the muzzle-head in a single barrel or monobarrel radial discharge barrel-assembly, wherein rotation is of one centered or axial barrel-tube, rather than two or more barrel-tubes radial to the central axis; single barrel turret-motor; single barrel turret-servomotor.
Tablet miniball—A spherule that consists of medication for ballistic implantation within the wall of a ductus. Can be unrelated to magnetic stenting (cf.).
Test shaft—A solid rod or tube (catheter) placed in a barrel-tube to take the place of an implant projectile in order to allow the penetrative force corresponding to the exit velocity to which the airgun is set to be evaluated.
[Thermal angioplasty] window—A sector of the muzzle-head body or outer shell cut from thin silver or copper sheet to allow heat generated within the muzzle-head to radiate against the diseased lumen wall in order to achieve thermal angioplasty; heating window.
Trapping [field] intensity; trapping field strength—the low or resting magnetic field strength of the miniball recovery tractive electromagnets used to trap any miniballs that fall into the lumen.
Trap-extraction magnet assembly—An electromagnet or pair of electromagnets mounted to the front of the muzzle-head, of which the individual or combined field strengths can be varied to prevent the escape of miniballs downstream or to retract miniballs that have already been implanted. It is unitized with and integral to every muzzle-head; miniball recovery and extraction tractive electromagnet assembly; recovery electromagnets, retrieval electromagnets, tractive electromagnets.
Trap-filter—A miniature filter having the form of a parachute, umbrella, trawling type fishing net, or windsock that is used with the side sweepers-scrapers to prevent distal embolization by intercepting any angioplasty produced debris that escapes and would otherwise pass down the bloodstream. The trap-filter is deployed from and withdrawn into a concavity in the muzzle-nose (cf.). It can be purchased as a finished product and adapted for incorporation into the nose of the muzzle-head; run-ahead filter, run-ahead trap-filter, filter trap.
[Muzzle-head] turret-motor—The motor used to rotate the muzzle-head at the rotary joint the barrel-catheter in a multiple barrel or multibarrel radial discharge barrel-assembly. Rotation of the off-center barrel-tubes twists these, so that the motor must be limited to an arc through which the barrel-tubes do not deform causing jams upon discharge; t-motor; turret-servomotor.
Twist-to-lock connector—A connector consisting of a male with sliding tabs stop-and-lock ring (cf.) mounted about the barrel-catheter at the distance from the end-plate (cf.) to which the barrel-catheter is to be inserted into the airgun barrel, and a female fitting mounted to the front of the airgun muzzle having slots and channels in which the tabs of the stop-and-lock ring are slid around beneath a compressive ceiling until stopped from further rotation. This connects the barrel-assembly to the airgun at the correct rotational angle. In an angioplasty barrel-assembly, the twist-to-lock connector serves as the proximal stop for the hand grip shaped battery pack when slid back to perform an angioplasty prior to inserting the barrel-assembly in the interventional airgun for stenting.
[Thermal angioplasty] windows, window-slits, window-slots—High heat conductivity areas of the shell or body of a muzzle-head for allowing the eccentrically directed flow of heat from the surge-current heated turret-motor and/or recovery electromagnets for thermal angioplasty.
Working arc—the range of muzzle-head rotation to either side of the center point of turret-motor rotation as limited by the inception of discharge obstructive deformation of the barrel-tubes in use, hence, the arc through which a given turret-motor is limited in rotating a specific muzzle-head.
Wrap-surround—A special bandage used to position magnets or ferromagnetic miniballs around a tubular anatomical structure. One mounting miniballs is a clasp-wrap (cf.), which is used when the ductus wall is incapable of being implanted with or retaining miniballs. It is in turn surrounded by a stent-jacket (cf.). One mounting magnets used to exert patenting tractive force upon the miniballs implanted in a neighboring, usually parallel, structure is a magnet-wrap (cf.), which serves in lieu of an immediate stent-jacket. A stent-jacket is not a kind of bandage or wrap-surround.

1. A catheter for extending the barrel of a gas-operated gun for introduction into, passage through, and discharge within the lumen of a tubular anatomical structure, thus allowing a spherule to be embedded within the wall of said tubular anatomical structure.

2. A spherule for projection by a catheter as defined in claim 1 wherein said spherule consists of ferromagnetic material, which is surrounded by an outer biocompatible layer that chemically isolates said material.

3. A spherule for projection by a catheter as defined in claim 1 which is further coated with medication.

4. A spherule for projection by a catheter as defined in claim 1 which consists of medication.

5. A spherule for projection by a catheter as defined in claim 1 wherein said spherule consists of a radiation-emitting seed.

6. A spherule for projection by a catheter as defined in claim 1 wherein said spherule consists of a radiation-emitting seed as its core with an outer coating of medication.

7. A catheter as defined in claim 1 wherein at least one tractive electromagnet is mounted proximal to the point of spherule discharge so that energizing said electromagnet allows a misplaced spherule to be retrieved.

8. A catheter as defined in claim 7 wherein the current conducted through the coil of said electromagnetic actuator can be increased causing the temperature of said coil to increase to levels suitable for thermal angioplasty and ablation.

9. A longitudinally extendable linkage for supporting the extracorporeal proximal length of a catheter as defined in claim 1 so that when said catheter is advanced through the passageway of said tubular anatomical structure, said catheter does not bend.

10. A shaft for replacing the spherule projectile discharged through a catheter as defined in claim 1, said rod extending to the muzzle of said gas-operated gun, so that when discharged, the effect upon a lumen wall of the shaft allows the effect upon the wall of a spherule that would be discharged with equivalent momentum to be predetermined.

11. An apparatus for stenting a tubular anatomical structure whereby a gas-operated gun catheter as defined in claim 1 is used to implant ferromagnetic spherules beneath the lumen lining of said tubular structure so that said spherules generate magnetic tractive force in relation to material of the opposite magnetic attracting-attracted state, said material of the opposite magnetic state being mounted about the external surface of a length of elastic tubing which is placed in surrounding relation to and thus draws radially outwards the wall of said tubular anatomical structure.

12. An apparatus for reducing the muzzle velocity of a gas-operated gun for use with a catheter as defined in claim 1 by introducing an adjustable pressure relief slot into the valve body of said gas-operated gun.

13. A gas-operated gun for use with a catheter as defined in claim 1 with not less than two points of control for regulating the muzzle velocity of the spherules discharged by said gas-operated gull.

14. A gas-operated gun for use with a catheter as defined in claim 1 wherein an electromagnetic actuator is used to depress the pin in the valve body of said gas-operated gun to admit into the chamber of said gas-operated gun the gas that is used to expel the spherules.

15. A gas-operated gun as defined in claim 14 wherein said electromagnetic actuator is a push-type solenoid.

16. A gas-operated gun as defined in claim 15 wherein the time that the plunger of said push-type solenoid is held in the extended stroke position is adjustable.

17. A base for a gas-operated gun that uses a catheter as defined in claim 1 which base consists of a linear positioning table that allows the muzzle of said catheter to be accurately moved along the lumen of a tubular anatomical structure in small increments.

18. A base for a gas-operated gun that uses a catheter as defined in claim 1 which base consists of a linear positioning table which allows the muzzle of said catheter to be accurately moved along the lumen of a tubular anatomical structure in small increments and that mounts said gas-operated gun for rotation about the longitudinal axis passing through the barrel of said gas-operated gun so that when said catheter discharges radially, the direction of discharge can be rotated.

19. A rotary magazine clip for loading a gas-operated gun that uses a catheter as defined in claim 1 wherein groups of holes for holding sets of spherule projectiles to be discharged simultaneously are separated by angular distances about said clip, each said hole having a rib that continues entirely around its interior surface for retaining said spherule projectiles pending discharge.

20. In a rotary magazine clip used to load a gas-operated gun as defined in claim 19, the running of a tacky substance into and along the circular gap separating a spherule from the surrounding edge of the hole wherein said spherule is positioned in order thereby to cause said spherule to resist discharge.

21. A catheter as defined in claim 1 which is smaller in diameter and axially conducted through a larger outer catheter.

22. A catheter as defined in claim 21 wherein said smaller catheter has gas pressure relief perforations along portions of its length so that pressure developed during discharge is expelled through said perforations into the space between the outer surface of the smaller catheter and the internal surface of said larger outer catheter.

23. A catheter as defined in claim 21 wherein only said larger catheter is divided at a rotary joint, its proximal segment clamped within a collar just proximal and coaxial to the through-bore rotor of a rotary actuator, and its distal segment journaled within the bore of said rotor and on emerging distal thereto, attached to a shell that extends forward to envelop and mount the distal muzzle exit end of said smaller catheter for termination through an exit hole on the side of said shell so that said rotary actuator can be used to adjust the angle at which a spherule is discharged.

24. A catheter as defined in claim 21 that mounts aristae in a brush block for radial extension and thus allows the internal surface of a tubular anatomical structure to be swept.

25. A catheter as defined in claim 21 wherein said brush block is pushed up guideways at its sides when electrical current is sent to a thermal expansion wire upon which said block rests.

26. A catheter as defined in claim 21 wherein said larger outer catheter includes a convoluted segment.

27. A larger outer catheter as defined in claim 21 wherein said catheter of smaller diameter is used as the barrel of a gas-operated gun that courses eccentrically through the larger outer catheter to allow a rotational atherectomy burr cable to extend longitudinally through the center of said larger outer catheter and terminate just beyond the nose at the front end of said larger outer catheter.

28. A larger outer catheter as defined in claim 21 wherein said catheter of smaller diameter is used as the barrel of a gas-operated gun that courses eccentrically through the larger outer catheter to allow an excimer laser cable to extend longitudinally through the center of said larger outer catheter and terminate so that when not deployed, the distal tip of said burr is flush to the nose at the front end of said larger outer catheter.

29. A catheter as defined in claim 23 wherein said rotary actuator is a subminiature through-bore torque motor and the rotor of said rotary actuator remains connected as an electromechanical positioning device but can be switched into an alternative circuit that is used to send current through and thus heat said rotor to 90 degrees centrigrade for performing a thermal angioplasty and to other temperatures for ablating other tissues.

30. A strip for implantion beneath the outer surface of a tubular anatomical structure.

31. A hand tool for inserting a strip as defined in claim 30.

32. A strip as defined in claim 30 which consists of medication.

33. A strip as defined in claim 30 which consists of ferromagnetic material and is overlain with a coating of a biocompatible material to chemically isolate said ferromagnetic material from surrounding tissue.

34. A stent consisting of a plurality of strips as defined in claim 33 which are used to generate magnetic tractive force in relation to material of the opposite magnetic attracting-attracted state, which material of the opposite magnetic state is mounted about the external surface of a length of elastic tubing which is placed in surrounding relation to and thus draws radially outwards the wall of said tubular anatomical structure.

35. A strip as defined in claim 33 having an additional coating of medication at its surface.

36. A strip as defined in claim 30 which consists of a radiation-emitting seed.

37. A strip as defined in claim 36 wherein said radiation-emitting seed is given an outer coating of medication.

38. An elastic sleeve with a slit along one side so that said sleeve can be opened to mantle about a tubular anatomical structure without restraining physiological changes in the caliber of said tubular anatomical structure.

39. An elastic sleeve as defined in claim 38 wherein said slit is edged with a dissoluble slit-expanding insert such that upon dissolution of said insert, the edges of the sleeve slit come together.

40. A tool for expanding the slit in an elastic sleeve as defined in claim 38 and thus allowing said sleeve to be placed in surrounding relation to said tubular anatomical structure, said tool configured as a tweezers with spatula-shaped blades directed inward at the distal ends.

41. A tool for expanding an elastic as defined in claim 38 and thus allowing said sleeve to be placed in surrounding relation to said tubular anatomical structure, said tool configured as a forceps with spatula-shaped blades directed inward at the distal ends.

42. A mounting for a ferromagnetic body for use with a length of elastic tubing as defined in claim 11, where said mounting consists of a base made of a nonmagnetic material having prongs directed downward and inward that end in sharp points for undercutting and so attaching to subjacent tissue.

43. A bandage with ferromagnetic bodies interleaved between layers of nonallergenic sterile gauze on one side and a nonallergenic sterile stretchable fabric on the other side for tying about and so fastening said bodies around a tubular anatomical structure.

44. A bandage made of a nonallergenic sterile stretchable fabric having ferromagnetic clasps with sharp points mounted to the side of said bandage to be in contact with a tubular anatomical structure, said clasps arranged in at least two formations in facing relation so that stretching said fabric allows said clasps to undercut and so attach to the outer tissue of a tubular anatomical structure.

45. A mounting for a ferromagnetic body for use with a bandage having ferromagnetic clasps as defined in claim 44, where said mounting consists of a base made of a nonmagnetic material having prongs directed downward and inward that end in sharp points for undercutting and so attaching to subjacent tissue.
